(19) United States
(12) Patent Application Publication
(10) Pub. No.: US 2022/0282285 A1
(43) Pub. Date: Sep. 8, 2022

(54) GENETICALLY-EDITED IMMUNE CELLS AND METHODS OF THERAPY

(71) Applicant: REGENTS OF THE UNIVERSITY OF MINNESOTA, Minneapolis, MN (US)

(72) Inventors: Beau WEBBER, Andover, MN (US); Branden MORIARITY, Shoreview, MN (US)

(21) Appl. No.: 17/700,880

(22) Filed: Mar. 22, 2022

Related U.S. Application Data
(63) Continuation of application No. PCT/US2020/052295, filed on Jun. 23, 2020.
(60) Provisional application No. 62/904,299, filed on Sep. 23, 2019.
(60) Provisional application No. 62/915,436, filed on Oct. 15, 2019.

(51) Int. Cl. C12N 15/90; A61K 35/17; C07K 14/725; C12N 5/0783; C12N 9/22

(52) U.S. Cl. CPC C12N 15/907; A61K 35/17; C07K 14/7051; C12N 5/0636; C12N 9/22; A61K 2039/5156

(57) ABSTRACT

The disclosure provides genetically-edited immune cells, methods of generating genetically-edited immune cells, and methods of therapy. In some embodiments, the methods described herein comprise contacting a plurality of mammalian cells with a polynucleic acid construct that comprises an insert sequence flanked by homology arms, wherein said homology arms comprise a sequence homologous to at most 400 consecutive nucleotides of a sequence adjacent to a target site in the genome of said plurality of mammalian cells.

### CROSS REFERENCE

This application is a continuation of International Application No. PCT/US2020/52295, filed Sep. 23, 2020, which claims benefit to U.S. Provisional Application Nos. 62/904,299, filed Sep. 23, 2019 and 62/915,436, filed Oct. 15, 2019, each of which is entirely incorporated herein by reference for all purposes.

### SEQUENCE LISTING

The instant application contains a Sequence Listing which has been submitted electronically in ASCII format and is hereby incorporated by reference in its entirety. Said ASCII copy, created on Mar. 21, 2022, is named 199827748601_SL.txt and is 5,341,564 bytes in size.

### BACKGROUND

Genetically-edited immune cells hold great promise as potential therapies for a range of disorders, including cancers, autoimmune disorders, inflammatory disorders, and infectious diseases. To realize this potential, techniques are needed to introduce desired modifications into the immune cell genome efficiently, while preserving cellular viability.

### INCORPORATION BY REFERENCE

Each patent, publication, and non-patent literature cited in the application is hereby incorporated by reference in its entirety as if each was incorporated by reference individually.

### SUMMARY

In one aspect, provided herein are methods of generating a population of engineered mammalian cells, comprising: (a) contacting a plurality of mammalian cells with a polynucleic acid construct that comprises an insert sequence flanked by homology arms, wherein each of said homology arms comprise a sequence homologous to at most 400 consecutive nucleotides of a sequence adjacent to a target site in the genome of said plurality of mammalian cells; (b) cleaving said polynucleic acid construct; and (c) inserting said insert sequence in said target site, to thereby generate a population of engineered mammalian cells.

In some embodiments, the method further comprises expanding said population of genetically engineered mammalian cells.

In some embodiments, the method further comprises contacting said plurality of mammalian cells with a DNase.

In some embodiments, said contacting said plurality of mammalian cells with said DNase results in an increase in the percentage of cells in said population of engineered mammalian cells that express a transgene encoded by said insert sequence as compared to a comparable population of engineered mammalian cells in which said contacting is not performed.

In some embodiments, said contacting said plurality of mammalian cells with said DNase results in an increase in the percentage of viable cells in said population of engineered mammalian cells as compared to a comparable population of engineered mammalian cells in which said contacting is not performed.

In some embodiments, said contacting said plurality of mammalian cells with said DNase results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said DNase is selected from the group consisting of DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase is DNase I. In some embodiments, said DNase is present at a concentration from about 5 μg/ml to about 15 μg/ml.

In some embodiments, the method further comprises contacting said plurality of mammalian cells with an exogenous immunostimulatory agent.

In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said exogenous immunostimulatory agent is B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, or truncated CD19.

In some embodiments, said exogenous immunostimulatory agent is configured to stimulate expansion of at least a portion of said plurality of mammalian cells. In some embodiments, the concentration of said immunostimulatory agent is from about 50 IU/ml to about 1000 IU/ml.

In some embodiments, the method further comprises contacting said plurality of mammalian cells with an exogenous agent that modulates DNA double strand break repair. In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed. In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said agent comprises NAC or an anti-IFNAR2 antibody. In some embodiments, said agent comprises a protein involved in DNA double strand break repair. In some embodiments, said protein involved in DNA double strand break repair is selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, and Scr7.

In some embodiments, said plurality of mammalian cells are cultured in vitro or ex vivo in a culture medium, wherein said culture medium is substantially antibiotic free.

In some embodiments, said insert sequence is introduced into said plurality of mammalian cells using a plasmid, a minicircle vector, a linearized double stranded DNA construct, or a viral vector.

In some embodiments, said insert sequence comprises a sequence encoding an exogenous receptor. In some embodiments, said exogenous receptor is a T cell receptor (TCR), a chimeric antigen receptor (CAR), a B cell receptor (BCR), a natural killer cell (NK cell) receptor, a cytokine receptor, or a chemokine receptor. In some embodiments, said exogenous receptor is an immune receptor with specificity for a disease-associated antigen. In some embodiments, said exogenous receptor is an immune receptor that specifically binds to a cancer antigen. In some embodiments, said exogenous receptor is an immune receptor that specifically binds an autoimmune antigen.

In some embodiments, said insert sequence comprises a promoter sequence, an enhancer sequence, or both a promoter sequence and an enhancer sequence.

In some embodiments, said method further comprises cleaving said target site in the genome of said plurality of mammalian cells. In some embodiments said cleaving said target site comprises cleaving with an endonuclease. In some embodiments, said cleaving said polynucleic acid construct comprises cleaving with an endonuclease. In some embodiments, said endonuclease is a CRISPR-associated endonuclease. In some embodiments, said endonuclease is a Cas9. In some embodiments, (a) further comprises introducing into said plurality of mammalian cells a first guide RNA (gRNA) or a polynucleic acid encoding said first gRNA. In some embodiments, (a) further comprises introducing into said plurality of mammalian cells a second guide RNA (gRNA) or a polynucleic acid encoding said second gRNA. In some embodiments, said first guide RNA targets said endonuclease to produce at least one double stranded break in the genome of said plurality of mammalian cells. In some embodiments, said first guide RNA targets said endonuclease to produce at least one double stranded break in the polynucleic acid construct.

In some aspects, a first gRNA and a second guide RNA comprise a sequence that comprises at least 60%, 70%, 80%, 85%, 90%, 95%, 97%, or 99% sequence identity with at least a portion of SEQ ID NO: 79 or SEQ ID NO: 82. In some cases, a first gRNA is capable of binding to an endogenous gene (such as one selected from Table 1, an immune checkpoint, and/or safe harbor gene) and a second gRNA is capable of binding a xenogeneic sequence or synthetic sequence (such as a targeting sequence of a universal gRNA provided herein).

In some embodiments, said first guide RNA targets said endonuclease to produce at least one double stranded break in the genome of said plurality of mammalian cells and at least one double stranded break in said polynucleic acid construct. In some embodiments, said double stranded break in the genome of said plurality of mammalian cells is introduced in a safe harbor locus. In some embodiments, said double stranded break in the genome of said plurality of mammalian cells is introduced in an immunomodulatory gene locus. In some embodiments, said double stranded break in the genome of said plurality of mammalian cells is introduced in an immune checkpoint gene locus. In some embodiments, said double stranded break in the genome of said plurality of mammalian cells is introduced in a gene that codes for an receptor. In some embodiments, said double stranded break in the genome of said plurality of mammalian cells is introduced in a gene that codes for a T cell receptor component. In some embodiments, said double stranded break in the genome of said plurality of mammalian cells is introduced in a TRAC or TCRB locus.

In some embodiments, expression of said endogenous protein encoded by said TRAC or TCRB locus is disrupted.

In some embodiments, said mammalian cells are human cells. In some embodiments, said mammalian cells are primary cells. In some embodiments, said mammalian cells are immune cells. In some embodiments, said immune cells are T cells, NK cells, NKT cells, B cells, tumor infiltrating lymphocytes (TIL), B cells, macrophages, dendritic cells, or neutrophils. In some embodiments, said plurality of mammalian cells comprises human T cells, NK cells, NKT cells, tumor infiltrating lymphocytes (TIL), B cells, macrophages, dendritic cells, or neutrophils. In some embodiments, said plurality of mammalian cells comprises human T cells.

In some embodiments, (c) comprises producing two double stranded breaks in said polynucleic acid construct.

In some embodiments, (b) comprises producing two double stranded breaks in the genome of said plurality of mammalian cells, wherein said insertion sequence is inserted into the genome of said plurality of mammalian cells and bridges said two double stranded breaks in the genome of said plurality of mammalian cells.

In some embodiments, at least 1, 2, 3, 4, 5, 6, 7, 8, 9, or 10 nucleotides are deleted from the mammalian cell genome.

In some embodiments, said homology arms comprises a number of nucleotides that is a multiple of three or four. In some embodiments, said homology arms comprise at most 5-100 base pairs. In some embodiments, said homology arms comprise at most 50 base pairs. In some embodiments, said homology arms flank the sequence for insertion. In some embodiments, said homology arms are flanked by a sequence targeted by a guide RNA. In some embodiments, said homology arms are different or identical. In some cases, the homology arms are different. In some cases, the homology arms are identical. In some cases, at least one of said homology arms is flanked by a sequence targeted by a guide RNA. In some cases, both homology arms are flanked by a sequence targeted by a guide RNA. In some embodiments, said homology arms flank the sequence for insertion. In some embodiments, the homology arms comprise sequences homologous to sequences in a TRAC or TCRB locus.

In some cases, homology arms can comprise a sequence homologous to 30-70, 35-65, 40-60, 45-55, 45-50, 60-80, 60-100, 50-200, 100-400, 200-600, or 500-1000 bases in length. In some cases, homology arms comprise a sequence homologous to 48 bases in length. In some cases, the sequence is an endogenous gene sequence, for example in Table 1, an immune checkpoint sequence, and/or a safe harbor sequence.

In some embodiments, the method further comprises disrupting one or more additional genes in the mammalian cell genome.

In some embodiments, the method further comprises introducing one or more additional polynucleic acid constructs comprising sequences for insertion in (a), producing double stranded breaks at additional sites in the mammalian cell genome in (b), producing double stranded breaks in the one or more additional polynucleic acid constructs in (c), and inserting the one or more additional sequences for insertion into the additional sites in the mammalian cell genome.

In one aspect, provided herein are methods of generating a population of engineered mammalian cells, comprising: (a) contacting a plurality of mammalian cells with a polynucleic acid construct comprising an insert sequence flanked by homology arms, wherein said homology arms comprise a sequence homologous to a sequence adjacent to a target site in the genome of said plurality of mammalian cells; (b) cleaving said polynucleic acid construct; and (c) inserting said insert sequence in said target site, wherein said inserting is at least 10% more efficient than a method that does not comprise (b), to thereby generate a population of engineered mammalian cells.

In some embodiments, the method further comprises expanding said population of genetically engineered mammalian cells.

In some embodiments, the method further comprises contacting said plurality of mammalian cells with a DNase.

In some embodiments, said contacting said plurality of mammalian cells with said DNase results in an increase in the percentage of cells in said population of engineered mammalian cells that express a transgene encoded by said insert sequence as compared to a comparable population of engineered mammalian cells in which said contacting is not performed.

In some embodiments, said contacting said plurality of mammalian cells with said DNase results in an increase in the percentage of viable cells in said population of engineered mammalian cells as compared to a comparable population of engineered mammalian cells in which said contacting is not performed.

In some embodiments, said contacting said plurality of mammalian cells with said DNase results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, at least 60%, 65%, 70%, 75%, 80%, or 90% of the cells in said population of engineered mammalian cells express said transgene encoded by said insert sequence, as measured by detection of said transgene by flow cytometry 7 days after said plurality of mammalian cells is contacted with said polynucleic acid construct.

In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase is DNase I. In some embodiments, said DNase is present at a concentration from about 5 μg/ml to about 15 μg/ml.

In some embodiments, the method further comprises contacting said plurality of mammalian cells with an exogenous immunostimulatory agent.

In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said exogenous immunostimulatory agent is B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, or truncated CD19.

In some embodiments, said exogenous immunostimulatory agent is configured to stimulate expansion of at least a portion of said plurality of mammalian cells. In some embodiments, the concentration of said immunostimulatory agent is from about 50 IU/ml to about 1000 IU/ml.

In some embodiments, the contacting of a plurality of mammalian cells with a polynucleic acid construct that comprises an insert sequence flanked by a homology arms occurs from 30 hrs-36 hrs after said contacting with said exogenous immunostimulatory agent. In some embodiments the contacting of a plurality of mammalian cells with a polynucleic acid construct that comprises an insert sequence flanked by homology arms occurs 36 hours after said contacting with said exogenous immunostimulatory agent.

In some embodiments, the method further comprises contacting said plurality of mammalian cells with an exogenous agent that modulates DNA double strand break repair. In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed. In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said agent comprises NAC or an anti-IFNAR2 antibody. In some embodiments, said agent comprises a protein involved in DNA double strand break repair. In some embodiments, said protein involved in DNA double strand break repair is selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, and Scr7.

In some embodiments, said plurality of mammalian cells are cultured in vitro or ex vivo in a culture medium, wherein said culture medium is substantially antibiotic free.

In some embodiments, said insert sequence is introduced into said plurality of mammalian cells using a plasmid, a minicircle vector, a linearized double stranded DNA construct, or a viral vector.

In some embodiments, said insert sequence or transgene comprises a sequence encoding an exogenous receptor. In some embodiments, said exogenous receptor is a T cell receptor (TCR), a chimeric antigen receptor (CAR), a B cell receptor (BCR), a natural killer cell (NK cell) receptor, a cytokine receptor, or a chemokine receptor. In some embodiments, said exogenous receptor is an immune receptor with specificity for a disease-associated antigen. In some embodiments, said exogenous receptor is an immune receptor that specifically binds to a cancer antigen. In some embodiments, said exogenous receptor is an immune receptor that specifically binds an autoimmune antigen.

In some cases, an exogenous receptor can be a TCR. In other cases, an exogenous receptor can be a CAR. A CAR can be coded by a polypeptide sequence that comprises at least 60%, 70%, 80%, 90%, 95%, 98%, or 100% identity with the polypeptide of SEQ ID NO: 91. In some cases, a polynucleic acid construct comprises at least 60%, 70%, 80%, 85%, 90%, 95%, 97%, or 99% sequence identity with at least a portion of SEQ ID NO: 90. In some cases, a polynucleic acid construct comprises SEQ ID NO: 90, or modified versions thereof.

In some embodiments, said insert sequence comprises a promoter sequence, an enhancer sequence, or both a promoter sequence and an enhancer sequence.

In some embodiments, said method further comprises cleaving said target site in the genome of said plurality of mammalian cells. In some embodiments, said cleaving said target site comprises cleaving with an endonuclease. In some embodiments, said cleaving said polynucleic acid construct comprises cleaving with an endonuclease. In some embodiments, said endonuclease is a CRISPR-associated endonuclease. In some embodiments, said endonuclease is a Cas9. In some embodiments, (a) further comprises introducing into said plurality of mammalian cells a first guide RNA (gRNA) or a polynucleic acid encoding said first gRNA. In some embodiments, (a) further comprises introducing into said plurality of mammalian cells a second guide RNA (gRNA) or a polynucleic acid encoding said second gRNA. In some embodiments, said first guide RNA targets said endonuclease to produce at least one double stranded break in the genome of said plurality of mammalian cells. In some embodiments, said first guide RNA targets said endonuclease to produce at least one double stranded break in the polynucleic acid construct.

In some embodiments, said first guide RNA targets said endonuclease to produce at least one double stranded break in the genome of said plurality of mammalian cells and at least one double stranded break in said polynucleic acid construct. In some embodiments, said double stranded break in the genome of said plurality of mammalian cells is introduced in a safe harbor locus. In some embodiments, said double stranded break in the genome of said plurality of mammalian cells is introduced in an immunomodulatory gene locus. In some embodiments, said double stranded break in the genome of said plurality of mammalian cells is introduced in an immune checkpoint gene locus. In some embodiments, said double stranded break in the genome of said plurality of mammalian cells is introduced in a gene that codes for a receptor. In some embodiments, said double stranded break in the genome of said plurality of mammalian cells is introduced in a gene that codes for a T cell receptor component. In some embodiments, said double stranded break in the genome of said plurality of mammalian cells is introduced in a TRAC or TCRB locus.

In some embodiments, expression of said endogenous protein encoded by said TRAC or TCRB locus is disrupted. In some cases, a double stranded break in a genome of a plurality of mammalian cells is introduced in the TRAC locus. In some cases, the double stranded break in the genome of the plurality of mammalian cells is introduced in exon 1 of the TRAC locus. In some cases, the double stranded break in the genome of the plurality of mammalian cells is introduced in exon 1 of TRAC, and comprises at least a portion of SEQ ID NO: 80 or a sequence at least about 1000 bases on either side, 5′ or 3′, of SEQ ID NO: 80.

In some embodiments, said mammalian cells are human cells. In some embodiments, said mammalian cells are primary cells. In some embodiments, said mammalian cells are immune cells. In some embodiments, said immune cells are T cells, NK cells, NKT cells, B cells, tumor infiltrating lymphocytes (TIL), B cells, macrophages, dendritic cells, or neutrophils. In some embodiments, said plurality of mammalian cells comprises human T cells, NK cells, NKT cells, tumor infiltrating lymphocytes (TIL), B cells, macrophages, dendritic cells, or neutrophils. In some embodiments, said plurality of mammalian cells comprises human T cells.

In some embodiments, (c) comprises producing two double stranded breaks in said polynucleic acid construct.

In some embodiments, (b) comprises producing two double stranded breaks in the genome of said plurality of mammalian cells, wherein said insertion sequence is inserted into the genome of said plurality of mammalian cells and bridges said two double stranded breaks in the genome of said plurality of mammalian cells.

In some embodiments, at least 1, 2, 3, 4, 5, 6, 7, 8, 9, or 10 nucleotides are deleted from the mammalian cell genome.

In some embodiments, said homology arms comprise a number of nucleotides that are multiples of three or four. In some embodiments, said homology arms comprise at most 5-100 base pairs. In some embodiments, said homology arms comprise at most 50 base pairs. In some embodiments, said homology arms comprise at most 75 base pairs. In some embodiments, said homology arms flank the sequence for insertion. In some embodiments, said homology arms are flanked by a sequence targeted by a guide RNA. In some embodiments, said polynucleic acid construct comprises identical or different homology arms. In some embodiments, said homology arms flank the sequence for insertion. In some embodiments, the homology arms comprise sequences homologous to sequences in a TRAC or TCRB locus.

In some embodiments, the method further comprises disrupting one or more additional genes in the mammalian cell genome.

In some embodiments, the method further comprises introducing one or more additional polynucleic acid constructs comprising sequences for insertion in (a), producing double stranded breaks at additional sites in the mammalian cell genome in (b), producing double stranded breaks in the one or more additional polynucleic acid constructs in (c), and inserting the one or more additional sequences for insertion into the additional sites in the mammalian cell genome.

In one aspect, provided herein are methods of generating a population of engineered mammalian cells, comprising: (a) contacting a plurality of mammalian cells with a polynucleic acid construct that comprises an insert sequence of at least 1000 base pairs flanked by homology arms, wherein said homology arms comprise a sequence homologous to at most 400 consecutive nucleotides of a sequence adjacent to a target site in the genome of said plurality of mammalian cells; (b) cleaving said polynucleic acid construct; and (c) inserting said insert sequence in said target site, wherein said inserting is at least 10% more efficient than a method wherein the homology arms comprise a sequence homologous to at least 500 consecutive nucleotides of said sequence adjacent to said target site, to thereby generate a population of engineered mammalian cells.

In some embodiments, the method further comprises expanding said population of genetically engineered mammalian cells.

In some embodiments, the method further comprises contacting said plurality of mammalian cells with a DNase.

In some embodiments, said contacting said plurality of mammalian cells with said DNase results in an increase in the percentage of cells in said population of engineered mammalian cells that express a transgene encoded by said insert sequence as compared to a comparable population of engineered mammalian cells in which said contacting is not performed.

In some embodiments, said contacting said plurality of mammalian cells with said DNase results in an increase in the percentage of viable cells in said population of engineered mammalian cells as compared to a comparable population of engineered mammalian cells in which said contacting is not performed.

In some embodiments, said contacting said plurality of mammalian cells with said DNase results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said DNase is selected from the group consisting of DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase is DNase I. In some embodiments, said DNase is present at a concentration from about 5 μg/ml to about 15 μg/ml.

In some embodiments, the method further comprises contacting said plurality of mammalian cells with an exogenous immunostimulatory agent.

In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said exogenous immunostimulatory agent is B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, or truncated CD19.

In some embodiments, said exogenous immunostimulatory agent is configured to stimulate expansion of at least a portion of said plurality of mammalian cells. In some embodiments, the concentration of said immunostimulatory agent is from about 50 IU/ml to about 1000 IU/ml.

In some embodiments, the method further comprises contacting said plurality of mammalian cells with an exogenous agent that modulates DNA double strand break repair. In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed. In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said agent comprises NAC or an anti-IFNAR2 antibody. In some embodiments, said agent comprises a protein involved in DNA double strand break repair. In some embodiments, said protein involved in DNA double strand break repair is selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, and Scr7.

In some embodiments, said plurality of mammalian cells are cultured in vitro or ex vivo in a culture medium, wherein said culture medium is substantially antibiotic free.

In some embodiments, said insert sequence is introduced into said plurality of mammalian cells using a plasmid, a minicircle vector, a linearized double stranded DNA construct, or a viral vector.

In some embodiments, said insert sequence comprises a sequence encoding an exogenous receptor. In some embodiments, said exogenous receptor is a T cell receptor (TCR), a chimeric antigen receptor (CAR), a B cell receptor (BCR), a natural killer cell (NK cell) receptor, a cytokine receptor, or a chemokine receptor. In some embodiments, said exogenous receptor is an immune receptor with specificity for a disease-associated antigen. In some embodiments, said exogenous receptor is an immune receptor that specifically binds to a cancer antigen. In some embodiments, said exogenous receptor is an immune receptor that specifically binds an autoimmune antigen.

In some embodiments, said insert sequence comprises a promoter sequence, an enhancer sequence, or both a promoter sequence and an enhancer sequence.

In some embodiments, said method further comprises cleaving said target site in the genome of said plurality of mammalian cells. In some embodiments, said cleaving said target site comprises cleaving with an endonuclease. In some embodiments, said cleaving said polynucleic acid construct comprises cleaving with an endonuclease. In some embodiments, said endonuclease is a CRISPR-associated endonuclease. In some embodiments, said endonuclease is a Cas9. In some embodiments, (a) further comprises introducing into said plurality of mammalian cells a first guide RNA (gRNA) or a polynucleic acid encoding said first gRNA. In some embodiments, (a) further comprises introducing into said plurality of mammalian cells a second guide RNA (gRNA) or a polynucleic acid encoding said second gRNA. In some embodiments, said first guide RNA targets said endonuclease to produce at least one double stranded break in the genome of said plurality of mammalian cells. In some embodiments, said first guide RNA targets said endonuclease to produce at least one double stranded break in the polynucleic acid construct.

In some embodiments, said first guide RNA targets said endonuclease to produce at least one double stranded break in the genome of said plurality of mammalian cells and at least one double stranded break in said polynucleic acid construct. In some embodiments, said double stranded break in the genome of said plurality of mammalian cells is introduced in a safe harbor locus. In some embodiments, said double stranded break in the genome of said plurality of mammalian cells is introduced in an immunomodulatory gene locus. In some embodiments, said double stranded break in the genome of said plurality of mammalian cells is introduced in an immune checkpoint gene locus. In some embodiments, said double stranded break in the genome of said plurality of mammalian cells is introduced in a gene that codes for a receptor. In some embodiments, said double stranded break in the genome of said plurality of mammalian cells is introduced in a gene that codes for a T cell receptor component. In some embodiments, said double stranded break in the genome of said plurality of mammalian cells is introduced in a TRAC or TCRB locus.

In some embodiments, expression of said endogenous protein encoded by said TRAC or TCRB locus is disrupted.

In some embodiments, said mammalian cells are human cells. In some embodiments, said mammalian cells are primary cells. In some embodiments, said mammalian cells are immune cells. In some embodiments, said immune cells are T cells, NK cells, NKT cells, B cells, tumor infiltrating lymphocytes (TIL), B cells, macrophages, dendritic cells, or neutrophils. In some embodiments, said plurality of mammalian cells comprises human T cells, NK cells, NKT cells, tumor infiltrating lymphocytes (TIL), B cells, macrophages, dendritic cells, or neutrophils. In some embodiments, said plurality of mammalian cells comprises human T cells.

In some embodiments, (c) comprises producing two double stranded breaks in said polynucleic acid construct.

In some embodiments, (b) comprises producing two double stranded breaks in the genome of said plurality of mammalian cells, wherein said insertion sequence is inserted into the genome of said plurality of mammalian cells and bridges said two double stranded breaks in the genome of said plurality of mammalian cells.

In some embodiments, at least 1, 2, 3, 4, 5, 6, 7, 8, 9, or 10 nucleotides are deleted from the mammalian cell genome.

In some embodiments, said homology arms comprises a number of nucleotides that is a multiple of three or four. In some embodiments, said homology arms comprise at most 5-100 base pairs. In some embodiments, said homology arms comprise at most 50 base pairs. In some embodiments, said homology arms comprise at most 75 base pairs. In some embodiments, said homology arms flank the sequence for insertion. In some embodiments, said homology arms are flanked by a sequence targeted by a guide RNA. In some embodiments, said polynucleic acid construct comprises identical or different homology arms. In some embodiments, said homology arms flank the sequence for insertion. In some embodiments, the homology arms comprise sequences homologous to sequences in a TRAC or TCRB locus.

In some embodiments, the method further comprises disrupting one or more additional genes in the mammalian cell genome.

In some embodiments, the method further comprises introducing one or more additional polynucleic acid constructs comprising sequences for insertion in (a), producing double stranded breaks at additional sites in the mammalian cell genome in (b), producing double stranded breaks in the one or more additional polynucleic acid constructs in (c), and inserting the one or more additional sequences for insertion into the additional sites in the mammalian cell genome.

In one aspect, provided herein are methods of generating a population of engineered mammalian cells, comprising: (a) contacting a plurality of mammalian cells with a polynucleic acid construct comprising an insert sequence flanked by homology arms, wherein said homology arms comprise a sequence homologous to at most 400 consecutive nucleotides of a sequence adjacent to a target site in the genome of said plurality of mammalian cells; (b) cleaving said polynucleic acid construct; (c) generating a first double stranded break in the genome of said plurality of mammalian cells at said target site and generating a second double stranded break in the genome of said plurality of mammalian cells at a second site; and (d) inserting said insert sequence in said target site, to thereby generate a population of engineered mammalian cells.

In some embodiments, the method further comprises expanding said population of genetically engineered mammalian cells.

In some embodiments, the method further comprises contacting said plurality of mammalian cells with a DNase.

In some embodiments, said contacting said plurality of mammalian cells with said DNase results in an increase in the percentage of cells in said population of engineered mammalian cells that express a transgene encoded by said insert sequence as compared to a comparable population of engineered mammalian cells in which said contacting is not performed.

In some embodiments, said contacting said plurality of mammalian cells with said DNase results in an increase in the percentage of viable cells in said population of engineered mammalian cells as compared to a comparable population of engineered mammalian cells in which said contacting is not performed.

In some embodiments, said contacting said plurality of mammalian cells with said DNase results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said DNase is selected from the group consisting of DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase is DNase I. In some embodiments, said DNase is present at a concentration from about 5 μg/ml to about 15 μg/ml.

In some embodiments, the method further comprises contacting said plurality of mammalian cells with an exogenous immunostimulatory agent.

In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells as compared to a comparable population of engineered mammalian cells in which said contacting is not performed.

In some embodiments, said contacting said plurality of cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed.

In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said exogenous immunostimulatory agent is B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, or truncated CD19.

In some embodiments, said exogenous immunostimulatory agent is configured to stimulate expansion of at least a portion of said plurality of mammalian cells. In some embodiments, the concentration of said immunostimulatory agent is from about 50 IU/ml to about 1000 IU/ml.

In some embodiments, the method further comprises contacting said plurality of mammalian cells with an exogenous agent that modulates DNA double strand break repair. In some embodiments, said contacting said plurality of mammalian cells with said exogenous immunostimulatory agent results in an increase in the percentage of cells in said population of engineered mammalian cells that express a transgene encoded by said insert sequence as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells as compared to a comparable population of engineered mammalian cells in which said contacting is not performed. In some embodiments, said contacting said plurality of mammalian cells with said exogenous immunostimulatory agent results in an increase in the percentage of viable cells in said population of engineered mammalian cells that express a transgene encoded by said insert sequence as compared to a comparable population of engineered mammalian cells in which said contacting is not performed. In some embodiments, at least 60% of the cells in said population of engineered mammalian cells express a transgene encoded by said insert sequence, as measured by detection of said transgene by flow cytometry 7 days after said plurality of mammalian cells is contacted with said polynucleic acid construct.

In some embodiments, said agent comprises NAC or an anti-IFNAR2 antibody. In some embodiments, said agent comprises a protein involved in DNA double strand break repair. In some embodiments, said protein involved in DNA double strand break repair is selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, and Scr7.

In some embodiments, said plurality of mammalian cells are cultured in vitro or ex vivo in a culture medium, wherein said culture medium is substantially antibiotic free.

In some embodiments, said insert sequence is introduced into said plurality of mammalian cells using a plasmid, a minicircle vector, a linearized double stranded DNA construct, or a viral vector.

In some embodiments, said insert sequence comprises a sequence encoding an exogenous receptor. In some embodiments, said exogenous receptor is a T cell receptor (TCR), a chimeric antigen receptor (CAR), a B cell receptor (BCR), a natural killer cell (NK cell) receptor, a cytokine receptor, or a chemokine receptor. In some embodiments, said exogenous receptor is an immune receptor with specificity for a disease-associated antigen. In some embodiments, said exogenous receptor is an immune receptor that specifically binds to a cancer antigen. In some embodiments, said exogenous receptor is an immune receptor that specifically binds an autoimmune antigen.

In some embodiments, said insert sequence comprises a promoter sequence, an enhancer sequence, or both a promoter sequence and an enhancer sequence.

In some embodiments, said method further comprises cleaving said target site in the genome of said plurality of mammalian cells. In some embodiments, said cleaving said target site comprises cleaving with an endonuclease. In some embodiments, said cleaving said polynucleic acid construct comprises cleaving with an endonuclease. In some embodiments, said endonuclease is a CRISPR-associated endonuclease. In some embodiments, said endonuclease is a Cas9. In some embodiments, (a) further comprises introducing into said plurality of mammalian cells a first guide RNA (gRNA) or a polynucleic acid encoding said first gRNA. In some embodiments, (a) further comprises introducing into said plurality of mammalian cells a second guide RNA (gRNA) or a polynucleic acid encoding said second gRNA. In some embodiments, said first guide RNA targets said endonuclease to produce at least one double stranded break in the genome of said plurality of mammalian cells. In some embodiments, said first guide RNA targets said endonuclease to produce at least one double stranded break in the polynucleic acid construct.

In some embodiments, said first guide RNA targets said endonuclease to produce at least one double stranded break in the genome of said plurality of mammalian cells and at least one double stranded break in said polynucleic acid construct. In some embodiments, said double stranded break in the genome of said plurality of mammalian cells is introduced in a safe harbor locus. In some embodiments, said double stranded break in the genome of said plurality of mammalian cells is introduced in an immunomodulatory gene locus. In some embodiments, said double stranded break in the genome of said plurality of mammalian cells is introduced in an immune checkpoint gene locus. In some embodiments, said double stranded break in the genome of said plurality of mammalian cells is introduced in a gene that codes for a receptor. In some embodiments, said double stranded break in the genome of said plurality of mammalian cells is introduced in a gene that codes for a T cell receptor component. In some embodiments, said double stranded break in the genome of said plurality of mammalian cells is introduced in a TRAC or TCRB locus.

In some embodiments, expression of said endogenous protein encoded by said TRAC or TCRB locus is disrupted.

In some embodiments, said mammalian cells are human cells. In some embodiments, said mammalian cells are primary cells. In some embodiments, said mammalian cells are immune cells. In some embodiments, said immune cells are T cells, NK cells, NKT cells, B cells, tumor infiltrating lymphocytes (TIL), B cells, macrophages, dendritic cells, or neutrophils. In some embodiments, said plurality of mammalian cells comprises human T cells, NK cells, NKT cells, tumor infiltrating lymphocytes (TIL), B cells, macrophages, dendritic cells, or neutrophils. In some embodiments, said plurality of mammalian cells comprises human T cells.

In some embodiments, (c) comprises producing two double stranded breaks in said polynucleic acid construct.

In some embodiments, (b) comprises producing two double stranded breaks in the genome of said plurality of mammalian cells, wherein said insertion sequence is inserted into the genome of said plurality of mammalian cells and bridges said two double stranded breaks in the genome of said plurality of mammalian cells.

In some embodiments, at least 1, 2, 3, 4, 5, 6, 7, 8, 9, or 10 nucleotides are deleted from the mammalian cell genome.

In some embodiments, said homology arms comprise a number of nucleotides that is a multiple of three or four. In some embodiments, said homology arms comprise at most 5-100 base pairs. In some embodiments, said homology arms comprise at most 50 base pairs. In some embodiments, said homology arms comprise at most 75 base pairs. In some embodiments, said homology arms flank the sequence for insertion. In some embodiments, said homology arms are flanked by a sequence targeted by a guide RNA. In some embodiments, said polynucleic acid construct comprises identical or different homology arms. In some embodiments, said homology arms flank the sequence for insertion. In some embodiments, the homology arms comprise sequences homologous to sequences in a TRAC or TCRB locus.

In some embodiments, the method further comprises disrupting one or more additional genes in the mammalian cell genome.

In some embodiments, the method further comprises introducing one or more additional polynucleic acid constructs comprising sequences for insertion in (a), producing double stranded breaks at additional sites in the mammalian cell genome in (b), producing double stranded breaks in the one or more additional polynucleic acid constructs in (c), and inserting the one or more additional sequences for insertion into the additional sites in the mammalian cell genome.

In one aspect, provided herein are methods of making an engineered T cell comprising: (a) providing a primary T cell from a human subject; (b) introducing, ex vivo, into the primary T cell: (i) a nuclease or a polynucleic acid encoding the nuclease, wherein the nuclease is a CRISPR-associated nuclease; (ii) a first guide RNA or polynucleic acid encoding the first guide RNA, wherein the first guide RNA targets a sequence in a TRAC or TCRB locus of the primary T cell; (iii) a second guide RNA or a polynucleic acid encoding the second guide RNA; and (iv) a polynucleic acid construct comprising a sequence for insertion, wherein the sequence for insertion comprises a sequence encoding an exogenous T cell receptor or chimeric antigen receptor, wherein the polynucleic acid construct comprises a first short homology arm and a second short homology arm that flank the sequence for insertion, wherein the first short homology arm and the second short homology arm comprise sequences homologous to sequences in the TRAC or TCRB locus of the primary T cell, wherein the first short homology arm is less than 50 base pairs and the second short homology arm is less than 50 base pairs, wherein the first short homology arm and the second short homology arm are flanked by sequences targeted by the second guide RNA; (c) producing a double stranded break in the TRAC or TCRB locus of the genome of the primary T cell, wherein double stranded break in the TRAC or TCRB locus is produced by the CRISPR-associated nuclease and the first guide RNA, wherein the double stranded break is between a first sequence homologous to the first short homology arm and a second sequence homologous to the second short homology arm; and (d) producing two double stranded breaks in the polynucleic acid construct, thereby generating a cleaved polynucleic acid construct, wherein the cleaved polynucleic acid construct comprises the first short homology arm at a first end and the second short homology arm at a second end, wherein the two double stranded breaks are produced by the CRISPR-associated nuclease and the second guide RNA; (e) inserting the sequence encoding the exogenous T cell receptor into the primary T cell genome at the site of the double stranded break in the TRAC or TCRB locus by homology mediated end joining.

In some cases, the introducing of (b) occurs from 30 hrs. to 36 hrs. after a contacting with an exogenous immunostimulatory agent. In other cases, the introducing of (b) occurs 36 hrs. after the contacting with the exogenous immunostimulatory agent. In some cases, an exogenous immunostimulatory agent is B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, or truncated CD19.

In one aspect, provided herein are methods of treating cancer in a subject in need thereof, comprising administering to said subject a composition described herein. In some embodiments, said cancer is bladder cancer, epithelial cancer, bone cancer, brain cancer, breast cancer, esophageal cancer, gastrointestinal cancer, leukemia, liver cancer, lung cancer, lymphoma, myeloma, ovarian cancer, prostate cancer, sarcoma, stomach cancer, thyroid cancer, acute lymphocytic cancer, acute myeloid leukemia, alveolar rhabdomyosarcoma, anal canal, rectal cancer, ocular cancer, cancer of the neck, gallbladder cancer, pleural cancer, oral cancer, cancer of the vulva, colon cancer, cervical cancer, fibrosarcoma, gastrointestinal carcinoid tumor, Hodgkin lymphoma, kidney cancer, mesothelioma, mastocytoma, melanoma, multiple myeloma, nasopharynx cancer, non-Hodgkin lymphoma, pancreatic cancer, peritoneal cancer, renal cancer, skin cancer, small intestine cancer, soft tissue cancer, solid tumors, stomach cancer, testicular cancer, or thyroid cancer. In some embodiments, said cancer is gastrointestinal cancer, breast cancer, lymphoma, or prostate cancer. In some embodiments, said population of engineered mammalian cells are allogenic or autologous to said subject.

In one aspect, provided herein are mammalian cells, comprising: (a) a polynucleic acid construct comprising an exogenous sequence flanked by homology arms, wherein each of the homology arms comprise a sequence homologous to at most 400 consecutive nucleotides of a sequence adjacent to a target site in the genome of the mammalian cell, wherein the polynucleic acid has been cleaved and comprises a resected end; and (b) a double stranded break in the genome of the mammalian cell, wherein at least one end exposed by the double stranded break is resected.

In some embodiments, said mammalian cell are human cells. In some embodiments, said mammalian cell are primary cells. In some embodiments, said mammalian cell are immune cells. In some embodiments, said immune cells are T cells, NK cell, NKT cells, B cells, tumor infiltrating lymphocytes (TIL), macrophages, dendritic cells, or neutrophils. In some embodiments, said immune cell is a T cell.

In one aspect, provided herein are mammalian cells, comprising: (a) a polynucleic acid construct that comprises an insert sequence of at least 1000 base pairs flanked by homology arms, wherein each of the homology arms comprise a sequence homologous to at most 400 consecutive nucleotides of a sequence adjacent to a target site in the genome of said plurality of mammalian cells; and (b) a double stranded break in the genome of the mammalian cell, wherein at least one end exposed by the double stranded break is resected. In some cases, homology arms comprise a sequence homologous to 30-70, 35-65, 40-60, 45-55, or 45-50 bases in length In some cases, homology arms comprise a sequence homologous to 48 bases in length.

In some embodiments, said mammalian cell are human cells. In some embodiments, said mammalian cell are primary cells. In some embodiments, said mammalian cell are immune cells. In some embodiments, said immune cells are T cells, NK cell, NKT cells, B cells, tumor infiltrating lymphocytes (TIL), macrophages, dendritic cells, or neutrophils. In some embodiments, said immune cell is a T cell.

In one aspect, provided herein are mammalian cells made by the method described herein.

In one aspect, provided herein is a population of mammalian cells made by the method described herein.

In one aspect, provided herein are pharmaceutical compositions comprising a mammalian cell made by a method described herein.

In one aspect, provided herein are pharmaceutical compositions comprising a population of mammalian cells made by a method described herein.

In one aspect, provided herein are compositions comprising: a cell population that has been contacted with a polynucleic acid encoding a transgene; and a DNase in a concentration from about 5 μg/ml to about 15 μg/ml; wherein in the presence of said DNase at least 60% of said cell population expresses said transgene as measured by detection of said transgene by flow cytometry 7 days after said cell population is contacted with said polynucleic acid.

In some embodiments, the nuclei of at least a portion of said cell population comprise at least one exogenously added modulator of DNA double strand break repair. In some embodiments, the composition is substantially antibiotic-free media.

In some embodiments, said cell population comprises primary cells. In some embodiments, said cell population comprises primary immune cells. In some embodiments, said composition further comprises at least one exogenously-added immune stimulatory agent. In some embodiments, said at least one exogenously-added immune stimulatory agent is present at a concentration from about 50 IU/ml to about 1000 IU/ml.

In some embodiments, said DNase is present at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are compositions comprising a genetically modified cell population, wherein said cell population comprises a cell, a nucleus of which comprises: a polynucleic acid encoding a transgene; and at least one exogenously-added modulator of DNA double strand break repair.

In some embodiments, the composition further comprises a DNase. In some embodiments, the composition is substantially antibiotic-free media.

In some embodiments, the nuclei of at least a portion of said cell population comprise at least one exogenously added modulator of DNA double strand break repair.

In some embodiments, said cell population comprises primary cells. In some embodiments, said cell population comprises primary immune cells. In some embodiments, said composition further comprises at least one exogenously-added immune stimulatory agent. In some embodiments, said at least one exogenously-added immune stimulatory agent is present at a concentration from about 50 IU/ml to about 1000 IU/ml.

In some embodiments, said DNase is present at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are compositions comprising: genetically modified cells; DNase; and a substantially antibiotic-free media.

In some embodiments, the nuclei of said genetically modified cells comprise at least one exogenously added modulator of DNA double strand break repair. In some embodiments, the composition is substantially antibiotic-free media.

In some embodiments, said cell population comprises primary cells. In some embodiments, said cell population comprises primary immune cells. In some embodiments, said composition further comprises at least one exogenously-added immune stimulatory agent. In some embodiments, said at least one exogenously-added immune stimulatory agent is present at a concentration from about 50 IU/ml to about 1000 IU/ml.

In some embodiments, said DNase is present at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided here are compositions comprising: genetically modified primary immune cells; DNase; and at least one exogenously-added immune stimulatory agent.

In some embodiments, the nuclei of at least a portion of said cell population comprise at least one exogenously added modulator of DNA double strand break repair. In some embodiments, the composition is substantially antibiotic-free media.

In some embodiments, said cell population comprises primary cells. In some embodiments, said cell population comprises primary immune cells. In some embodiments, said composition further comprises at least one exogenously-added immune stimulatory agent. In some embodiments, said at least one exogenously-added immune stimulatory agent is present at a concentration from about 50 IU/ml to about 1000 IU/ml.

In some embodiments, said DNase is present at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are compositions comprising: genetically modified primary immune cells; DNase; and at least one exogenously-added immune stimulatory agent at a concentration from about 50 IU/ml to about 1000 IU/ml.

In some embodiments, the nuclei of at least a portion of said cell population comprise at least one exogenously added modulator of DNA double strand break repair. In some embodiments, the composition is substantially antibiotic-free media.

In some embodiments, said cell population comprises primary cells. In some embodiments, said cell population comprises primary immune cells. In some embodiments, said composition further comprises at least one exogenously-added immune stimulatory agent. In some embodiments, said at least one exogenously-added immune stimulatory agent is present at a concentration from about 50 IU/ml to about 1000 IU/ml.

In some embodiments, said DNase is present at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of increasing transgene expression of engineered cells comprising: introducing to a population of primary immune cells an exogenous polynucleic acid that encodes a transgene thereby generating a population of modified primary immune cells; and contacting said population of modified primary immune cells with a DNase and an immune stimulatory agent; wherein said contacting results in an increase in a percentage of cells that express said transgene encoded by said exogenous polynucleic acid as compared to a comparable population of modified primary immune cells to which only one of said DNase or said immune stimulatory agent is contacted.

In some embodiments, the nuclei of at least a portion of said cell population comprise at least one exogenously added modulator of DNA double strand break repair. In some embodiments, the composition is substantially antibiotic-free media.

In some embodiments, said cells are primary cells. In some embodiments, said at least one exogenously-added immune stimulatory agent is present at a concentration from about 50 IU/ml to about 1000 IU/ml.

In some embodiments, said DNase is added at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both. In some embodiments, said endogenous gene comprises a T cell receptor gene. In some embodiments, said endogenous gene comprises TRAC, TCRB, or both. In some embodiments, said endogenous gene comprises a T cell receptor and an immune checkpoint gene.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of increasing viability of engineered cells comprising: introducing to a population of primary immune cells an exogenous polynucleic acid that encodes a transgene thereby generating a population of modified primary immune cells; and contacting said population of modified primary immune cells with a DNase and an immune stimulatory agent; wherein said contacting results in an increase in a percentage of viable cells that express said transgene encoded by said exogenous polynucleic acid as compared to a comparable population of modified primary immune cells to which only one of said DNase or said immune stimulatory agent is contacted.

In some embodiments, contacting with said DNase and with said immune stimulatory agent take place simultaneously.

In some embodiments, the nuclei of at least a portion of said cell population comprise at least one exogenously added modulator of DNA double strand break repair. In some embodiments, the composition is substantially antibiotic-free media.

In some embodiments, said cells are primary cells. In some embodiments, said at least one exogenously-added immune stimulatory agent is present at a concentration from about 50 IU/ml to about 1000 IU/ml.

In some embodiments, said DNase is added at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, TRAC, TCRB, or any combination thereof.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of increasing cellular viability of engineered cells comprising: introducing to a population of primary immune cells an exogenous polynucleic acid that encodes a transgene thereby generating a population of modified primary immune cells; and contacting said population of modified primary immune cells with a DNase; wherein said contacting results in an increase in a percentage of viable cells that express said transgene as encoded by said exogenous polynucleic acid in said population as compared to a comparable population of modified primary immune cells to which said introducing but not said contacting is performed.

In some embodiments, the nuclei of at least a portion of said cell population comprise at least one exogenously added modulator of DNA double strand break repair. In some embodiments, the composition is substantially antibiotic-free media.

In some embodiments, said cells are primary cells. In some embodiments, said at least one exogenously-added immune stimulatory agent is present at a concentration from about 50 IU/ml to about 1000 IU/ml.

In some embodiments, said DNase is added at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, TRAC, TCRB, or any combination thereof.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of increasing transgene expression of engineered cells comprising: introducing to a population of primary immune cells an exogenous polynucleic acid that encodes a transgene thereby generating a population of modified primary immune cells; and contacting said population of primary immune cells with a DNase; wherein said contacting results in an increase in a percentage of cells that express said transgene encoded by said exogenous polynucleic acid as compared to a comparable population of modified primary immune cells to which said introducing but not said contacting is performed.

In some embodiments, the nuclei of at least a portion of said cell population comprise at least one exogenously added modulator of DNA double strand break repair. In some embodiments, the composition is substantially antibiotic-free media.

In some embodiments, said cells are primary cells. In some embodiments, said at least one exogenously-added immune stimulatory agent is present at a concentration from about 50 IU/ml to about 1000 IU/ml.

In some embodiments, said DNase is added at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both. In some embodiments, said endogenous gene comprises CISH, PD-1, TRAC, TCRB, or a combination thereof.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of increasing cellular viability of engineered cells comprising: introducing to a population of cells a minicircle vector or a linearized double stranded DNA construct that encodes a transgene thereby generating a population of modified cells; and contacting said population of modified cells with a DNase; wherein said contacting results in an increase in a percentage of viable cells in said population of modified cells as compared to a comparable population of modified cells to which said introducing but not said contacting is performed.

In some embodiments, the nuclei of at least a portion of said cell population comprise at least one exogenously added modulator of DNA double strand break repair. In some embodiments, the composition is substantially antibiotic-free media.

In some embodiments, said cells are primary cells. In some embodiments, said at least one exogenously-added immune stimulatory agent is present at a concentration from about 50 IU/ml to about 1000 IU/ml.

In some embodiments, said DNase is added at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both. In some embodiments, said endogenous gene comprises CISH, PD-1, TRAC, TCRB, or a combination thereof.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of increasing integration efficiency of engineered cells comprising: introducing to a population of cells an minicircle vector or a linearized double stranded DNA construct that encodes a transgene thereby generating a population of modified cells; and contacting said population of modified cells with a DNase; wherein said contacting results in an increase in a percentage of cells that express said transgene encoded by said minicircle vector or said linearized double stranded DNA construct as compared to a comparable population of modified cells to which said introducing but not said contacting is performed.

In some embodiments, said introducing comprises electroporating said population of cells with said exogenous polynucleic acid or said minicircle vector or said linearized double stranded DNA construct.

In some embodiments, the nuclei of at least a portion of said cell population comprise at least one exogenously added modulator of DNA double strand break repair. In some embodiments, the composition is substantially antibiotic-free media.

In some embodiments, said cells are primary cells. In some embodiments, said at least one exogenously-added immune stimulatory agent is present at a concentration from about 50 IU/ml to about 1000 IU/ml.

In some embodiments, said DNase is added at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, or both. In some embodiments, said endogenous gene comprises CISH, PD-1, TRAC, TCRB, or a combination thereof.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of genomically editing a population of primary cells comprising: introducing to said population of primary cells an exogenous polynucleic acid that encodes a transgene into a double strand break thereby generating a population of modified primary cells; and introducing into said population of modified primary cells a modulator of DNA double strand break repair; wherein said contacting increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said cell population comprises primary immune cells.

In some embodiments, the nuclei of at least a portion of said cell population comprise at least one exogenously added modulator of DNA double strand break repair. In some embodiments, the composition is substantially antibiotic-free media.

In some embodiments, said cells are primary cells. In some embodiments, said at least one exogenously-added immune stimulatory agent is present at a concentration from about 50 IU/ml to about 1000 IU/ml.

In some embodiments, said DNase is added at a concentration from about 5 μg/ml to about 15 μg/ml. In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said at least one exogenously added modulator of DNA double strand break repair comprises NAC, anti-IFNAR2 antibody, or both. In some embodiments, said at least one exogenously added modulator of DNA double strand break repair comprises a protein involved in DNA double strand break repair. In some embodiments, the protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said at least one exogenously added protein involved in DNA double strand break repair comprises RS-1, RAD51, or both.

In some embodiments, said at least one exogenously-added immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said at least one exogenously-added immune stimulatory agent comprises IL-2, IL-7, IL-15, or any combination thereof. In some embodiments, said at least one exogenously-added immune stimulatory agent is configured to stimulate expansion of at least a portion of said cell population or said cells.

In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells.

In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells.

In some embodiments, said primary T cells comprise a gamma delta T cell, a helper T cell, a memory T cell, a natural killer T cell, an effector T cell, or any combination thereof.

In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, the composition further comprises antigen-presenting cells (APCs). In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

In some embodiments, said genetically modified cells comprise disruption of one or more genomic sites. In some embodiments, said genetically modified cells comprise a modification or deletion of one or more endogenous gene. In some embodiments, said endogenous gene comprises an immune checkpoint gene. In some embodiments, said endogenous gene comprises CISH, PD-1, TRAC, TCRB, or a combination thereof.

In some embodiments, nuclei of said genetically modified cells comprise a transgene.

In some embodiments, said transgene codes for a protein selected from the group consisting of: a cellular receptor, an immunological checkpoint protein, a cytokine, and any combination thereof. In some embodiments, said transgene codes for a cellular receptor selected from the group consisting of: a T cell receptor (TCR), a B cell receptor (BCR), a chimeric antigen receptor (CAR), or any combination thereof. In some embodiments, said transgene codes for a T cell receptor.

In one aspect, provided herein are methods of genomically editing a population of primary immune cells comprising: electroporating said population of primary immune cells to introduce: a guide polynucleic acid; a guided nuclease; and a minicircle vector or a linearized double stranded DNA construct that encodes a transgene thereby generating a population of modified primary immune cells; contacting said population of modified primary immune cells with a DNase and an immune stimulatory agent; wherein said contacting results in an increase in a percentage of viable cells that express said transgene in said population of modified primary immune cells as compared to a comparable population of modified primary immune cells to which said electroporating but not said contacting is performed.

In some embodiments, said electroporating comprises contacting said cells with a polynucleic acid that codes for said guided-nuclease. In some embodiments, said polynucleic acid comprises DNA. In some embodiments, said polynucleic acid comprises mRNA. In some embodiments, said electroporating comprises contacting said cells with said guided-nuclease. In some embodiments, said guided-nuclease comprises Cas proteins, Zinc finger nuclease, TALEN, meganucleases, homologues thereof, or modified versions thereof, or any combination thereof.

In some embodiments, said guided-nuclease comprises a Cas protein.

A Cas protein can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild type exemplary Cas protein. Variants or fragments can comprise at least about 5%, 10%, 20%, 30%, 40%, 50%, 60%, 70%, 80%, 90%9, 91%, 92%, 93%, 94%, 95%, 96%, 97%, 98%, 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

In some embodiments, said Cas protein comprises Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof.

In some embodiments, said guide polynucleic acid comprises DNA that codes for a guide RNA. In some embodiments, said guide polynucleic acid comprises a guide RNA.

In some embodiments, said electroporating comprise contacting said population primary human cells with a guided-ribonucleoprotein complex that comprises said guide polynucleic acid and said guided-nuclease.

In some embodiments, said guide RNA comprises a CRISPR RNA (crRNA) and a transactivating crRNA (tracrRNA).

In some embodiments, said DNase is present at a concentration from about 5 μg/ml to about 15 μg/ml.

In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said contacting further comprises contacting said population of modified primary cells with an immune stimulatory agent. In some embodiments, said contacting with said immune stimulatory agent increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said immune stimulatory agent comprises IL-2, IL-7, IL-15, or any combination thereof. In some embodiments, said immune stimulatory agent is present at a concentration from about 50 IU/ml to about 1000 IU/ml. In some embodiments, said immune stimulatory agent is configured to stimulate expansion of at least a portion of said cell population or said cells. In some embodiments, said contacting further comprises introducing into said population of modified cells a modulator of DNA double strand break repair.

In some embodiments, said introducing said modulator of DNA double strand break repair increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said modulator of DNA double strand break repair comprises NAC, anti-IFNAR2 antibody, or both. In some embodiments, said modulator of DNA double strand break repair comprises a protein involved in DNA double strand repair. In some embodiments, said protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said protein involved in DNA comprises RS-1, RAD51, or both.

In some embodiments, said contacting comprising contacting said population of modified primary cells in a substantially antibiotics-free media. In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells. In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells. In some embodiments, said primary T cells comprise a gamma delta T cell, a helper T cell, a memory T cell, a natural killer T cell, an effector T cell, or any combination thereof. In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, said contacting comprises contacting said TILs at the presence of co-cultured APCs. In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof. In some embodiments, said introducing comprises disrupting one or more genomic sites of at least a portion of said population of primary cells, resulting in said population of modified primary cells.

In some embodiments, said transgene codes for a protein selected from the group consisting of: a cellular receptor, an immunological checkpoint protein, a cytokine, and any combination thereof. In some embodiments, said transgene codes for a T cell receptor.

In some embodiments, said introducing comprises modifying or deleting one or more endogenous gene of at least a portion of said population of primary cells, resulting in said population of modified primary cells. In some embodiments, said endogenous gene comprise an immune checkpoint gene. In some embodiments, said endogenous gene comprises PD-1.

In one aspect, provided herein are methods of genomically editing a population of primary immune cells comprising: a) electroporating said population of primary human cells to introduce: a guide polynucleic acid; a guided-nuclease; and a minicircle vector or a linearized double stranded DNA construct that encodes a transgene thereby generating a population of modified primary immune cells; and contacting said population of modified primary immune cells with a DNase and an immune stimulatory agent; wherein said contacting results in an increase in a percentage of cells that express said transgene encoded by said minicircle vector or said linearized double stranded DNA construct as compared to a comparable population of modified primary immune cells to which said electroporating but not said contacting is performed.

In some embodiments, said electroporating comprises contacting said cells with a polynucleic acid that codes for said guided-nuclease. In some embodiments, said polynucleic acid comprises DNA. In some embodiments, said polynucleic acid comprises mRNA. In some embodiments, said electroporating comprises contacting said cells with said guided-nuclease. In some embodiments, said guided-nuclease comprises Cas proteins, Zinc finger nuclease, TALEN, meganucleases, homologues thereof, or modified versions thereof, or any combination thereof.

In some embodiments, said guided-nuclease comprises a Cas protein. In some embodiments, said Cas protein comprises Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof.

In some embodiments, said guide polynucleic acid comprises DNA that codes for a guide RNA. In some embodiments, said guide polynucleic acid comprises a guide RNA.

In some embodiments, said electroporating comprise contacting said population primary human cells with a guided-ribonucleoprotein complex that comprises said guide polynucleic acid and said guided-nuclease.

In some embodiments, said guide RNA comprises a CRISPR RNA (crRNA) and a transactivating crRNA (tracrRNA).

In some embodiments, said DNase is present at a concentration from about 5 μg/ml to about 15 μg/ml.

In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said contacting further comprises contacting said population of modified primary cells with an immune stimulatory agent. In some embodiments, said contacting with said immune stimulatory agent increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said immune stimulatory agent comprises IL-2, IL-7, IL-15, or any combination thereof. In some embodiments, said immune stimulatory agent is present at a concentration from about 50 IU/ml to about 1000 IU/ml. In some embodiments, said immune stimulatory agent is configured to stimulate expansion of at least a portion of said cell population or said cells. In some embodiments, said contacting further comprises introducing into said population of modified cells a modulator of DNA double strand break repair.

In some embodiments, said introducing said modulator of DNA double strand break repair increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said modulator of DNA double strand break repair comprises NAC, anti-IFNAR2 antibody, or both. In some embodiments, said modulator of DNA double strand break repair comprises a protein involved in DNA double strand repair. In some embodiments, said protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said protein involved in DNA comprises RS-1, RAD51, or both.

In some embodiments, said contacting comprising contacting said population of modified primary cells in a substantially antibiotics-free media. In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells. In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells. In some embodiments, said primary T cells comprise a gamma delta T cell, a helper T cell, a memory T cell, a natural killer T cell, an effector T cell, or any combination thereof. In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, said contacting comprises contacting said TILs at the presence of co-cultured APCs. In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof. In some embodiments, said introducing comprises disrupting one or more genomic sites of at least a portion of said population of primary cells, resulting in said population of modified primary cells.

In some embodiments, said transgene codes for a protein selected from the group consisting of: a cellular receptor, an immunological checkpoint protein, a cytokine, and any combination thereof. In some embodiments, said transgene codes for a T cell receptor.

In some embodiments, said introducing comprises modifying or deleting one or more endogenous gene of at least a portion of said population of primary cells, resulting in said population of modified primary cells. In some embodiments, said endogenous gene comprise an immune checkpoint gene. In some embodiments, said endogenous gene comprises PD-1.

In one aspect, provided herein are methods of electroporating cells comprising: a first electroporation step to introduce a guided-nuclease to said cells; and a second electroporation step comprising introducing: a guide polynucleic acid comprising a region complementary to at least a portion of a gene; and an exogenous polynucleic acid comprising a cellular receptor sequence thereby generating modified cells; wherein said modified cells have at least one of: an increase in a percentage of integration of said exogenous polynucleic acid comprising a cellular receptor sequence; or an increase in a percentage of viability as compared to comparable cells comprising a single electroporation consisting of a) and b).

In some embodiments, said first electroporation step comprises contacting said cells with a polynucleic acid that codes for said guided-nuclease. In some embodiments, said polynucleic acid comprises DNA. In some embodiments, said polynucleic acid comprises mRNA. In some embodiments, said first electroporation step comprises contacting said cells with said guided-nuclease.

In some embodiments, said electroporating comprises contacting said cells with a polynucleic acid that codes for said guided-nuclease. In some embodiments, said polynucleic acid comprises DNA. In some embodiments, said polynucleic acid comprises mRNA. In some embodiments, said electroporating comprises contacting said cells with said guided-nuclease. In some embodiments, said guided-nuclease comprises Cas proteins, Zinc finger nuclease, TALEN, meganucleases, homologues thereof, or modified versions thereof, or any combination thereof.

In some embodiments, said guided-nuclease comprises a Cas protein. In some embodiments, said Cas protein comprises Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof.

In some embodiments, said guide polynucleic acid comprises DNA that codes for a guide RNA. In some embodiments, said guide polynucleic acid comprises a guide RNA.

In some embodiments, said electroporating comprise contacting said population primary human cells with a guided-ribonucleoprotein complex that comprises said guide polynucleic acid and said guided-nuclease.

In some embodiments, said guide RNA comprises a CRISPR RNA (crRNA) and a transactivating crRNA (tracrRNA).

In some embodiments, said DNase is present at a concentration from about 5 μg/ml to about 15 μg/ml.

In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said contacting further comprises contacting said population of modified primary cells with an immune stimulatory agent. In some embodiments, said contacting with said immune stimulatory agent increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said immune stimulatory agent comprises IL-2, IL-7, IL-15, or any combination thereof. In some embodiments, said immune stimulatory agent is present at a concentration from about 50 IU/ml to about 1000 IU/ml. In some embodiments, said immune stimulatory agent is configured to stimulate expansion of at least a portion of said cell population or said cells. In some embodiments, said contacting further comprises introducing into said population of modified cells a modulator of DNA double strand break repair.

In some embodiments, said introducing said modulator of DNA double strand break repair increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said modulator of DNA double strand break repair comprises NAC, anti-IFNAR2 antibody, or both. In some embodiments, said modulator of DNA double strand break repair comprises a protein involved in DNA double strand repair. In some embodiments, said protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said protein involved in DNA comprises RS-1, RAD51, or both.

In some embodiments, said contacting comprising contacting said population of modified primary cells in a substantially antibiotics-free media. In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells. In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells. In some embodiments, said primary T cells comprise a gamma delta T cell, a helper T cell, a memory T cell, a natural killer T cell, an effector T cell, or any combination thereof. In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, said contacting comprises contacting said TILs at the presence of co-cultured APCs. In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof. In some embodiments, said introducing comprises disrupting one or more genomic sites of at least a portion of said population of primary cells, resulting in said population of modified primary cells.

In some embodiments, said transgene codes for a protein selected from the group consisting of: a cellular receptor, an immunological checkpoint protein, a cytokine, and any combination thereof. In some embodiments, said transgene codes for a T cell receptor.

In some embodiments, said introducing comprises modifying or deleting one or more endogenous gene of at least a portion of said population of primary cells, resulting in said population of modified primary cells. In some embodiments, said endogenous gene comprise an immune checkpoint gene. In some embodiments, said endogenous gene comprises PD-1.

In one aspect, provided herein are methods of electroporating cells comprising: a first electroporation step to introduce a guided-ribonucleoprotein complex to said cells; and a second electroporation step comprising to introduce an exogenous polynucleic acid, thereby generating modified cells; wherein said modified cells have at least one of: an increase in a percentage of integration of said exogenous polynucleic acid comprising a cellular receptor sequence; or an increase in a percentage of viability as compared to comparable cells comprising a single electroporation consisting of a) and b).

In some embodiments, said exogenous polynucleic acid comprise a linearized double-strand DNA. In some embodiments, said electroporating comprises contacting said cells with a polynucleic acid that codes for said guided-nuclease. In some embodiments, said polynucleic acid comprises DNA. In some embodiments, said polynucleic acid comprises mRNA. In some embodiments, said electroporating comprises contacting said cells with said guided-nuclease. In some embodiments, said guided-nuclease comprises Cas proteins, Zinc finger nuclease, TALEN, meganucleases, homologues thereof, or modified versions thereof, or any combination thereof.

In some embodiments, said guided-nuclease comprises a Cas protein. In some embodiments, said Cas protein comprises Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof.

In some embodiments, said guide polynucleic acid comprises DNA that codes for a guide RNA. In some embodiments, said guide polynucleic acid comprises a guide RNA.

In some embodiments, said electroporating comprise contacting said population primary human cells with a guided-ribonucleoprotein complex that comprises said guide polynucleic acid and said guided-nuclease.

In some embodiments, said guide RNA comprises a CRISPR RNA (crRNA) and a transactivating crRNA (tracrRNA).

In some embodiments, said DNase is present at a concentration from about 5 μg/ml to about 15 μg/ml.

In some embodiments, said DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof. In some embodiments, said DNase comprises DNase I.

In some embodiments, said contacting further comprises contacting said population of modified primary cells with an immune stimulatory agent. In some embodiments, said contacting with said immune stimulatory agent increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said immune stimulatory agent comprises B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, truncated CD19, derivative thereof, or any combination. In some embodiments, said immune stimulatory agent comprises IL-2, IL-7, IL-15, or any combination thereof. In some embodiments, said immune stimulatory agent is present at a concentration from about 50 IU/ml to about 1000 IU/ml. In some embodiments, said immune stimulatory agent is configured to stimulate expansion of at least a portion of said cell population or said cells. In some embodiments, said contacting further comprises introducing into said population of modified cells a modulator of DNA double strand break repair.

In some embodiments, said introducing said modulator of DNA double strand break repair increases at least one of: a percent of viability; or a percent of expression of said transgene encoded by said exogenous polynucleic acid; in said population of modified primary cells as compared to a comparable population of modified primary cells to which said introducing but not said contacting is performed. In some embodiments, said modulator of DNA double strand break repair comprises NAC, anti-IFNAR2 antibody, or both. In some embodiments, said modulator of DNA double strand break repair comprises a protein involved in DNA double strand repair. In some embodiments, said protein involved in DNA double strand break repair comprises a protein selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some embodiments, said protein involved in DNA comprises RS-1, RAD51, or both.

In some embodiments, said contacting comprising contacting said population of modified primary cells in a substantially antibiotics-free media. In some embodiments, said primary immune cells comprises a cell selected from the group consisting of: a B cell, a basophil, a dendritic cell, an eosinophil, a gamma delta T cell, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a natural killer T cell, a neutrophil, a precursor cell, a plasma cell, a progenitor cell, a regulatory T-cell, a T cell, a thymocyte, any differentiated or de-differentiated cell thereof, or any mixture or combination of cells thereof. In some embodiments, said primary immune cells comprise primary T cells. In some embodiments, said primary T cells are isolated from a blood sample of a subject. In some embodiments, said subject is a human. In some embodiments, said blood sample is a whole blood sample or a fractioned blood sample. In some embodiments, said blood sample comprises isolated peripheral blood mononuclear cells. In some embodiments, said primary T cells comprise a gamma delta T cell, a helper T cell, a memory T cell, a natural killer T cell, an effector T cell, or any combination thereof. In some embodiments, said primary immune cells comprise CD3+ cells. In some embodiments, said primary immune cells comprise tumor infiltrating lymphocytes (TILs). In some embodiments, the TILs comprise T cells, B cells, natural killer cells, macrophages, differentiated or de-differentiated cell thereof, or any combination thereof.

In some embodiments, said contacting comprises contacting said TILs at the presence of co-cultured APCs. In some embodiments, said APCs are configured to stimulate expansion of said TILs. In some embodiments, said APCs express B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof. In some embodiments, said introducing comprises disrupting one or more genomic sites of at least a portion of said population of primary cells, resulting in said population of modified primary cells.

In some embodiments, said transgene codes for a protein selected from the group consisting of: a cellular receptor, an immunological checkpoint protein, a cytokine, and any combination thereof. In some embodiments, said transgene codes for a T cell receptor.

In some embodiments, said introducing comprises modifying or deleting one or more endogenous gene of at least a portion of said population of primary cells, resulting in said population of modified primary cells. In some embodiments, said endogenous gene comprise an immune checkpoint gene. In some embodiments, said endogenous gene comprises PD-1.

In one aspect, provided herein are methods of treating cancer comprising administering a composition described herein or a population of modified cells generated by a method described herein to a subject in need thereof. In some embodiments, said subject is in need of said treatment. In some embodiments, said subject has been diagnosed with a cancer or a tumor. In some embodiments, said subject is a human. In some embodiments, said administering comprises transfusing said composition or said population of modified cells into blood vessels of said subject.

Provided herein is an engineered polynucleotide that comprises a sequence that comprises at least 60%, 70%, 80%, 85%, 90%, 95%, 97%, or 99% sequence identity with at least a portion of SEQ ID NO: 81 or SEQ ID NO: 84 as determined by BLAST.

Provided herein is also an engineered polynucleotide that comprises at least 60%, 70%, 80%, 85%, 90%, 95%, 97%, or 99% sequence identity with at least a portion of SEQ ID NO: 79 or SEQ ID NO: 82 as determined by BLAST.

Provided herein is a ribonucleoprotein (RNP) that comprises an engineered polynucleotide. An RNP can further comprise an endonuclease. In some aspects, an endonuclease comprises a CRISPR endonuclease.

Provided herein is a cell that comprises an engineered polynucleotide and/or an RNP.

Provided herein is a population of cells that comprises an engineered cell.

Provided herein is also a kit that comprises an engineered polynucleotide and/or a ribonucleoprotein in a container.

### BRIEF DESCRIPTION OF THE FIGURES

FIGS. 1A-C provide a schematic for introducing an insert sequence into an immune cell genome. FIG. 1A illustrates a polynucleotide construct. C1 and C2 represent sequences targeted for cleavage by a nuclease, for example, a sequence targeted by a guide RNA for cleavage by a CRISPR-associated nuclease. C1 and C2 can be the same sequence or different sequences. H1 and H2 represent homology arm sequences. “Insert” represents a sequence to be inserted in the genome. The construct is designed for insertion at a target site in the genome represented in FIG. 1B. C3 represents a sequence targeted for cleavage by a nuclease, which can be the same sequence as C1 and/or C2 or a different sequence. H1 and H2 in FIG. 1B represent sequences in the genome homologous to H1 and H2 in the polynucleotide construct. FIG. 1C illustrates the genome after introduction of the insert sequence by the methods of the disclosure.

FIG. 2 provides the results of an experiment demonstrating that an insert TCR sequence is not integrated into the genome or expressed by cells in experimental conditions without a nuclease or guide RNA. Each column represents a condition. Each row represents a sample derived from a different donor. The y-axes represent fluorescence from CD3 staining, and the X-axes represent fluorescence from staining for the insert TCR. The numbers represent the percentage of live cells that fall within the quadrant. Condition one is mock-treated cells. Condition 2 is cells receiving a DNA minicircle vector with 1000 bp homology arms. Condition 3 is cells receiving a DNA minicircle vector with 48 bp homology arms.

FIG. 3 illustrates that higher proportions and numbers of cells express an insert TCR in the experimental conditions with 48 base pair homology arms and minicircle-targeting guide RNAs (conditions 6 & 7) compared to the experimental conditions with the 1000 base pair homology arms (conditions 4 & 5). Each column represents a condition. Each row represents a sample derived from a different donor. The y-axes represent fluorescence from CD3 staining, and the X-axes represent fluorescence from staining for the insert TCR. The numbers represent the percentage of live cells that fall within the quadrant.

FIG. 4 provides the percentage of live cells that express insert TCR from various experimental conditions. Data are presented for samples processed from two donors, with two technical replicates per donor. The results illustrate that higher proportions and numbers of cells express the insert TCR in the experimental conditions with 48 base pair homology arms and minicircle-targeting guide RNAs (conditions 6 & 7) compared to the experimental conditions with the 1000 base pair homology arms (conditions 4 & 5). Condition 1 is mock-treated cells. Condition 2 is cells receiving a DNA minicircle vector with 1000 bp homology arms, but no guide RNA or nuclease. Condition 3 is cells receiving a DNA minicircle vector with 48 bp homology arms, but not guide RNA or nuclease.

FIG. 5 provides the percentage of live cells that express a GFP reporter from various experimental conditions. Data are presented for samples processed from two donors, with three technical replicates per donor. Condition 1 is mock-treated cells. Condition 2 is cells receiving a DNA minicircle vector with 1000 bp homology arms, but no guide RNA or nuclease. Condition 3 is cells receiving a DNA minicircle vector with 48 bp homology arms, but not guide RNA or nuclease. Conditions 4 & 5 are cells that received a DNA minicircle vector with 1000 bp homology arms, minicircle-targeting guide RNAs, and nuclease. Conditions 6&7 are cells that received a DNA minicircle vector with 48 bp homology arms, minicircle-targeting guide RNAs, and nuclease. The results illustrate efficient immune cell genome editing using methods that comprise single strand annealing.

FIG. 6A-FIG. 6E provide a schematic for editing an immune cell genome with methods of the disclosure comprising a polynucleotide construct with two homology arms and two cleavage sites. FIG. 6A illustrates a polynucleotide construct. C1 and C2 represent sequences targeted for cleavage by a nuclease, for example, sequences targeted by a guide RNA for cleavage by a CRISPR-associated nuclease. C1 and C2 can be the same sequence or different sequences. H1 and H2 represent homology arm sequences. “(insert)” represents an intervening sequence between the two homology arms, that can be present or absent. The construct is designed for insertion at a target site in the genome represented in FIG. 6B. FIG. 6B illustrates a target site in the immune cell genome. C3 represents a sequence targeted for cleavage by a nuclease, which can be the same sequence as C1 and/or C2 or a different sequence. H1 and H2 in FIG. 6B represent sequences in the genome homologous to H1 and H2 in the polynucleotide construct. FIG. 6C represents the polynucleotide construct of FIG. 6A that has been cleaved at C1 and C2 and undergone 5′ resection from the sites of the double stranded breaks, exposing single stranded sequences of the H1 and H2 homology arms. FIG. 6D represents the site in the immune cell genome from FIG. 6A that has been cleaved at C3. Each end exposed by the double-stranded break has undergone 5′ resection, exposing single stranded sequences homologous to the sequences in the H1 and H2 homology arms. FIG. 6E represents the genome after repair of the genome using the polynucleic acid construct or a part thereof as a repair template (e.g., repair via a pathway comprising single strand annealing, homology-mediated end joining, microhomology-mediated end joining, alternative end joining, homology-directed repair, homologous recombination, or a combination thereof).

FIG. 7A-FIG. 7E provide a schematic for editing an immune cell genome with methods of the disclosure comprising a polynucleotide construct with one homology arms and one cleavage site. FIG. 7A illustrates a polynucleotide construct. C1 represents a sequence targeted for cleavage by a nuclease, for example, a sequence targeted by a guide RNA for cleavage by a CRISPR-associated nuclease. H1 represents a homology arm sequences. “(insert)” represents an intervening sequence between the two homology arms, that can be present or absent. The construct is designed for insertion at a target site in the genome represented in FIG. 7B. FIG. 7B illustrates a target site in the immune cell genome. C2 represents a sequence targeted for cleavage by a nuclease, which can be the same sequence as C1 or a different sequence. H1 in FIG. 7B represents a sequence in the genome homologous to H1 in the polynucleotide construct. FIG. 7C represents the polynucleotide construct of FIG. 7A that has been cleaved at C1 and undergone 5′ resection from the sites of the double stranded break, exposing a single stranded sequence of the H1 homology arm. FIG. 7D represents the site in the immune cell genome from FIG. 7A that has been cleaved at C2. The end exposed by the double-stranded break has undergone 5′ resection, exposing single stranded sequences homologous to the sequence in the H1 homology arm. FIG. 7E represents the genome after repair of the genome using the polynucleic acid construct or a part thereof as a repair template (e.g., repair via a pathway comprising single strand annealing, homology-mediated end joining, microhomology-mediated end joining, alternative end joining, homology-directed repair, homologous recombination, or a combination thereof).

FIG. 8A-FIG. 8E provide a schematic for editing an immune cell genome with methods of the disclosure comprising a polynucleotide construct with two homology arms and two cleavage sites and introducing two double-stranded breaks in the immune cell genome (e.g., to facilitate a large deletion). FIG. 8A illustrates a polynucleotide construct. C1 and C2 represent sequences targeted for cleavage by a nuclease, for example, sequences targeted by a guide RNA for cleavage by a CRISPR-associated nuclease. C1 and C2 can be the same sequence or different sequences. H1 and H2 represent homology arm sequences. “(i)” represents an intervening sequence between the two homology arms, that can be present or absent. The construct is to bridge two target sites in the immune cell when inserted, thereby generating a deletion in the immune cell genome, with or without an insertion. FIG. 8B illustrates the two target sites in the immune cell genome. C3 and C3 represent sequences targeted for cleavage by a nuclease, each of which can be the same sequence as C1 and/or C2 or different sequence(s). H1 and H2 in FIG. 8B represent sequences in the genome homologous to H1 and H2 in the polynucleotide construct. FIG. 8C represents the polynucleotide construct of FIG. 8A that has been cleaved at C1 and C2 and undergone 5′ resection from the sites of the double stranded breaks, exposing single stranded sequences of the H1 and H2 homology arms. FIG. 8D represents the site in the immune cell genome from FIG. 8A that has been cleaved at C3 and C4. Each end exposed by the double-stranded breaks has undergone 5′ resection, exposing single stranded sequences homologous to the sequences in the H1 and H2 homology arms. FIG. 8E represents the genome after repair of the genome using the polynucleic acid construct or a part thereof as a repair template (e.g., repair via a pathway comprising single strand annealing, homology-mediated end joining, microhomology-mediated end joining, alternative end joining, homology-directed repair, homologous recombination, or a combination thereof).

FIG. 9A-FIG. 9C provide a schematic for introducing an insert sequence into an immune cell genome using a polynucleotide construct that comprises one homology arm and one cleavage site. FIG. 9A illustrates a polynucleotide construct. C1 represents a sequence targeted for cleavage by a nuclease, for example, a sequence targeted by a guide RNA for cleavage by a CRISPR-associated nuclease. H1 represents a homology arm sequence. “Insert” represents a sequence to be inserted in the genome. The construct is designed for insertion at a target site in the genome represented in FIG. 9B. C2 represents a sequence targeted for cleavage by a nuclease, which can be the same sequence as C1 or a different sequence. H1 in FIG. 9B represents a sequence in the genome homologous to H1 in the polynucleotide construct. FIG. 9C illustrates the genome after introduction of the insert sequence by the methods of the disclosure.

FIG. 10A-FIG. 10C provide a schematic for editing an immune cell genome of the disclosure (e.g., introducing a small INDEL). FIG. 10A illustrates a polynucleotide construct. C1 and C2 represent sequences targeted for cleavage by a nuclease, for example, a sequence targeted by a guide RNA for cleavage by a CRISPR-associated nuclease. C1 and C2 can be the same sequence or different sequences. H1 and H2 represent homology arm sequences. The construct is designed to act as a repair template for a target site in the genome represented in FIG. 10B. C3 represents a sequence targeted for cleavage by a nuclease, which can be the same sequence as C1 and/or C2 or a different sequence. H1 and H2 in FIG. 10B represent sequences in the genome homologous to H1 and H2 in the polynucleotide construct. FIG. 10C illustrates the genome after introduction of the insert sequence by the methods of the disclosure.

FIG. 11A-FIG. 11C provide a schematic for editing an immune cell genome of the disclosure (e.g., introducing a small INDEL) using a polynucleotide construct that comprises one homology arm and one cleavage site. FIG. 11A illustrates a polynucleotide construct. C1 represents a sequence targeted for cleavage by a nuclease, for example, a sequence targeted by a guide RNA for cleavage by a CRISPR-associated nuclease. H1 represents a homology arm sequence. The construct is designed to act as a repair template for a target site in the immune cell genome represented in FIG. 11B. C2 represents a sequence targeted for cleavage by a nuclease, which can be the same sequence as C1 or a different sequence. H1 in FIG. 11B represents a sequence in the genome homologous to H1 in the polynucleotide construct. FIG. 11C illustrates the genome after introduction of the insert sequence by the methods of the disclosure.

FIG. 12A-FIG. 12C provide a schematic for introducing an insert sequence into an immune cell genome with methods of the disclosure comprising a polynucleotide construct with two homology arms and two cleavage sites and introducing two double-stranded breaks in the immune cell genome (e.g., to facilitate a deletion). FIG. 12A illustrates a polynucleotide construct. C1 and C2 represent sequences targeted for cleavage by a nuclease, for example, a sequence targeted by a guide RNA for cleavage by a CRISPR-associated nuclease. C1 and C2 can be the same sequence or different sequences. H1 and H2 represent homology arm sequences. “Insert” represents a sequence to be inserted in the genome. The construct is designed for insertion at a target site in the genome represented in FIG. 12B. C3 and C4 represents sequences targeted for cleavage by a nuclease, each of which can be the same sequence as C1 and/or C2 or a different sequence. H1 and H2 in FIG. 12B represent sequences in the genome homologous to H1 and H2 in the polynucleotide construct. FIG. 12C illustrates the genome after introduction of the insert sequence and deletion of the sequence spanning H1 and H2 by the methods of the disclosure.

FIG. 13A-FIG. 13C provide a schematic for generating a deletion in an immune cell genome with methods of the disclosure comprising a polynucleotide construct with two homology arms and two cleavage sites and introducing two double-stranded breaks in the immune cell genome (e.g., to facilitate a deletion). FIG. 13A illustrates a polynucleotide construct. C1 and C2 represent sequences targeted for cleavage by a nuclease, for example, a sequence targeted by a guide RNA for cleavage by a CRISPR-associated nuclease. C1 and C2 can be the same sequence or different sequences. H1 and H2 represent homology arm sequences. The construct is designed to act as a repair template for target sites in the immune cell represented in FIG. 13B. C3 and C4 represents sequences targeted for cleavage by a nuclease, each of which can be the same sequence as C1 and/or C2 or a different sequence. H1 and H2 in FIG. 13B represent sequences in the genome homologous to H1 and H2 in the polynucleotide construct. FIG. 13C illustrates the genome after use of the polynucleotide construct as a repair template to generate a deletion of the sequence spanning H1 and H2 in the immune cell genome by the methods of the disclosure.

FIG. 14 shows an image taken 24 hours following electroporation of an activated T cell culture with plasmid donor vector in 6-well dish in the presence or absence of DNase in the culture medium. The figure clearly demonstrates cell clumping in the absence of DNase, while no cell clumps were visible in the culture that has DNase.

FIG. 15A shows percent recovery of transfected cells 24 hours post-electroporation of a plasmid in the presence or absence of DNase in the culture medium, FIG. 15B is a graph quantifying the percent recovery in each condition. Both figures demonstrate lymphocyte survival after transfection was increased in the culture that has DNase as compared in the culture without DNase. FIG. 15C shows percent expression of GFP+ cells on day 14 post electroporation of a plasmid donor on day 0 or day 1, with or without DNase treatment. FIG. 15D shows percent expression of mTCR+ cells on day 14 post electroporation of a plasmid donor on day 0 or day 1, with or without DNase treatment. Both FIGS. 15B and 15D both demonstrate transgene integration was increased in the culture that has DNase as compared in the culture without DNase.

FIG. 16A shows percent expression of GFP+ cells on day 14 post electroporation of a plasmid donor on day 0 or day 1, Cas9, gRNA, in the presence or absence of RS1, DNase, or RS1 and DNase. FIG. 16B shows percent expression of mTCR+ cells on day 14 post electroporation of a plasmid donor on day 0 or day 1, Cas9, gRNA, in the presence or absence of RS1, DNase, or RS1 and DNase. Results show increased transgene expression with RS1, and/or Dnase treatment.

FIG. 17A shows day 7 percent GFP expression of T cells electroporated on day 0 post stimulation with pulse (control), Cas9 and gRNA, donor (GFP), donor and DNase, or donor, DNase, and RS-1. FIG. 17B shows day 7 percent mTCR expression of T cells electroporated on day 0 post stimulation with pulse (control), Cas9 and gRNA, donor (GFP), donor and DNase, or donor, DNase, and RS-1 (post-transfection only). FIG. 17C shows day 7 percent GFP expression of T cells electroporated on day 1 post stimulation with pulse (control), Cas9 and gRNA, donor (GFP), donor and DNase, or donor, DNase, and RS-1 (post-transfection or both pre- and post-transfection). FIG. 17D shows day 7 percent mTCR expression of T cells electroporated on day 1 post stimulation with pulse (control), Cas9 and gRNA, donor (GFP), donor and DNase, or donor, DNase, and RS-1 (post-transfection or both pre- and post-transfection). Results show increased transgene expression with RS1, and/or DNase treatment.

FIG. 18A shows day 14 percent GFP or mTCR expression of T cells electroporated on day 0 post stimulation with pulse (control), Cas9 and gRNA, donor (GFP or mTCR), donor and DNase, or donor, DNase, and RS-1 (post-transfection only). FIG. 18B shows day 14 percent GFP or mTCR expression of T cells electroporated on day 1 post stimulation with pulse (control), Cas9 and gRNA, donor (GFP or mTCR), donor and DNase, or donor, DNase, and RS-1 (post-transfection or both pre- and post-transfection). Results show increased stable transgene expression 14 days post-transfection with RS1, and/or DNase treatment.

FIG. 19 shows FACs analysis of electroporation efficiency for donor 055330 electroporated with or without RS-1, or DNase and a mTCR at 36 hours post stimulation or 36 hours post stimulation and 6 hours post initial electroporation. Results show increased transgene expression with RS1, and/or DNase treatment at both time points, suggesting the lasting effect of the treatment.

FIG. 20 shows FACs analysis of electroporation efficiency for donor 119866 electroporated with or without RS-1, or DNase and a mTCR at 36 hours post stimulation or 36 hours post stimulation and 6 hours post initial electroporation. Results show increased transgene expression with RS1, and/or DNase treatment at both time points, suggesting the lasting effect of the treatment.

FIG. 21A shows FACs analysis of electroporation efficiency for donors 055330 and 119866 electroporated with or without RS-1, or DNase and a mTCR at 36 hours post stimulation and 24 hours post initial electroporation. FIG. 21B shows FACs analysis of electroporation efficiency for donor 120534 electroporated with or without RS-1, or DNase and a mTCR at 36 hours post stimulation or 36 hours post stimulation and 6 hours post initial electroporation. Results show increased transgene expression with RS1, and/or DNase treatment at both time points, suggesting the lasting effect of the treatment

FIG. 22A shows graphs of viable cell count (number of viable cells) on day 2 post-electroporation with or without N-acetyl-cysteine (NAC), Akt VIII inhibitor (Akt Inh), or anti-IFNAR2 antibody (IFN Ab). FIG. 22B shows graphs of viable cell count (number of viable cells) on day 5 post-electroporation with or without N-acetyl-cysteine (NAC), Akt VIII inhibitor (Akt Inh), or anti-IFNAR2 antibody (IFN Ab). FIG. 22C shows graphs of viable cell count (number of viable cells) on day 7 post-electroporation with or without N-acetyl-cysteine (NAC), Akt VIII inhibitor (Akt Inh), or anti-IFNAR2 antibody (IFN Ab).

FIG. 23A shows graphs of viable cell count (percentage of viable cells) on day 2 post-electroporation with or without N-acetyl-cysteine (NAC), Akt VIII inhibitor (Akt Inh), or anti-IFNAR2 antibody (IFN Ab). FIG. 23B shows graphs of viable cell count (percentage of viable cells) on day 5 post-electroporation with or without N-acetyl-cysteine (NAC), Akt VIII inhibitor (Akt Inh), or anti-IFNAR2 antibody (IFN Ab). FIG. 23C shows graphs of viable cell count (percentage of viable cells) on day 7 post-electroporation with or without N-acetyl-cysteine (NAC), Akt VIII inhibitor (Akt Inh), or anti-IFNAR2 antibody (IFN Ab).

FIG. 24 shows a graph of percentage of mTCR positive cells on day 7 post-electroporation with or without N-acetyl-cysteine (NAC), Akt VIII inhibitor (Akt Inh), or anti-IFNAR2 antibody (IFN Ab). Figure demonstrates that transgene expression was increased in the culture that contains IFN Ab as compared to in the control culture when 30 or 50 μg exogenous donor DNA was used.

FIG. 25A shows cytoflex results of total live cells that have undergone a second stimulation post electroporation utilizing an AAVS1-GFP donor comprising homology arms (HR) or single strand annealing (SSA) that target AAVS1. FIG. 25B shows percent GFP post electroporation and a secondary stimulation of the same cells electroporated with the AAVS1-GFP donor. GFP was measured at day 7 post electroporation. The second stimulation was added about 30 minutes after the electroporation.

FIG. 26A shows flow cytometry plots of HCT1116 cells comprising a knockout of RAD52, Exo1, RAD54B, Lig3, BRD, or PolQ. Knocked out HCT1116 cells were electroporated with an AAVS1 SA-GFP donor via SSA or HR, results were acquired on day 10 post electroporation and normalized to control. FIG. 26B shows percent change in GFP expression of HR donor templates normalized to wild type (WT). FIG. 26C shows percent change in GFP expression of SSA donor templates normalized to wild type (WT).

FIG. 27 is a schematic of an exemplary strategy to knock in a transgene, such as a transgene that comprises a cellular receptor such as a CAR or TCR into an exemplary gene, such as an immune checkpoint and/or TCR, provided in Table 1.

FIG. 28A shows percent of T cells in S phase of the cell cycle at 24 hrs., 36 hr., 48 hrs., or 72 hrs. post electroporation with either control (pulse only) or an HR transgene donor.

FIG. 28B shows percent GFP on day 7 post electroporation with control (pulse only), HR SA-GFP donor, or SSA SA GFP minicircle (MC). FIG. 28C shows percent CAR (CD34+) on day 7 post electroporation with control (pulse only) or SSA anti-mesothelin CAR minicircle (MC). Percent GFP and CAR were compared against cells electroporated at 24 hrs., 36 hrs., 48 hrs., or 72 hrs.

FIG. 29A shows fold change above baseline of DNA sensors, their timing, and expression after 36 hrs. This aligns with the cell cycle mapped on the X axis. A transfection zone around 36 hrs. is shown as a shaded box. FIG. 29B shows percent of T cells in S-phase of two T cell donors at 24, 36, 48, and 72 hrs. post stimulation. FIG. 29C shows percent GFP in T cells stimulated using anti-CD3 and anti-CD28 coated beads, comprising anti-CD3 and -CD28, and electroporated with the SA-GFP plasmid alone (plasmid control) or the SA-Donor in combination with Cas9 and AAVS1 gRNA (HR) at 24 hrs., 36 hrs., 48 hrs., or 72 hrs., post-stimulation.

FIG. 30A shows perfect GFP expression in T cells stimulated with anti-CD3 and anti-CD28 coated beads for 36 hours and electroporated with the donor plasmid alone or in combination with the CRISPR Cas9 reagents. Both the HR and HMEJ cargo is the SA-GFP construct integrated at AAVS1. Plasmid was delivered alone or in combination with Cas9 mRNA and AAVS1 gRNA (HR), or for HMEJ Cas9 mRNA and AAVS1 gRNA and universal gRNA. Constructs contain a 1 kb insert cargo. FIG. 30B shows percent expression of a murine TCR (KRAS G12D TCR) insert transfected via an HR-mTCR or SSA-mTCR (HMEJ) as compared to plasmid control. Briefly, T cells were stimulated with anti-CD3 and anti-CD28 coated beads for 36 hours and electroporated with the donor plasmid alone or in combination with the CRISPR Cas9 reagents. Both the HR and HMEJ cargo is the MND-anti-KRAS TCR with 1 kb homology (for HR) and with 48 bp homology (HMEJ). Plasmid was delivered alone or in combination with Cas9 mRNA and AAVS1 gRNA (HR), or for HMEJ Cas9 mRNA, AAVS1 gRNA and Universal gRNA.

FIG. 31A shows an exemplary workflow of non-viral cellular manufacturing. (1) T cells are isolated and purified (2) T cells are activated via addition of beads and/or suitable stimulatory antibodies (3) Activation beads are removed (4) Activated T cells are electroporated and (5) Modified cells are expanded. FIG. 31B shows fold expansion of cells manufactured using the exemplary workflow of FIG. 31A and electroporated with a plasmid control, HR, or HMEJ construct. FIG. 31C shows an exemplary optional workflow comprising additional stimulation, as denoted by the second bead addition after electroporation.

FIG. 32A shows fold expansion of T cells electroporated with a murine TCR (KRAS G12D TCR) insert delivered via an HR-mTCR or SSA-mTCR (HMEJ) transgene as compared to plasmid control. Also shown are re-stimulated SSA-mTCR (HMEJ) cells. FIG. 32B shows percent anti-mesothelin CAR expression (CD34 expression) of cells transfected with the SSA-mTCR (HMEJ) transgene or SSA-mTCR (HMEJ) transgene and also restimulated as compared to control (pulse only). FIG. 32C shows luminescence data of the same cells.

FIG. 33 shows GFP expression of CD4 and CD8 cells electroporated with plasmid only, plasmid, Cas9 mRNA, and AAVS1 gRNA (HR), or plasmid, cas9 mRNA, AAVS1 gRNA and Universal gRNA for HMEJ.

FIG. 34A shows percent knock in of T cells electroporated with donor only (control), SA-eGFP-pA (HR), or SA-eGFP-pA (HMEJ) constructs comprising homology arms from 48, 100, 250, 500, 750, or 1000 base pairs in length. FIG. 34B shows a bar graph showing targeted integration rates using the SA-eGFP-pA (HR), or SA-eGFP-pA (HMEJ) constructs with increasing homology arm length, as described in FIG. 34A.

FIG. 35A cell expansion following targeted integration using donor only (control), SA-eGFP-pA (HR), or SA-eGFP-pA (HMEJ) constructs, comprising increasing homology arm length, with or without additional stimulation. FIG. 35B shows a bar graph of the same data as described in FIG. 35A.

FIG. 36 shows an exemplary clinical workflow. The provided workflow can be modified to include an additional stimulation of the T cells as described herein.

### DETAILED DESCRIPTION

### Introduction

Genetically-edited immune cells hold great promise as potential therapies for a range of disorders, including cancers, autoimmune disorders, inflammatory disorders, and infectious diseases. To realize this potential, techniques are needed to introduce desired modifications into the immune cell genome efficiently, while preserving cellular viability. Disclosed herein, in some embodiments, are genetically-edited immune cells, improved methods of genetically editing immune cells, and methods of therapy. Modifications that can be introduced into the immune cell genome include, for example, insertions, deletions, sequence replacement, (e.g., substitutions), and combinations thereof.

A number of existing methods of genetically editing immune cells rely on homologous recombination pathways. For example, a double-stranded break can be introduced into the genome, and a repair template provided to direct repair of the double-stranded break via homologous recombination. To direct repair via homologous recombination, repair templates can require long homology arms (e.g., about 500-1500 base pair homology arms). Methods that rely on repair via homologous recombination can have limitations, for example, because of the size of the homology arms required, because of the efficiency of repair, or a combination thereof. In the methods disclosed herein, double-stranded breaks can be introduced in the repair template as well as the target site in the genome. This can allow integration of the repair template via alternate or additional repair pathways, for example, pathways that comprise end resection, pathways that require only short homology arms in the repair template, or a combination thereof. Non-limiting examples of alternate or additional repair pathways that can be utilized include pathways comprising single strand annealing, homology-mediated end joining, microhomology-mediated end joining, alternative end joining, and combinations thereof.

The methods disclosed herein can have advantages over existing methods of editing immune cells, for example, higher editing efficiency, higher viability of edited cells, the ability to generate larger populations of edited cells, the ability to generate edited cells with enhanced proliferative capacity and/or effector functions, the ability to use smaller repair template constructs (e.g., comprising shorter homology arms), the ability to introduce larger sequences into the immune cell genome (e.g., at higher efficiency), the ability to introduce multiple modifications into the immune cell genome (e.g., insertions, deletions, substitutions, and/or or other modifications), and combinations thereof.

### Genetically-Modified Cells

Disclosed herein, in some embodiments, are genetically-edited cells, and methods of editing cells. In some embodiments, the cells comprise kidney cells, liver cells, pancreatic cells, blood cells, immune cells, lymphocytes, heart cells, lung cells, stem cells, ovary cells, prostate cells, muscle cells, tendon cells, ligament cells, cardiac cells, bone cells, bone marrow cells, cornea cells, retinal cells, cartilage cells, endothelial cells, cervical cells, breast cells, nervous system cells, spinal cord cells, brain cells, neurons, skin cells, epithelial cells, gastrointestinal cells, hormone secreting cells, pancreatic R cells, thyroid cells, thymus cells, exocrine cells, and parathyroid cells.

Disclosed herein, in some embodiments, are genetically-edited immune cells, and methods of editing immune cells. In some embodiments, the immune cells comprise lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (T_{EM}), central memory T cells (T_{CM}), resident memory T cells (T_{RM}), Natural killer T cells (NKTs), tumor-infiltrating lymphocytes (TILs), Natural killer cells (NKs), Innate Lymphoid Cells (ILCs), B cells, B1 cells, B1a cells, B1b cells, B2 cells, plasma cells, B regulatory cells, antigen presenting cells (APCs), monocytes, macrophages, M1 macrophages, M2 macrophages, dendritic cells, plasmacytoid dendritic cells, neutrophils, mast cells, or a combination thereof.

In some embodiments, the immune cells are a cell line. For example, a cell line can be a population of cells that have undergone mutation and gained the ability to proliferate extensively in culture.

Immune cells of the disclosure can be human mammalian cells. Immune cells of the disclosure can be human immune cells. Immune cells of the disclosure can be mouse immune cells. Immune cells of the disclosure can be rat immune cells. Immune cells of the disclosure can be rabbit immune cells. Immune cells of the disclosure can be goat immune cells. Immune cells of the disclosure can be non-human primate immune cells. Immune cells of the disclosure can be pig immune cells. Immune cells of the disclosure can be llama immune cells. Immune cells of the disclosure can be goat immune cells. Immune cells of the disclosure can be immune cells from a genetically-modified animal.

In some embodiments, the immune cells are primary cells. In some embodiments, genetic editing of immune cells can be conducted ex vivo or in vitro. For example, primary cells can be harvested from a donor organism, genetically-edited, and infused into a recipient organism or back into the donor organism. In some embodiments, genetic editing of primary cells can be conducted within an organism (e.g., in vivo).

### Polynucleic Acid Constructs

Disclosed herein, in some embodiments, are methods of genetically editing immune cells, for example, introducing insertions, deletions, sequence replacements, and combinations thereof in the immune cell. Polynucleic acid constructs can be used in the methods of the disclosure, for example, used to provide a repair template to direct the repair of a double-stranded break in the immune cell genome. A repair template can favor a certain outcome of the repair process, for example, a repaired genome comprising an insertion, deletion, replaced sequence, or any combination thereof.

Polynucleic acid constructs can comprise, for example, one or more homology arms and one or more cleavage sites that can be targeted for cleavage by a nuclease (e.g., targeted by a guide RNA and Cas9). In some embodiments, polynucleic acids constructs comprise an insert sequence.

In the methods disclosed herein, double-stranded breaks can be introduced in the repair template as well as the target site in the genome. This can allow integration of the repair template via alternate or additional repair pathways, for example, pathways that comprise end resection, pathways that require only short homology arms in the repair template, or a combination thereof. Non-limiting examples of alternate or additional repair pathways that can be utilized include pathways comprising single strand annealing, homology-mediated end joining, microhomology-mediated end joining, alternative end joining, and combinations thereof.

A polynucleic acid construct can comprise DNA, RNA, chemically-modified nucleotides, or a combination thereof. In some embodiments, the polynucleic acid construct comprises DNA. In some embodiments, the polynucleic acid comprises RNA. In some embodiments, the polynucleic acid comprises RNA and can be reverse transcribed into complementary DNA. In some embodiments, the polynucleic acid comprises a DNA minicircle. In some embodiments, the polynucleic acid construct comprises a plasmid. In some embodiments, the polynucleic acid comprises a linear DNA, e.g., a PCR product, a linear DNA liberated from a DNA minicircle or plasmid, or a synthetically-produced DNA. In some embodiments, the polynucleic acid construct comprises a circular RNA. In some embodiments, polynucleic acid construct comprises chemical modifications (e.g., as disclosed herein).

In some embodiments, the polynucleic acid construct is contained in a viral vector. Exemplary viral vectors include, but are not limited to, lentiviral vectors, retroviral vectors, adeno-associated viral vectors (AAV), adenoviral vectors, herpes simplex viral vectors, alphaviral vectors, flaviviral vectors, rhabdoviral vectors, measles viral vectors, Newcastle disease viral vectors, poxviral vectors, and picornaviral vectors. In some embodiments, the polynucleic acid construct is contained in an AAV viral vector.

Disclosed herein, in some embodiments, are methods of introducing a plurality of modifications in an immune cell genome (e.g., an insertion and a deletion, multiple insertions, multiple deletions, an insertion and multiple deletions, multiple insertions and a deletion, or multiple insertions and multiple deletions).

### Insert Sequence

In some embodiments, the methods disclosed herein allow for or comprise insertion of an insert sequence into the genome of an immune cell. In some embodiments, the insert sequence is a polynucleic acid, e.g., a DNA sequence. In some embodiments, polynucleic acid constructs comprise an insert sequence.

In some embodiments, the insert sequence is at least 10 bp, 20 bp, 30 bp, 40 bp, 50 bp, 60 bp, 70 bp, 80 bp, 90 bp, 100 bp, 150 bp, 200 bp, 250 bp, 300 bp, 400 bp, 500 bp, 600 bp, 700 bp, 800 bp, 900 bp, 1 kb, 2 kb, 3 kb, 4 kb, 5 kb, 10 kb, 20 kb, 50 kb, 100 kb, 200 kb, 300 kb, 400 kb, 500 kb, 600 kb, 700 kb, 800 kb, 900 kb, 1000 kb, or more. In some embodiments, the insert sequence is greater than 0.5 kb, 1 kb, 2 kb, 3 kb, 4 kb, 5 kb, 10 kb, 20 kb, 50 kb, 100 kb, 200 kb, 300 kb, 400 kb, 500 kb, 600 kb, 700 kb, 800 kb, 900 kb, or 1000 kb.

In some embodiments, the insert sequence is from about 500 bp to 500 kb, 500 bp to 400 kb, 500 bp to 300 kb, 500 bp to 200 kb, 500 bp to 100 kb, 500 bp to 50 kb, 500 bp to 40 kb, 500 bp to 30 kb, 500 bp to 20 kb, 500 bp to 10 kb, 500 bp to 9 kb, 500 bp to 8 kb, 500 bp to 7 kb, 500 bp to 6 kb, 500 bp to 5 kb, 500 bp to 4 kb, 500 bp to 3 kb, 500 bp to 2 kb, or 500 bp to 1 kb. In some embodiments, the insert sequence is from about 1 kb to 500 kb, 1 kb to 400 kb, 1 kb to 300 kb, 1 kb to 200 kb, 1 kb to 200 kb, 1 kb to 100 kb, 1 kb to 90 kb, 1 kb to 80 kb, 1 kb to 70 kb, 1 kb to 60 kb, 1 kb to 50 kb, 1 kb to 40 kb, 1 kb to 30 kb, 1 kb to 20 kb, 1 kb to 10 kb, 1 kb to 9 kb, 1 kb to 8 kb, 1 kb to 7 kb, 1 kb to 6 kb, 1 kb to 5 kb, 1 kb to 4 kb, 1 kb to 3 kb, or 1 kb to 2 kb. In some embodiments, the insert sequence is from about 2 kb to 500 kb, 2 kb to 400 kb, 2 kb to 300 kb, 2 kb to 200 kb, 2 kb to 200 kb, 2 kb to 100 kb, 2 kb to 90 kb, 2 kb to 80 kb, 2 kb to 70 kb, 2 kb to 60 kb, 2 kb to 50 kb, 2 kb to 40 kb, 2 kb to 30 kb, 2 kb to 20 kb, 1 kb to 10 kb, 2 kb to 9 kb, 2 kb to 8 kb, 2 kb to 7 kb, 2 kb to 6 kb, 2 kb to 5 kb, 2 kb to 4 kb, or 2 kb to 3 kb. In some embodiments, the insert sequence is from about 3 kb to 500 kb, 3 kb to 400 kb, 3 kb to 300 kb, 3 kb to 200 kb, 3 kb to 200 kb, 3 kb to 100 kb, 3 kb to 90 kb, 3 kb to 80 kb, 3 kb to 70 kb, 3 kb to 60 kb, 3 kb to 50 kb, 3 kb to 40 kb, 3 kb to 30 kb, 3 kb to 20 kb, 1 kb to 10 kb, 3 kb to 9 kb, 3 kb to 8 kb, 3 kb to 7 kb, 3 kb to 6 kb, 3 kb to 5 kb, or 3 kb to 4 kb. In some embodiments, the insert sequence is from about 4 kb to 500 kb, 4 kb to 400 kb, 4 kb to 300 kb, 4 kb to 200 kb, 4 kb to 200 kb, 4 kb to 100 kb, 4 kb to 90 kb, 4 kb to 80 kb, 4 kb to 70 kb, 4 kb to 60 kb, 4 kb to 50 kb, 4 kb to 40 kb, 4 kb to 30 kb, 4 kb to 20 kb, 1 kb to 10 kb, 4 kb to 9 kb, 4 kb to 8 kb, 4 kb to 7 kb, 4 kb to 6 kb, or 4 kb to 5 kb. In some embodiments, the insert sequence is from about 5 kb to 500 kb, 5 kb to 400 kb, 5 kb to 300 kb, 5 kb to 200 kb, 5 kb to 200 kb, 5 kb to 100 kb, 5 kb to 90 kb, 5 kb to 80 kb, 5 kb to 70 kb, 5 kb to 60 kb, 5 kb to 50 kb, 5 kb to 40 kb, 5 kb to 30 kb, 5 kb to 20 kb, 1 kb to 10 kb, 5 kb to 9 kb, 5 kb to 8 kb, 5 kb to 7 kb, or 5 kb to 6 kb. In some embodiments, the insert sequence is from about 10 kb to 500 kb, 10 kb to 400 kb, 10 kb to 300 kb, 10 kb to 200 kb, 10 kb to 200 kb, 10 kb to 100 kb, 10 kb to 90 kb, 10 kb to 80 kb, 10 kb to 70 kb, 10 kb to 60 kb, 10 kb to 50 kb, 10 kb to 40 kb, 10 kb to 30 kb, or 10 kb to 20 kb. In some embodiments, the insert sequence is from about 30 kb to 500 kb, 30 kb to 400 kb, 30 kb to 300 kb, 30 kb to 200 kb, 30 kb to 200 kb, 30 kb to 100 kb, 30 kb to 90 kb, 30 kb to 80 kb, 30 kb to 70 kb, 30 kb to 60 kb, 30 kb to 50 kb, or 30 kb to 40 kb.

In some embodiments, the insert sequence does not encode a protein. In some embodiments, the insert sequence is less than 500 bp, 400 bp, 300 bp, 200 bp, 100 bp, 50 bp, 40 bp, 30 bp, 20 bp, 10 bp 5 bp, 4 bp, 3 bp, or 2 bp.

An insert sequence can comprise, for example, a non-coding sequence, a sequence that encodes an RNA, a sequence that encodes a protein, or a combination thereof. In some embodiments, the insert sequence does not encode for a functional protein. In some embodiments, the insert sequence encodes for a protein. In some embodiments, the insert sequence encodes for a functional protein.

In some embodiments, the insert sequence encodes at least one protein. In some embodiments, the insert sequence encodes a membrane protein. In some embodiments, the insert sequence encodes a transmembrane protein. In some embodiments, the insert sequence encodes a transmembrane receptor protein. In some embodiments, the insert sequence encodes an intracellular protein (e.g., a cytoplasmic or nuclear protein). In some embodiments, the insert sequence encodes a secreted protein. In some embodiments, the insert sequence encodes a chimeric protein. In some embodiments, the insert sequence encodes a fusion protein.

In some embodiments, the insert sequence encodes a receptor expressed on the surface of an immune cell (for example, a receptor expressed on the surface of a T cell, CD4+ T cell, CD8+ T cell, alpha-beta T cell, gamma-delta T cell, T regulatory cell (Treg), cytotoxic T lymphocyte, memory T cell, effector T cell, effector-memory T cell (T_{EM}), central memory T cell (T_{CM}), resident memory T cell (T_{RM}), naïve T cell, B cell, plasma cell, NK cell, NK T cell, monocyte, macrophage, dendritic cell, antigen presenting cell, neutrophil, or tumor infiltrating lymphocyte).

In some embodiments, the insert sequence encodes a T cell receptor (TCR) or a functional portion thereof. In some embodiments, the insert sequence encodes a chimeric antigen receptor (CAR) or a functional portion thereof. In some embodiments, the insert sequence encodes a B cell receptor or a functional portion thereof. In some embodiments, the insert sequence encodes a chemokine receptor. In some embodiments, the insert sequence encodes a cytokine receptor. In some embodiments, the insert sequence encodes a fusion protein comprising one or more antigen recognition domains (e.g., an antigen recognition domain of a TCR, BCR, antibody or antigen-binding fragment thereof, DARPin etc.), one or more transmembrane domains, and one or more signaling domains (e.g., a signaling domain from a TCR, BCR, immune co-receptor, cytokine receptor, chemokine receptor, immunoreceptor tyrosine-based inhibitory domain (ITIM), immunoreceptor tyrosine-based activation domain (ITAM), immune checkpoint gene, or a combination thereof).

In some embodiments, the insert sequence encodes a receptor that specifically binds to an antigen or neoantigen expressed by a cancer cell. In some embodiments, the insert sequence encodes a receptor that specifically binds to an antigen or neoantigen expressed or presented on the surface of a cancer cell. In some embodiments, the antigen or neoantigen is from an oncogene or tumor suppressor gene (e.g., a mutated tumor suppressor gene). In some embodiments, the antigen comprises a T cell epitope. In some embodiments, the cancer is a solid tumor, hematological cancer, or soft tissue cancer. In some embodiments, the cancer cell is selected from the group consisting of bladder cancer, epithelial cancer, bone cancer, brain cancer, breast cancer, colorectal cancer, esophageal cancer, gastrointestinal cancer, leukemia, liver cancer, lung cancer, lymphoma, myeloma, ovarian cancer, prostate cancer, sarcoma, stomach cancer, thyroid cancer, acute lymphocytic cancer, acute myeloid leukemia, alveolar rhabdomyosarcoma, anal canal, rectal cancer, ocular cancer, cancer of the neck, gallbladder cancer, pleural cancer, oral cancer, cancer of the vulva, colon cancer, cervical cancer, fibrosarcoma, gastrointestinal carcinoid tumor, Hodgkin lymphoma, kidney cancer, mesothelioma, mastocytoma, melanoma, multiple myeloma, myeloma, nasopharynx cancer, non-Hodgkin lymphoma, pancreatic cancer, peritoneal cancer, renal cancer, skin cancer, small intestine cancer, stomach cancer, testicular cancer, and thyroid cancer. In some embodiments, the cancer cell is selected from the group consisting of gastrointestinal cancer, breast cancer, lymphoma, and prostate cancer.

In some embodiments, the insert sequence encodes a protein that specifically binds to an antigen expressed by a pathogen. In some embodiments, the insert sequence encodes a receptor (e.g., immune receptor) that specifically binds to an antigen expressed by a pathogen. In some embodiments, the antigen comprises a T cell epitope. In some embodiments, the pathogen is a bacterium, virus, fungus, yeast, parasite (e.g., single-celled or multicellular eukaryotic parasite), or other microorganism.

In some embodiments, the insert sequence encodes a protein that specifically binds to an antigen associated with a disease (e.g., an inflammatory or autoimmune disease). In some embodiments, the insert sequence encodes a receptor (e.g., immune receptor) that specifically binds to an antigen associated with a disease. In some embodiments, the antigen comprises a T cell epitope. In some embodiments, the disease is acute disseminated encephalomyelitis, acute motor axonal neuropathy, Addison's disease, adiposis dolorosa, adult-onset still's disease, alopecia areata, ankylosing spondylitis, anti-glomerular basement membrane nephritis, anti-neutrophil cytoplasmic antibody-associated vasculitis, anti-n-methyl-d-aspartate receptor encephalitis, antiphospholipid syndrome, antisynthetase syndrome, aplastic anemia, autoimmune angioedema, autoimmune encephalitis, autoimmune enteropathy, autoimmune hemolytic anemia, autoimmune hepatitis, autoimmune inner ear disease, autoimmune lymphoproliferative syndrome, autoimmune neutropenia, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy, autoimmune thrombocytopenic purpura, autoimmune thyroiditis, autoimmune urticaria, autoimmune uveitis, balo concentric sclerosis, behçet's disease, bickerstaffs encephalitis, bullous pemphigoid, celiac disease, chronic fatigue syndrome, chronic inflammatory demyelinating polyneuropathy, churg-strauss syndrome, cicatricial pemphigoid, cogan syndrome, cold agglutinin disease, complex regional pain syndrome, crest syndrome, crohn's disease, dermatitis herpetiformis, dermatomyositis, diabetes mellitus type 1, discoid lupus erythematosus, endometriosis, enthesitis, enthesitis-related arthritis, eosinophilic esophagitis, eosinophilic fasciitis, epidermolysis bullosa acquisita, erythema nodosum, essential mixed cryoglobulinemia, evans syndrome, felty syndrome, fibromyalgia, gastritis, gestational pemphigoid, giant cell arteritis, goodpasture syndrome, graves' disease, graves ophthalmopathy, guillain-barré syndrome, hashimoto's encephalopathy, hashimoto thyroiditis, henoch-schonlein purpura, hidradenitis suppurativa, idiopathic dilated cardiomyopathy, idiopathic inflammatory demyelinating diseases, IgA nephropathy, IgG4-related systemic disease, inclusion body myositis, inflamatory bowel disease, intermediate uveitis, interstitial cystitis, juvenile arthritis, kawasaki's disease, lambert-eaton myasthenic syndrome, leukocytoclastic vasculitis, lichen planus, lichen sclerosus, ligneous conjunctivitis, linear IgA disease, lupus nephritis, lupus vasculitis, lyme disease (chronic), ménière's disease, microscopic colitis, microscopic polyangiitis, mixed connective tissue disease, mooren's ulcer, morphea, mucha-habermann disease, multiple sclerosis, myasthenia gravis, myocarditis, myositis, neuromyelitis optica, neuromyotonia, opsoclonus myoclonus syndrome, optic neuritis, ord's thyroiditis, palindromic rheumatism, paraneoplastic cerebellar degeneration, parry romberg syndrome, parsonage-turner syndrome, pediatric autoimmune neuropsychiatric disorder associated with Streptococcus, pemphigus vulgaris, pernicious anemia, Pityriasis lichenoides et varioliformis acuta, poems syndrome, polyarteritis nodosa, polymyalgia rheumatica, polymyositis, postmyocardial infarction syndrome, postpericardiotomy syndrome, primary biliary cirrhosis, primary immunodeficiency, primary sclerosing cholangitis, progressive inflammatory neuropathy, psoriasis, psoriatic arthritis, pure red cell aplasia, pyoderma gangrenosum, raynaud's phenomenon, reactive arthritis, relapsing polychondritis, restless leg syndrome, retroperitoneal fibrosis, rheumatic fever, rheumatoid arthritis, rheumatoid vasculitis, sarcoidosis, schnitzler syndrome, scleroderma, sjogren's syndrome, stiff person syndrome, subacute bacterial endocarditis, susac's syndrome, sydenham chorea, sympathetic ophthalmia, systemic lupus erythematosus, systemic scleroderma, thrombocytopenia, tolosa-hunt syndrome, transverse myelitis, ulcerative colitis, undifferentiated connective tissue disease, urticaria, urticarial vasculitis, vasculitis, or vitiligo.

In some embodiments, the insert sequence encodes a cytokine receptor or a functional portion thereof (e.g., a cytokine recognition domain or a signaling domain). In some embodiments the insert sequence encodes a receptor for 4-1BBL, APRIL, CD153, CD154, CD178, CD70, G-CSF, GITRL, GM-CSF, IFN-α, IFN-β, IFN-γ, IL-1RA, IL-1α, IL-1β, IL-2, IL-3, IL-4, IL-5, IL-6, IL-7, IL-9, IL-10, IL-11, IL-12, IL-13, IL-14, IL-15, IL-16, IL-17, IL-18, IL-20, IL-23, LIF, LIGHT, LT-β, M-CSF, MSP, OSM, OX40L, SCF, TALL-1, TGF-β, TGF-β1, TGF-β2, TGF-β3, TNF-α, TNF-β, TRAIL, TRANCE, TWEAK, a functional portion thereof, or a combination thereof. In some embodiments, the insert sequence encodes a common gamma chain receptor, a common beta chain receptor, an interferon receptor, a TNF family receptor, a TGF-B receptor, a functional portion thereof, or a combination thereof. In some embodiments, the insert sequence encodes Apo3, BCMA, CD114, CD115, CD116, CD117, CD118, CD120, CD120a, CD120b, CD121, CD121a, CD121b, CD122, CD123, CD124, CD126, CD127, CD130, CD131, CD132, CD212, CD213, CD213a1, CD213a13, CD213a2, CD25, CD27, CD30, CD4, CD40, CD95 (Fas), CDw119, CDw121b, CDw125, CDw131, CDw136, CDw137 (41BB), CDw210, CDw217, GITR, HVEM, IL-11R, IL-11Rα, IL-14R, IL-15R, IL-15Ra, IL-18R, IL-18Rα, IL-18Rβ, IL-20R, IL-20Rα, IL-20Rβ, IL-9R, LIFR, LTβR, OPG, OSMR, OX40, RANK, TACI, TGF-βR1, TGF-βR2, TGF-βR3, TRAILR1, TRAILR2, TRAILR3, TRAILR4, a functional portion thereof, or a combination thereof.

In some embodiments, the insert sequence encodes a chemokine, or a functional portion thereof (e.g., a portion that binds to a chemokine receptor). In some embodiments the insert sequence encodes ACT-2, AMAC-a, ATAC, ATAC, BLC, BCA-1−, BRAK−, CCL1, CCL11, CCL13, CCL14, CCL15, CCL16, CCL17, CCL18, CCL19, CCL2, CCL20, CCL21, CCL22, CCL23, CCL24, CCL25, CCL26, CCL27, CCL3, CCL4, CCL5, CCL7, CCL8, CKb-6, CKb-8, CTACK, CX3CL1, CXCL1, CXCL10, CXCL11, CXCL12, CXCL13, CXCL14, CXCL2, CXCL3, CXCL4, CXCL5, CXCL6, CXCL7, CXCL8, CXCL9, DC-CK1, ELC, ENA-78+, eotaxin, eotaxin-2, eotaxin-3, Eskine, exodus-1, exodus-2, exodus-3, fractalkine, GCP-2+, GROa, GROb, GROg, HCC-1, HCC-2, HCC-4, I-309, IL-8, ILC, IP-10−, I-TAC−, LAG-1, LARC, LCC-1, LD78α, LEC, Lkn-1, LMC, lymphotactin, lymphotactin b, MCAF, MCP-1, MCP-2, MCP-3, MCP-4, MDC, MDNCF+, MGSA-a, MGSA-b, MGSA-g, Mig−, MIP-1d, MIP-1α, MIP-1β, MIP-2a+, MIP-2b+, MIP-3, MIP-3α, MIP-3β, MIP-4, MIP-4a, MIP-5, MPIF-1, MPIF-2, NAF, NAP-1, NAP-2, oncostatin A−, PARC, PF4, PPBP+, RANTES, SCM-1a, SCM-1b, SDF-1α/β−, SLC, STCP-1, TARC, TECK, XCL1, XCL2, a functional portion thereof, or a combination thereof. In some embodiments, the insert sequence encodes CCR1, CCR2, CCR3, CCR4, CCR5, CCR6, CCR7, CCR8, CCR9, CCR10, CX3CR1, CXCR1, CXCR2, CXCR3, CXCR4, CXCR5, XCR1, XCR1, a functional portion thereof, or a combination thereof.

In some embodiments, the insert sequence encodes a transcription factor (e.g., a transcription factor that affects expression of immune genes, immune cell function, immune cell differentiation, or a combination thereof). Examples of transcription factors that can be encoded by an insert sequence of the disclosure include, but are not limited to, AP-1, Bcl6, E2A, EBF, Eomes, FoxP3, GATA3, Id2, Ikaros, IRF, IRF1, IRF2, IRF3, IRF3, IRF7, NFAT, NFkB, Pax5, PLZF, PU.1, ROR-gamma-T, STAT, STAT1, STAT2, STAT3, STAT4, STAT5, STAT5A, STAT5B, STAT6, T-bet, TCF7, and ThPOK.

In some embodiments, the insert sequence encodes a transcription factor encodes a fusion protein comprising a drug-responsive domain (e.g., a protein that can be activated or inactivated by a drug). In some embodiments, the insert sequence encodes an enzyme.

In some embodiments, the insert sequence encodes an antibody, antigen-binding protein, or a functional portion thereof. For example, an insert sequence can encode an antibody heavy chain, light chain, or a combination thereof (for example, a heavy or light chain from an IgM, IgG, IgD, IgE, IgA, IgG1, IgG2, IgG3, IgG4, IgA1 or IgA2). An insert sequence can encode an antibody with constant regions or Fc regions that are selected or modified to provide suitable antibody characteristics, for example, suitable characteristics for treating a disease or condition as disclosed herein. In some embodiments, IgG1 can be used, for example, to promote immune activation effector functions (e.g., ADCC, ADCP, CDC, ITAM signaling, cytokine induction, or a combination thereof for the treatment of a cancer). In some embodiments, IgG4 can be used, for example, in cases where antagonistic properties of the antibody in the absence of immune effector functions are desirable.

An insert sequence can encode a non-antibody product that can bind a target antigen, for example, a designed ankyrin repeat protein (DARPin) or an aptamer.

In some embodiments, an insert sequence can encode a functional portion of an antibody or an antibody-derived protein. For example, an insert sequence can encode a protein comprising one or more complementarity determining regions (CDRs). An insert sequence can encode a protein comprising one or more variable regions derived from an antibody. Non-limiting examples of functional portions of antibodies and antibody-derived proteins include Fab, Fab′, F(ab′)₂, dimers and trimers of Fab conjugates, Fv, scFv, minibodies, dia-, tria-, and tetrabodies, linear antibodies. Fab and Fab′ are antigen-binding fragments that can comprise the VH and CHI domains of the heavy chain linked to the VL and CL domains of the light chain via a disulfide bond. A F(ab′)₂ can comprise two Fab or Fab′ that are joined by disulfide bonds. A Fv can comprise the VH and VL domains held together by non-covalent interactions. A scFv (single-chain variable fragment) is a fusion protein that can comprise the VH and VL domains connected by a peptide linker. Manipulation of the orientation of the VH and VL domains and the linker length can be used to create different forms of molecules that can be monomeric, dimeric (diabody), trimeric (triabody), or tetrameric (tetrabody).

An insert sequence can encode a fusion protein comprising one or more antigen-binding regions. An insert sequence can encode a fusion protein comprising two or more antigen-binding regions. For example, an insert sequence can encode a multi-specific antigen binding protein. In some embodiments, a multi-specific antigen binding protein can bind a cancer antigen and an immune cell antigen, thereby directing the immune cell to the cancer cell. An immune cell antigen can be present on, for example, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (T_{EM}), central memory T cells (T_{CM}), resident memory T cells (T_{RM}), Natural killer T cells (NKTs), tumor-infiltrating lymphocytes (TILs), Natural killer cells (NKs), Innate Lymphoid Cells (ILCs), B cells, B1 cells, B1a cells, B1b cells, B2 cells, plasma cells, B regulatory cells, antigen presenting cells (APCs), monocytes, macrophages, M1 macrophages, M2 macrophages, dendritic cells, plasmacytoid dendritic cells, neutrophils, mast cells, or a combination thereof. A multi-specific antigen-binding protein can comprise, for example, two, three, four, five, six, seven, eight, nine, ten, 11, 12, 13, 14, 15, 16, 17, 18, 19, or 20 antigen binding sites, or more. A multi-specific antigen-binding protein can comprise binding specificity for, for example, two, three, four, five, six, seven, eight, nine, or ten different target antigens.

In some embodiments, the insert sequence encodes a T cell receptor, a B cell receptor, cytokine receptor, chemokine receptor, NK cell receptor, NK T cell receptor, dendritic cell receptor, macrophage receptor, or monocyte receptor. In some embodiments, the insert sequence encodes a chimeric antigen receptor (CAR). In some embodiments, the insert sequence encodes a TCR or CAR.

In some cases, the insert sequence encodes for a CAR. In an aspect, a CAR comprises a CD3 zeta-chain (sometimes referred to as a 1st generation CAR). In another aspect, a CAR comprises a CD-3 zeta-chain and a single co-stimulatory domain (for example, CD28 or 4-1BB) (sometimes referred to as a 2nd generation CAR). In another aspect, a CAR comprises a CD-3 zeta-chain and two co-stimulatory domains (CD28/OX40 or CD28/4-1BB) (sometimes referred to as a 3rd generation CAR). Together with co-receptors such as CD8, these various signaling chains can produce downstream activation of kinase pathways, which support gene transcription and functional cellular responses.

A CAR can comprise an extracellular targeting domain, a transmembrane domain, and an intracellular signaling domain. A CAR can comprise at least a first binding moiety. Non-limiting examples of a binding moiety include, but are not limited to, a monoclonal antibody, a polyclonal antibody, a recombinant antibody, a human antibody, a humanized antibody, or a functional derivative, variant or fragment thereof, including, but not limited to, a Fab, a Fab′, a F(ab′)₂, an Fv, a single-chain Fv (scFv), minibody, a diabody, and a single-domain antibody such as a heavy chain variable domain (VH), a light chain variable domain (VL) and any combination thereof. A CAR may generally comprise a targeting domain derived from single chain antibody, hinge domain (H) or spacer, transmembrane domain (TM) providing anchorage to plasma membrane and signaling domains responsible of T-cell activation.

In an aspect, a receptor provided herein, such as a CAR, further comprises a hinge. A hinge can be located at any region of a CAR. In an aspect, a hinge is located between a binding moiety and a transmembrane region. In another aspect, a subject CAR comprises a hinge or a spacer. The hinge or the spacer can refer to a segment between the binding moiety and the transmembrane domain. In some embodiments, a hinge can be used to provide flexibility to a targeting moiety, e.g., scFv. In some embodiments, a hinge can be used to detect the expression of a CAR on the surface of a cell, for example when antibodies to detect the scFv are not functional or available. In some cases, the hinge is derived from an immunoglobulin molecule and may require optimization depending on the location of the first epitope or second epitope on the target. In some cases, a hinge may not belong to an immunoglobulin molecule but instead to another molecule such the native hinge of a CD8 alpha molecule. A CD8 alpha hinge can contain cysteine and proline residues which many play a role in the interaction of a CD8 co-receptor and MHC molecule. In some embodiments, a cysteine and proline residue can influence the performance of a CAR and may therefore be engineered to influence a CAR performance. I

In some embodiments, a hinge provided herein can be of any suitable length. In some embodiments, a hinge, for example used in a CAR, can be size tunable and can compensate, to some extent, in normalizing the orthogonal synapse distance between a CAR-expressing cell and a target cell. This topography of the immunological synapse between the CAR-expressing cell and target cell can also define a distance that cannot be functionally bridged by a CAR due to a membrane-distal epitope on a cell-surface target molecule that, even with a short hinge CAR, cannot bring the synapse distance in to an approximation for signaling. Likewise, membrane-proximal CAR target antigen epitopes have been described for which signaling outputs are only observed in the context of a long hinge CAR. A hinge disclosed herein can be tuned according to the single chain variable fragment region that can be used. In some embodiments, a hinge is from CD28, IgG1, and/or CD8α.

In some cases, a binding moiety of a CAR can be linked to an intracellular signaling domain via a transmembrane domain. A transmembrane domain can be a membrane spanning segment. A transmembrane domain of a CAR can anchor the CAR to the plasma membrane of a cell, for example an immune cell. In some embodiments, the membrane spanning segment comprises a polypeptide. The membrane spanning polypeptide linking the targeting moiety and the intracellular signaling domain of the CAR can have any suitable polypeptide sequence. In some cases, the membrane spanning polypeptide comprises a polypeptide sequence of a membrane spanning portion of an endogenous or wild-type membrane spanning protein. In some embodiments, the membrane spanning polypeptide comprises a polypeptide sequence having at least 1 (e.g., at least 2, 3, 4, 5, 6, 7, 8, 9, 10 or greater) of an amino acid substitution, deletion, and insertion compared to a membrane spanning portion of an endogenous or wild-type membrane spanning protein. In some embodiments, the membrane spanning polypeptide comprises a non-natural polypeptide sequence, such as the sequence of a polypeptide linker. The polypeptide linker may be flexible or rigid. The polypeptide linker can be structured or unstructured. In some embodiments, the membrane spanning polypeptide transmits a signal from an extracellular targeting moiety to an intracellular region. In an aspect, a subject CAR can comprise a transmembrane region that connects the targeting moiety to the intracellular region. A transmembrane region can be from or derived from an exogenous cellular transmembrane region. Various transmembrane regions are known in the art and can be from immune cell receptors. In an aspect, a transmembrane domain is from an alpha chain of a T cell receptor (TCR), beta chain of a TCR, CD3 epsilon, CD8, CD4, CD5, CD9, CD16, CD22, CD28, CD33, CD37, CD45, CD64, CD86, CD134, CD137, PD-1, and/or CD152. In some instances, a variety of human hinges can be employed as well including the human Ig (immunoglobulin) hinge. A native transmembrane portion of CD28 can be used in a CAR. In other cases, a native transmembrane portion of CD8 alpha can also be used in a subject CAR. In an aspect, the transmembrane domain is from an alpha chain of a TCR. In an aspect, the transmembrane domain is from CD8 and is CD8α. In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The intracellular signaling domain of a CAR of a subject fusion protein can comprise a signaling domain, or any derivative, variant, or fragment thereof, involved in immune cell signaling. The intracellular signaling domain of a CAR can induce activity of an immune cell comprising the CAR. The intracellular signaling domain can transduce the effector function signal and direct the cell to perform a specialized function. The signaling domain can comprise signaling domains of other molecules. While usually the signaling domain of another molecule can be employed in a CAR, in many cases it is not necessary to use the entire chain. In some cases, a truncated portion of the signaling domain is used in a CAR of the subject fusion protein.

In some embodiments, the intracellular signaling domain comprises multiple signaling domains involved in immune cell signaling, or any derivatives, variants, or fragments thereof. For example, the intracellular signaling domain can comprise at least 2 immune cell signaling domains, e.g., at least 2, 3, 4, 5, 7, 8, 9, or 10 immune cell signaling domains. An immune cell signaling domain can be involved in regulating primary activation of the TCR complex in either a stimulatory way or an inhibitory way. The intracellular signaling domain may be that of a TCR complex. The intracellular signaling domain of a subject CAR in a subject fusion protein can comprise a signaling domain of an Fcγ receptor (FcγR), an Fcε receptor (FcεR), an Fcα receptor (FcαR), neonatal Fc receptor (FcRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247ζ, CD247η, DAP10, DAP12, FYN, LAT, Lck, MAPK, MHC complex, NFAT, NF-κB, PLC-γ, iC3b, C3dg, C3d, and Zap70. In some embodiments, the signaling domain includes an immunoreceptor tyrosine-based activation motif or ITAM. A signaling domain comprising an ITAM can comprise two repeats of the amino acid sequence YxxL/I separated by 6-8 amino acids, wherein each x is independently any amino acid, producing the conserved motif YxxL/Ix₍₆₋₈₎YxxL/I. A signaling domain comprising an ITAM can be modified, for example, by phosphorylation when the targeting moiety is bound to an epitope. A phosphorylated ITAM can function as a docking site for other proteins, for example proteins involved in various signaling pathways. In some embodiments, the primary signaling domain comprises a modified ITAM domain, e.g., a mutated, truncated, and/or optimized ITAM domain, which has altered (e.g., increased or decreased) activity compared to the native ITAM domain.

In some embodiments, the intracellular signaling domain of a CAR in a subject fusion protein comprises an FcγR signaling domain (e.g., ITAM). The FcγR signaling domain can be selected from FcγRI (CD64), FcγRIIA (CD32), FcγRIIB (CD32), FcγRIIIA (CD16a), and FcγRIIIB (CD16b). In some embodiments, the intracellular signaling domain comprises an FcεR signaling domain (e.g., ITAM). The FcεR signaling domain can be selected from FcεRI and FcεRII (CD23). In some embodiments, the intracellular signaling domain comprises an FcαR signaling domain (e.g., ITAM). The FcαR signaling domain can be selected from FcαRI (CD89) and Fcα/μR. In some embodiments, the intracellular signaling domain comprises a CD3ζ (signaling domain. In some embodiments, the primary signaling domain comprises an ITAM of CD3ζ.

In some embodiments, an intracellular signaling domain of a subject CAR comprises an immunoreceptor tyrosine-based inhibition motif or ITIM. A signaling domain comprising an ITIM can comprise a conserved sequence of amino acids (S/I/V/LxYxxI/V/L) that is found in the cytoplasmic tails of some inhibitory receptors of the immune system. A primary signaling domain comprising an ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SHP-1 and SHP-2, the inositol-phosphatase called SHIP, and proteins having one or more SH2 domains (e.g., ZAP70). A intracellular signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1 (IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LIR8), leukocyte-associated immunoglobulin-like receptor 1 (LAIR-1), mast cell function-associated antigen (MAFA), NKG2A, natural cytotoxicity triggering receptor 2 (NKp44), NTB-A, programmed cell death protein 1 (PD-1), PILR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLEC5 or CD170), sialic acid binding Ig like lectin 6 (SIGLEC6), sialic acid binding Ig like lectin 7 (SIGLEC7), sialic acid binding Ig like lectin 10 (SIGLEC10), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT). In some embodiments, the intracellular signaling domain comprises a modified ITIM domain, e.g., a mutated, truncated, and/or optimized ITIM domain, which has altered (e.g., increased or decreased) activity compared to the native ITIM domain.

In some embodiments, the intracellular signaling domain comprises at least 2 ITAM domains (e.g., at least 3, 4, 5, 6, 7, 8, 9, or 10 ITAM domains). In some embodiments, the intracellular signaling domain comprises at least 2 ITIM domains (e.g., at least 3, 4, 5, 6, 7, 8, 9, or 10 ITIM domains) (e.g., at least 2 primary signaling domains). In some embodiments, the intracellular signaling domain comprises both ITAM and ITIM domains. In an aspect, an intracellular signaling domain of subject CAR is from an Fcγ receptor (FcγR), an Fcε receptor (FcεR), an Fcα receptor (FcαR), neonatal Fc receptor (FcRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247ζ, CD247η, DAP10, DAP12, FYN, LAT, Lck, MAPK, MHC complex, NFAT, NF-κB, PLC-γ, iC3b, C3dg, C3d, and Zap70. In another aspect, the intracellular signaling domain of a subject CAR is from CD3, CD3ζ, CD3γ, CD3δ, and/or CD3ε.

In some cases, a fusion protein provided herein comprises an intracellular signaling domain that comprises a co-stimulatory domain. In an aspect, a costimulatory domain can be part of a subject CAR of a fusion protein provided herein. In some embodiments, a co-stimulatory domain, for example from a cellular co-stimulatory molecule, can provide co-stimulatory signals for immune cell signaling, such as signaling from ITAM and/or ITIM domains, e.g., for the activation and/or deactivation of immune cell activity. In some embodiments, a costimulatory domain is operable to regulate a proliferative and/or survival signal in the immune cell. In some embodiments, a co-stimulatory signaling domain comprises a signaling domain of a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some embodiments, the costimulatory domain comprises a signaling domain of a molecule selected from the group consisting of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD5, CD53, CD58/LFA-3, CD69, CD7, CD8α, CD8β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6. In some embodiments, the intracellular signaling domain comprises multiple costimulatory domains, for example at least two, e.g., at least 3, 4, or 5 costimulatory domains. In an aspect, a receptor provided herein, such as a CAR, comprises at least 2 or 3 co-stimulatory domains. In an aspect, a receptor comprises at least 2 costimulatory domains, and wherein the at least 2 costimulatory domains are CD28 and CD137. In an aspect, the receptor comprises at least 3 costimulatory domains, and wherein the at least 3 costimulatory domains are CD28, CD137, and OX40L. Co-stimulatory signaling regions may provide a signal synergistic with the primary effector activation signal and can complete the requirements for activation of a T cell. In some embodiments, the addition of co-stimulatory domains to the CAR can enhance the efficacy and persistence of the immune cells provided herein.

In some cases, the insert sequence encodes a TCR or functional fragment thereof. A TCR refers to a molecule on the surface of a T cell or T lymphocyte that is responsible for recognizing an antigen. A TCR is a heterodimer which can be composed of two different protein chains. In some embodiments, the TCR of the present disclosure consists of an alpha (α) chain and a beta (β) chain and is referred as αβ TCR. αβ TCR recognizes antigenic peptides degraded from protein bound to major histocompatibility complex molecules (MHC) at the cell surface. In some embodiments, the TCR of the present disclosure consists of a gamma (γ) and a delta (δ) chain and is referred as γδ TCR. γδ TCR recognizes peptide and non-peptide antigens in a MHC-independent manner. γδ T cells have shown to play a prominent role in recognizing lipid antigens. In particular, the γ chain of TCR includes but is not limited to Vγ2, Vγ3, Vγ4, Vγ5, Vγ8, Vγ9, Vγ10, a functional variant thereof, and a combination thereof; and the 6 chain of TCR includes but is not limited to δ1, δ2, δ3, a functional variant thereof, and a combination thereof. In some embodiments, the γδ TCR may be Vγ2/Vδ1TCR, Vγ2/Vδ2 TCR, Vγ2/Vδ3 TCR, Vγ3/Vδ1 TCR, Vγ3/Vδ2 TCR, Vγ3/Vδ3 TCR, Vγ4/Vδ1 TCR, Vγ4/Vδ2 TCR, Vγ4/Vδ3 TCR, Vγ5/Vδ1 TCR, Vγ5/Vδ2 TCR, Vγ5/Vδ3 TCR, Vγ8/Vδ1 TCR, Vγ8/Vδ2 TCR, Vγ8/Vδ3 TCR, Vγ9/Vδ1 TCR, Vγ9/Vδ2 TCR, Vγ9/Vδ3 TCR, Vγ10/Vδ1 TCR, Vγ10/Vδ2 TCR, and/or Vγ10/Vδ3 TCR. In some examples, the γδ TCR may be Vγ9/Vδ2 TCR, Vγ10/Vδ2 TCR, and/or Vγ2/Vδ2 TCR.

In some cases, an insert sequence encodes for a TCR that comprises a TCR previously identified. In some cases, the TCR can be identified using whole-exomic sequencing. For example, a TCR can target a neoantigen or neoepitope that is identified by whole-exomic sequencing of a target cell. Alternatively, the TCR can be identified from autologous, allogenic, or xenogeneic repertoires. Autologous and allogeneic identification can entail a multistep process. In both autologous and allogeneic identification, dendritic cells (DCs) can be generated from CD14-selected monocytes and, after maturation, pulsed or transfected with a specific peptide. Peptide-pulsed DCs can be used to stimulate autologous or allogeneic immune cells, such as T cells. Single-cell peptide-specific T cell clones can be isolated from these peptide-pulsed T cell lines by limiting dilution. Subject TCRs of interest can be identified and isolated. Alpha, beta, gamma, and delta chains of a TCR of interest can be cloned, codon optimized, and encoded into a vector, for instance a lentiviral vector. In some embodiments, portions of the TCR can be replaced. For example, constant regions of a human TCR can be replaced with the corresponding murine regions. Replacement of human constant regions with corresponding murine regions can be performed to increase TCR stability. The TCR can also be identified with high or supraphysiologic avidity ex vivo. In some cases, a method of identifying a TCR can include immunizing transgenic mice that express the human leukocyte antigen (HLA) system with human tumor proteins to generate T cells expressing TCRs against human antigens (see e.g., Stanislawski et al., Circumventing tolerance to a human MDM2-derived tumor antigen by TCR gene transfer, Nature Immunology 2, 962-970 (2001)). An alternative approach can be allogeneic TCR gene transfer, in which tumor-specific T cells are isolated from a subject experiencing tumor remission and reactive TCR sequences can be transferred to T cells from another subject that shares the disease but may be non-responsive (de Witte, M. A., et al., Targeting self-antigens through allogeneic TCR gene transfer, Blood 108, 870-877 (2006)). In some cases, in vitro technologies can be employed to alter a sequence of a TCR, enhancing their tumor-killing activity by increasing the strength of an interaction (avidity) of a weakly reactive tumor-specific TCR with target antigen (Schmid, D. A., et al., Evidence for a TCR affinity threshold delimiting maximal CD8 T cell function. J. Immunol. 184, 4936-4946 (2010)).

In some embodiments, the insert sequence encodes a protein expressed on an immune cell that specifically binds an antigen expressed on a cancer cell. In some embodiments, the insert sequence encodes a protein expressed on an immune cell that specifically binds a neoantigen expressed on a cancer cell. In some embodiments, the insert sequence encodes a protein expressed on an immune cell that specifically binds a cancer associated antigen. In some embodiments, the insert sequence encodes a protein expressed on an immune cell that specifically binds an antigen expressed on a cancer cell. In some embodiments, the insert sequence encodes a protein expressed on an immune cell that specifically binds an antigen associated with an autoimmune disease. In some embodiments, the insert sequence encodes a protein expressed on an immune cell that specifically binds an antigen expressed on a pathogen (e.g., a microorganism, e.g., a virus, bacterium, parasite, fungus, or yeast).

In some embodiments, the insert sequence encodes a protein expressed on an immune cell and specifically binds carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, c-Met, mesothelin, GD3, HERV-K, IL-11Ralpha, kappa chain, lambda chain, CSPG4, ERBB2, EGFRvIII, VEGFR2, carbonic anhydrase IX, alphafetoprotein (AFP), α-actinin-4, ART-4, A1847, Ba 733, BAGE, BCMA, BrE3-antigen, CA125, CAMEL, CAP-1, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD3ε, CD40, CD40L, CD44, CD45, CD46, CD52, CD54, CD55, CD56, CD59, CD64, CD66a-e, CD67, CD70, CD7OL, CD74, CD79a, CD80, CD83, CD95, CD126, CD123, CD132, CD133, CD138, CD147, CD154, CDC27, CDK-4/m, CDKN2A, CTLA-4, CXCR4, CXCR7, CXCL12, HIF-1α, colon-specific antigen-p (CSAp), CEA (CEACAM5), CEACAM6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG), HER2/neu, HMGB-1, hypoxia inducible factor (HIF-1), HSP70-2 M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KS1-4, Le-Y, LDR/FUT, macrophage migration inhibitory factor (MIF), MAGE, MAGE-3, MART-1, MART-2, NY-ESO-1, TRAG-3, CRP, MDA-MB-231, MCP-1, MIP-1A, MIP-1B, MUC1, MUC2, MUC3, MUC4, MUC5ac, MUC13, MUC16, MUM-1/2, MUM-3, Mesothelin, NCA66, NCA95, NCA90, pancreatic cancer mucin, PD1, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, T101, SAGE, 5100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factor C3, complement factor C3a, complement factor C3b, complement factor C5a, complement factor C5, 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD3ε, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R1701, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

In some cases, a cellular receptor provided in an insert can be capable of binding to a neoantigen and/or neoepitope. Neoantigens and neoepitopes generally refer to tumor-specific mutations that in some cases trigger an antitumor T cell response. For example, these endogenous mutations can be identified using a whole-exomic-sequencing approach. Tran E, et al., “Cancer immunotherapy based on mutation-specific CD4+ T cells in a patient with epithelial cancer,” Science 344: 641-644 (2014). An antigen binding domain, for example, that of a subject CAR or a modified TCR complex can exhibit specific binding to a tumor-specific neo-antigen. Neoantigens bound by antigen binding domains the modified TCR complex can be expressed on a target cell, and for example, are neoantigens and neoepitopes encoded by mutations in any endogenous gene. In some cases, the two or more antigen binding domains bind a neoantigen or neoepitope encoded by a mutated gene. The gene can be selected from the group consisting of: ABL1, ACOl 1997, ACVR2A, AFP, AKT1, ALK, ALPPL2, ANAPC1, APC, ARID1A, AR, AR-v7, ASCL2, β2 M, BRAF, BTK, C15ORF40, CDH1, CLDN6, CNOT1, CT45A5, CTAG1B, DCT, DKK4, EEF1B2, EEF1DP3, EGFR, EIF2B3, env, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGEC1, MEK, MLANA, MLL2, MMP13, MSH3, MSH6, MYC, NDUFC2, NRAS, NY-ESO, PAGE2, PAGE5, PDGFRa, PIK3CA, PMEL, pol protein, POLE, PTEN, RAC1, RBM27, RNF43, RPL22, RUNX1, SEC31A, SEC63, SF3B1, SLC35F5, SLC45A2, SMAP1, SMAP1, SPOP, TFAM, TGFBR2, THAP5, TP53, TTK, TYR, UBR5, VHL, and XPOT.

In some embodiments, a cellular receptor provided in an insert can bind an antigen or epitope that may be present on a stroma. Stroma generally refers to tissue which, among other things, provides connective and functional support of a biological cell, tissue, or organ. A stroma can be that of the tumor microenvironment. The epitope may be present on a stromal antigen. Such an antigen can be on the stroma of the tumor microenvironment. Neoantigens and neoepitopes, for example, can be present on tumor endothelial cells, tumor vasculature, tumor fibroblasts, tumor pericytes, tumor stroma, and/or tumor mesenchymal cells. Example antigens include, but are not limited to, CD34, MCSP, FAP, CD31, PCNA, CD117, CD40, MMP4, and Tenascin.

### Homology Arms

A polynucleic acid construct can comprise a homology arm or homology arms. A homology arm can comprise a sequence with a degree of homology to a sequence in the genome of the immune cell to be edited, for example, to direct the repair of a double stranded break in the immune cell genome using the polynucleic acid construct or a part thereof as a repair template (e.g., repair via a pathway comprising single strand annealing, homology-mediated end joining, microhomology-mediated end joining, alternative end joining, homology-directed repair, homologous recombination, or a combination thereof). A homology arm can target a polynucleic acid construct or a part thereof to a desired site in the immune cell genome, e.g., a site adjacent to a double stranded break. A polynucleic acid construct can comprise one homology arm. A polynucleic acid construct of the disclosure can comprise two homology arms. Two homology arms in a polynucleic acid construct can flank a sequence to be inserted into the immune cell genome (e.g., a transgene). Two homology arms in a polynucleic acid construct can be directly adjacent to each other (e.g., for generating a deletion in the immune cell genome). A polynucleic acid construct of the disclosure can comprise three or more homology arms.

Homology arms of the disclosure can be single stranded DNA (ssDNA). In other aspects, homology arms are double stranded (dsDNA). In some aspects, a homology arm or homology arms are 100 nt and can flank each side of a donor insert sequence. In some aspects, a homology arm or homology arms are ssDN A of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, or 200 nt and can flank each side of a donor insert sequence. In some aspects, a homology arm or homology arms are ssDNA of about 100 nt and can flank each side of a donor insert sequence.

A homology arm can comprise a sequence with about or at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%9, 90%, 91%, 92%, 93%, 94%, 95%, 95.5%, 96%, 96.5%, 97%, 97.5%, 98%, 98.5%, 99%, 99.1%, 99.1%, 99.3%, 99.4%, 99.5%, 99.6%, 99.7%, 99.8%, 99.9%, 99.95%, 99.99%, or 100% sequence identity with a sequence in the genome of the immune cell to be edited. A homology arm can comprise a sequence with a degree of homology that is sufficient to allow the polynucleic acid construct or a part thereof to be used as a repair template for a double-stranded break in the immune cell genome. In some embodiments, a homology arm can contain one or more nucleotides that do not match the homologous sequence in the immune cell genome (e.g., for correction of one or more single nucleotide polymorphisms (SNPs) or for introduction of one or more SNPs). In some embodiments, two or more homology arms in a polynucleic acid construct contain the same degree of homology to corresponding sites in the immune cell genome. In some embodiments, two or more homology arms in a polynucleic acid construct contain different degrees of homology to corresponding sites in the immune cell genome. A homology arm can contain a nucleic acid sequence that is homologous to nucleotides in a gene, nucleotides in an open reading frame, nucleotides in a non-coding region or a combination thereof.

In some embodiments, a homology arm is about 24 nucleotides in length. In some embodiments, a homology arm is about 48 nucleotides in length. A homology arm can be, for example, about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 520, 540, 560, 580, 600, 620, 640, 660, 680, 700, 720, 740, 760, 780, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 nucleotides in length.

In some embodiments, a homology arm of the disclosure is at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 520, 540, 560, 580, 600, 620, 640, 660, 680, 700, 720, 740, 760, 780, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 nucleotides in length.

In some embodiments, a homology arm of the disclosure is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 520, 540, 560, 580, 600, 620, 640, 660, 680, 700, 720, 740, 760, 780, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 nucleotides in length.

A homology arm can be a short homology arm. A short homology arm can be, for example, at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 200, 210, 220, 230, 240, 250, 260, 270, 280, 290, 300, 310, 320, 330, 340, 350, 360, 370, 380, 390, or 400 nucleotides in length.

A short homology arm can be, for example, about 3-400, 5-300, 5-200, 5-100, 5-90, 5-80, 5-70, 5-60, 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-7, 5-16, 5-15, 5-14, 5-13, 5-12, 5-11, 5-10, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-29, 20-28, 20-27, 20-26, 20-25, 20-24, 24-50, 24-49, 24-48, 24-47, 24-46, 24-45, 24-44, 24-43, 24-42, 24-41, 24-40, 24-39, 24-38, 24-37, 24-36, 24-35, 24-34, 24-33, 24-32, 24-31, 24-30, 24-29, or 24-28 nucleotides in length.

A homology arm can be a long homology arm. A long homology arm can be, for example, at least 400, 410, 420, 430, 440, 450, 460, 470, 480, 490, 500, 520, 540, 560, 580, 600, 620, 640, 660, 680, 700, 720, 740, 760, 780, 800, 850, 900, 950, 1000, 1050, 1100, 1150, 1200, 1250, 1300, 1350, 1400, 1450, or 1500 nucleotides in length.

In some embodiments, a homology arm contains a number of nucleotides that is a multiple of three. In some embodiments, a homology arm contains a number of nucleotides that is not a multiple of three. In some embodiments, a homology arm contains a number of nucleotides that is a multiple of four. In some embodiments, a homology arm contains a number of nucleotides that is not a multiple of four.

A polynucleic acid construct of the disclosure can comprise, for example, 1, 2, 3, 4, 5, 6, 7, 8, 9, 10, 11, 12, 13, 14, 15, 16, 17, 18, 19, 20 homology arms, or more. In some embodiments, two or more homology arms in a polynucleic acid construct are the same length. In some embodiments, two or more homology arms in a polynucleic acid construct are different lengths.

In some embodiments, the homology arm comprises a nucleotide sequence that is at least about 70%, 75%, 80%, 85%, 90%, 91%, 92%, 93%, 94%, 95%, 96%, 97%, 98%, 99%, or 100% complementary to a genomic locus adjacent to a target site. In some embodiments, the homology arm comprises a nucleotide sequence that is from about 70%-100%, 80%-100%, 90%-100, 95%-100%, 96%-100%, 97%-100%, 98%-100%, 99%-100% complementary to a genomic locus adjacent to a target site. In some embodiments, the homology arm comprises a nucleotide sequence that is about 70%, 75%, 80%, 85%, 90%, 91%, 92%, 93%, 94%, 95%, 96%, 97%, 98%, 99%, or 100% complementary to a genomic locus adjacent to a target site.

In some embodiments, homology arms can comprise a sequence that is about 70%, 75%, 80%, 85%, 90%, 91%, 92%, 93%, 94%, 95%, 96%, 97%, 98%, 99%, or 100% complementary to a gene from Table 1, an immune checkpoint gene, a safe harbor gene, or any combination thereof.

### Cleavage Sites

In some embodiments, the one or more (e.g., two) homology arms in a polynucleic acid construct that flank an insert sequence are flanked by a cleavage site. For example, in some embodiments, a polynucleic acid construct comprises two homology arms, one on each end of an insert sequence (e.g., transgene), and each homology arm is flanked by a cleavage site. For example, from 5′ to 3′ the polynucleic acid can comprise a first cleavage site, a first homology arm, an insert sequence (e.g., a transgene), a second homology arm, and a second cleavage site. In some embodiments, a polynucleic acid construct contains one homology arm. For example, a polynucleic acid can comprise from 5′ to 3′ a cleavage site, a homology arm, an insert sequence (e.g., a transgene); or an insert sequence, a homology arm, and a cleavage site; or a cleavage site, an insert sequence, homology arm, and a cleavage site; or a cleavage site, a homology arm, an insert sequence, and a cleavage site.

In some embodiments, said cleavage site is adjacent to a targeted sequence recognized by a guide RNA (gRNA). In some embodiments, the targeted sequence is recognized by a gRNA (e.g., a sgRNA) that directs an endonuclease to the cleavage site. In some embodiments, said endonuclease is a CRISPR system endonuclease (e.g., a Cas endonuclease), TALEN endonuclease, or zinc finger endonuclease. In some embodiments, said endonuclease is an endonuclease described herein.

In some embodiments, the cleavage site is a CRISPR system cleavage site. In some embodiments, the CRISPR system cleavage site comprises a PAM motif and a sequence at least 80%, 85%, 90%, 95%, 96%, 97%, 98%, 99%, or 100% complementary to a gRNA. In some embodiments, said gRNA binds to said sequence.

In some embodiments, the CRISPR system cleavage site comprises a PAM motif. In some embodiments, the polynucleic acid construct comprises a spacer between the PAM motif and the homology arm. In some embodiments, the spacer is at least 1, 2, 3, 4, 5, 6, 7, 8, 9, or 10 bp. In some embodiments, the spacer is from about 1-10 bp, 1-9 bp, 1-8 bp, 1-7 bp, 1-6 bp, 1-5 bp, 1-4 bp, 1-3 bp, or 1-2 bp. In some embodiments, the spacer is about 1, 2, 3, 4, 5, 6, 7, 8, 9, or 10 bp. In some embodiments, the spacer is about 3 bp. In some embodiments, the spacer is at least 1, 2, 3, 4, 5, 6, 7, 8, 9, or 10 nucleotides. In some embodiments, the spacer is from about 1-10 nucleotides, 1-9 nucleotides, 1-8 nucleotides, 1-7 nucleotides, 1-6 nucleotides, 1-5 nucleotides, 1-4 nucleotides, 1-3 nucleotides, or 1-2 nucleotides. In some embodiments, the spacer is about 1, 2, 3, 4, 5, 6, 7, 8, 9, or 10 nucleotides. In some embodiments, the spacer is about 3 nucleotides.

### Promoters and Enhancers

In some embodiments, said polynucleic acid construct comprises a promoter. A suitable promoter can be selected by a person of ordinary skill in the art. Expression of a transgene can be controlled by at least one promoter. Exemplary promoters include, but are not limited to, CMV, U6, MND, PKG, MND, or EF1a.

The promoter can be a ubiquitous, constitutive (unregulated promoter that allows for continual transcription of an associated gene), tissue-specific promoter or an inducible promoter. Exemplary ubiquitous promoters include, but are not limited to, a CAGGS promoter, an hCMV promoter, a PGK promoter, an SV40 promoter, or a ROSA26 promoter.

The promoter can be endogenous or exogenous. For example, one or more transgenes can be inserted adjacent or near to an endogenous or exogenous ROSA26 promoter. Further, a promoter can be specific to a T cell. For example, one or more transgenes can be inserted adjacent or near to a porcine ROSA26 promoter.

Tissue specific promoter or cell-specific promoters can be used to control the location of expression. For example, one or more transgenes can be inserted adjacent or near to a tissue-specific promoter. Tissue-specific promoters can be a FABP promoter, a Lck promoter, a CamKII promoter, a CD19 promoter, a Keratin promoter, an Albumin promoter, an aP2 promoter, an insulin promoter, an MCK promoter, an MyHC promoter, a WAP promoter, or a Col2A promoter.

Inducible promoters can be used as well. These inducible promoters can be turned on and off when desired, by adding or removing an inducing agent. It is contemplated that an inducible promoter can be, but is not limited to, a Lac, tac, trc, trp, araBAD, phoA, recA, proU, cst-1, tetA, cadA, nar, PL, cspA, T7, VHB, Mx, and/or Trex.

In some embodiments, the insert sequence comprises an enhancer. In some embodiments, the enhancer is tissue specific. In some embodiments, the insert sequence comprises multiple enhancers (e.g., at least 2).

### Methods of Genetically Modifying Cells

Provided herein are methods of making genomically modified cells, e.g., immune cells, e.g., immune cells described herein. In some embodiments, the methods comprise introducing into a cell (e.g., an immune cell) (e.g., ex vivo) an endonuclease system (e.g., a CRISPR system that comprises a gRNA and a Cas nuclease) that introduces a genomic disruption in a targeted gene sequence, and introducing into the cell a polynucleic acid construct (e.g., described herein) that comprises at least one (e.g., 2) cleavage sequences, at least one (e.g., two homology arms) and an insert sequence (e.g., a transgene), wherein the transgene is inserted into the genomic disruption. In some embodiments an endonuclease introduces a double strand break at the least one cleavage site. In some embodiments, a single endonuclease is introduced. In some embodiments, at least two endonucleases are used. In some embodiments, the insert sequence is incorporated into the genome through microhomology-mediated end joining. In some embodiments, the insert sequence is incorporated into the genome through single strand annealing. insert sequence is incorporated into the genome through homology mediated end joining.

### Cleavage of Polynucleic Acid Construct

In some embodiments, the one or more (e.g., two) homology arms in a polynucleic acid construct that flank an insert sequence are flanked by a cleavage site. For example, in some embodiments, a polynucleic acid construct comprises two homology arms, one on each end of an insert sequence (e.g., transgene), and each homology arm is flanked by a cleavage site. For example, from 5′ to 3′ the polynucleic acid can comprise a first cleavage site, a first homology arm, an insert sequence (e.g., a transgene), a second homology arm, and a second cleavage site. In some embodiments, a polynucleic acid construct contains one homology arm. For example, a polynucleic acid can comprise from 5′ to 3′ a cleavage site, a homology arm, an insert sequence (e.g., a transgene); or an insert sequence, a homology arm, and a cleavage site; or a cleavage site, an insert sequence, homology arm, and a cleavage site; or a cleavage site, a homology arm, an insert sequence, and a cleavage site.

In some embodiments, the cleavage site is recognized by an endonuclease. In some embodiments, the cleavage sites comprise a CRISPR, zinc finger, or TALEN system cleavage site, as described herein. In some embodiments, the cleavage site comprises a CRISPR system cleavage site. In some embodiments, the CRISPR system cleavage site comprises a PAM sequence (e.g., as described herein) and a targeting nucleic acid sequence recognized by a gRNA (e.g., as described herein).

In some embodiments, cleavage at the cleavage site is mediated by introducing an endonuclease into the cell. In some embodiments, cleavage at the cleavage site is mediated by introducing a CRISPR system (e.g., as described herein) into the cell. In some embodiments, the CRISPR system comprises an endonuclease (e.g., as described herein) and a gRNA (e.g., as described herein).

### Genome Target Site

In some embodiments, the insert sequence is inserted into an endogenous gene in the genome of the cell. In some embodiments, the gene is a safe harbor locus, e.g., AAVS (e.g., AAVS1, AAVS2), CCR5, hROSA26, albumin. or HPRT.

In some embodiments, the gene codes for a cell surface receptor (e.g., TCR, BCR).

In some embodiments, the gene codes for an inhibitory immune checkpoint protein. In some embodiments, the inhibitory immune checkpoint protein is A2AR, B7-H3, B7-H4, BTLA, CTLA-4, IDO, KIR, LAG3, PD-1, TIM-3, VISTA or CISH. In some embodiments, the gene codes for a gene in Table 1.

In some cases, a construct provided herein can comprise homology arms that target any one of the exemplary endogenous genes from Table 1 and or other comparable genes. For example, a construct can comprise homology arms that are specific to a region of a gene in Table 1. In some aspects, an exemplary endogenous gene can be disrupted with a transgene insert sequence provided herein. The disruption may be sufficient to reduce and/or eliminate expression of an RNA or protein encoded by the endogenous gene.

TABLE 1
Exemplary Endogenous Genes

| | | | | NCBI | | | |
|---|---|---|---|---|---|---|---|
| | | | | number | | | |
| SEQ | | | | (GRCh38.p2) | | | Location |
| ID | Gene | | | *AC010327.8 | Original | Original | in |
| NO | Symbol | Abbreviation | Name | **GRCh38.p7 | Start | Stop | genome |
| 1 | ADORA2A | A2aR; | adenosine A2a | 135 | 24423597 | 24442360 | 22q11.23 |
| | | RDC8; | receptor | | | | |
| | | ADORA2 | | | | | |
| 3 | CD276 | B7H3; B7- | CD276 molecule | 80381 | 73684281 | 73714518 | 15q23-q24 |
| | | H3; B7RP- | | | | | |
| | | 2; 4Ig-B7- | | | | | |
| | | H3 | | | | | |
| 4 | VTCN1 | B7X; | V-set domain | 79679 | 117143587 | 117270368 | 1p13.1 |
| | | B7H4; | containing T cell | | | | |
| | | B7S1; B7- | activation inhibitor | | | | |
| | | H4; B7h.5; | 1 | | | | |
| | | VCTN1; | | | | | |
| | | PRO1291 | | | | | |
| 5 | BTLA | BTLA1; | B and T lymphocyte | 151888 | 112463966 | 112499702 | 3q13.2 |
| | | CD272 | associated | | | | |
| 6 | CTLA4 | GSE; | cytotoxic T- | 1493 | 203867788 | 203873960 | 2q33 |
| | | GRD4; | lymphocyte- | | | | |
| | | ALPS5; | associated protein 4 | | | | |
| | | CD152; | | | | | |
| | | CTLA-4; | | | | | |
| | | IDDM12; | | | | | |
| | | CELIAC3 | | | | | |
| 7 | IDO1 | IDO; | indoleamine 2,3- | 3620 | 39913809 | 39928790 | 8p12-p11 |
| | | INDO; | dioxygenase 1 | | | | |
| | | IDO-1 | | | | | |
| 8 | KIR3DL1 | KIR; | killer cell | 3811 | 54816438 | 54830778 | 19q13.4 |
| | | NKB1; | immunoglobulin- | | | | |
| | | NKAT3; | like receptor, three | | | | |
| | | NKB1B; | domains, long | | | | |
| | | NKAT-3; | cytoplasmic tail, 1 | | | | |
| | | CD158E1; | | | | | |
| | | KIR3DL2; | | | | | |
| | | KIR3DL1/ | | | | | |
| | | S1 | | | | | |
| 9 | LAG3 | LAG3; | lymphocyte- | 3902 | 6772483 | 6778455 | 12p13.32 |
| | | CD223 | activation gene 3 | | | | |
| 10 | PDCD1 | PD1; PD- | programmed cell | 5133 | 241849881 | 241858908 | 2q37.3 |
| | | 1; CD279; | death 1 | | | | |
| | | SLEB2; | | | | | |
| | | hPD-1; | | | | | |
| | | hPD-1; | | | | | |
| | | hSLE1 | | | | | |
| 11 | HAVCR2 | TIM3; | hepatitis A virus | 84868 | 157085832 | 157109237 | 5q33.3 |
| | | CD366; | cellular receptor 2 | | | | |
| | | KIM-3; | | | | | |
| | | TIMD3; | | | | | |
| | | Tim-3; | | | | | |
| | | TIMD-3; | | | | | |
| | | HAVcr-2 | | | | | |
| 12 | VISTA | C10orf54, | V-domain | 64115 | 71747556 | 71773580 | 10q22.1 |
| | | differentiation | immunoglobulin | | | | |
| | | of | suppressor of T-cell | | | | |
| | | ESC-1 | activation | | | | |
| | | (Dies1); | | | | | |
| | | platelet | | | | | |
| | | receptor | | | | | |
| | | Gi24 | | | | | |
| | | precursor; | | | | | |
| | | PD1 | | | | | |
| | | homolog | | | | | |
| | | (PD1H) | | | | | |
| | | B7H5; | | | | | |
| | | GI24; B7- | | | | | |
| | | H5; | | | | | |
| | | SISP1; | | | | | |
| | | PP2135 | | | | | |
| 13 | CD244 | 2B4; 2B4; | CD244 molecule, | 51744 | 160830158 | 160862902 | 1q23.3 |
| | | NAIL; | natural killer cell | | | | |
| | | Nmrk; | receptor 2B4 | | | | |
| | | NKR2B4; | | | | | |
| | | SLAMF4 | | | | | |
| 14 | CISH | CIS; G18; | cytokine inducible | 1154 | 50606454 | 50611831 | 3p21.3 |
| | | SOCS; | SH2-containing | | | | |
| | | CIS-1; | protein | | | | |
| | | BACTS2 | | | | | |
| 15 | HPRT1 | HPRT; | hypoxanthine | 3251 | 134452842 | 134500668 | Xq26.1 |
| | | HGPRT | phosphoribosyltrans | | | | |
| | | | ferase 1 | | | | |
| 16 | AAV*S1 | AAV | adeno-associated | 14 | 7774 | 11429 | 19q13 |
| | | | virus integration site | | | | |
| | | | 1 | | | | |
| 17 | CCR5 | CKR5; | chemokine (C-C | 1234 | 46370142 | 46376206 | 3p21.31 |
| | | CCR-5; | motif) receptor 5 | | | | |
| | | CD195; | (gene/pseudogene) | | | | |
| | | CKR-5; | | | | | |
| | | CCCKR5; | | | | | |
| | | CMKBR5; | | | | | |
| | | IDDM22; | | | | | |
| | | CC-CKR-5 | | | | | |
| 18 | CD160 | NK1; | CD160 molecule | 11126 | 145719433 | 145739288 | 1q21.1 |
| | | BY55; | | | | | |
| | | NK28 | | | | | |
| 19 | TIGIT | VSIG9; | T-cell | 201633 | 114293986 | 114310288 | 3q13.31 |
| | | VSTM3; | immunoreceptor | | | | |
| | | WUCAM | with Ig and ITIM | | | | |
| | | | domains | | | | |
| 20 | CD96 | TACTILE | CD96 molecule | 10225 | 111542079 | 111665996 | 3q13.1 |
| | | | | | | | 3q13.2 |
| 21 | CRTAM | CD355 | cytotoxic and | 56253 | 122838431 | 122872643 | 11q24.1 |
| | | | regulatory T-cell | | | | |
| | | | molecule | | | | |
| 22 | LAIR1 | CD305; | leukocyte | 3903 | 54353624 | 54370556 | 19q13.4 |
| | | LAIR-1 | associated | | | | |
| | | | immunoglobulin | | | | |
| | | | like receptor 1 | | | | |
| 23 | SIGLEC7 | p75; | sialic acid binding | 27036 | 51142294 | 51153526 | 19q13.3 |
| | | QA79; | Ig like lectin 7 | | | | |
| | | AIRM1; | | | | | |
| | | CD328; | | | | | |
| | | CDw328; | | | | | |
| | | D-siglec; | | | | | |
| | | SIGLEC7; | | | | | |
| | | SIGLECP2; | | | | | |
| | | SIGLEC19P; | | | | | |
| | | p75/AIRM1 | | | | | |
| 24 | SIGLEC9 | CD329; | sialic acid binding | 27180 | 51124880 | 51141020 | 19q13.41 |
| | | CDw329; | Ig like lectin 9 | | | | |
| | | FOAP-9; | | | | | |
| | | siglec-9; | | | | | |
| | | OBBP- | | | | | |
| | | LIKE | | | | | |
| 25 | TNFRSF10B | DR5; | tumor necrosis | 8795 | 23006383 | 23069187 | 8p22-p21 |
| | | CD262; | factor receptor | | | | |
| | | KILLER; | superfamily | | | | |
| | | TRICK2; | member 10b | | | | |
| | | TRICKB; | | | | | |
| | | ZTNFR9; | | | | | |
| | | TRAILR2; | | | | | |
| | | TRICK2A; | | | | | |
| | | TRICK2B; | | | | | |
| | | TRAIL- | | | | | |
| | | R2; | | | | | |
| | | KILLER/ | | | | | |
| | | DR5 | | | | | |
| 26 | TNFRSF10A | DR4; | tumor necrosis | 8797 | 23191457 | 23225167 | 8p21 |
| | | APO2; | factor receptor | | | | |
| | | CD261; | superfamily | | | | |
| | | TRAILR1; | member 10a | | | | |
| | | TRAILR-1 | | | | | |
| 27 | CASP8 | CAP4; | caspase 8 | 841 | 201233443 | 201287711 | 2q33-q34 |
| | | MACH; | | | | | |
| | | MCH5; | | | | | |
| | | FLICE; | | | | | |
| | | ALPS2B; | | | | | |
| | | Casp-8 | | | | | |
| 28 | CASP10 | MCH4; | caspase 10 | 843 | 201182898 | 201229406 | 2q33-q34 |
| | | ALPS2; | | | | | |
| | | FLICE2 | | | | | |
| 29 | CASP3 | CPP32; | caspase 3 | 836 | 184627696 | 184649475 | 4q34 |
| | | SCA-1; | | | | | |
| | | CPP32B | | | | | |
| 30 | CASP6 | MCH2 | caspase 6 | 839 | 109688628 | 109713904 | 4q25 |
| 31 | CASP7 | MCH3; | caspase 7 | 840 | 113679162 | 113730909 | 10q25 |
| | | CMH-1; | | | | | |
| | | LICE2; | | | | | |
| | | CASP-7; | | | | | |
| | | ICE-LAP3 | | | | | |
| 32 | FADD | GIG3; | Fas associated via | 8772 | 70203163 | 70207402 | 11q13.3 |
| | | MORT1 | death domain | | | | |
| 33 | FAS | APT1; | Fas cell surface | 355 | 88969801 | 89017059 | 10q24.1 |
| | | CD95; | death receptor | | | | |
| | | FAS1; | | | | | |
| | | APO-1; | | | | | |
| | | FASTM; | | | | | |
| | | ALPS1A; | | | | | |
| | | TNFRSF6 | | | | | |
| 34 | TGFBRII | AAT3; | transforming | 7048 | 30606493 | 30694142 | 3p22 |
| | | FAA3; | growth factor beta | | | | |
| | | LDS2; | receptor II | | | | |
| | | MFS2; | | | | | |
| | | RIIC; | | | | | |
| | | LDS1B; | | | | | |
| | | LDS2B; | | | | | |
| | | TAAD2; | | | | | |
| | | TGFR-2; | | | | | |
| | | TGFbeta- | | | | | |
| | | RII | | | | | |
| 35 | TGFBR1 | AAT5; | transforming | 7046 | 99104038 | 99154192 | 9q22 |
| | | ALK5; | growth factor beta | | | | |
| | | ESS1; | receptor I | | | | |
| | | LDS1; | | | | | |
| | | MSSE; | | | | | |
| | | SKR4; | | | | | |
| | | ALK-5; | | | | | |
| | | LDS1A; | | | | | |
| | | LDS2A; | | | | | |
| | | TGFR-1; | | | | | |
| | | ACVRLK4; | | | | | |
| | | tbetaR-I | | | | | |
| 36 | SMAD2 | JV18; | SMAD family | 4087 | 47833095 | 47931193 | 18q21.1 |
| | | MADH2; | member 2 | | | | |
| | | MADR2; | | | | | |
| | | JV18-1; | | | | | |
| | | hMAD-2; | | | | | |
| | | hSMAD2 | | | | | |
| 37 | SMAD3 | LDS3; | SMAD family | 4088 | 67065627 | 67195195 | 15q22.33 |
| | | LDS1C; | member 3 | | | | |
| | | MADH3; | | | | | |
| | | JV15-2; | | | | | |
| | | HSPC193; | | | | | |
| | | HsT17436 | | | | | |
| 38 | SMAD4 | JIP; | SMAD family | 4089 | 51030213 | 51085042 | 18q21.1 |
| | | DPC4; | member 4 | | | | |
| | | MADH4; | | | | | |
| | | MYHRS | | | | | |
| 39 | SKI | SGS; SKV | SKI proto-oncogene | 6497 | 2228695 | 2310213 | 1p36.33 |
| 40 | SKIL | SNO; | SKI-like proto- | 6498 | 170357678 | 170396849 | 3q26 |
| | | SnoA; | oncogene | | | | |
| | | SnoI; | | | | | |
| | | SnoN | | | | | |
| 41 | TGIF1 | HPE4; | TGFB induced | 7050 | 3411927 | 3458411 | 18p11.3 |
| | | TGIF | factor homeobox 1 | | | | |
| 42 | IL10RA | CD210; | interleukin 10 | 3587 | 117986391 | 118001483 | 11q23 |
| | | IL10R; | receptor subunit | | | | |
| | | CD210a; | alpha | | | | |
| | | CDW210A; | | | | | |
| | | HIL- | | | | | |
| | | 10R; IL- | | | | | |
| | | 10R1 | | | | | |
| 43 | IL10RB | CRFB4; | interleukin 10 | 3588 | 33266360 | 33297234 | 21q22.11 |
| | | CRF2-4; | receptor subunit | | | | |
| | | D21S58; | beta | | | | |
| | | D21S66; | | | | | |
| | | CDW210B; | | | | | |
| | | IL- | | | | | |
| | | 10R2 | | | | | |
| 44 | HMOX2 | HO-2 | heme oxygenase 2 | 3163 | 4474703 | 4510347 | 16p13.3 |
| 45 | IL6R | IL6Q; | interleukin 6 | 3570 | 154405193 | 154469450 | 1q21 |
| | | gp80; | receptor | | | | |
| | | CD126; | | | | | |
| | | IL6RA; | | | | | |
| | | IL6RQ; | | | | | |
| | | IL-6RA; | | | | | |
| | | IL-6R-1 | | | | | |
| 46 | IL6ST | CD130; | interleukin 6 signal | 3572 | 55935095 | 55994993 | 5q11.2 |
| | | GP130; | transducer | | | | |
| | | CDW130; | | | | | |
| | | IL-6RB | | | | | |
| 47 | CSK | CSK | c-src tyrosine kinase | 1445 | 74782084 | 74803198 | 15q24.1 |
| 48 | PAG1 | CBP; PAG | phosphoprotein | 55824 | 80967810 | 81112068 | 8q21.13 |
| | | | membrane anchor | | | | |
| | | | with | | | | |
| | | | glycosphingolipid | | | | |
| | | | microdomains 1 | | | | |
| 49 | SIT1 | SIT1 | signaling threshold | 27240 | 35649298 | 35650950 | 9p13- |
| | | | regulating | | | | p12 |
| | | | transmembrane | | | | |
| | | | adaptor 1 | | | | |
| 50 | FOXP3 | JM2; | forkhead box P3 | 50943 | 49250436 | 49269727 | Xp11.23 |
| | | AIID; | | | | | |
| | | IPEX; | | | | | |
| | | PIDX; | | | | | |
| | | XPID; | | | | | |
| | | DIETER | | | | | |
| 51 | PRDM1 | BLIMP1; | PR domain 1 | 639 | 106086320 | 106109939 | 6q21 |
| | | PRDI-BF1 | | | | | |
| 52 | BATF | SFA2; B- | basic leucine zipper | 10538 | 75522441 | 75546992 | 14q24.3 |
| | | ATF; | transcription factor, | | | | |
| | | BATF1; | ATF-like | | | | |
| | | SFA-2 | | | | | |
| 53 | GUCY1A2 | GC-SA2; | guanylate cyclase 1, | 2977 | 106674012 | 107018445 | 11q21- |
| | | GUC1A2 | soluble, alpha 2 | | | | q22 |
| 54 | GUCY1A3 | GUCA3; | guanylate cyclase 1, | 2982 | 155666568 | 155737062 | 4q32.1 |
| | | MYMY6; | soluble, alpha 3 | | | | |
| | | GC-SA3; | | | | | |
| | | GUC1A3; | | | | | |
| | | GUCSA3; | | | | | |
| | | GUCY1A1 | | | | | |
| 55 | GUCY1B2 | GUCY1B2 | guanylate cyclase 1, | 2974 | 50994511 | 51066157 | 13q14.3 |
| | | | soluble, beta 2 | | | | |
| | | | (pseudogene) | | | | |
| 56 | GUCY1B3 | GUCB3; | guanylate cyclase 1, | 2983 | 155758973 | 155807642 | 4q31.3- |
| | | GC-SB3; | soluble, beta 3 | | | | q33 |
| | | GUC1B3; | | | | | |
| | | GUCSB3; | | | | | |
| | | GUCY1B1; | | | | | |
| | | GC-S-beta-1 | | | | | |
| 57 | TRA | IMD7; | T-cell receptor | 6955 | 21621904 | 22552132 | 14q11.2 |
| | | TCRA; | alpha locus | | | | |
| | | TCRD; | | | | | |
| | | TRAalpha; | | | | | |
| | | TRAC | | | | | |
| 58 | TRB | TCRB; | T cell receptor beta | 6957 | 142299011 | 142813287 | 7q34 |
| | | TRBbeta | locus | | | | |
| 59 | EGLN1 | HPH2; | egl-9 family | 54583 | 231363751 | 231425044 | 1q42.1 |
| | | PHD2; | hypoxia-inducible | | | | |
| | | SM20; | factor 1 | | | | |
| | | ECYT3; | | | | | |
| | | HALAH; | | | | | |
| | | HPH-2; | | | | | |
| | | HIFPH2; | | | | | |
| | | ZMYND6; | | | | | |
| | | C1orf12; | | | | | |
| | | HIF-PH2 | | | | | |
| 60 | EGLN2 | EIT6; | egl-9 family | 112398 | 40799143 | 40808441 | 19q13.2 |
| | | PHD1; | hypoxia-inducible | | | | |
| | | HPH-1; | factor 2 | | | | |
| | | HPH-3; | | | | | |
| | | HIFPH1; | | | | | |
| | | HIF-PH1 | | | | | |
| 61 | EGLN3 | PHD3; | egl-9 family | 112399 | 33924215 | 33951083 | 14q13.1 |
| | | HIFPH3; | hypoxia-inducible | | | | |
| | | HIFP4H3 | factor 3 | | | | |
| 62 | PPP1R12C** | p84; p85; | protein phosphatase | 54776 | 55090913 | 55117600 | 19q13.42 |
| | | LENG3; | 1 regulatory subunit | | | | |
| | | MBS85 | 12C | | | | |

In some embodiments, the gene codes for a cell surface receptor that comprises an ITIM. In some embodiments, the endogenous gene is TRAC, TCRB, adenosine A2a receptor (ADORA), CD276, V-set domain containing T cell activation inhibitor 1 (VTCN1), B and T lymphocyte associated (BTLA), cytotoxic T-lymphocyte-associated protein 4 (CTLA4), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), programmed cell death 1 (PD-1), hepatitis A virus cellular receptor 2 (HAVCR2), V-domain immunoglobulin suppressor of T-cell activation (VISTA), natural killer cell receptor 2B34 (CD244), cytokine inducible SH2-containing protein (CISH), hypoxanthine phosphoribosyltransferase 1 (HPRT), adeno-associated virus integration site (AAVS (e.g., AAVS1, AAVS2)), or chemokine (C—C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1 (TGIF1), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), guanylate cyclase 1, soluble, beta 3 (GUCY1B3), prolyl hydroxylase domain (PHD1, PHD2, PHD3) family of proteins, A2AR, B7-H3, B7-H4, IDO, KIR, LAG3, TIM-3, VISTA, CD27, CD40, CD122, OX40, GITR, CD137, CD28, ICOS, A2AR, B7-H3, B7-H4, or PPP1R12C.

In some embodiments, multiple (e.g., at least 2, 3, 4, 5, 6, or more) target genes are disrupted in the host genome. In some embodiments, the genomic disruptions are double strand DNA. In some embodiments, one double strand break is introduced into a target site in the host genome. In some embodiments, at least two double strand breaks are introduced into two different target sites in the host genome. In some embodiments, two double strand breaks are introduced into two different target sites in the host genome in order to mediate deletion of a large section of DNA. In some embodiments, two double strand breaks are introduced into a single gene in the host genome in order to mediate deletion of a large section of DNA. In some embodiments, the genomic disruption suppresses expression of a protein encoded by the gene comprising the genomic disruption. In some embodiments, the genomic disruption suppresses expression of a functional protein encoded by the gene comprising the genomic disruption.

### DNA Repair Pathways

In some embodiments, provided herein are methods of resolving introduced double-stranded breaks in the genome of call using a repair template with at least one double strand break introduced. The introduction of at least one double stand break in the repair template permits the use of alternate or additional repair pathways, for example, pathways that comprise end resection, pathways that require only short homology arms in the repair template, or a combination thereof, for insertion of an insert sequence into the genome. Non-limiting examples of alternate or additional repair pathways that can be utilized include pathways comprising single strand annealing, homology-mediated end joining, microhomology-mediated end joining, alternative end joining, and combinations thereof.

In some embodiments, the methods provided herein exhibit an increased integration efficiency compared to a comparable method using a repair template that does not have the at least one double strand break.

In some embodiments, the methods described herein provide for an increase in percentage of cells which incorporate the insert sequence relative to a comparable population using a repair template that does not have the at least one double strand break. In some embodiments, at least 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, or 95% of the cells in population of cells described herein comprise an insert sequence. In some embodiments, at least 10% of the cells in population of cells described herein comprise an insert sequence. In some embodiments, at least 20% of the cells in population of cells described herein comprise an insert sequence. In some embodiments, at least 30% of the cells in population of cells described herein comprise an insert sequence. In some embodiments, at least 40% of the cells in population of cells described herein comprise an insert sequence. In some embodiments, at least 50% of the cells in population of cells described herein comprise an insert sequence. In some embodiments, at least 60% of the cells in population of cells described herein comprise an insert sequence. In some embodiments, at least 70% of the cells in population of cells described herein comprise an insert sequence. In some embodiments, at least 80% of the cells in population of cells described herein comprise an insert sequence. In some embodiments, at least 90% of the cells in population of cells described herein comprise an insert sequence. In some embodiments, at least 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, or 95% of the cells in population of cells described herein comprise an insert sequence and are viable. In some embodiments, at least 10% of the cells in population of cells described herein comprise an insert sequence and are viable. In some embodiments, at least 20% of the cells in population of cells described herein comprise an insert sequence and are viable. In some embodiments, at least 30% of the cells in population of cells described herein comprise an insert sequence and are viable. In some embodiments, at least 40% of the cells in population of cells described herein comprise an insert sequence and are viable. In some embodiments, at least 50% of the cells in population of cells described herein comprise an insert sequence and are viable. In some embodiments, at least 60% of the cells in population of cells described herein comprise an insert sequence and are viable. In some embodiments, at least 70% of the cells in population of cells described herein comprise an insert sequence and are viable. In some embodiments, at least 80% of the cells in population of cells described herein comprise an insert sequence and are viable. In some embodiments, at least 90% of the cells in population of cells described herein comprise an insert sequence and are viable. In some embodiments, integration of a transgene is measured 1-30, 1-21, 1-14, 1-7, 1-5, 1-4, 1-3, 1-2 days post introduction of said transgene. In some embodiments, cell viability is measured 1-30, 1-21, 1-14, 1-7, 1-5, 1-4, 1-3, 1-2 days post introduction of said transgene.

In some embodiments, efficiency of insert sequence integration is a function of the efficiency of the introduction of at least one double strand break in the polynucleic acid construct that comprises the insert sequence. In some embodiments, efficiency of insert sequence integration is a function of the efficiency of the excision of transgene from the polynucleic acid construct that comprises the insert sequence.

In some embodiments, the cells comprising the integrated transgene are expanded. In some embodiments, the cells comprising the integrated transgene are selectively expanded. In some embodiments, the cells comprising the integrated transgene are selectively expanded in vitro.

### Cell Viability and Integration Efficiency

Provided herein are methods of enhancing genomic transplantation. In some cases, methods provided herein increase cell viability. In some cases, methods provided herein increase transgene integration efficiency (also termed “transfection efficiency”). In some cases, methods provided herein increase both cell viability and transgene integration efficiency.

In some cases, cell viability is measured by cell counting via various approaches. In some cases, cell counting can be aided by staining of live cells. In some cases, cell counting can be automated, for instance, by flow cytometry or object recognition algorithm. In some cases, cell counting can be performed manually. In some cases, cell viability can be directly observed, for example, cells in culture dish tend to clump when dying. In some cases, cell viability can be measured by a viability assay, which can measure for instance, but not limited to, cytolysis, membrane leakage, mitochondrial activity or caspase expression, certain cellular function, expression of certain genes, genome integrity. In some cases, cell viability can be measured by viability dye staining. In some cases, the viability dye staining can be followed by flow cytometry. Viability dyes can differentiate live or dead cells or dying cells. The differential staining of the cells can be detected, e.g., by flow cytometry or microscopy.

Integration efficiency can be measured by detecting genomic insertion of transgene in the cells. In some cases, integration efficiency can be measured by detecting transgene product. For example, the exogenous polynucleic acid can comprise a reporter gene, e.g. a fluorescent protein, e.g. GFP, YFP, or mCherry. In some cases, the integration efficiency can be measured by examining the expression of the reporter gene, for example, by flow cytometry, which can count the cells expressing the fluorescent protein. In some cases, integration efficiency can be measured by assessing the genomic sequences of the electroporated cells directly, for instance, by examining the insertion of the transgene via sequencing.

In some embodiments, the methods provided herein exhibit an increased integration efficiency compared to a comparable method using a repair template that does not have the at least one double strand break.

In some embodiments, the methods described herein provide for an increase in percentage of cells which incorporate the insert sequence relative to a comparable population using a repair template that does not have the at least one double strand break. In some embodiments, at least 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, or 95% of the cells in population of cells described herein comprise an insert sequence. In some embodiments, at least 10% of the cells in population of cells described herein comprise an insert sequence. In some embodiments, at least 20% of the cells in population of cells described herein comprise an insert sequence. In some embodiments, at least 30% of the cells in population of cells described herein comprise an insert sequence. In some embodiments, at least 40% of the cells in population of cells described herein comprise an insert sequence. In some embodiments, at least 50% of the cells in population of cells described herein comprise an insert sequence. In some embodiments, at least 60% of the cells in population of cells described herein comprise an insert sequence. In some embodiments, at least 70% of the cells in population of cells described herein comprise an insert sequence. In some embodiments, at least 80% of the cells in population of cells described herein comprise an insert sequence. In some embodiments, at least 90% of the cells in population of cells described herein comprise an insert sequence. In some embodiments, at least 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90%, or 95% of the cells in population of cells described herein comprise an insert sequence and are viable. In some embodiments, at least 10% of the cells in population of cells described herein comprise an insert sequence and are viable. In some embodiments, at least 20% of the cells in population of cells described herein comprise an insert sequence and are viable. In some embodiments, at least 30% of the cells in population of cells described herein comprise an insert sequence and are viable. In some embodiments, at least 40% of the cells in population of cells described herein comprise an insert sequence and are viable. In some embodiments, at least 50% of the cells in population of cells described herein comprise an insert sequence and are viable. In some embodiments, at least 60% of the cells in population of cells described herein comprise an insert sequence and are viable. In some embodiments, at least 70% of the cells in population of cells described herein comprise an insert sequence and are viable. In some embodiments, at least 80% of the cells in population of cells described herein comprise an insert sequence and are viable. In some embodiments, at least 90% of the cells in population of cells described herein comprise an insert sequence and are viable. In some embodiments, integration of a transgene is measured 1-30, 1-21, 1-14, 1-7, 1-5, 1-4, 1-3, 1-2 days post introduction of said transgene. In some embodiments, cell viability is measured 1-30, 1-21, 1-14, 1-7, 1-5, 1-4, 1-3, 1-2 days post introduction of said transgene.

In some embodiments, efficiency of insert sequence integration is a function of the efficiency of the introduction of at least one double strand break in the polynucleic acid construct that comprises the insert sequence. In some embodiments, efficiency of insert sequence integration is a function of the efficiency of the excision of transgene from the polynucleic acid construct that comprises the insert sequence.

In some embodiments, the cells comprising the integrated transgene are expanded. In some embodiments, the cells comprising the integrated transgene are selectively expanded. In some embodiments, the cells comprising the integrated transgene are selectively expanded in vitro.

### Nuclease Treatment

Provided herein are methods of improving overall yield from cell engineering, including, for instance, improving cell viability after cell engineering, and/or improving transfection efficiency, comprising contacting genetically modified cells with a sufficient amount of at least one nuclease. In some instances, contacting with a sufficient amount of at least one nuclease for a sufficient period of time can increase cell viability. In some cases, contacting with a sufficient amount of at least one nuclease for a period of time can increase transfection efficiency. In some instances, contacting with a sufficient amount of at least one nuclease for a sufficient period of time can increase both cell viability and transfection efficiency.

Without wishing to be bound by a particular theory, as one of skills in the art would understand, during transfection, at least one point of the cell membrane is broken to allow exogenous nucleic acids, and/or other agent to enter the cell, causing invasive damage to the cell integrity and potentially with a lasting effect despite of the reversible nature of the membrane's open up. Moreover, as the exogenous agents are introduced to the intracellular environment, cells are not necessarily tolerant to their intracellular presence. Another potential adverse effect may come from the exogenous agents that can be trapped between the lipid bilayer of the cell membrane as the membrane reseals after the temporary open up.

In some instances, the method of promoting cell viability can comprise contacting the cells with a nuclease, which, by definition, can catalyze the hydrolytic cleavage of phosphodiester linkages (hydrolysis or digestion) in polynucleic acids with selectivity. The nuclease can include deoxyribonuclease (DNase), ribonuclease (RNase), or both. DNase can specifically digest DNAs, while RNase can digest RNAs specifically. Nucleases can also be classified as endonucleases or exonucleases. An exonuclease can refer to any of a group of enzymes that catalyze the hydrolysis of a polynucleic acid molecule from its' 5′, 3′, both ends. An endonuclease can refer to any of a group of enzymes that catalyze the hydrolysis of a polynucleic acid molecule between nucleic acids in the interior of a polynucleic acid molecule. Some enzymes can have both exonuclease and endonuclease properties. In addition, some enzymes are able to digest both DNA and RNA sequences.

Contacting with a nuclease can lead to an increase in viability percentage about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 125%, about 150%, about 175%, about 200%, about 250%, about 300%, or even more. In some cases, an increase in viability percentage can be from about 50% to about 200%. Contacting with a nuclease can lead to an increase in integration efficiency can be about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 125%, about 150%, about 175%, about 200%, about 250%, about 300%, or even more. In some cases, an increase in integration efficiency can be from about 50% to about 200%.

Non-limiting examples of the DNase in connection with the subject matter described herein can include DNase I, Benzonase, Exonuclease I, Exonuclease III, Mung Bean Nuclease, Nuclease BAL 31, RNase I, S1 Nuclease, Lambda Exonuclease, RecJ, T7 exonuclease, and various restriction enzymes that are specialized in breaking phosphodiester linkages in their respective recognition sequences. Non-limiting examples of the RNase in connection with the subject matter disclosed herein can include RNase A, RNase H, RNase III, RNase L, RNase P, RNase PhyM, RNase T1, RNase T1, RNase U2, RNase V, Polynucleotide Phosphorylase, RNase PH, RNase R, RNase D, RNase I, RNase II, RNase T, Oligoribonuclease, Exoribonuclease I, and Exoribonuclease II. Appropriate nuclease can be chosen depending on the property of the polynucleic acid being introduced into the cell and the type of the cell being transfected.

In some cases, the nuclease can be applied after the cell transfection. In some cases, the nuclease can be introduced immediately after the cell transfection is completed. In some cases, the nuclease can be introduced while the cell transfection is being conducted, for instance, applied while electroporation is being performed, or applied while the cell is still being exposed to transfection reagents. In some cases, the nuclease can be introduced up to several minutes to several hours post-transfection. The time delay between the completion of cell transfection and application of the nuclease can be about 30 sec, about 1 min, about 2 min, about 3 min, about 4 min, about 5 min, about 6 min, about 7 min, about 8 min, about 9 min, about 10 min, about 15 min, about 30 min, about 45 min, about 60 min, about 1.5 hrs, about 2 hrs, about 3 hrs, about 4 hrs, about 5 hrs, about 7.5 hrs, about 8 hrs, about 10 hrs, about 12 hrs, about 20 hrs, about 30 hrs, about 40 hrs, about 50 hrs, about 60 hrs, about 70 hrs, about 80 hrs, about 90 hrs, or about 1 week. In some cases, the time delay can be even longer.

In some cases, the nuclease can be applied before the cell transfection. A nuclease can be present in the cell culture through a period of time before the transfection. In some cases, the “pre-treatment” of the nuclease can promote the general health of the target cells. For instance, in many cases, it can promote the survival of the cells after isolation from the living organ of an organism. The nuclease can be present both before and after the cell transfection.

The nuclease can be supplied in the culture medium at a concentration about 1 μg/ml, 10 μg/ml, 50 μg/ml, 100 μg/ml, 200 μg/ml, 300 μg/ml, 400 μg/ml, 500 μg/ml, 600 μg/ml, 700 μg/ml, 800 μg/ml, 900 μg/ml, 950 μg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 20 mg/ml, 30 mg/ml, 40 mg/ml, 50 mg/ml, 100 mg/ml, 200 mg/ml, 500 mg/ml, or about a value between any two of these values. The nuclease can be supplied in the culture medium at a concentration about 1 mg/mL. The nuclease can be supplied in the culture medium, and the culture medium containing the nuclease can be replaced once about every 3 hr, 6 hr, 12 hr, 20 hr, 21 hr, 22 hr, 23 hr, 24 hr, 26 hr, 28 hr, 30 hr, 32 hr, 34 hr, 36 hr, 40 hr, 44 hr, 48 hr, 50 hr, 60 hr. The frequent replacement of the culture medium can maintain the concentration of the nuclease at a certain level.

As those skilled in the art would appreciate, the choice of the nuclease, the concentration of the nuclease, and the timing and the duration for the incubation of the nuclease can vary depending on many parameters of the particular application of the subject matter described herein. The various parameters can include, but not limited to, the cell type, the properties of the polynucleic acids to be transferred, the overall health of the cells, the expected viability to achieve, and the intended use of the transfected cells.

In some cases, the cells can be treated/incubated with the nuclease for a period of time. The incubation time can be at least about 1 min, at least about 2 min, at least about 3 min, at least about 4 min, at least about 5 min, at least about 10 min, at least about 20 min, at least about 30 min, at least about 45 min, or at least about 60 min. The incubation time can be at least about 1 hr, at least about 2 hrs, at least about 3 hrs, at least about 4 hrs, at least about 5 hrs, at least about 7.5 hrs, at least about 8 hrs, at least about 10 hrs, at least about 12 hrs, at least about 20 hrs, at least about 30 hrs, at least about 40 hrs, at least about 50 hrs, at least about 60 hrs, at least about 70 hrs, at least about 80 hrs, at least about 90 hrs, or at least about 1 week. In some cases, the incubation time can be at least 1 week, at least 2 weeks, at least 3 weeks, or even longer.

In some instances, the cells can be exposed to the nuclease for 1 to 30 min at 18-25° C. in a mixture. In some examples, the mixture can comprise PBS, FBS, magnesium, and DNase.

### Immune Stimulatory Agent

Provided herein are methods of improving overall yield from cell engineering, including, for instance, improving cell viability after cell engineering, and/or improving transfection efficiency, comprising contacting genetically modified cells with a sufficient amount of at least one immune stimulatory agent. In some instances, contacting with a sufficient amount of at least one immune stimulatory agent for a sufficient period of time can increase cell viability. In some cases, contacting with a sufficient amount of at least one immune stimulatory agent for a period of time can increase transfection efficiency. In some instances, contacting with a sufficient amount of at least one immune stimulatory agent for a sufficient period of time can increase both cell viability and transfection efficiency.

Immune stimulatory agent can include any type of reagent that can stimulate an immune cell. For example, an immune stimulatory agent can comprise a cytokine. In some cases, an immune stimulatory agent can comprise an antibody against or a ligand of an immune cell receptor.

Cytokines refer to proteins (e.g., chemokines, interferons, lymphokines, interleukins, and tumor necrosis factors) released by cells which can affect cell behavior. Cytokines are produced by a broad range of cells, including immune cells such as macrophages, B lymphocytes, T lymphocytes and mast cells, as well as endothelial cells, fibroblasts, and various stromal cells. A given cytokine can be produced by more than one type of cell. Cytokines can be involved in producing systemic or local immunomodulatory effects. Exemplary cytokines include, but are not limited to, IL-2, IL-7, IL-12, IL-15, IL-21, or any combination thereof.

In some cases, an aAPC may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83. In some cases, modified target cells of present disclosure can comprise immune cells, e.g., T cells or B cells. Immune cells can be stimulated by immune stimulatory agent to expand. For example, T cells can be expanded by contact with a surface having attached thereto an agent that can stimulate a CD3 TCR complex associated signal and a ligand that can stimulate a co-stimulatory molecule on the surface of the T cells. In particular, T cell populations can be stimulated such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. For co-stimulation of an accessory molecule on the surface of the T cells, a ligand that binds the accessory molecule can be used. For example, a population of T cells can be contacted with an anti-CD3 antibody and an anti-CD28 antibody, under conditions that can stimulate proliferation of the T cells. In some cases, 4-1BB can be used to stimulate cells. For example, cells can be stimulated with 4-1BB and IL-21 or another cytokine.

To stimulate proliferation of either CD4 T cells or CD8 T cells, an anti-CD3 antibody and an anti-CD28 antibody can be used. For example, the agents providing a signal may be in solution or coupled to a surface. In some cases, the cells, such as T cells, can be combined with agent-coated beads. Each bead can be coated with either anti-CD3 antibody or an anti-CD28 antibody, or in some cases, a combination of the two. Any bead to cell ratio can be utilized. In some cases, a ratio is 5:1; 2.5:1; 1:1; 1:2; 1:5; 1:2.5; or 2:1 bead:cells. Immune stimulatory agents that are appropriate for modified T cell proliferation and viability include, but not limited to, interleukin-2 (IL-2), IFN-g, IL-4, IL-7, GM-CSF, IL-10, IL-21, IL-15, TGF beta, and TNF alpha or any derivatives thereof.

In some cases, an additional stimulation protocol can be utilized during preparation of modified cells. An additional stimulation can comprise an initial stimulation utilizing an immune stimulatory agent provided herein. Stimulation can be timed such that cells are stimulated prior, concurrent, and/or after electroporation. In some cases, cells may be subject to one or more stimulations. In some cases, cells may be subject to 1, 2, 3, 4, 5, or up to about 6 stimulations utilizing any of the antibodies, antibody fragments thereof, and/or any beads displaying stimulatory antibodies or fragments thereof. In some cases, an additional stimulation comprises continuous stimulation. For example, after an electroporation, cells may be continuously stimulated thereafter using any of the compositions and methods provided herein, for example anti-CD3 and/or anti-CD28. An additional stimulation may increase cellular expansion as compared to a comparable method that lacks the additional stimulation. In some cases, the additional stimulation, such as a second stimulation, is performed after cellular electroporation. In some cases, a second stimulation is performed immediately after electroporation. In other cases, a second stimulation is performed from about 5 min, 10 min, 20 min, 30 min, 40 min, 50 min, 1 hr, 2 hrs, 4 hrs, 6 hrs, 8 hrs, 10 hrs, 12 hrs, 14 hrs, 16 hrs, 18 hrs, or up to about 20 hrs after an electroporation. The additional stimulation may be performed for any length of time. For example, the additional stimulation may be performed for about 2 hrs, 4 hrs, 6 hrs, 8 hrs, 10 hrs, 12 hrs, 14 hrs, 16 hrs, 18 hrs, 20 hrs, 22 hrs, 24 hrs, 26 hrs, 28 hrs, 30 hrs, 32 hrs, 34 hrs, 36 hrs, 38 hrs, 40 hrs, 42 hrs, 44 hrs, 46 hrs, 48 hrs, or up to about 50 hrs. In some cases, the additional stimulation is from about 24-48 hrs. or from about 30-40 hrs. In some cases, a stimulation comprises a first stimulation prior to electroporation followed by a second stimulation after electroporation. The electroporated cells can be stimulated with beads at the previously described ratios, for example at 2:1 or 1:2.5 (beads per cell).

In some cases, target cells or modified target cells can be activated or expanded by co-culturing with tissue or cells. A cell can be an antigen presenting cell or an artificial antigen presenting cell. Antigen presenting cells (APCs) can include, but not limited to, dendritic cells, macrophages, B cells, and other non-professional APCs. An APC can express a number of immune stimulatory molecules on its surface, such as, but not limited to, B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, membrane-bound IL-15, membrane-bound IL-17, membrane-bound IL-21, membrane-bound IL-2, truncated CD19, derivative thereof, or any combination thereof.

An artificial antigen presenting cells (aAPCs) can express ligands for T cell receptor and costimulatory molecules and can activate and expand T cells for transfer, while improving their potency and function in some cases. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal.

An aAPC can be a bead. A spherical polystyrene bead can be coated with antibodies against CD3 and CD28 and be used for T cell activation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a 4, poly(lactic-co-glycolic acid) (PLGA) microsphere, a nonspherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof.

In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells. In some cases, the use of aAPCs can be combined with exogenously introduced cytokines for T cell activation, expansion, or any combination. Cells can also be expanded in vivo, for example in the subject's blood after administration of modified cells into a subject.

In some cases, methods and compositions provided herein can include substantially antibiotics-free cell culture media. Antibiotics, e.g., penicillin and streptomycin, can be included only in experimental cultures, possibly not in cultures of cells that are to be infused into a subject. The term “substantially antibiotics-free medium” can refer to a medium having no or almost no antibiotics therein, for instance, a medium having 0 g/ml antibiotics, or a medium having at most 1 μg/ml, at most 0.5 μg/ml, at most 0.2 μg/ml, at most 100 ng/ml, at most 50 ng/ml, at most 20 ng/ml, at most 10 ng/ml, at most 5 ng/ml, at most 2 ng/ml, at most 1 ng/ml, at most 500 μg/ml, at most 200 μg/ml, at most 100 μg/ml, at most 50 μg/ml, at most 20 μg/ml, at most 10 μg/ml, at most 5 μg/ml, at most 2 μg/ml, at most 1 μg/ml, at most 500 fg/ml, at most 200 fg/ml, at most 100 fg/ml, at most 50 fg/ml, at most 20 fg/ml, at most 10 fg/ml, or at most 1 fg/ml antibiotics.

In some cases, the immune stimulatory agent can be applied after the cell transfection. In some cases, the immune stimulatory agent can be introduced immediately after the cell transfection is completed. In some cases, the immune stimulatory agent can be introduced while the cell transfection is being conducted, for instance, applied while electroporation is being performed, or applied while the cell is still being exposed to transfection reagents. In some cases, the immune stimulatory agent can be introduced up to several minutes to several hours post-transfection. The time delay between the completion of cell transfection and application of the immune stimulatory agent can be about 30 sec, about 1 min, about 2 min, about 3 min, about 4 min, about 5 min, about 6 min, about 7 min, about 8 min, about 9 min, about 10 min, about 15 min, about 30 min, about 45 min, about 60 min, about 1.5 hrs, about 2 hrs, about 3 hrs, about 4 hrs, about 5 hrs, about 7.5 hrs, about 8 hrs, about 10 hrs, about 12 hrs, about 20 hrs, about 30 hrs, about 40 hrs, about 50 hrs, about 60 hrs, about 70 hrs, about 80 hrs, about 90 hrs, or about 1 week. In some cases, the time delay can be even longer.

In some cases, the immune stimulatory agent can be applied before the cell transfection. In certain cases, the immune stimulatory agent can be present in the cell culture through a period of time before the transfection. The immune stimulatory agent can be present both before and after the cell transfection.

The immune stimulatory agent can be supplied in the culture medium at a concentration about 20 μg/ml, 50 μg/ml, 100 μg/ml, 200 μg/ml, 300 μg/ml, 400 μg/ml, 500 μg/ml, 600 μg/ml, 700 μg/ml, 800 μg/ml, 900 μg/ml, 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 12 ng/ml, 15 ng/ml, 20 ng/ml, 25 ng/ml, 30 ng/ml, 35 ng/ml, 40 ng/ml, 45 ng/ml, 50 ng/ml, 75 ng/ml, 100 ng/ml, 200 ng/ml, 500 ng/ml, 750 ng/ml, 1 μg/ml, 5 μg/ml, 10 μg/ml, 50 μg/ml, or about a value between any two of these values. The immune stimulatory agent can be supplied in the culture medium at a concentration about 5 ng/mL. The immune stimulatory agent can be supplied in the culture medium, and the culture medium containing the immune stimulatory agent can be replaced once about every 6 hr, 12 hr, 20 hr, 21 hr, 22 hr, 23 hr, 24 hr, 26 hr, 28 hr, 30 hr, 32 hr, 34 hr, 36 hr, 40 hr, 44 hr, 48 hr, 50 hr, 60 hr. The frequent replacement of the culture medium can maintain the concentration of the immune stimulatory agent at a certain level.

As those skilled in the art would appreciate, the choice of the immune stimulatory agent, the concentration of the immune stimulatory agent, and the timing and the duration for the incubation of the immune stimulatory agent can vary depending on many parameters as discussed above.

In some cases, the cells can be treated/incubated with the immune stimulatory agent for a period of time. The incubation time can be at least about 1 min, at least about 2 min, at least about 3 min, at least about 4 min, at least about 5 min, at least about 10 min, at least about 20 min, at least about 30 min, at least about 45 min, or at least about 60 min. The incubation time can be at least about 1 hr, at least about 2 hrs, at least about 3 hrs, at least about 4 hrs, at least about 5 hrs, at least about 7.5 hrs, at least about 8 hrs, at least about 10 hrs, at least about 12 hrs, at least about 20 hrs, at least about 30 hrs, at least about 40 hrs, at least about 50 hrs, at least about 60 hrs, at least about 70 hrs, at least about 80 hrs, at least about 90 hrs, or at least about 1 week. In some cases, the incubation time can be at least 1 week, at least 2 weeks, at least 3 weeks, or even longer.

### Modulator of Double Strand Break Repair

Provided herein are methods of improving overall yield from cell engineering, including, for instance, improving cell viability after cell engineering, and/or improving transfection efficiency, comprising contacting genetically modified cells with a sufficient amount of at least one modulator of DNA double strand break repair. In some instances, contacting with a sufficient amount of at least one modulator of DNA double strand break repair for a sufficient period of time can increase cell viability. In some cases, contacting with a sufficient amount of at least one modulator of DNA double strand break repair for a period of time can increase transfection efficiency. In some instances, contacting with a sufficient amount of at least one modulator of DNA double strand break repair for a sufficient period of time can increase both cell viability and transfection efficiency.

In some cases, a modulator of DNA double strand break repair can comprise a protein involved in DNA double strand break repair. In some case, a modulator of DNA double strand break repair can comprise a chemical compound. A modulator of double strand break repair can be human, non-human, and/or synthetic. In some cases, a modulator of double strand break repair is human. In some cases, a modulator of double strand break repair is non-human. Suitable non-human sources include any one of the following, non-limiting species: rat, mouse, donkey, pig, cow, dog, cat, ferret, monkey, goat, sheep, fish, or any combination thereof.

Non-limiting examples of a protein involved in DNA double strand break repair that can be used for improving genome editing can include Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, homologs and derivatives thereof, Scr7, and any combination thereof. In some cases, a protein involved in DNA double strand break repair that can be used for improving genome editing can comprise RAD51. In some cases, a protein involved in DNA double strand break repair that can be used for improving genome editing can comprise RS-1. A protein of RS-1 or RAD51 can be used. A polynucleotide encoding RS-1 or RAD-51 can also be used. An mRNA of RS-1 or RAD-51 can also be used.

In some cases, genome editing as described herein can comprise insertion of a transgene. A transgene is typically not identical to the genomic sequence where it is placed. Insertion of a transgene typically involves excision of target genomic sequence, thereby DNA double strand break. In some cases, nonhomologous end-joining (NHEJ pathway), involving proteins like Ku70 and Ku80, as well as homologous recombination pathway, involving proteins like BRCA, BRCA2 and Rad51 (HR pathway), are activated during a double strand break event in a cell. In some cases, a modulator of DNA double strand break repair as described herein can comprise a HR enhancer. A HR enhancer can promote homologous recombination mediated DNA double strand break repair. In some cases, a HR enhancer can inhibit NHEJ mediated DNA double strand break repair. In some cases, a modulator of DNA double strand break repair as described herein can comprise a NHEJ enhancer. A NHEJ enhancer can promote NHEJ mediated DNA double strand break repair. In some cases, a NHEJ enhancer can inhibit HR mediated DNA double strand break repair.

A transgene as described herein can be introduced to a genome by homologous recombination. In some cases, a transgene can be flanked by homology arms. In some cases, homology arms can comprise complementary regions that target a transgene to a desired integration site. In some cases, a donor transgene can contain a non-homologous sequence flanked by two regions of homology to allow for efficient HDR at the location of interest. Additionally, transgene sequences can comprise a vector molecule containing sequences that are not homologous to the region of interest in cellular chromatin. A transgene can contain several, discontinuous regions of homology to cellular chromatin. For example, for targeted insertion of sequences not normally present in a region of interest, a sequence can be present in a donor nucleic acid molecule and flanked by regions of homology to sequence in the region of interest.

A transgene can be flanked by homology arms where the degree of homology between the arm and its complementary sequence is sufficient to allow homologous recombination between the two. For example, the degree of homology between the arm and its complementary sequence can be 50% or greater. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. Any other gene, e.g., the genes described herein, can be used to generate a recombination arm.

A transgene can also be flanked by engineered sites that are complementary to the targeted double strand break region in a genome. In some cases, engineered sites are not homology arms. Engineered sites can have homology to a double strand break region. Engineered sites can have homology to a gene. Engineered sites can have homology to a coding genomic region. Engineered sites can have homology to a non-coding genomic region. In some cases, a transgene can be excised from a polynucleic acid so it can be inserted at a double strand break region without homologous recombination. A transgene can integrate into a double strand break without homologous recombination.

In some cases, a homologous recombination HR enhancer can be used to suppress non-homologous end-joining (NHEJ). Non-homologous end-joining can result in the loss of nucleotides at the end of double stranded breaks; non-homologous end-joining can also result in frameshift. In some cases, homology-directed repair can be a more attractive mechanism to use when knocking in genes. To suppress non-homologous end-joining, a HR enhancer can be delivered. In some cases, more than one HR enhancer can be delivered. A HR enhancer can inhibit proteins involved in non-homologous end-joining, for example, KU70, KU80, and/or DNA Ligase IV. In some cases, a Ligase IV inhibitor, such as Scr7, can be delivered. In some cases, the HR enhancer can be L755507. In some cases, a different Ligase IV inhibitor can be used. In some cases, a HR enhancer can be an adenovirus 4 protein, for example, E1B55K and/or E4 orf6. In some cases, a chemical inhibitor can be used.

Non-homologous end-joining molecules such as KU70, KU80, and/or DNA Ligase IV can be suppressed by using a variety of methods. For example, non-homologous end-joining molecules such as KU70, KU80, and/or DNA Ligase IV can be suppressed by gene silencing. For example, non-homologous end-joining molecules KU70, KU80, and/or DNA Ligase IV can be suppressed by gene silencing during transcription or translation of factors. Non-homologous end-joining molecules KU70, KU80, and/or DNA Ligase IV can also be suppressed by degradation of factors. Non-homologous end-joining molecules KU70, KU80, and/or DNA Ligase IV can be also be inhibited. Inhibitors of KU70, KU80, and/or DNA Ligase IV can comprise E1B55K and/or E4 orf6. Non-homologous end-joining molecules KU70, KU80, and/or DNA Ligase IV can also be inhibited by sequestration. Gene expression can be suppressed by knock out, altering a promoter of a gene, and/or by administering interfering RNAs directed at the factors.

In some cases, the insertion can comprise homology directed repair. In some cases, an enhancer of HR can be used, such as RS-1. RS-1 can be added to the media of a cellular culture. RS-1 can increase the efficiency of nuclease-mediated integration of an exogenous polynucleic acid into a genome. For example, RS-1 can improve the efficiency of integration of a TCR sequence into the genome of a cell by homologous recombination. RS-1 can also increase the viability of cells post cellular engineering. RS-1 protein or portion thereof can be introduced to a population of cells at a concentration from about 3 μM to about 12 μM. RS-1 protein or portion thereof can be introduced to a population of cells at a concentration from about 7 μM to about 8 μM. In some cases, RS-1 protein or portion thereof can be introduced to a population of cells at a concentration from about 3 μM, 4 μM, 5 μM, 6 μM, 7 μM, 8 μM, 9 μM, 10 μM, 11 μM, or up to about 12 μM. In some cases, a downstream factor in the RS-1 pathway may be utilized. RS-1 (3-((benzylamino) sulfonyl)-4-bromo-N-(4-bromophenyl)benzamide) can stimulate RAD51, a player in the HR complex. In some cases, modulating a RAD51-interacting factor such as PALB2 (partner and localizer of BRCA2), Nap1 (nucleosome assembly protein 1), p400 ATPase, EVL (Ena/Vasp-like) and the like may also lead to enhanced integration frequencies in nuclease-mediated gene targeting. For example, RAD51 may be introduced to a cellular culture to improve integration of an exogenous sequence into a cellular genome.

Rad51 may assist HR through a variety of methods. For example, HR can be dependent on the availability of a template, synthesized during the S-phase of the cell cycle. The breast cancer susceptibility gene (BRCA2) and Rad51, a structural and functional homolog of bacterial RecA recombinase, can be used for the error-free repair of DSB by HR. Following detection of DSB, BRCA2 recruits Rad51 to the junction of DSBs. Rad 51 protein or portion thereof can be introduced to a population of cells at a concentration from about 100 ng to about 20 μg in some cases. For example, Rad 51 can be introduced to a population of cells from about 100 ng, 200 ng, 300 ng, 400 ng, 500 ng, 600 ng, 700 ng, 800 ng, 900 ng, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, or up to about 20 μg.

In some cases, an enhancer of homologous recombination can be n-acetyl-cysteine (NAC). NAC can be a thiol-containing compound that nonenzymatically interacts with and detoxifies reactive electrophiles and free radicals. NAC can be introduced to a cellular culture in some cases. For example, NAC may be introduced prior to electroporation, during electroporation, or after an electroporation. In other cases, NAC may be cultured with cells during an expansion step. In some cases, a vector encoding NAC may be introduced to a cell. NAC can be supplied in the culture medium at a concentration about 1 μM, 5 μM, 10 μM, 20 μM, 50 μM, 75 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 15 mM, 16 mM, 18 mM, 20 mM, 22 mM, 24 mM, 25 mM, 26 mM, 28 mM, 30 mM, 35 mM, 40 mM, 45 mM, 50 mM, 75 mM, 100 mM, 200 mM, 500 mM, 750 mM, 1 M, 10 M, 100 M, or about a value between any two of these values. NAC can be supplied in the culture medium at a concentration about 10 mM.

An enhancer can be a protein involved in double strand break repair. Proteins involved in double strand break repair can be MRE11, RAD50, NBS1 (XRS2) complex, BRCA1, histone H2AX, PARP-1, RAD18, DNA-dependent protein kinase catalytic subunit (DNA-PKcs), and ATM. In some cases, an enhancer can be AKT or be involved in an AKT pathway. AKT can be involved in NHEJ-mediated double strand break repair. In some cases, AKT1 can inhibit HR by inducing the cytoplasmic translocation of Brca1 and Rad51. AKT, also known as protein kinase B (PKB), belongs to the cAMP-dependent, cGMP-dependent, protein kinase C kinase family. The AKT family can have 3 evolutionarily conserved isoforms: AKT1 (PKBα) (including 3 splice variants), AKT2 (PKBβ), and AKT3 (PKBγ) (including 2 splice variants). Growth factors and cytokines, such as IL-2, can bind to a transmembrane receptor and stimulate the activity of lipid enzyme phosphatidyl-inositol 3-kinase (PI3K) family members, which can phosphorylate phosphatidyl-inositol di-phosphate (PIP2) to generate PIP3 at the plasma membrane. PIP3 can constitute binding sites for proteins that contain a pleckstrin homology (PH) domain, such as AKT and PDK1, recruiting them to the membrane. In some cases, a PI3K family members may be introduced to a cell to enhance integration of an exogenous sequence.

In some cases, AKT can be inhibited. Inhibition of AKT by selective chemical inhibitors or AKT siRNA can restore the DNA damage-induced recruitment of RPA, CtIP, Rad51, and Chk1 activation. In some cases, blockage of growth factors, cytokines, or both, can inhibit AKT pathway. In some cases, blockage of growth factors, cytokines, or both, e.g., anti-IFNAR2 antibody (antibody against human Interferon (alpha, beta, and omega) Receptor 2), can promote HR mediated DNA double strand break repair. IFNAR2 antibody can be supplied in the culture medium at a concentration about 100 μg/ml, 500 μg/ml, 1 ng/ml, 5 ng/ml, 10 ng/ml, 20 ng/ml, 50 ng/ml, 75 ng/ml, 100 ng/ml, 200 ng/ml, 500 ng/ml, 750 ng/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 12 μg/ml, 14 μg/ml, 15 μg/ml, 16 μg/ml, 18 μg/ml, 20 μg/ml, 22 μg/ml, 25 μg/ml, 30 μg/ml, 40 μg/ml, 50 μg/ml, 60 μg/ml, 70 μg/ml, 80 μg/ml, 90 μg/ml, 1 mg/ml, 5 mg/ml, 10 mg/ml, 20 mg/ml, 50 mg/ml, 100 mg/ml, 200 mg/ml, 500 mg/ml, 1 g/ml, or about a value between any two of these values. IFNAR2 antibody can be supplied in the culture medium at a concentration about 10 μg/ml.

A HR enhancer that suppresses non-homologous end-joining can be delivered with plasmid DNA. Sometimes, the plasmid can be a double stranded DNA molecule. The plasmid molecule can also be single stranded DNA. The plasmid can also carry at least one gene. The plasmid can also carry more than one gene. At least one plasmid can also be used. More than one plasmid can also be used. A HR enhancer that suppresses non-homologous end-joining can be delivered with plasmid DNA in conjunction with CRISPR-Cas, primers, and/or a modifier compound. A modifier compound can reduce cellular toxicity of plasmid DNA and improve cellular viability. An HR enhancer and a modifier compound can be introduced to a cell before genomic engineering. The HR enhancer can be a small molecule. In some cases, the HR enhancer can be delivered to a T cell suspension. An HR enhancer can improve viability of cells transfected with double stranded DNA.

A HR enhancer that suppresses non-homologous end-joining can be delivered with an HR substrate to be integrated. A substrate can be a polynucleic acid. A polynucleic acid can comprise a TCR transgene. A polynucleic acid can be delivered as mRNA. A polynucleic acid can comprise homology arms to an endogenous region of the genome for integration of a TCR transgene. A polynucleic acid can be a vector. A vector can be inserted into another vector (e.g., viral vector) in either the sense or anti-sense orientation. Upstream of the 5′ LTR region of the viral genome a T7, T3, or other transcriptional start sequence can be placed for in vitro transcription of the viral cassette. This vector cassette can be then used as a template for in vitro transcription of mRNA. For example, when this mRNA is delivered to any cell with its cognate reverse transcription enzyme, delivered also as mRNA or protein, then the single stranded mRNA cassette can be used as a template to generate hundreds to thousands of copies in the form of double stranded DNA (dsDNA) that can be used as a HR substrate for the desired homologous recombination event to integrate a transgene cassette at an intended target site in the genome. This method can circumvent the need for delivery of toxic plasmid DNA for CRISPR mediated homologous recombination. Additionally, as each mRNA template can be made into hundreds or thousands of copies of dsDNA, the amount of homologous recombination template available within the cell can be very high. The high amount of homologous recombination template can drive the desired homologous recombination event. Further, the mRNA can also generate single stranded DNA. Single stranded DNA can also be used as a template for homologous recombination, for example with recombinant AAV (rAAV) gene targeting. mRNA can be reverse transcribed into a DNA homologous recombination HR enhancer in situ. This strategy can avoid the toxic delivery of plasmid DNA. Additionally, mRNA can amplify the homologous recombination substrate to a higher level than plasmid DNA and/or can improve the efficiency of homologous recombination. In the event that only robust reverse transcription of the single stranded DNA occurs in a cell, mRNAs encoding both the sense and anti-sense strand of the viral vector can be introduced. In this case, both mRNA strands can be reverse transcribed within the cell and/or naturally anneal to generate dsDNA.

A HR enhancer that suppresses non-homologous end-joining can be delivered as a chemical inhibitor. For example, a HR enhancer can act by interfering with Ligase IV-DNA binding. A HR enhancer can also activate the intrinsic apoptotic pathway. A HR enhancer can also be a peptide mimetic of a Ligase IV inhibitor. A HR enhancer can also be co-expressed with the Cas9 system. A HR enhancer can also be co-expressed with viral proteins, such as E1B55K and/or E4 orf6. A HR enhancer can also be SCR7, L755507, or any derivative thereof. A HR enhancer can be delivered with a compound that reduces toxicity of exogenous DNA insertion.

In some cases, a homologous recombination HR enhancer can be used to suppress non-homologous end-joining. In some cases, a homologous recombination HR enhancer can be used to promote homologous directed repair. In some cases, a homologous recombination HR enhancer can be used to promote homologous directed repair after a CRISPR-Cas double stranded break. In some cases, a homologous recombination HR enhancer can be used to promote homologous directed repair after a CRISPR-Cas double stranded break and the knock-in and knock-out of one of more genes.

Increase in HR efficiency with an HR enhancer can be or can be about 10%, 20%, 30%, 40%, 50%, 60%, 70%, 80%, 90%, or 100%. Decrease in NHEJ with an HR enhancer can be or can be about 10%, 20%, 30%, 40%, 50%, 60%, 70%, 80%, 90%, or 100%.

Contacting with modulator of DNA double strand break repair can lead to an increase in cell viability about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 125%, about 150%, about 175%, about 200%, about 250%, about 300%, or even more. In some cases, an increase in cell viability can be from about 50% to about 200%. Contacting with modulator of DNA double strand break repair can lead to an increase in integration efficiency about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 125%, about 150%, about 175%, about 200%, about 250%, about 300%, or even more. In some cases, an increase in integration efficiency can be from about 50% to about 200%.

In some cases, the modulator of DNA double strand break repair can be applied after the cell transfection. In some cases, the modulator of DNA double strand break repair can be introduced immediately after the cell transfection is completed. In some cases, the modulator of DNA double strand break repair can be introduced while the cell transfection is being conducted, for instance, applied while electroporation is being performed, or applied while the cell is still being exposed to transfection reagents. In some cases, the modulator of DNA double strand break repair can be introduced up to several minutes to several hours post-transfection. The time delay between the completion of cell transfection and application of the modulator of DNA double strand break repair can be about 30 sec, about 1 min, about 2 min, about 3 min, about 4 min, about 5 min, about 6 min, about 7 min, about 8 min, about 9 min, about 10 min, about 15 min, about 30 min, about 45 min, about 60 min, about 1.5 hrs, about 2 hrs, about 3 hrs, about 4 hrs, about 5 hrs, about 7.5 hrs, about 8 hrs, about 10 hrs, about 12 hrs, about 20 hrs, about 30 hrs, about 40 hrs, about 50 hrs, about 60 hrs, about 70 hrs, about 80 hrs, about 90 hrs, or about 1 week. In some cases, the time delay can be even longer.

In some cases, the modulator of DNA double strand break repair can be applied before the cell transfection. In certain cases, the modulator of DNA double strand break repair can be present in the cell culture through a period of time before the transfection. The modulator of DNA double strand break repair can be present both before and after the cell transfection.

The modulator of DNA double strand break repair can be supplied in the culture medium, and the culture medium containing the modulator of DNA double strand break repair can be replaced once about every 6 hr, 12 hr, 20 hr, 21 hr, 22 hr, 23 hr, 24 hr, 26 hr, 28 hr, 30 hr, 32 hr, 34 hr, 36 hr, 40 hr, 44 hr, 48 hr, 50 hr, 60 hr. The frequent replacement of the culture medium can maintain the concentration of the modulator of DNA double strand break repair at a certain level.

As those skilled in the art would appreciate, the choice of the modulator of DNA double strand break repair, the concentration of the modulator of DNA double strand break repair, and the timing and the duration for the incubation of the modulator of DNA double strand break repair can vary depending on many parameters as discussed above.

In some cases, the cells can be treated/incubated with the modulator of DNA double strand break repair for a period of time. The incubation time can be at least about 1 min, at least about 2 min, at least about 3 min, at least about 4 min, at least about 5 min, at least about 10 min, at least about 20 min, at least about 30 min, at least about 45 min, or at least about 60 min. The incubation time can be at least about 1 hr, at least about 2 hrs, at least about 3 hrs, at least about 4 hrs, at least about 5 hrs, at least about 7.5 hrs, at least about 8 hrs, at least about 10 hrs, at least about 12 hrs, at least about 20 hrs, at least about 30 hrs, at least about 40 hrs, at least about 50 hrs, at least about 60 hrs, at least about 70 hrs, at least about 80 hrs, at least about 90 hrs, or at least about 1 week. In some cases, the incubation time can be at least 1 week, at least 2 weeks, at least 3 weeks, or even longer.

### Minicircle and Linearized Double Stranded DNA Construct

Provided herein are methods of improving overall yield from cell engineering, including, for instance, improving cell viability after cell engineering, and/or improving transfection efficiency, comprising contacting a population of cells a minicircle vector that encodes a transgene thereby generating a population of modified cells.

Also provided herein is a method of improving overall yield from cell engineering, including, for instance, improving cell viability after cell engineering, and/or improving transfection efficiency, comprising contacting a population of cells a linearized double stranded DNA construct that encodes a transgene thereby generating a population of modified cells.

One aspect of the present disclosure provides a method of genomically editing, comprising introducing to a population of cells a minicircle vector that encodes a transgene thereby generating a population of modified cells. One aspect of the present disclosure provides a method of genomically editing, comprising introducing to a population of cells a linearized double stranded DNA construct that encodes a transgene thereby generating a population of modified cells.

In some cases, the exogenous polynucleic acid, e.g., a transgene, can be introduced to the cell in a minicircle vector. The term “minicircle” as used herein can refer to small circular plasmid derivative that is free of most, if not all, prokaryotic vector parts (e.g. control sequences and other non-functional sequences of prokaryotic origin). With wishing to be bound by a certain theory, minimizing the size of exogenous nucleic acid can reduce cell toxicity and potentially promote the integration efficiency. In some cases, a method provided herein comprising introducing to a cell a minicircle vector that encodes a transgene can increase cell viability. In some cases, a method provided herein comprising introducing to a cell a minicircle vector that encodes a transgene can increase integration efficiency.

A minicircle vector can have a size of about 1.5 kb, about 2 kb, about 2.2 kb, about 2.4 kb, about 2.6 kb, about 2.8 kb, about 3 kb, about 3.2 kb, about 3.4 kb, about 3.6 kb, about 3.8 kb, about 4 kb, about 4.2 kb, about 4.4 kb, about 4.6 kb, about 4.8 kb, about 5 kb, about 5.2 kb, about 5.4 kb, about 5.6 kb, about 5.8 kb, about 6 kb, about 6.5 kb, about 7 kb, about 8 kb, about 9 kb, about 10 kb, about 12 kb, about 25 kb, about 50 kb, or a value between any two of these numbers. Sometimes, a mini-circle as provided herein can have a size at most 2.1 kb, at most 3.1 kb, at most 4.1 kb, at most 4.5 kb, at most 5.1 kb, at most 5.5 kb, at most 6.5 kb, at most 7.5 kb, at most 8.5 kb, at most 9.5 kb, at most 11 kb, at most 13 kb, at most 15 kb, at most 30 kb, or at most 60 kb.

A minicircle vector concentration can be from about 0.5 nanograms (ng) to about 50 μg. A minicircle vector concentration can be from about 0.5 ng to about 50 μg, from about 1 ng to about 25 μg, from about 5 ng to about 10 μg, from about 10 ng to about 5 μg, from about 20 ng to about 1 μg, from about 50 ng to 500 ng, or from about 100 ng to 250 ng.

In some cases, the exogenous polynucleic acid, e.g., a transgene, can be introduced to the cell in a linearized double stranded DNA (dsDNA) construct. In some cases, a method provided herein comprising introducing to a cell a linearized dsDNA construct that encodes a transgene can increase cell viability. In some cases, a method provided herein comprising introducing to a cell a linearized dsDNA construct that encodes a transgene can increase integration efficiency.

A linearized dsDNA construct can have a size of at least 500 bp, at least 750 bp, at least 1 kb, at least 1.1 kb, at least 1.2 kb, at least 1.3 kb, at least 1.4 kb, at least 1.5 kb, at least 1.6 kb, at least 1.7 kb, at least 1.8 kb, at least 1.9 kb, at least 2 kb, or even larger size. A linearized dsDNA construct can have a size of about 500 bp, about 750 bp, about 1 kb, about 1.1 kb, about 1.2 kb, about 1.3 kb, about 1.4 kb, about 1.5 kb, about 1.6 kb, about 1.7 kb, about 1.8 kb, about 1.9 kb, or about 2 kb.

A linearized dsDNA construct concentration can be from about 0.5 nanograms (ng) to about 50 μg. A linearized dsDNA construct concentration can be from about 0.5 ng to about 50 μg, from about 1 ng to about 25 μg, from about 5 ng to about 10 μg, from about 10 ng to about 5 μg, from about 20 ng to about 1 μg, from about 50 ng to 500 ng, or from about 100 ng to 250 ng.

A minicircle vector or a double-stranded linearized construct can contain a transgene as discussed above. A minicircle or a double-stranded linearized construct can comprise any nucleotide sequence, e.g., any gene of interest. A minicircle or a double-stranded linearized construct can comprise a transgene that encodes a cellular receptor. A cellular receptor can include, but not limited to, a TCR, BCR, CAR, and any combination thereof. A minicircle or a double-stranded linearized construct can comprise a transgene that encodes a TCR as discussed above.

### Nuclease Systems

Gene editing can be performed using a nuclease, including CRISPR associated proteins (Cas proteins, e.g., Cas9), Zinc finger nuclease (ZFN), Transcription Activator-Like Effector Nuclease (TALEN), or meganucleases. Nucleases (e.g., endonucleases) can be naturally existing nucleases, genetically modified, and/or recombinant. Gene editing can also be performed using a transposon-based system (e.g. PiggyBac, Sleeping beauty). For example, gene editing can be performed using a transposase.

### CRISPR System

In some embodiments, methods described herein use a CRISPR system. There are at least five types of CRISPR systems which all incorporate RNAs and Cas proteins. Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute proteins; any derivative thereof; any variant thereof; and any fragment thereof.

The general mechanism and recent advances of CRISPR system is discussed in Cong, L. et al., “Multiplex genome engineering using CRISPR systems,” Science, 339(6121): 819-823 (2013); Fu, Y. et al., “High-frequency off-target mutagenesis induced by CRISPR-Cas nucleases in human cells,” Nature Biotechnology, 31, 822-826 (2013); Chu, V T et al. “Increasing the efficiency of homology-directed repair for CRISPR-Cas9-induced precise gene editing in mammalian cells,” Nature Biotechnology 33, 543-548 (2015); Shmakov, S. et al., “Discovery and functional characterization of diverse Class 2 CRISPR-Cas systems,” Molecular Cell, 60, 1-13 (2015); Makarova, K S et al., “An updated evolutionary classification of CRISPR-Cas systems,”, Nature Reviews Microbiology, 13, 1-15 (2015). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). For example, an engineered cell can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

### Cas Protein

A vector can be operably linked to an enzyme-coding sequence encoding a CRISPR enzyme, such as a Cas protein (CRISPR-associated protein). Non-limiting examples of Cas proteins include, but are not limited to, Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof. In some embodiments, the Cas enzyme is unmodified. In some embodiments, the CRISPR enzyme directs cleavage of one or both strands at a target sequence, such as within a target sequence and/or within a complement of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. In some embodiments, the CRISPR enzyme is mutated with respect to a corresponding wild-type enzyme such that the mutated CRISPR enzyme lacks the ability to cleave one or both strands of a target polynucleotide containing a target sequence can be used. In some embodiments, the Cas protein is a high fidelity cas protein such as Cas9HiFi.

Cas9 refers to the wild type or a modified form of the Cas9 protein that can comprise an amino acid change such as a deletion, insertion, substitution, variant, mutation, fusion, chimera, or any combination thereof. In some embodiments, a polynucleotide encoding an endonuclease (e.g., a Cas protein such as Cas9) is codon optimized for expression in particular cells, such as eukaryotic cells. This type of optimization can entail the mutation of foreign-derived (e.g., recombinant) DNA to mimic the codon preferences of the intended host organism or cell while encoding the same protein. In some embodiments, an endonuclease comprises an amino acid sequence having at least or at least about 50%, 60%, 70%, 75%, 80%, 85%, 90%, 95%, 99%, or 100%, amino acid sequence identity to the nuclease domain of a wild type exemplary site-directed polypeptide (e.g., Cas9 from S. pyogenes).

Any functional concentration of Cas protein can be introduced to a cell. For example, 15 micrograms of Cas mRNA can be introduced to a cell. In other cases, a Cas mRNA can be introduced from 0.5 micrograms to 100 micrograms. A Cas mRNA can be introduced from 0.5, 5, 10, 15, 20, 25, 30, 35, 40, 45, 50, 55, 60, 65, 70, 75, 80, 85, 90, 95, or 100 micrograms.

In some embodiments, a vector that encodes a CRISPR enzyme comprises one or more nuclear localization sequences (NLSs), such as more than or more than about 1, 2, 3, 4, 5, 6, 7, 8, 9, 10, NLSs can be used. For example, a CRISPR enzyme can comprise more than or more than about 1, 2, 3, 4, 5, 6, 7, 8, 9, 10, NLSs at or near the ammo-terminus, more than or more than about 1, 2, 3, 4, 5, 6, 7, 8, 9, 10, NLSs at or near the carboxyl-terminus, or any combination of these (e.g., one or more NLS at the ammo-terminus and one or more NLS at the carboxyl terminus). When more than one NLS is present, each can be selected independently of others, such that a single NLS can be present in more than one copy and/or in combination with one or more other NLSs present in one or more copies. In some embodiments, CRISPR enzymes used in the methods comprise NLSs. The NLS can be located anywhere within the polypeptide chain, e.g., near the N- or C-terminus. For example, the NLS can be within or within about 1, 2, 3, 4, 5, 10, 15, 20, 25, 30, 40, 50 amino acids along a polypeptide chain from the N- or C-terminus. Sometimes the NLS can be within or within about 50 amino acids or more, e.g., 100, 200, 300, 400, 500, 600, 700, 800, 900, or 1000 amino acids from the N- or C-terminus.

Non-limiting examples of NLSs include an NLS sequence derived from: the NLS of the SV40 virus large T-antigen, having the amino acid sequence PKKKRKV (SEQ ID NO: 2); the NLS from nucleoplasmin (e.g. the nucleoplasmin bipartite NLS with the sequence KRPAATKKAGQAKKKK (SEQ ID NO: 63)); the c-myc NLS having the amino acid sequence PAAKRVKLD (SEQ ID NO: 64) or RQRRNELKRSP (SEQ ID NO: 65); the hRNPA1 M9 NLS having the sequence NQSSNFGPMKGGNFGGRSSGPYGGGGQYFAKPRNQGGY (SEQ ID NO: 66); the sequence RMRIZFKNKGKDTAELRRRRVEVSVELRKAKKDEQILKRRNV (SEQ ID NO: 67) of the IBB domain from importin-alpha; the sequences VSRKRPRP (SEQ ID NO: 68) and PPKKARED (SEQ ID NO: 69) of the myoma T protein; the sequence PQPKKKPL (SEQ ID NO: 70) of human p53; the sequence SALIKKKKKMAP (SEQ ID NO: 71) of mouse c-abl IV; the sequences DRLRR (SEQ ID NO: 72) and PKQKKRK (SEQ ID NO: 73) of the influenza virus NS1; the sequence RKLKKKIKKL (SEQ ID NO: 74) of the Hepatitis virus delta antigen; the sequence REKKKFLKRR (SEQ ID NO: 75) of the mouse M×1 protein; the sequence KRKGDEVDGVDEVAKKKSKK (SEQ ID NO: 76) of the human poly(ADP-ribose) polymerase; and the sequence RKCLQAGMNLEARKTKK (SEQ ID NO: 77) of the steroid hormone receptors (human) glucocorticoid.

### Guide RNA

As used herein, the term “guide RNA (gRNA)”, and its grammatical equivalents refers to a RNA which can be specific for a target DNA and can form a complex with a Cas protein. A guide RNA can comprise a guide sequence, or spacer sequence, that specifies a target site and guides a RNA/Cas complex to a specified target DNA for cleavage. Site-specific cleavage of a target DNA occurs at locations determined by both 1) base-pairing complementarity between a guide RNA and a target DNA (also called a protospacer) and 2) a short motif in a target DNA referred to as a protospacer adjacent motif (PAM).

The methods disclosed herein can comprise introducing into a cell or embryo at least one guide RNA or nucleic acid, e.g., DNA encoding at least one guide RNA. A guide RNA can interact with a RNA-guided endonuclease to direct the endonuclease to a specific target site, at which site the 5′ end of the guide RNA base pairs with a specific protospacer sequence in a chromosomal sequence.

In some embodiments, a guide RNA comprises two RNAs, e.g., CRISPR RNA (crRNA) and transactivating crRNA (tracrRNA). In some embodiments, a guide RNA comprises a single-guide RNA (sgRNA) formed by fusion of a portion (e.g., a functional portion) of crRNA and tracrRNA. A guide RNA can also be a dual RNA comprising a crRNA and a tracrRNA. A guide RNA can comprise a crRNA and lack a tracrRNA. Furthermore, a crRNA can hybridize with a target DNA or protospacer sequence.

As discussed above, a guide RNA can be an expression product. For example, a DNA that encodes a guide RNA can be a vector comprising a sequence coding for the guide RNA. A guide RNA can be transferred into a cell or organism by transfecting the cell or organism with an isolated guide RNA or plasmid DNA comprising a sequence coding for the guide RNA and a promoter. A guide RNA can also be transferred into a cell or organism in other way, such as using virus-mediated gene delivery.

A guide RNA can be isolated. For example, a guide RNA can be transfected in the form of an isolated RNA into a cell or organism. A guide RNA can be prepared by in vitro transcription using any in vitro transcription system. A guide RNA can be transferred to a cell in the form of isolated RNA rather than in the form of plasmid comprising encoding sequence for a guide RNA.

In some embodiments, the guide RNA comprises a DNA-targeting segment and a protein binding segment. A DNA-targeting segment (or DNA-targeting sequence, or spacer sequence) comprises a nucleotide sequence that can be complementary to a specific sequence within a target DNA (e.g., a protospacer). A protein-binding segment (or protein-binding sequence) can interact with a site-directed modifying polypeptide, e.g. an RNA-guided endonuclease such as a Cas protein. By “segment” it is meant a segment/section/region of a molecule, e.g., a contiguous stretch of nucleotides in an RNA. A segment can also mean a region/section of a complex such that a segment may comprise regions of more than one molecule. For example, in some cases a protein-binding segment of a DNA-targeting RNA is one RNA molecule and the protein-binding segment therefore comprises a region of that RNA molecule. In other cases, the protein-binding segment of a DNA-targeting RNA comprises two separate molecules that are hybridized along a region of complementarity.

In some embodiments, the guide RNA comprises two separate RNA molecules or a single RNA molecule. An exemplary single molecule guide RNA comprises both a DNA-targeting segment and a protein-binding segment.

An exemplary two-molecule DNA-targeting RNA can comprise a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A first RNA molecule can be a crRNA-like molecule (targeter-RNA), that can comprise a DNA-targeting segment (e.g., spacer) and a stretch of nucleotides that can form one half of a double-stranded RNA (dsRNA) duplex comprising the protein-binding segment of a guide RNA. A second RNA molecule can be a corresponding tracrRNA-like molecule (activator-RNA) that can comprise a stretch of nucleotides that can form the other half of a dsRNA duplex of a protein-binding segment of a guide RNA. In other words, a stretch of nucleotides of a crRNA-like molecule can be complementary to and can hybridize with a stretch of nucleotides of a tracrRNA-like molecule to form a dsRNA duplex of a protein-binding domain of a guide RNA. As such, each crRNA-like molecule can be said to have a corresponding tracrRNA-like molecule. A crRNA-like molecule additionally can provide a single stranded DNA-targeting segment, or spacer sequence. Thus, a crRNA-like and a tracrRNA-like molecule (as a corresponding pair) can hybridize to form a guide RNA. A subject two-molecule guide RNA can comprise any corresponding crRNA and tracrRNA pair.

In some embodiments, the DNA-targeting segment or spacer sequence of a guide RNA is complementary to sequence at a target site in a chromosomal sequence, e.g., protospacer sequence) such that the DNA-targeting segment of the guide RNA can base pair with the target site or protospacer. In some cases, a DNA-targeting segment of a guide RNA comprises from or from about 10 nucleotides to from or from about 25 nucleotides or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more than 25 nucleotides in length. In some embodiments, a first region of a guide RNA is about 19, 20, or 21 nucleotides in length.

In some embodiments, a guide RNA targets a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30 or more nucleotides. A target nucleic acid can be at most or at most about 5, 10, 15, 16, 17, 18, 19, 20, 21, 22, 23, 24, 25, 30 or more nucleotides. A target nucleic acid sequence can be or can be about 20 bases immediately 5′ of the first nucleotide of the PAM. A guide RNA can target the nucleic acid sequence.

A guide nucleic acid, for example, a guide RNA, can refer to a nucleic acid that can hybridize to another nucleic acid, for example, the target nucleic acid or protospacer in a genome of a cell. A guide nucleic acid can be RNA. A guide nucleic acid can be DNA. The guide nucleic acid can be programmed or designed to bind to a sequence of nucleic acid site-specifically. A guide nucleic acid can comprise a polynucleotide chain and can be called a single guide nucleic acid. A guide nucleic acid can comprise two polynucleotide chains and can be called a double guide nucleic acid.

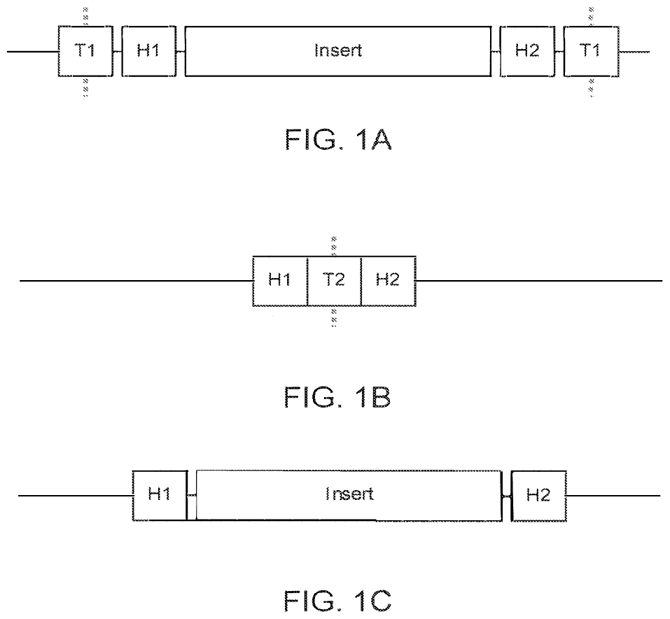

A guide nucleic acid can hybridize to a genomic site, such as an endogenous gene provided in Table 1. In other cases, a guide nucleic acid can hybridize to a construct that comprises an insert transgene, for example as exemplified in FIG. 1A-FIG. 1C. In some aspects, a guide nucleic acid hybridizes to a sequence that is non-human. For example, in cases where a guide nucleic acid hybridizes to a construct that comprises an insert it may be specific to a non-human sequence such as a xenogeneic sequence or a synthetic sequence. In some cases, a non-human sequence is xenogeneic. Xenogeneic sequences can be obtained from any non-human sources, including but not limited to, fish, cow, cat, goat, monkey, pig, dog, horse, sheep, bird, ferret, hamster, rabbit, snake, or combinations thereof. In some cases, a xenogeneic sequence is from a fish and the fish is a zebrafish.

In other cases, to simplify targeting construct design and/or allow for consistent, reproducible liberation of a donor transgene cargo in vivo by a CRISPR nuclease, for example Cas9, a universal guide RNA sequence, UgRNA can be utilized and described in Wierson et al., 2019. In some cases, a universal guide can comprise optimal base composition using CRISPRScan for example as provided in Moreno-Mateos et al., 2015. An exemplary universal UgRNA may not comprise predicted targets in a xenogeneic genome such as the zebra-fish, pig, or human genome. When utilized a universal guide can show efficient double strand break induction and homology mediated repair at a target site, for example of a guide polynucleic acid and/or in a fluorescent reporter integrated into the zebrafish noto gene (Wierson et al., 2019a).

A guide nucleic acid can comprise one or more modifications to provide a nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides.

A guide nucleic acid can comprise a nucleotide sequence (e.g., a spacer), for example, at or near the 5′ end or 3′ end, that can hybridize to a sequence in a target nucleic acid (e.g., a protospacer). A spacer of a guide nucleic acid can interact with a target nucleic acid in a sequence-specific manner via hybridization (i.e., base pairing). A spacer sequence can hybridize to a target nucleic acid that is located 5′ or 3′ of a protospacer adjacent motif (PAM). The length of a spacer sequence can be at least or at least about 5, 10, 15, 16, 17, 18, 19, 20, 21, 22, 23, 24, 25, 30 or more nucleotides. The length of a spacer sequence can be at most or at most about 5, 10, 15, 16, 17, 18, 19, 20, 21, 22, 23, 24, 25, 30 or more nucleotides.

A guide RNA can also comprise a dsRNA duplex region that forms a secondary structure. For example, a secondary structure formed by a guide RNA can comprise a stem (or hairpin) and a loop. A length of a loop and a stem can vary. For example, a loop can range from about 3 to about 10 nucleotides in length, and a stem can range from about 6 to about 20 base pairs in length. A stem can comprise one or more bulges of 1 to about 10 nucleotides. The overall length of a second region can range from about 16 to about 60 nucleotides in length. For example, a loop can be or can be about 4 nucleotides in length and a stem can be or can be about 12 base pairs. A dsRNA duplex region can comprise a protein-binding segment that can form a complex with an RNA-binding protein, such as an RNA-guided endonuclease, e.g. Cas protein.

A guide RNA can also comprise a tail region at the 5′ or 3′ end that can be essentially single-stranded. For example, a tail region is sometimes not complementarity to any chromosomal sequence in a cell of interest and is sometimes not complementarity to the rest of a guide RNA. Further, the length of a tail region can vary. A tail region can be more than or more than about 4 nucleotides in length. For example, the length of a tail region can range from or from about 5 to from or from about 60 nucleotides in length.

A guide RNA can be introduced into a cell or embryo as an RNA molecule. For example, an RNA molecule can be transcribed in vitro and/or can be chemically synthesized. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. An RNA coding sequence can be operably linked to a promoter sequence that is recognized by RNA polymerase III (Pol III).

A DNA molecule encoding a guide RNA can also be linear. A DNA molecule encoding a guide RNA can also be circular. A DNA sequence encoding a guide RNA can also be part of a vector. Some examples of vectors can include plasmid vectors, phagemids, cosmids, artificial/mini-chromosomes, transposons, and viral vectors. For example, a DNA encoding an RNA-guided endonuclease is present in a plasmid vector. Other non-limiting examples of suitable plasmid vectors include pUC, pBR322, pET, pBluescript, and variants thereof. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

When both a RNA-guided endonuclease and a guide RNA are introduced into a cell as DNA molecules, each can be part of a separate molecule (e.g., one vector containing fusion protein coding sequence and a second vector containing guide RNA coding sequence) or both can be part of a same molecule (e.g., one vector containing coding (and regulatory) sequence for both a fusion protein and a guide RNA).

A Cas protein, such as a Cas9 protein or any derivative thereof, can be pre-complexed with a guide RNA to form a ribonucleoprotein (RNP) complex. The RNP complex can be introduced into primary immune cells. Introduction of the RNP complex can be timed. The cell can be synchronized with other cells at GI, S, and/or M phases of the cell cycle. The RNP complex can be delivered at a cell phase such that HDR is enhanced. The RNP complex can facilitate homology directed repair.

A guide RNA can also be modified. The modifications can comprise chemical alterations, synthetic modifications, nucleotide additions, and/or nucleotide subtractions. The modifications can also enhance CRISPR genome engineering. A modification can alter chirality of a gRNA. In some cases, chirality may be uniform or stereopure after a modification. A guide RNA can be synthesized. The synthesized guide RNA can enhance CRISPR genome engineering. A guide RNA can also be truncated. Truncation can be used to reduce undesired off-target mutagenesis. The truncation can comprise any number of nucleotide deletions. For example, the truncation can comprise 1, 2, 3, 4, 5, 10, 15, 20, 25, 30, 40, 50 or more nucleotides. A guide RNA can comprise a region of target complementarity of any length. For example, a region of target complementarity can be less than 20 nucleotides in length. A region of target complementarity can be more than 20 nucleotides in length.

In some cases, a modification is on a 5′ end, a 3′ end, from a 5′ end to a 3′ end, a single base modification, a 2′-ribose modification, or any combination thereof. A modification can be selected from a group consisting of base substitutions, insertions, deletions, chemical modifications, physical modifications, stabilization, purification, and any combination thereof.

In some cases, a modification is a chemical modification. A modification can be selected from 5′ adenylate, 5′ guanosine-triphosphate cap, 5′N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3 phosphorothioate or any combinations thereof.

In some cases, a modification is a 2-O-methyl 3 phosphorothioate addition. A 2-O-methyl 3 phosphorothioate addition can be performed from 1 base to 150 bases. A 2-O-methyl 3 phosphorothioate addition can be performed from 1 base to 4 bases. A 2-O-methyl 3 phosphorothioate addition can be performed on 2 bases. A 2-O-methyl 3 phosphorothioate addition can be performed on 4 bases. A modification can also be a truncation. A truncation can be a 5-base truncation.

In some cases, a dual nickase approach may be used to introduce a double stranded break. Cas proteins can be mutated at known amino acids within either nuclease domains, thereby deleting activity of one nuclease domain and generating a nickase Cas protein capable of generating a single strand break. A nickase along with two distinct guide RNAs targeting opposite strands may be utilized to generate a DSB within a target site (often referred to as a “double nick” or “dual nickase” CRISPR system). This approach may dramatically increase target specificity, since it is unlikely that two off-target nicks will be generated within close enough proximity to cause a DSB.

A gRNA can be introduced at any functional concentration. For example, a gRNA can be introduced to a cell at 10 micrograms. In other cases, a gRNA can be introduced from 0.5 micrograms to 100 micrograms. A gRNA can be introduced from 0.5, 5, 10, 15, 20, 25, 30, 35, 40, 45, 50, 55, 60, 65, 70, 75, 80, 85, 90, 95, or 100 micrograms.

In some cases, a GUIDE-Seq analysis can be performed to determine the specificity of engineered guide RNAs. The general mechanism and protocol of GUIDE-Seq profiling of off-target cleavage by CRISPR system nucleases is discussed in Tsai, S. et al., “GUIDE-Seq enables genome-wide profiling of off-target cleavage by CRISPR system nucleases,” Nature, 33: 187-197 (2015).

In some cases, one or more guides are introduced into a cell. In other cases, two or more guides are introduced into a cell. The two or more guide nucleic acids can be simultaneously present on the same expression vector or introduced as naked guides. The two or more guide nucleic acids can be under the same transcriptional control. In some embodiments, two or more (e.g., 3 or more, 4 or more, 5 or more, 10 or more, 15 or more, 20 or more, 25 or more, 30 or more, 35 or more, 40 or more, 45 or more, or 50 or more) guide nucleic acids are simultaneously expressed in a target cell (from the same or different vectors). In some cases, guide nucleic acids can be differently recognized by dead Cas proteins (e.g., dCas9 proteins from different bacteria, such as S. pyogenes, S. aureus, S. thermophilus, L. innocua, and N. meningitides).

### Inhibition of Non-Homologous Recombination

Non-homologous end-joining molecules such as KU70, KU80, and/or DNA Ligase IV can be suppressed by using a variety of methods. For example, non-homologous end-joining molecules such as KU70, KU80, and/or DNA Ligase IV can be suppressed by gene silencing (e.g., during transcription or translation). Non-homologous end-joining molecules KU70, KU80, and/or DNA Ligase IV can also be suppressed by degradation of the protein. Non-homologous end-joining molecules KU70, KU80, and/or DNA Ligase IV can be also be inhibited. Inhibitors of KU70, KU80, and/or DNA Ligase IV can comprise E1B55K and/or E4 orf6. Non-homologous end-joining molecules KU70, KU80, and/or DNA Ligase IV can also be inhibited by sequestration. An agent that suppresses non-homologous end-joining can be a small molecule.

### Delivery Systems

Conventional viral and non-viral based gene transfer methods can be used to introduce nucleic acids encoding an endonuclease (e.g., CRISPR, TALEN, transposon-based, ZEN, meganuclease, or Mega-TAL molecules), a polynucleic acid construct (e.g., comprising an insert sequence), and gRNAs, to cells in vitro, ex vivo, or in vivo.

Exemplary viral vector delivery systems include DNA and RNA viruses, which have either episomal or integrated genomes after delivery to the cell. Viral vectors can be introduced into a cell using transduction methods known to the person of ordinary skill in the art. Exemplary non-viral vector delivery systems include DNA plasmids, mini-circle DNA, naked nucleic acid, mRNA, and nucleic acid complexed with a delivery vehicle such as a liposome or poloxamer.

Methods of non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Additional exemplary nucleic acid delivery systems include those provided by AMAXA® Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. (see for example U.S. Pat. No. 6,008,336). Lipofection reagents are sold commercially (e.g., TRANSFECTAM® and LIPOFECTIN®), sonoporation using, e.g., the Sonitron 2000 system (Rich-Mar).

In some embodiments, the endonuclease is introduced into a cell using an mRNA molecule encoding said endonuclease. In some embodiments, the endonuclease is introduced into a cell using a viral vector. In some embodiments, the gRNA is introduced into a cell using a synthetic RNA molecule. In some embodiments, the polynucleic acid construct is introduced into the cell using a DNA plasmid. In some embodiments, the polynucleic acid construct is introduced into the cell using a minicircle DNA plasmid. In some embodiments, the polynucleic acid construct is introduced into the cell using a viral vector. In some embodiments, the polynucleic acid construct is introduced into the cell using an AAV vector.

In some cases, a polynucleic acid construct described herein is introduced into a cell for via RNA, e.g., messenger RNA (mRNA). In some embodiments, the mRNA polynucleic acid can be introduced into a cell with a reverse transcriptase (RT) (either in protein form or a polynucleic acid encoding for a RT). Exemplary RT include, but are not limited to, those derived from Avian Myeloblastosis Virus Reverse Transcriptase (AMV RT), Moloney murine leukemia virus (M-MLV RT), human immunodeficiency virus (HIV) reverse transcriptase (RT), derivatives thereof or combinations thereof. Once transfected, a reverse transcriptase may transcribe the engineered mRNA polynucleic acid into a double strand DNA (dsNDA). A reverse transcriptase (RT) can be an enzyme used to generate complementary DNA (cDNA) from an RNA template. In some cases, an RT enzyme can synthesize a complementary DNA strand initiating from a primer using RNA (cDNA synthesis) or single-stranded DNA as a template.

### Electroporation Schemes

Provided herein are methods of improving overall yield from cell engineering, including, for instance, improving cell viability after cell engineering, and/or improving transfection efficiency. One aspect of the present disclosure provides a method of genomically editing, comprising a first electroporation step and a second electroporation step. In some instances, a sequential electroporation scheme as provided herein can increase cell viability. In some cases, a sequential electroporation scheme as provided herein can increase transfection efficiency. In some instances, a sequential electroporation scheme as provided herein can increase both cell viability and transfection efficiency.

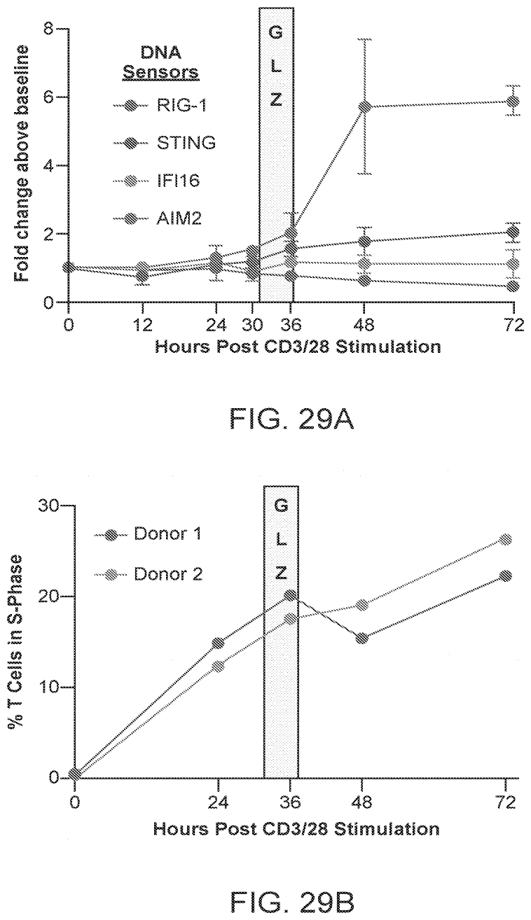

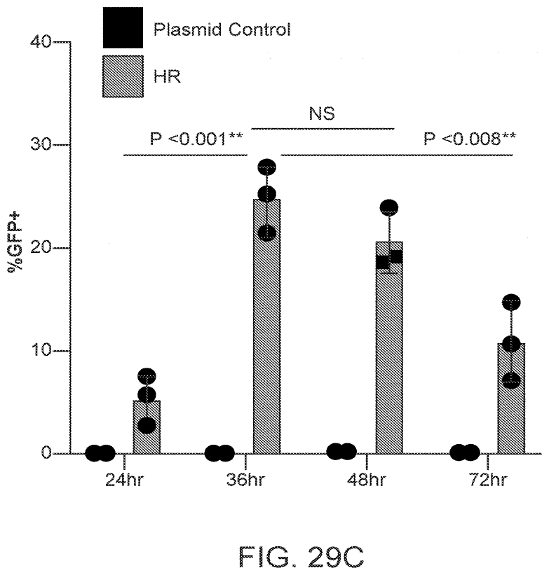

In some case, A first electroporation step can comprise introducing a guided-nuclease into the cells. A second electroporation step can comprise introducing into the cells a guide polynucleic acid comprising a region complementary to at least a portion of a gene. A second electroporation step can further comprise introducing into the cells an exogenous polynucleic acid. A method can generate modified cells. A first electroporation can be performed at any time. In some cases, an electroporation is performed after stimulation, such as with anti-CD3 and/or anti-CD28. Any number of cytokines or interleukins can also be used in combination with the anti-CD3 or anti-CD28 for stimulation. Electroporation can be performed from about 0 hr., 2 hr., 4 hr., 6 hr., 8 hr., 10 hr., 12 hr., 14 hr., 16 hr., 18 hr., 20 hr., 22 hr., 24 hr., 26 hr., 28 hr., 30 hr., 32 hr., 34 hr., 36 hr., 38 hr., 40 hr., 42 hr., 44 hr., 46 hr., 48 hr., 50 hr., 52 hr., 54 hr., 56 hr., 58 hr., 60 hr., 62 hr., 64 hr., 66 hr., 68 hr., 70 hr., 72 hr., 74 hr., 76 hr., 78 hr., 80 hr., 82 hr., 84 hr., 86 hr., 88 hr., 90 hr., 92 hr., 94 hr., 96 hr., 98 hr., or up to about 100 hrs after an electroporation. In some cases, an electroporation is performed from about 30 hrs.-40 hrs. after stimulation. In some cases, an electroporation is performed at 36 hrs. post stimulation. In some cases, transfection is timed based on the S-phase of a cellular population, see for example, FIG. 29A and FIG. 29B showing expression levels of various DNA sensors as a function of hours post stimulation.

In some cases, a first electroporation step can comprise introducing a guided-ribonucleoprotein complex into the cells. A second electroporation step can comprise introducing into the cells an exogenous polynucleic acid. A method can generate modified cells.

A method provided herein can comprise sequential electroporation of the cells to be modified. In some cases, a method can comprise a first electroporation step and a second electroporation step. In some cases, the first and second electroporation steps are conducted with an interval. The interval between the first and second electroporation steps can be from about 10 min to about 48 hr, from about 30 min to about 44 hr, from about 1 hr to about 40 hr, from about 2 hr to about 36 hr, from 3 hr to about 32 hr, from about 4 hr to about 30 hr, from about 5 hr to about 28 hr, from about 5.5 hr to about 26 hr, from about 6 hr to about 24 hr, from about 6.5 hr to about 22 hr, from about 7 hr to about 20 hr, from about 8 hr to about 16 hr, from about 9 hr to about 12 hr, or from about 10 to about 11 hr. In some cases, the interval between the first and second electroporation steps can be about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, or about 24 hr.

An interval between the first and second electroporation steps can be beneficial to the cell viability. A method comprising a first and a second electroporation steps as provided herein can promote an increase in a percentage of viability as compared to comparable cells comprising a single electroporation consisting of both first and second electroporation steps. An increase in viability percentage can be about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 125%, about 150%, about 175%, about 200%, about 250%, about 300%, or even more. In some cases, an increase in viability percentage can be from about 50% to about 200%.

An interval between the first and second electroporation steps can be beneficial to the transgene integration efficiency. A method comprising a first and a second electroporation steps as provided herein can promote an increase in a percentage of integration efficiency as compared to comparable cells comprising a single electroporation consisting of both first and second electroporation steps. An increase in integration efficiency can be about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 125%, about 150%, about 175%, about 200%, about 250%, about 300%, or even more. In some cases, an increase in integration efficiency can be from about 50% to about 200%.

A first electroporation step can comprise introducing to the cells a guided-nuclease. As provided herein, a guided-nuclease can comprise CRISPR associated proteins (Cas proteins, e.g., Cas9), Zinc finger nuclease (ZFN), Transcription Activator-Like Effector Nuclease (TALEN), transposases, and meganucleases. Guided-nucleases can be naturally existing nucleases, genetically modified, and/or recombinant. Guided-nucleases can be introduced to the target cell in any form that may result in functional presence of the guided-nucleases inside the cell. In some cases, the guided-nucleases can be transfected into the cells in the form of a DNA. In some cases, the guided-nucleases can be transfected in the form of an mRNA. In some cases, the guided-nucleases can be delivered into the cells in the form of a protein or protein complex. In some cases, a guided-nuclease can comprise a Cas protein. Non-limiting examples of Cas protein that can be used for the method provided herein included Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, or modified versions thereof.

A second electroporation step can comprise introducing to the cells a guide polynucleic acid comprising a region complementary to at least a portion of a gene. A second electroporation step can comprise introducing to the cells an exogenous polynucleic acid. A second electroporation step can comprise introducing to the cells a guide polynucleic acid comprising a region complementary to at least a portion a gene and an exogenous polynucleic acid. In some cases, the guide polynucleic acid comprising a region complementary to at least a portion of a gene can comprise a guide RNA as used in CRISPR system. A guide RNA can comprise a crRNA and a tracrRNA. In some cases, the guide polynucleic acid comprising a region complementary to at least a portion of a gene and the exogenous polynucleic acid can be present on a single polynucleotide molecule, for instance, on a single DNA plasmid.

An exogenous polynucleic acid that can be used for a method provided herein can comprise any nucleotide sequence. In some cases, an exogenous polynucleic acid can comprise a transgene. A transgene can be any gene or derivative thereof. In some cases, a transgene can comprise a cellular receptor, such as, a T cell receptor (TCR), a B cell receptor (BCR), a chimeric antigen receptor (CAR), or a combination thereof.

### Therapeutic Applications

Genetically-edited immune cells of the disclosure can be used in methods of therapy, for example, therapies for a cancer, inflammatory disorder, autoimmune disorder, or infectious disease. Modifications that can be introduced into the immune cell genome include, for example, insertions, deletions, sequence replacement, (e.g., substitutions), and combinations thereof. One or more sequences can be inserted into the genome, for example, to allow expression of an exogenous gene product (e.g., a T cell receptor or chimeric antigen receptor of known antigen-specificity, an immunoglobulin of known specificity, a cytokine or cytokine receptor, a chemokine or chemokine receptor, or a protein comprising a drug-responsive domain). A promoter sequence can be inserted into the genome, for example, to allow for regulated or constitutive expression of and endogenous gene product or an exogenous (inserted) gene product. One or more genes can be disrupted, for example, to disrupt expression of a product that contributes to the pathogenesis of a disease (e.g., an immune checkpoint gene that decreases an anti-cancer or anti-pathogen immune response, or a pro-inflammatory gene that contributes to an inflammatory disorder or autoimmune disorder). A defined sequence can be deleted from the genome, for example, to alter the function of a gene product (e.g., deletion of an exon or deletion of one or more domains of a protein). A sequence in the genome can be replaced by another sequence, for example, to replace a disease-associated sequence (e.g., SNP or mutation) with a normal sequence, or to alter the function of a gene product (e.g., binding affinity for an antigen, ligand, agonist, antagonist etc.).

Exemplary cancers include, but are not limited to, acute lymphocytic cancer, acute myeloid leukemia, alveolar rhabdomyosarcoma, bladder cancer, bone cancer, brain cancer, breast cancer, anal cancer, anal canal cancer, rectum cancer, eye cancer, intrahepatic bile duct cancer, joint cancer, neck cancer, gallbladder cancer, pleura cancer, nose cancer, nasal cavity cancer, middle ear cancer, oral cavity cancer, vulva cancer, chronic lymphocytic leukemia, chronic myeloid cancer, colon cancer, esophageal cancer, cervical cancer, fibrosarcoma, gastrointestinal cancer, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum cancer, omentum cancer, mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer.

In some embodiments, the cancer is bladder cancer, epithelial cancer, bone cancer, brain cancer, breast cancer, esophageal cancer, gastrointestinal cancer, leukemia, liver cancer, lung cancer, lymphoma, myeloma, ovarian cancer, prostate cancer, sarcoma, stomach cancer, thyroid cancer, acute lymphocytic cancer, acute myeloid leukemia, alveolar rhabdomyosarcoma, anal canal, rectal cancer, ocular cancer, cancer of the neck, gallbladder cancer, pleural cancer, oral cancer, cancer of the vulva, colon cancer, cervical cancer, fibrosarcoma, gastrointestinal carcinoid tumor, Hodgkin lymphoma, kidney cancer, mesothelioma, mastocytoma, melanoma, multiple myeloma, nasopharynx cancer, non-Hodgkin lymphoma, pancreatic cancer, peritoneal cancer, renal cancer, skin cancer, small intestine cancer, soft tissue cancer, solid tumors, stomach cancer, testicular cancer, or thyroid cancer. In some embodiments, the cancer is gastrointestinal cancer, breast cancer, lymphoma, or prostate cancer.

Exemplary autoimmune diseases include, but are not limited to, achalasia, Addison's disease, adult still's disease, agammaglobulinemia, alopecia areata, amyloidosis, ankylosing spondylitis, anti-GBM/Anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy, baló disease, behcet's disease, benign mucosal pemphigoid, bullous pemphigoid, castleman disease, celiac disease, chagas disease, chronic inflammatory demyelinating polyneuropathy, chronic recurrent multifocal osteomyelitis, churg-strauss syndrome, eosinophilic granulomatosis, cicatricial pemphigoid, cogan's syndrome, cold agglutinin disease, congenital heart block, coxsackie myocarditis, CREST syndrome, crohn's disease, dermatitis herpetiformis, dermatomyositis, devic's disease (neuromyelitis optica), discoid lupus, dressler's syndrome, endometriosis, eosinophilic esophagitis, eosinophilic fasciitis, erythema nodosum, essential mixed cryoglobulinemia, evans syndrome, fibromyalgia fibrosing alveolitis, giant cell arteritis (temporal arteritis), giant cell myocarditis, glomerulonephritis, goodpasture's syndrome, granulomatosis with polyangiitis graves' disease, guillain-barre syndrome, hashimoto's thyroiditis, hemolytic anemia, henoch-schonlein purpura, herpes gestationis or pemphigoid gestationis, hidradenitis suppurativa, hypogammaglobulinemia, IgA nephropathy, IgG4-related sclerosing disease, immune thrombocytopenic purpura, inclusion body myositis, interstitial cystitis, juvenile arthritis, juvenile diabetes, juvenile myositis, kawasaki disease, lambert-eaton syndrome, leukocytoclastic vasculitis, lichen planus lichen sclerosis, ligneous conjunctivitis, linear IgA disease, lupus, lyme disease chronic, Meniere's disease, microscopic polyangiitis, mixed connective tissue disease, mooren's ulcer, mucha-habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, PANDAS, paraneoplastic cerebellar degeneration, paroxysmal nocturnal hemoglobinuria, parry romberg syndrome, pars planitis, parsonage-turner syndrome, pemphigus, peripheral neuropathy, perivenous encephalomyelitis, pernicious anemia, POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, subacute bacterial endocarditis, Susac's syndrome, sympathetic ophthalmia, Takayasu's arteritis, temporal arteritis/giant cell arteritis, thrombocytopenic purpura, Tolosa-Hunt syndrome, transverse myelitis, type 1 diabetes, ulcerative colitis, undifferentiated connective tissue disease, uveitis, vasculitis, vitiligo, and Vogt-Koyanagi-Harada disease.

The cells described herein can be administered to a subject in need thereof. In some embodiments, the cells are allogenic or autologous to the subject they are administered to. In some embodiments, the cells are administered as a single dose. In some embodiments, the cells are administered in multiple doses. In some embodiments, the cells are administered via intravenous infusion.

In some embodiments, target cells such as cancer cells can form a tumor. A tumor treated with the compositions and methods provided herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

Death of target cells such as cancer cells can be determined by any suitable method, including, but not limited to, counting cells before and after treatment, or measuring the level of a marker associated with live or dead cells (e.g. live or dead target cells). Degree of cell death can be determined by any suitable method. In some embodiments, degree of cell death is determined with respect to a starting condition. For example, an individual can have a known starting amount of target cells, such as a starting cell mass of known size or circulating target cells at a known concentration. In such cases, degree of cell death can be expressed as a ratio of surviving cells after treatment to the starting cell population. In some embodiments, degree of cell death can be determined by a suitable cell death assay. A variety of cell death assays are available, and can utilize a variety of detection methodologies. Examples of detection methodologies include, without limitation, the use of cell staining, microscopy, flow cytometry, cell sorting, and combinations of these. When a tumor is subject to surgical resection following completion of a therapeutic period, the efficacy of treatment in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%). In some embodiments, the necrosis percentage of the resected tissue is 100%, that is, no living tumor tissue is present or detectable.

Exposing a cancer cell to an immune cell or population of immune cells disclosed herein can be conducted either in vitro or in vivo. Exposing a target cell to an immune cell or population of immune cells generally refers to bringing the target cell in contact with the immune cell and/or in sufficient proximity such that an antigen of a target cell (e.g., membrane bound or non-membrane bound) can bind to the antigen interacting domain of the chimeric transmembrane receptor polypeptide expressed in the immune cell. Exposing a target cell to an immune cell or population of immune cells in vitro can be accomplished by co-culturing the target cells and the immune cells. Target cells and immune cells can be co-cultured, for example, as adherent cells or alternatively in suspension. Target cells and immune cells can be co-cultured in various suitable types of cell culture media, for example with supplements, growth factors, ions, etc. Exposing a target cell to an immune cell or population of immune cells in vivo can be accomplished, in some cases, by administering the immune cells to a subject, for example a human subject, and allowing the immune cells to localize to the target cell via the circulatory system. In some cases, an immune cell can be delivered to the immediate area where a target cell is localized, for example, by direct injection. Exposing can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

### Kits

Any of the compositions described herein may be comprised in a kit. In a non-limiting example, a transgene, a vector, a polynucleotide, a peptide, reagents to generate compositions provided herein, and any combination thereof may be comprised in a kit. In some cases, kit components are provided in suitable container means.

Kits may comprise a suitably aliquoted composition. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

In some embodiments, a kit can comprise an engineered guide RNA, a precursor engineered guide RNA, a vector comprising the engineered guide RNA or the precursor engineered guide RNA, or a nucleic acid of the engineered guide RNA or the precursor engineered guide RNA, an engineered cellular receptor, a polynucleotide encoding the engineered cellular receptor, or a pharmaceutical composition that comprises any of the above and a container. In some instances, a container can be plastic, glass, metal, or any combination thereof.

In some instances, a packaged product comprising a composition described herein can be properly labeled. In some instances, the pharmaceutical composition described herein can be manufactured according to good manufacturing practice (cGMP) and labeling regulations. In some cases, a pharmaceutical composition disclosed herein can be aseptic.

While preferred embodiments have been shown and described herein, it will be obvious to those skilled in the art that such embodiments are provided by way of example only. Numerous variations, changes, and substitutions will now occur to those skilled in the art. It should be understood that various alternatives to the embodiments described herein may be employed. It is intended that the following claims define the scope and that methods and structures within the scope of these claims and their equivalents be covered herein.

### EXAMPLES

### Example 1: Isolation of T Cells

Peripheral blood mononuclear cells (PBMCs) are isolated from whole blood or an apheresis unit using ammonium chloride-based RBC lysis and/or density gradient centrifugation (e.g., Ficoll-Paque). PBMCs are enumerated, cell density is adjusted to 5×10\hat{ }7 cells/mL in EasySep buffer or PBS with 2% FBS and 1 mM EDTA (Calcium and Magnesium-free), and up to 8 mL is transferred to a round bottom tube. An isolation cocktail from an EasySep Human T-cell Isolation kit (Cat #19051) is added to the cells at 50 uL/mL. The cells are mixed by pipetting and incubated for 5 minutes at room temperature. RapidSpheres are mixed by vortexing for 30 seconds, and added to the sample at 40 uL/mL. Samples are topped up to 5 mL or 10 mL and mixed gently by pipetting. The tube is placed into an EasySep magnet and incubated at room temperature for 3 minutes. Non-T cells are captured on the magnet, while T cells remain unbound. Isolated T cells are transferred to a new conical tube by carefully pipetting or pouring the supernatant in one continuous motion. T cells are counted, and purity validated by flow cytometry (e.g., validated for >90% CD3+ cells). Cells can be cultured, stimulated, or aliquoted and frozen for future use (e.g., with Cryostor CS10).

### Example 2: Expansion of T Cells

Isolated T cells are plated at a density of 1×10\hat{ }6 cells/mL in a 24 well plate in OpTmizer™ T-Cell Expansion Basal Medium with 2.6% OpTmizer™ T-Cell Expansion Supplement, 2.5% CTS™ Immune Cell Serum Replacement, 1% L-Glutamine, 1% Penicillin/Streptomycin, 10 mM N-Acetyl-L-cysteine, 300 IU/mL recombinant human IL-2, 5 ng/mL recombinant human IL-7, and 5 ng/mL recombinant human IL-15. If frozen cells frozen isolated T cells are used, the cells are rested for at least 4-5 h after thawing prior to stimulation.

Human T-Activator CD3/CD28 Dynabeads are washed with culturing media, collected using a magnet, and added to the isolated T cells at a ratio of 2 beads per cell or 1 bead for every 2.5 cells. The cells are incubated at 37° C., 5% CO2. After 12-24 hours, the sample is gently pipetted to redistribute the beads. After a total of 36 hours of incubation the beads are removed using a magnet.

### Example 3: Nucleofection of T Cells

Isolated T cells are stimulated as outlined in Example 2 and are electroporated using the Lonza 4D Nucleofector™ X Unit & Amaxa 4D-Nucleofector X Kits. Cells are pelleted, washed once with kit-provided buffer, resuspended in kit-provided buffer, and transferred to a cuvette according to kit instructions.

For 100 uL cuvettes, 5-15 ug Cas9 mRNA, and 5-25 ug gRNA-RNA is added per cuvette. If plasmid DNA is added to the 100 uL cuvette, 5-10 ug of plasmid DNA is added.

For 20 uL cuvettes, 1-3 ug Cas9 mRNA, and 1-5 ug gRNA-RNA is added per cuvette. If plasmid DNA is added to the 20 uL cuvette, 1-2 ug of plasmid DNA is added.

Nucleofection is performed according to kit instructions. The cells are rested for 15 minutes in the cuvette, then transferred to a recovery plate containing antibiotic-free culture media. Cells are handled gently with minimal pipetting. For 100 uL cuvettes, contents are transferred to 1 mL per well of a 6 well plate. For 20 uL cuvettes, contents are transferred to 300 uL per well of a 24 well plate. If plasmid DNA was added, 1 ug DNase is included in recovery wells. Cells are incubated for 30 minutes at 37° C., 5% CO2, then additional culture media is added to bring the cell concentration to 1×10\hat{ }6 cells/mL, and cultures are maintained at 37° C., 5% CO2. Growth and viability are monitored periodically, e.g., via trypan blue exclusion with an automated cell counter.

### Example 4: Genomic Editing of T Cells Comprising Single Strand Annealing

A DNA minicircle construct is designed and synthesized comprising the elements represented in FIG. 1A. The “Insert” box represents a DNA sequence comprising a promoter (MND promoter), and an open reading frame encoding a T Cell Receptor (an exogenous G12D KRAS-specific TCR comprising a mouse TCRb sequence recognizable by specific monoclonal antibodies), including a poly-A tail. T1 represents a sequence targeted for cleavage by a guide RNA (for example, a guide RNA that does not target the genome (e.g., zebrafish guide RNA or algorithmically-designed guide RNA), or a guide RNA that targets a disruption target site in the genome). H1 and H2 represent short homology arms with sequences homologous to chosen sites in the genome (48 base pair sequences within TRAC exon 1). As a control, a DNA minicircle construct is designed comprising the insert with 1000 base pair homology arms instead of 48 base pair homology arms.

The construct is designed for insertion at a TRAC target site in the genome represented in FIG. 1B. H1 and H2 represent sequences in the genome homologous to H1 and H2 in the DNA minicircle construct. C2 represents a sequence targeted for cleavage by a guide RNA (e.g., the same guide RNA that targets C1 or a different guide RNA).

Human T cells are isolated as in Example 1, and expanded as in Example 2, and electroporated using a Lonza 4D Nucleofector™ X Unit and Amaxa 4D-Nucleofector X Kit. Cells are pelleted, washed once with kit-provided buffer, resuspended in kit-provided buffer, and transferred to 20 uL cuvettes according to kit instructions.

DNA and/or RNA are added to the cuvettes in the amounts shown in Table 2.

TABLE 2

| | | DNA | | | |
|---|---|---|---|---|---|
| | DNA | minicircle | | | DNA |
| | minicircle | with 1000 | | TRAC | minicircle |
| | with 48 bp | bp | | (C2)- | (C1)- |
| | homology | homology | Cas9 | targeting | targeting |
| Condition # | arms | arms | mRNA | gRNA | gRNA |
| 1 | — | — | — | — | — |
| 2 | — | 1 ug | — | — | — |
| 3 | 1 ug | — | — | — | — |
| 4 | — | 1 ug | 1.5 ug | 1 ug | — |
| 5 | — | 1 ug | 3 ug | 1 ug | — |
| 6 | 1 ug | — | 1.5 ug | 1 ug | 1 ug |
| 7 | 1 ug | — | 3 ug | 1 ug | 1 ug |

Nucleofection is performed according to kit instructions. The cells are rested for 15 minutes in the cuvette, then transferred to a 24-well plate containing 300 uL of antibiotic-free culture medium per well, with 1 ug DNase. Cells are handled gently with minimal pipetting. Cells are incubated for 30 minutes at 37° C., 5% CO2, then additional culture media is added to bring the cell concentration to 1×10\hat{ }6 cells/mL. Cultures are maintained at 37° C., 5% CO2 for 7 days, with media changed and cultures split as needed.

On day 7 post-nucleofection, cells are analyzed by flow cytometry to determine the frequency and number of cells expressing the TCR encoded by the DNA minicircle constructs. 5×10≡cells per experimental condition are taken, pelleted, and stained with fluorescently-conjugated monoclonal antibodies specific for CD3 and the insert TCR. The cells are also stained with a viability dye. After staining, cells are subjected to flow cytometry, and live cells are analyzed for expression of CD3 and the insert TCR.

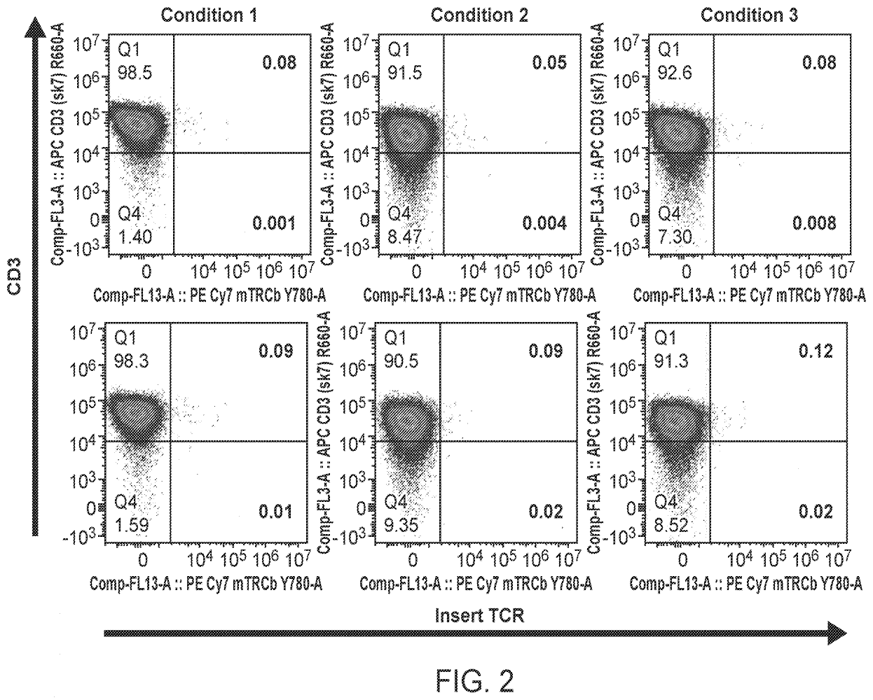

FIG. 2 presents the results for experimental conditions 1-3 and illustrates that in conditions without nuclease or guide RNA, the insert TCR is not expressed. Each column represents a condition. Each row represents a sample derived from a different donor. The y-axes represent fluorescence from CD3 staining, and the X-axes represent fluorescence from staining for the insert TCR. The numbers represent the percentage of live cells that fall within the quadrant.

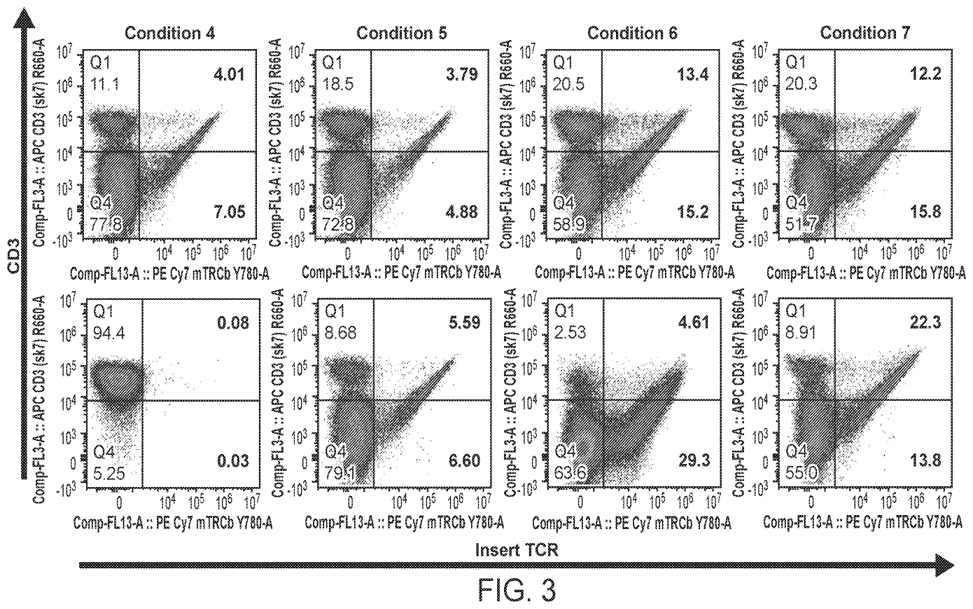

FIG. 3 presents the results from experimental conditions 4-7 and illustrates that higher proportions and numbers of cells express the insert TCR in the experimental conditions with 48 base pair homology arms and minicircle-targeting guide RNAs (conditions 6 & 7) compared to the experimental conditions with the 1000 base pair homology arms (conditions 4 & 5). Each column represents a condition. Each row represents a sample derived from a different donor. The y-axes represent fluorescence from CD3 staining, and the X-axes represent fluorescence from staining for the insert TCR. The numbers represent the percentage of live cells that fall within the quadrant. These results demonstrate improved efficiency of immune cell genome editing using methods that comprise single strand annealing compared to homologous recombination.

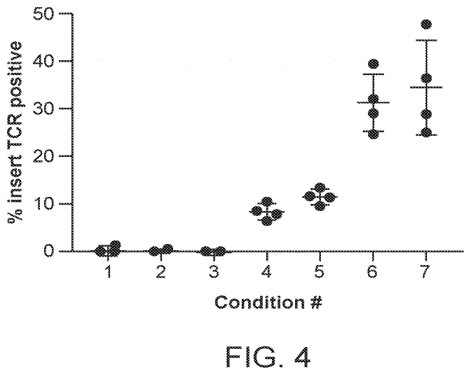

FIG. 4 provides the percentage of live cells that express the insert TCR from experimental conditions 1-7. Data are presented for samples processed from two donors, with two technical replicates per donor. The results illustrate that higher proportions and numbers of cells express the insert TCR in the experimental conditions with 48 base pair homology arms and minicircle-targeting guide RNAs (conditions 6 & 7) compared to the experimental conditions with the 1000 base pair homology arms (conditions 4 & 5). These results demonstrate improved efficiency of immune cell genome editing using methods that comprise single strand annealing compared to homologous recombination.

TABLE 3
Exemplary polynucleic acid constructs

| SEQ | | |
|---|---|---|
| ID | | |
| NO | Identity | Sequence |
| 78 | Anti-TRAC gRNA | mU*mC*mU* rCrUrC rArGrC |
| | (exemplary | rUrGrG rUrArC rArCrG |
| | modifications | rGrCrG rUrUrU rUrArG |
| | denoted) | rArGrC rUrArG rArArA |
| | | rUrArG rCrArA rGrUrU |
| | | rArArA rArUrA rArGrG |
| | | rCrUrA rGrUrC rCrGrU |
| | | rUrArU rCrArA rCrUrU |
| | | rGrArA rArArA rGrUrG |
| | | rGrCrA rCrCrG rArGrU |
| | | rCrGrG rUrGrC mU*mU*mU* |
| | | rU |
| 79 | Anti-TRAC gRNA | UCUCUCAGCUGGUACACGGCGU |
| | | UUUAGAGCUAGAAAUAGCAAGU |
| | | UAAAAUAAGGCUAGUCCGUUAU |
| | | CAACUUGAAAAAGUGGCACCGA |
| | | GUCGGUGCUUUU |
| 80 | Genomic target | GCCGTGTACCAGCTGAGAGA |
| | sequence of anti- | |
| | TRAC gRNA (sense | |
| | strand) | |
| 81 | Anti-TRAC gRNA | UCUCUCAGCUGGUACACGGC |
| | spacer sequence | |

### Example 5: Genomic Editing of T Cells Comprising Single Strand Annealing

A DNA minicircle construct is designed and synthesized comprising the elements represented in FIG. 1A. The “Insert” box represents a DNA sequence comprising a promoter and an open reading frame encoding green fluorescent protein (GFP). T1 represents a sequence targeted for cleavage by a guide RNA (for example, a guide RNA that does not target the genome (e.g., zebrafish guide RNA or algorithmically-designed guide RNA), or a guide RNA that targets a disruption target site in the genome). H1 and H2 represent short homology arms with sequences homologous to chosen sites in the genome (48 base pair sequences within the AAVS1 safe harbor locus). As a control, a DNA minicircle construct is designed comprising the insert with 1000 base pair homology arms instead of 48 base pair homology arms.

An exemplary gRNA that targets a xenogeneic sequence, such as a universal sequence, can comprise from about 50%, 60%, 70%, 80%, 85%, 90%, 95%, 97%, 98%, 99%, or 100% identity to: GGGAGGCGUUCGGGCCACAGGUUUUAGAGCUAGAAAUAGCAAGUUAAAAUAA GGCUAGUCCGUUAUCAACUUGAAAAAGUGGCACCGAGUCGGUGCUUUU (SEQ ID NO: 82). The exemplary aforementioned gRNA can also comprise modifications, such as those described in: mG*mG*mG*rArGrG rCrGrU rUrCrG rGrGrC rCrArC rArGrG rUrUrU rUrArG rArGrC rUrArG rArArA rUrArG rCrArA rGrUrU rArArA rArUrA rArGrG rCrUrA rGrUrC rCrGrU rUrArU rCrArA rCrUrU rGrArA rArArA rGrUrG rGrCrA rCrCrG rArGrU rCrGrG rUrGrC mU*mU*mU*rU (SEQ ID NO: 83). The spacer sequence of an exemplary universal guide (zebrafish) gRNA can comprise from about 50%, 60%, 70%, 80%, 85%, 90%, 95%, 97%, 98%, 99%, or 100% identity to: gggaggcguucgggccacag (SEQ ID NO: 84). A target sequence that can be bound by the aforementioned universal gRNA (exemplary universal sequence), T1, comprises: CTGTGGCCCGAACGCCTCCC (SEQ ID NO: 85).

The genomic target sequence that is bound by the AAVS1 gRNA comprises: CTAGGGACAGGATTGGTGAC (SEQ ID NO: 86). The AAVS1 gRNA can share the backbone, or region lacking the spacer sequence, of any one of SEQ ID NO: 79 or 82. For example, the AAVS1 gRNA can comprise the backbone of SEQ ID NO: 79, which correspond to residues 21-100.

The construct is designed for insertion at an AAVS1 target site in the genome represented in FIG. 1B. H1 and H2 represent sequences in the genome homologous to H1 and H2 in the DNA minicircle construct. T2 represents a sequence targeted for cleavage by a guide RNA (e.g., the same guide RNA that targets C1 or a different guide RNA).

Human T cells are isolated as in Example 1, and expanded as in Example 2, and electroporated using a Lonza 4D Nucleofector™ X Unit & Amaxa 4D-Nucleofector X Kit. Cells are pelleted, washed once with kit-provided buffer, resuspended in kit-provided buffer, and transferred to 20 uL cuvettes according to kit instructions.

DNA and/or RNA are added to the cuvettes in the amounts shown in Table 4.

TABLE 4

| | | DNA | | | |
|---|---|---|---|---|---|
| | DNA | minicircle | | | DNA |
| | minicircle | with 1000 | | AAVS1 | minicircle |
| | with 48 bp | bp | | (C2)- | (C1)- |
| | homology | homology | Cas9 | targeting | targeting |
| Condition # | arms | arms | mRNA | gRNA | gRNA |
| 1 | — | — | — | — | — |
| 2 | — | 1 ug | — | — | — |
| 3 | 1 ug | — | — | — | — |
| 4 | — | 1 ug | 1.5 ug | 1 ug | — |
| 5 | — | 1 ug | 3 ug | 1 ug | — |
| 6 | 1 ug | — | 1.5 ug | 1 ug | 1 ug |
| 7 | 1 ug | — | 3 ug | 1 ug | 1 ug |

Nucleofection is performed according to kit instructions. The cells are rested for 15 minutes in the cuvette, then transferred to a 24-well plate containing 300 uL of antibiotic-free culture medium per well, with 1 ug DNase. Cells are handled gently with minimal pipetting. Cells are incubated for 30 minutes at 37° C., 5% CO2, then additional culture media is added to bring the cell concentration to 1×10\hat{ }6 cells/mL. Cultures are maintained at 37° C., 5% CO2 for 7 days, with media changed and cultures split as needed.

On day 7 post-nucleofection, cells are analyzed by flow cytometry to determine the frequency of cells expressing the GFP reporter from the DNA minicircle constructs. 5×10≡cells per experimental condition are taken, pelleted, and stained with a viability dye. After staining, cells are subjected to flow cytometry, and live cells are analyzed for expression of GFP.

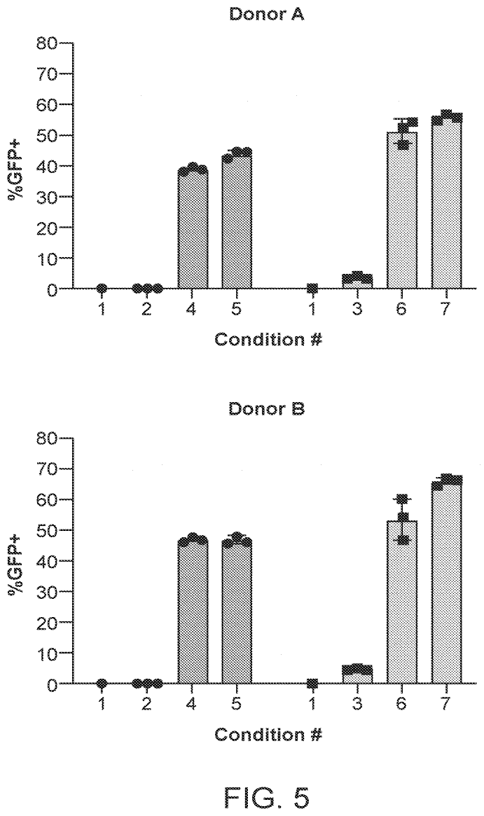

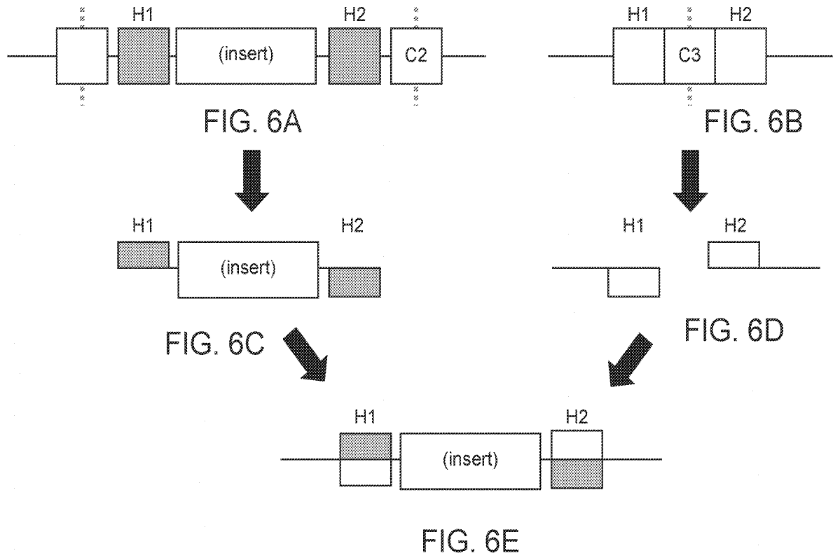

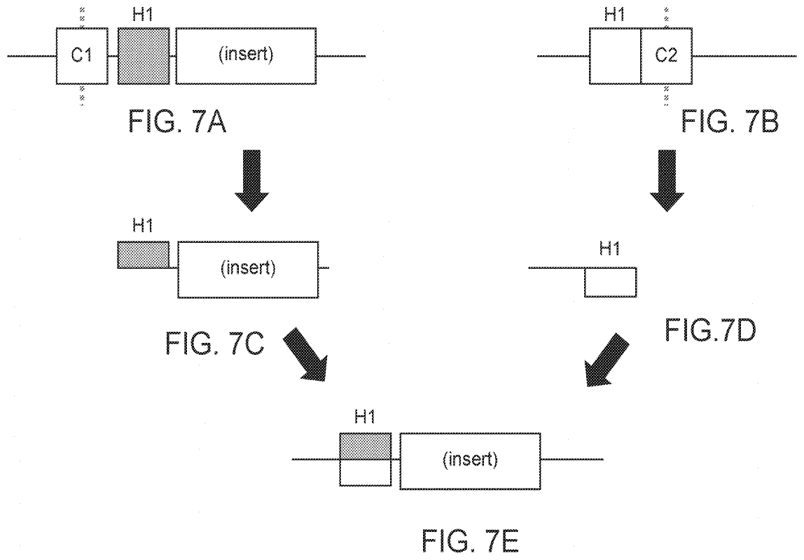

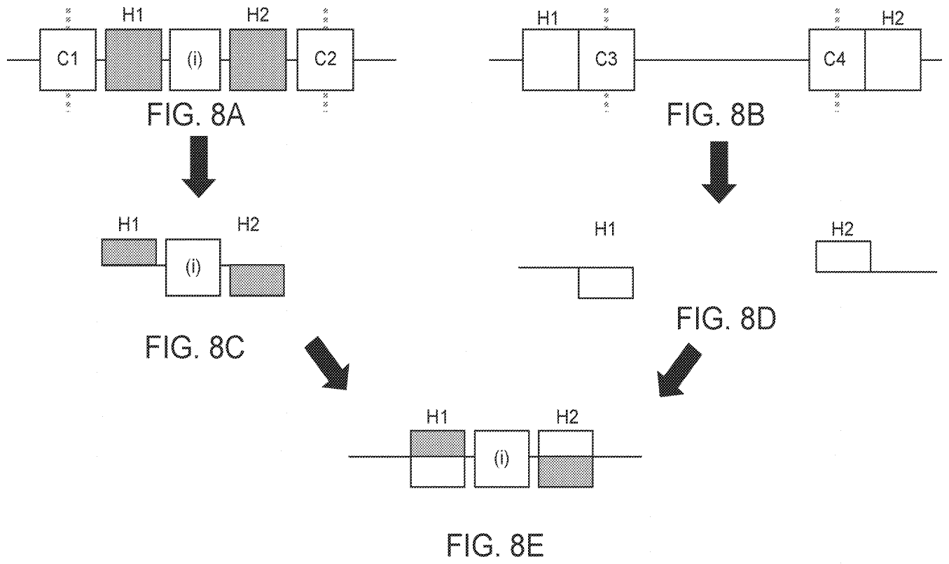

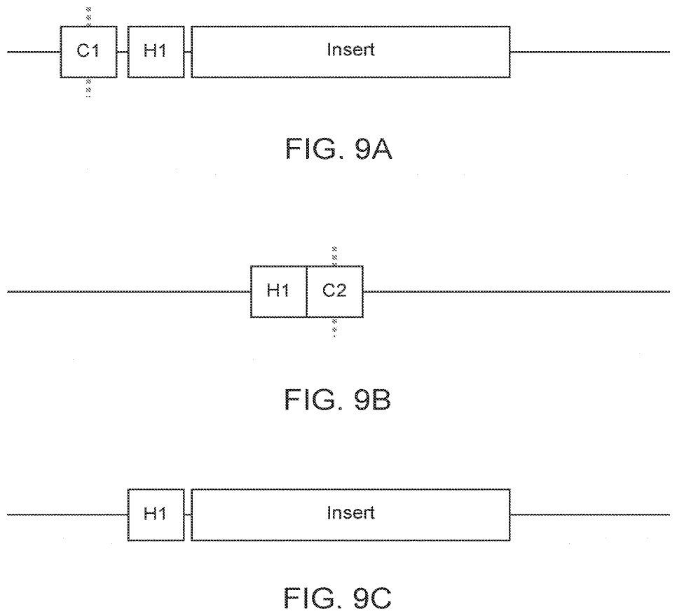

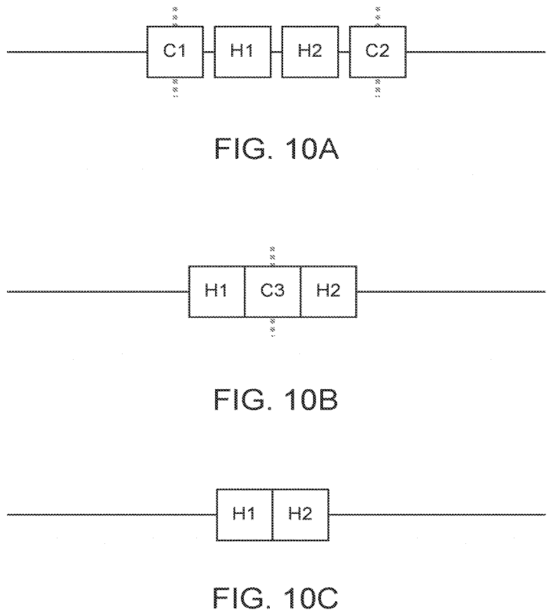

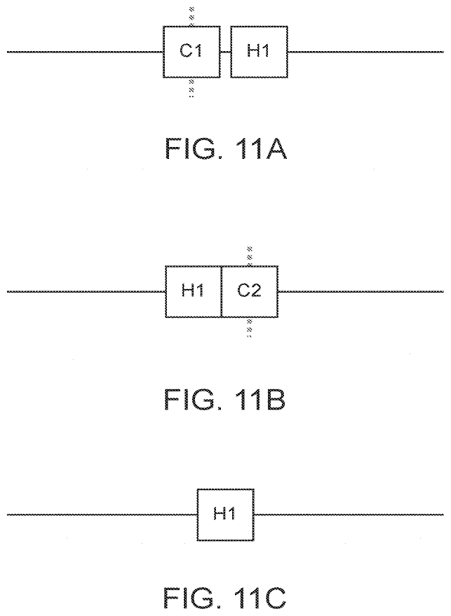

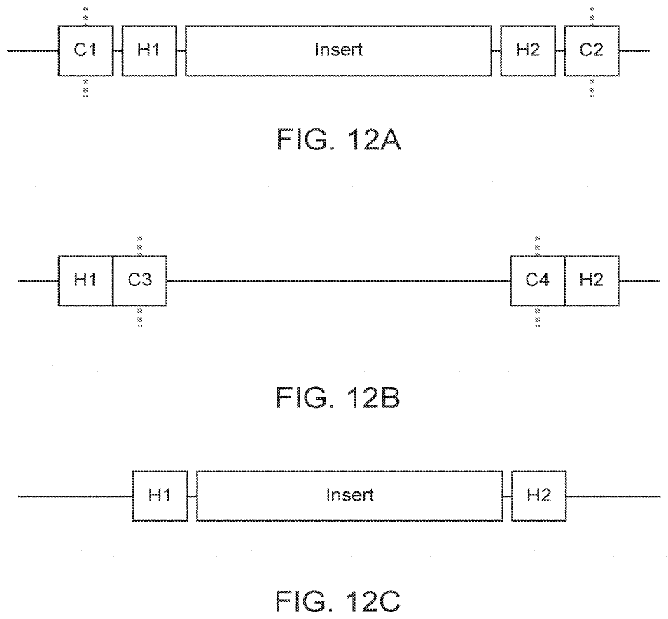

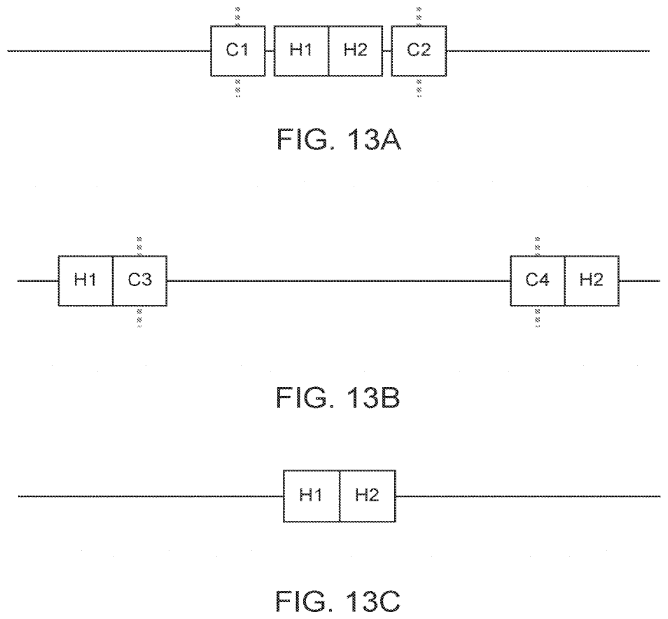

FIG. 5 provides the percentage of live cells that express the GFP reporter from experimental conditions 1-7. Data are presented for samples processed from two donors, with three technical replicates per donor. The results illustrate efficient immune cell genome editing using methods that comprise single strand annealing.

### Example 6: Materials and Methods for T Cell Modification

This Example provides materials and methods used in the examples 7-11 involving certain steps of primary T cell isolation, culture, transfection, and post-electroporation culture.

### Exemplary Protocol 1

Materials

Culturing Media:
X-VIVO 15 w/gentamicin, w/L-glutamine, w/transferrin, w/phenol red
X-VIVO 15 w/out gentamicin w/out phenol red, w/L-glutamine, w/transferrin
(*recovery media)
10% AB Human Serum
DNase I Solution (1 mg/ml)
3001 U/ml IL-2
5 ng/ml IL-7
5 ng/ml IL-15

Freezing Media:
Cryostor CS10

Cell Separation Reagents:
Human T-cell Isolation Kit
Ammonium chloride RBC lysis solution

Other Reagents:
Dynamag-2
Neon Kits

Antibody List:
Anti-Human CTLA4
Anti-human PD-1
Anti-human CD3

Methods

Isolation of Peripheral Blood Mononuclear Cells (PBMCs) from an Apheresis Unit (Leukopak) Using Ammonium Chloride Based RBC Lysis
(a) Measure volume of blood in leukopak
(b) Dispense leukopac into sterile 500 ml bottle and add equal volume of Ammonium chloride solution
(c) Mix by inverting several times
(d) Incubate on ice for 15 min
(e) Distribute sample evenly into 50 ml conicals and centrifuge at 500×g for 10 minutes.
(f) Carefully remove and discard supernatant.
(g) Top up the tube with 1×PBS+2% human AB serum and centrifuge at 150×g for 10 minutes with brake off.
(h) Repeat this wash step at least 1 time to remove platelets.
(i) Resuspend in appropriate media for T-cell purification using the EasySep Human T-cell Isolation kit.
   Isolation of Peripheral Blood Mononuclear Cells (PBMCs) from a Trima Cone Using Ammonium Chloride Based RBC Lysis
(a) Measure volume of blood in cone (usually −10 ml)
(b) Split volume into two 50 ml conicals
(c) Add 15 ml of 1×ACK lysis solution
(d) Incubate on ice for 20 min and quench with 20 ml 1×PBS+2% human AB Serum
(e) Centrifuge at 500×g for 10 minutes.
(f) Carefully remove and discard supernatant.
(g) Top up the tube with 1×PBS+2% human AB serum and centrifuge at 150×g for 10 minutes with brake off.
(h) Repeat this wash step at least 1 time to remove platelets.
(i) Resuspend in appropriate media for T-cell purification using the EasySep Human T-cell Isolation kit.

### Isolation of CD3+ T Cells Using EasySep Human T-Cell Isolation Kit (Cat #19051)

(a) Count Ficoll Separated PBMCs (*or washed apheresis unit/Leukopac) and adjust cell density to 1×10\hat{ }7 cells/mL.
(b) Transfer up to 45 mL to 50 ml conical tube (For use with Easy “50” magnet).
(c) Add 50 uL/mL of the Isolation Cocktail to the cells.
(d) Mix by pipetting and incubate for 10 minutes at room temperature.
(e) Vortex RapidSpheres for 30 seconds and add 50 uL/mL to the sample. Mix by pipetting up and down and incubate at RT for 10 minutes.
(f) Top up to 50 mL for samples >10 mLs.
(g) Place conical tube into Easy “50” magnet and incubate at room temperature for 10 minutes.
(h) Carefully pipette suspension out of conical in magnet and dispense in new 50 mL conical.
(i) Place conical back into magnet for second isolation and incubate for 5 minutes.
(j) Remove unbound T-cells by carefully removing supernatant in one round of pipetting using 50 ml pipette and transfer to new 50 ml conical.
(k) Count T-cells and validate purity by flow cytometry for % CD3+(>90%)
(l) Aliquot and freeze unused cells for future use (Crystor or 90% FBS:10% DMSO)

### Thawing Samples Originally Frozen in CryoStor CS10

(a) Thaw the cells in pre-warmed culture media (37° C.). Use the same type of media they will be cultured in.
(b) Add 1 mL of culture media to a sterile 15 mL conical tube.
(c) Thaw frozen vials in a 37° C. water bath until a single ice crystal remains. Immediately take the vials to a biosafety cabinet, spray with 70% ethanol and wipe.
(d) Open vials carefully. Gently pipet cell suspension dropwise from one vial into the 15 ml conical tube.
(e) Add an additional 1 ml of culture media dropwise and gently swirl.
(f) Add another 1 ml of culture media dropwise and gently swirl.
(g) Add additional 4 ml of culture media and gently mix.
(h) Centrifuge at 175 g for 10 min. Higher centrifugal forces will lead to cell death.
(i) Aspirate supernatant and suspend the cell pellet in culture medium.
(j) Cells are ready to be counted and tested or placed in culture. Do not delay getting the cells into culture medium and into the incubator.
   Stimulation of CD3+ T Cells with Dynabeads
(a) Plate isolated T-cells at a density of 1×10\hat{ }6 cells/mL in a 24 well plate in X-vivo media+10% human AB serum+300 IU/ml IL, 5 ng/ml IL-7, and 5 ng/ml of IL-15.
(b) Calculate the number of Dynabeads Human T-Activator CD3/CD28 beads (Gibco, Life Technologies) required to obtain 2:1 ratio (beads:cells) and wash with 1×PBS with 0.2% BSA, collecting beads using dynamagnet-2.
(c) Add washed beads at a 2:1 ratio or 1:2.5 (beads:cells) to the cells.
(d) Incubate cells for between 24-36 hours at 37° C. and 5% CO2.
(e) Remove beads using a dynamagnet-2.
(f) Culture cells without beads for at least 30 minutes before electroporation.

### Neon Transfection of CD3+ T Cells

(a) Stimulated T cells are electroporated using the Neon Transfection System (100 uL or 10 ul Kit, Invitrogen, Life Technologies).
(b) Pellet cells and wash once with PBS or T buffer.
(c) Resuspend cells at a density of 3×10≡cells in 10 uL of T buffer for 10 ul tip, and 1×10\hat{ }6 cells in 100 ul T buffer for 100 ul tips.
(d) Add specified mass of mRNA/DNA and electroporated at 1400 V, 10 ms, 3 pulses.
   a. For knockout using all mRNA:
      i. 100 ul tip: 15 ug Cas9 mRNA, 10 ug gRNA-RNA
      ii. 10 ul tip: 1.5 ug Cas9 mRNA, 1 ug gRNA-RNA
   b. When including plasmid donor for knock-in:
      i. 100 ul tip: 5-20 ug plasmid
      ii. 10 ul tip: 0.5-2 ug plasmid
   c. For sequential electroporations:
      On 0 hr timepoint deliver the amount of Cas9 specified in “a”
      ii. At time point 0 hr and 6-24 hr, deliver gRNA and plasmid together in amounts specified in “a” and “b”.
(e) After transfection, plate cells at 3000 cells/ul in antibiotic free culture media containing 10 ug/ml DNase I and incubate at 37° C. in 5% CO2 for ˜20 minutes.
(f) After recovery period, add 2 times volume of antibiotic containing media to well and culture at 37° C. in 5% CO2.
   rAAV Transduction of CD3+ T Cells
(a) Thaw rAAV on ice and mix well prior to addition to cells.
(b) Add specified MOI at the following timepoints post-electroporation
   a. For Cas9 mRNA edited cells:
      i. Add virus 4-6 hours post
   b. For Cas9 protein (RNP):
      i. Add virus 15 minutes post

### Post-Electroporation Culture of Primary T Cells

1. Observe media color post-electroporation as indicator for media addition. The timing will vary depending on the health of the cells for particular experiments/donors. When media begins to turn orange in color (as early as 48 hrs in some cases), double the volume of the culture media with culture media containing 2× concentration of cytokines (2× media). Continue this process as needed over the course of culture period.
2. In some cases, if cells are growing very rapidly (particularly around day 7-9) and media is become spent quickly the cells can be spun down and reconstituted in 2-3 times volume of 1× media.
3. In cases where cells are growing poorly and 3-4 days have passed without a need for media doubling, carefully remove ˜50% of the media by pipetting from the top being cautious to not disturb cells settled on the bottom of the flask and replace with equal volume 2× media.

### Exemplary Protocol 2 (Additional Stimulation): Modifications Over Exemplary Protocol 1

Reagents and Materials

(A) Culturing Media: 1 L OpTmizer™ T-Cell Expansion Basal Medium (Gibco Cat #A10221-01) with 2.6% OpTmizer™ T-Cell Expansion Supplement (Gibco Cat #A10484-02), 2.5% CTS™ Immune Cell Serum Replacement (Gibco Cat #A25961-01), 1% L-Glutamine (Gibco Cat #25030-081), 1% Penicillin/Streptomycin (Millipore Cat #TMS-AB2-C), 10 mM N-Acetyl-L-cysteine (Sigma Cat #A9165-256), 3001 U/ml Recombinant Human IL-2 (Peprotech Cat #200-02), 5 ng/ml Recombinant Human IL-7 (Peprotech Cat #200-07), 5 ng/ml Recombinant Human IL-15 (Peprotech Cat #200-15).
(B) Recovery Media: Culturing Media without Penicillin/Streptomycin.

### (C) Freezing Media: Cryostor CS10 (Stemcell Cat #07930).

(D) Separation Buffer: 1 L Phosphate Buffered Saline 1× (Hyclone Cat #SH302-56-01) with 0.2% Human AB Serum Heat Inactivated (Valley Biomedical Cat #HP1022HI), 1% Penicillin/Streptomycin (Millipore Cat #TMS-AB2-C) and 0.1 M EDTA pH 8.0 (Invitrogen Cat #AM9261)
(E) FACS Buffer: 500 mls Phosphate Buffered Saline 1× (Hyclone Cat #SH302-56-01) with 0.5% Penicillin/Streptomycin (Millipore Cat #TMS-AB2-C), 0.1% Human AB Serum Heat Inactivated (Valley Biomedical Cat #HP1022HI) and 0.1 M EDTA pH 8.0 (Invitrogen Cat #AM9261)

### (F) Cell Separation Reagents: Human T-cell Isolation Kit (Stem Cell Technologies Cat #17951) and ACK Lysing Buffer (Quality Biological Cat #118-156-101).

### (G) Additional Reagents: Dynabeads Human T-Activator CD3/CD28 (Gibco Cat #11132D), Amaxa 4D-Nucleofector X Kits (Lonza Cat #V4XP-3032, V4XP-3024), Stemcell EasySep Human T-cell Isolation kit (Cat #19051).

(H) Antibodies: APC Mouse Anti Human CD3 (BD Pharmingen Cat #555335), Anti Mouse TCRb PE CY 7 Clone H57-597 (eBioscience Cat #25-5961-80), Anti Mouse TCRb PE CY 7 Clone SK7 (BD Biosciences Cat #340440), and Fixable Viability Dye eFluor 780 (eBioscience Cat #65-0865-14).
(I) Materials: DynaMag™-2 magnet (ThermoFisher Scientific Cat #12321D), The Big Easy EasySep™ Magnet (Stemcell Cat #18001), and The Invitrogen™ Countess™ II FL Automated Cell Counter (ThermoFisher Scientific Cat #AMQAF1000).

Isolation of peripheral blood mononuclear cells (PBMCs) from a Trima cone using Ammonium Chloride based RBC lysis is performed as previously described.

Isolation of CD3+ T Cells Using EasySep Human T-Cell Isolation Kit (Cat #19051)

(A) Count Ficoll Separated PBMCs (*or washed apheresis unit/Leukopac) and adjust cell density to 1×10\hat{ }7 cells/ml.
(B) Transfer up to 8 mL to 14 ml round bottom tube (For use with “The Big Easy” EasySep magnet).
(C) Add 50 uL/mL of the Isolation Cocktail to the cells.
(D) Mix by pipetting and incubate for 5 minutes at room temperature.
(E) Vortex RapidSpheres for 30 seconds and add 40 uL/mL to the sample. Mix by pipetting up and down and incubate at RT for 3 minutes.
(F) Top up to 5 mL for samples <4 mLs, Top up to 10 ml for samples >4 mls.
(G) Remove unbound T-cells by carefully removing supernatant in one round of pipetting using sterile pipette to transfer to new conical tube.
(H) Count T-cells and validate purity by flow cytometry for % CD3+(>90%).
(I) Aliquot and freeze unused cells for future use with Cryostor CS10 (Stemcell Cat #07930).

Amaxa Nucleofection of CD3+ T Cells

Stimulated T cells are electroporated using the Lonza 4D Nucleofector™ X Unit & Amaxa 4D-Nucleofector X Kits using P3 buffer (V4XP-3032, V4XP-3024).
1) For the P3 kit the buffer solution Master Mix must be prepared beforehand and allowed to come to RT. Once mixed it will be good for 90 days stored at 4° C. so only make slightly more than is required for a given experiment.
   18 uL Supplement 1+82 uL P3 Primary Cell Nucleofector Solution
      100 ul cuvette: 90 uL P3 buffer mix/reaction
      20 ul cuvette: 18 uL P3 buffer mix/reaction
2) Mix and agitate cells well using a pipette to disrupt binding to the Dynabeads.
3) Remove beads using a dynamagnet-2.
4) Wash cells once with PBS at 400×g for 5 min.
5) Resuspend cells and count.
   For 100 ul cuvette you can use 2-20×10\hat{ }6 cells/condition.
   For 20 ul cuvette you can use 0.5-1×10\hat{ }6 cells/condition.
6) Move appropriate number of cells +1 extra reaction's worth to a new 50 mL conical (i.e. for 10 reactions, start with 11×10\hat{ }6 cells).
7) Fill conical to 50 mL with PBS and spin at 200×g for 10 min.
8) Aspirate the PBS as carefully as possible by slowly decanting the conical while the aspirator collects liquid. Do not move aspirator lower than the angled lip at the bottom of the tube as the pellet will be loose. We have found it best to simply hold it in this manner for 15-20 s.
9) Resuspend cells in the P3 Master Mix you have prepared.
   100 ul cuvette: 90 uL P3 buffer mix/reaction
   20 ul cuvette: 18 uL P3 buffer mix/reaction
10) Add desired volume of mRNA/DNA to 100 uL PCR tubes on ice in a sterile environment
   For knockout using all mRNA:
      100 ul cuvette: 5-15 ug Cas9 mRNA, 5-25 ug gRNA
      20 ul cuvette: 1-3 ug Cas9 mRNA, 1-5 ug gRNA
   When including plasmid donor/DNA:
      100 ul cuvette: 5-10 ug plasmid
      20 ul cuvette: 1-2 ug plasmid
   Nucleic acid amounts scale based on reaction volume not cell number in this system so large cuvettes should contain 5× optimized amounts of mRNA/gRNA/DNA from the small cuvettes whether using 2 or 20 million cells.
   Be sure to use concentrated nucleic acids for this protocol (1 ug/uL or greater) to ensure you are not diluting out the buffer reagents.
11) Add Master Mix containing cells to each tube prepared in step 10.
   100 ul cuvette: 90 uL P3 buffer mix with cells/reaction
   20 ul cuvette: 18 uL P3 buffer mix with cells/reaction
12) Mix each tube once with a pipette to incorporate all reagents and move total reaction mixture into the appropriate cuvette.
   Maximum loading volume of cuvettes—leave any extra in PCR tube
      100 ul cuvette: 120 uL
      20 ul cuvette: 24 uL
13) CAP IT, TAP IT, ZAP it
   Place cap on cuvette/s
   Tap lightly on a flat surface several times to ensure that any bubbles are removed
   Take to the Amaxa X module and electroporate the sample
      For all mRNA/gRNA zaps use program EO-115
      For all zaps containing DNA use program FI-115
14) After nucleofection allow cells to rest for 10-15 minutes in cuvette in hood.
15) During this incubation prepare a recovery plate containing 300 ul per well of 24 well plate if using 20 ul cuvette & 1 ml per well of 6 well plate if using 100 ul cuvette. Be sure to use recovery media for this (Culture Media with no antibiotics) *Include 1 ug DNASE in recovery wells if plasmid DNA is used*
16) After 15 minutes transfer to recovery plate by taking 80 uL of recovery media from the plate that you have set up and adding it to the cuvette.
17) Incubate at 37° C. and 5% CO2 for 30-60 minutes.
18) After 30 min incubation add additional regular media with antibiotics to bring cells up to 1×10\hat{ }6 cell/ml and culture at 37° C. in 5% CO2.
   700 uL for 1×10\hat{ }6 cells in a 24 well plate
   3 mL for 2-20×10\hat{ }6 cells in a 6 well plate
19) Culture cells, feed by carefully pulling old medium off top of wells and adding back in new or moving cells up to larger wells/plates as needed to culture.

Additional “Continuous” Stimulation of T Cells

(A) Calculate the # of Dynabeads Human T-Activator CD3/CD28 beads required to obtain 1:2 ratio (beads:cells) and wash with Culturing Media, collecting beads using dynamagnet-2. Utilize ¼ the amount used in the initial T cell activation.
(B) During the addition of media at step 18 in the Amaxa nucleofection protocol, add beads to the regular culture media before adding to cells.
(C) Add bead/media mixture to the cells and gently mix once.
(D) Culture cells normally, do not pipette wells to break up clumps of beads/cells.

Flow Cytometry

(A) Using cell counts, pull 0.5-1×10\hat{ }6 cells per sample to perform FACS.
(B) Prepare cells by adding 1×PBS to wash, spin at 1000×g for 3 minutes, decant supernatant off then add stain according to manufacturer recommendations for each antibody.
(C) Mix and let incubate 20-30 minutes in the dark.
(D) Following 30 minute incubation add 1 mL FACS Buffer to quench, spin again, and decant supernatant off.
(E) Repeat wash 1 more time with FACS Buffer.
(F) Resuspend pellet in 300 ul FACS Buffer to run FACS.

### Example 7: Examination of Transfection Efficiency by Flow Cytometry

Electroporated T cells are analyzed by flow cytometry ˜24-48 hours after transfection to test for expression of GFP or other fluorochrome (marker for transgene expression). For knockout experiments, analysis for loss of target protein is conducted between 7-9 days post transfection. For knock-in experiments, measure marker expression on day 7 and day 14. Cells are prepared by washing with chilled 1×PBS with 0.5% FBS and stain according to manufacturer recommendations for each antibody.

### Example 8: DNase Treatment Increased T Cell Survival after Electroporation

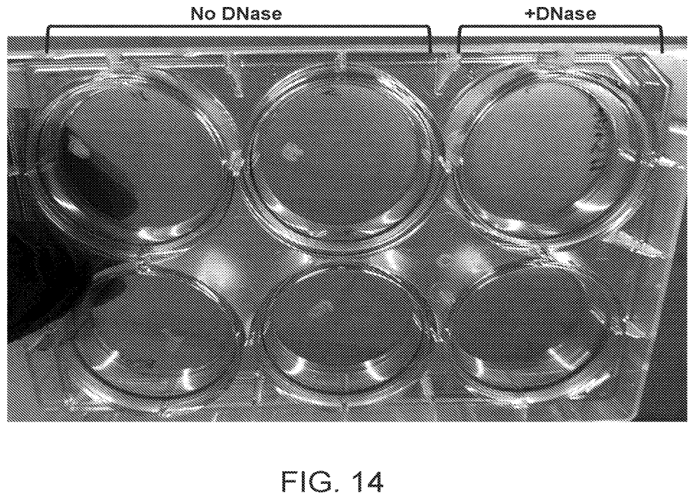

This example examined the effect of DNase on post-electroporation T cell survival. As shown in the representative photo in FIG. 14, 24 hours following electroporation with plasmid donor vector, activated T cell culture that was not treated with DNase showed cell clumping and dead cells floating on the medium, while T cell culture treated with DNase did not show cell clumping or floating cell corpses.

### Example 9: DNase Treatment Increased Viability and Transfection Efficiency of T Cells

This example examined the effect of DNase on post-electroporation T cell survival as well as transfection efficiency. Primary human T cells were cultured and stimulated with IL-2, IL-7, and IL-15. Later the T cells were either pulsed (control) or transfected with 1.5 μg of pMND-GFP plasmid (about 7.5 kb) at 36 hr or 48 hr post-stimulation. For comparison, DNase was added to recovery media of one group of transfected cells at 10 μg/ml. Cells were incubated in this recovery media following electroporation for 30 min. And after recovery, 2 times volume of complete media was added without any wash step (therefore diluted DNase remained in the media).

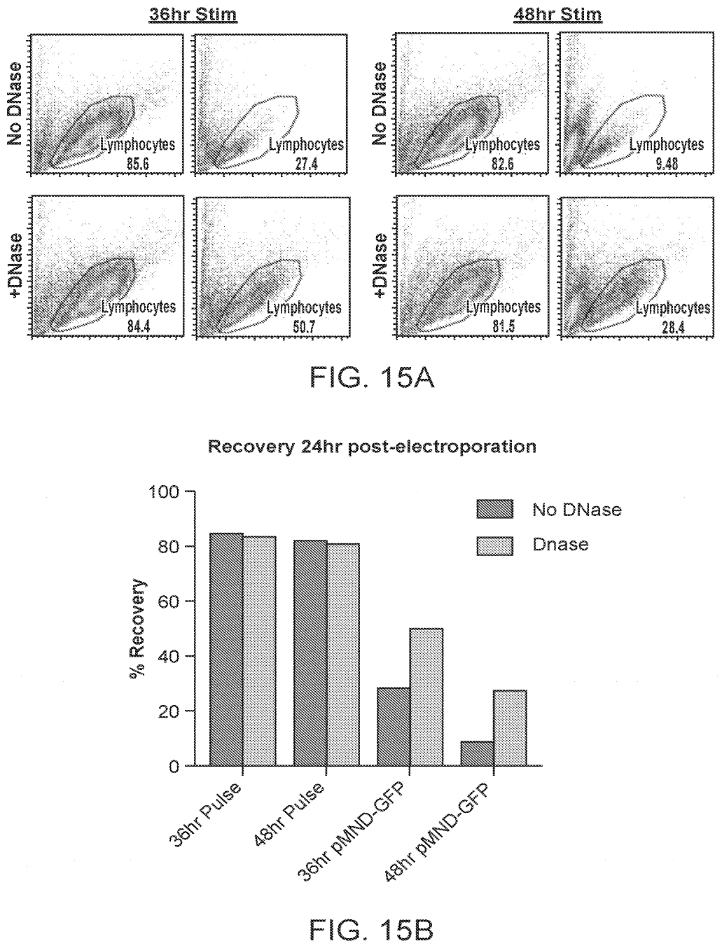

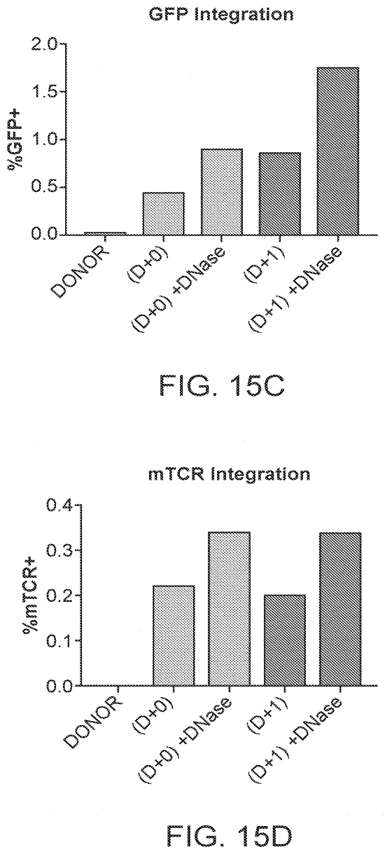

Cells were analyzed 24 hr post electroporation by flow cytometry to determine the percentage of recovered viable cells, as shown in FIG. 15A. FIG. 15B is a graph showing the percent recovery of the transfected cells in each group. DNase increased percent recovery in both “36 hr pMND-GFP” group, where cells were transfected with pMND-GFP plasmid at 36 hr post-stimulation, and “48 hr pMND-GFP” group, where cells were transfected with pMND-GFP at 48 hr post-stimulation.

Transfection efficiency was also assessed by examining the stable expression of the transgenes introduced by the plasmids. FIG. 15C is a graph showing percentage of GFP-expressing cells in each group of cells, and FIG. 15D is a graph showing percentage of mTCR-expressing cells in each group of cells. In these experiments, the primary T cells were transfected through electroporation with plasmid donor expressing GFP or mTCR on day 0 or 1, FACs was performed to examine the transgene expression on day 14 post electroporation. As shown in FIGS. 15C and 15D, DNase treatment increased integration efficiency for both GFP and mTCR under all tested conditions.

### Example 10. DNase and RS-1 Treatment Increased Transfection Efficiency of T Cells

This example examined the effects of treatment of electroporated T cells with DNase, RS-1, or both DNase and RS-1, on transfection efficiency. Primary T cells were transfected through electroporation with plasmid donor expressing GFP or mTCR on day 0 or 1, FACs was performed to examine the transgene expression on day 14 post electroporation.

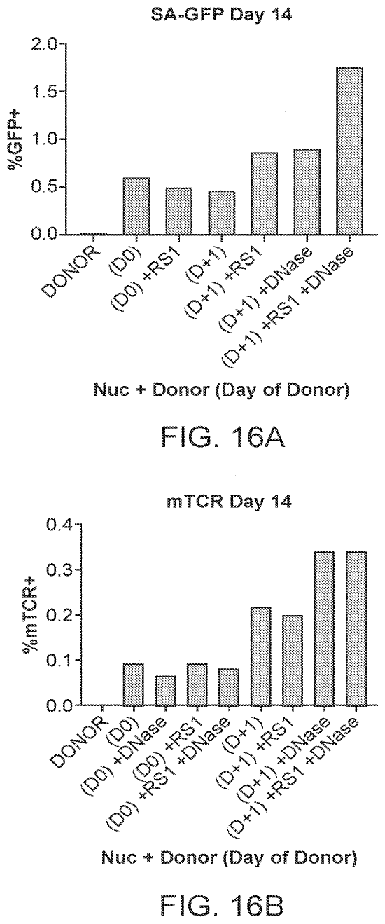

FIGS. 16A and 16B show percentage of GFP+ and mTCR+ cells, respectively. As shown in the figures, when T cells were transfected on day 1, treatment of DNase and RS-1 combined with DNase both promoted GFP expression and mTCR expression.

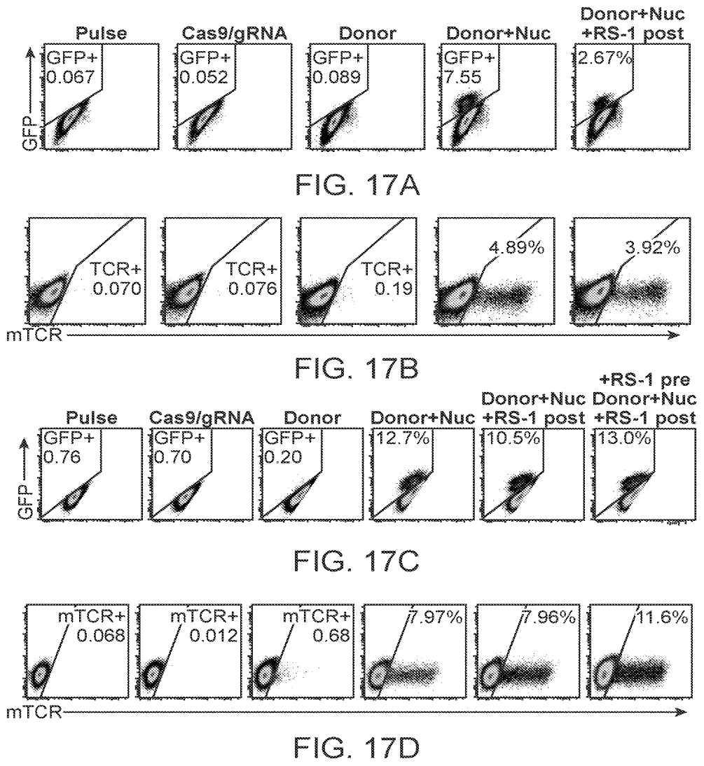

FIGS. 17A-17D are FACs density plots of T cells on day 7 post electroporation. FIG. 17A shows day 7 percent GFP expression of T cells that were electroporated on day 0 post stimulation with pulse (control), Cas9 and gRNA, donor (GFP), donor and DNase, or donor, DNase, and RS-1. FIG. 17B shows day 7 percent mTCR expression of T cells that were electroporated on day 0 post stimulation with pulse (control), Cas9 and gRNA, donor (GFP), donor and DNase, or donor, DNase, and RS-1. FIG. 17C shows day 7 percent GFP expression of T cells that were electroporated on day 1 post stimulation with pulse (control), Cas9 and gRNA, donor (GFP), donor and DNase, or donor, DNase, and RS-1. FIG. 17D shows day 7 percent mTCR expression of T cells that were electroporated on day 1 post stimulation with pulse (control), Cas9 and gRNA, donor (GFP), donor and DNase, or donor, DNase, and RS-1. Number in each plot shows the percentage of cells with positive GFP or mTCR signal.

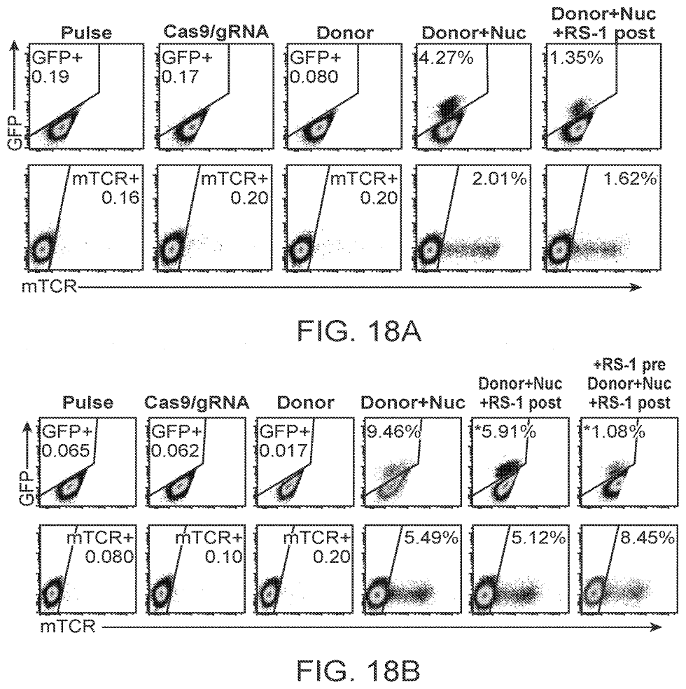

FIGS. 18A-18B are FACs density plots of T cells on day 14 post electroporation. FIG. 18A shows day 14 percent GFP and mTCR expression of T cells electroporated on day 0 post stimulation with pulse (control), Cas9 and gRNA, donor (GFP or mTCR), donor and DNase, or donor, DNase, and RS-1. FIG. 5B shows day 14 percent GFP and mTCR expression of T cells electroporated on day 1 post stimulation with pulse (control), Cas9 and gRNA, donor (GFP or mTCR), donor and DNase, or donor, DNase, and RS-1.

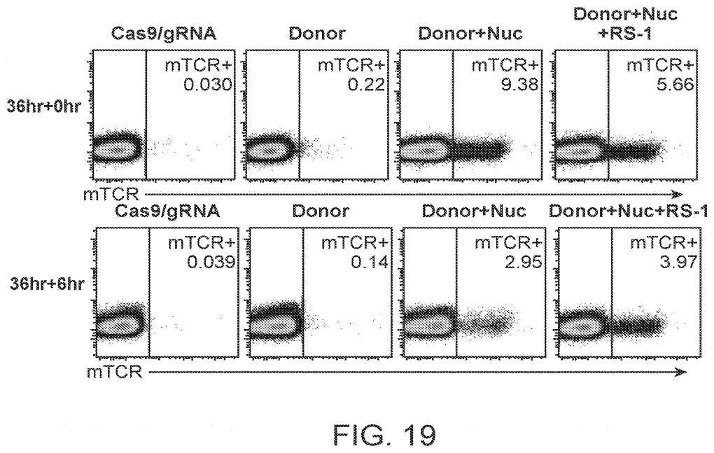

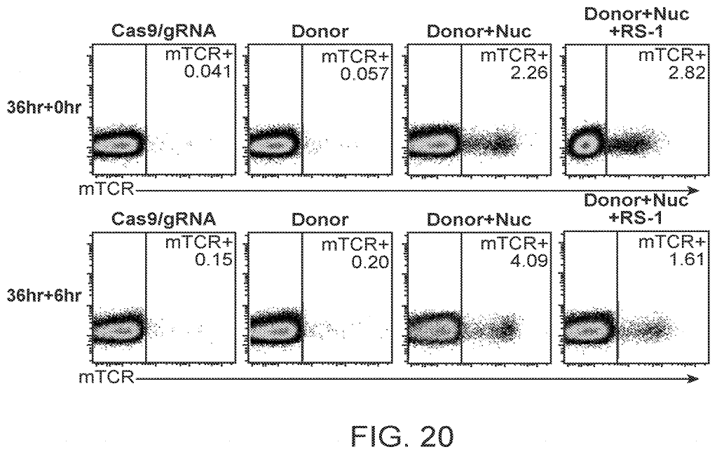

FIG. 19 shows FACs analysis of electroporation efficiency for T cells from donor 055330 electroporated with or without RS-1, or DNase and a mTCR at 36 hours post stimulation or 36 hours post stimulation and 6 hours post initial electroporation.

FIG. 20 shows FACs analysis of electroporation efficiency for T cells from donor 119866 electroporated with or without RS-1, or DNase and a mTCR at 36 hours post stimulation or 36 hours post stimulation and 6 hours post initial electroporation.

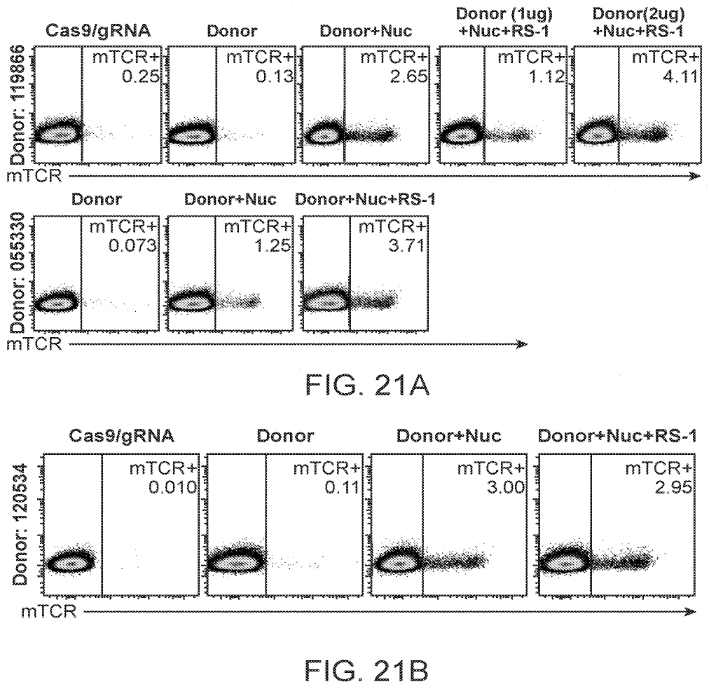

FIG. 21 shows FACs analysis of electroporation efficiency for T cells from donor 120534 electroporated with or without RS-1, or DNase and a mTCR at 36 hours post stimulation or 36 hours post stimulation and 6 hours post initial electroporation.

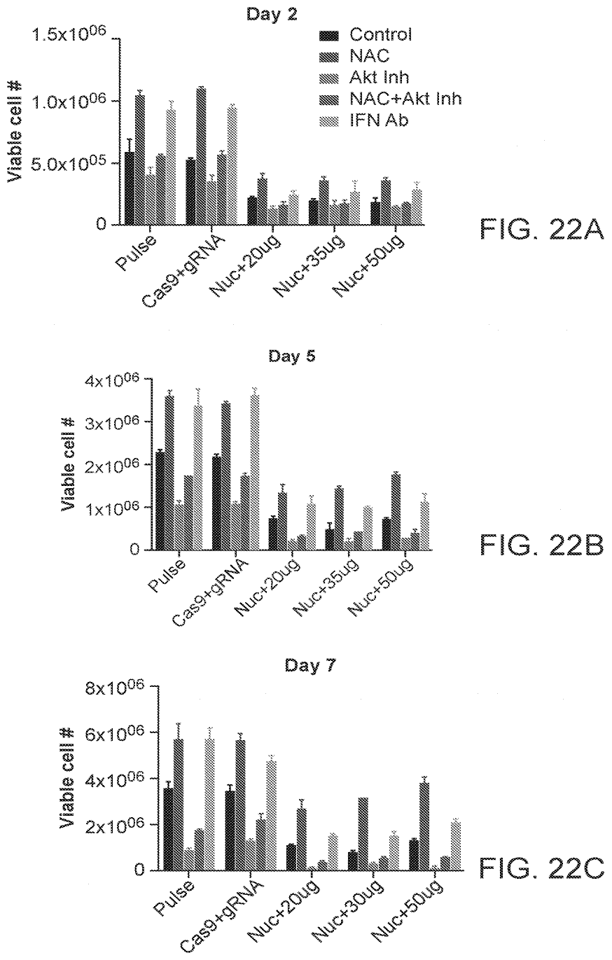

FIG. 22A shows FACs analysis of electroporation efficiency for T cells from donors 055330 and 119866 electroporated with or without RS-1, or DNase and a mTCR at 36 hours post stimulation and 24 hours post initial electroporation. FIG. 22B shows FACs analysis of electroporation efficiency for donor 120534 electroporated with or without RS-1, or DNase and a mTCR at 36 hours post stimulation and 24 hours post initial electroporation.

### Example 11. Effects of NAC, Akt Inhibitor, and Anti-IFNAR2 on Viability and Transfection Efficiency of T Cells

This example examined the effects of treatment with NAC, Akt VIII inhibitor, or anti-IFNAR2, on post-electroporation T cell survival as well as transfection efficiency. In these experiments, 2×10⁶ cells in 100 μl were electroporated non-sequentially at 36 hr post-stimulation. After electroporation, the cells were recovered for 15 min, and then split equally among 5 different supplement conditions, as listed in Table 5. NAC was added to the medium at 10 mM for duration, Akt VIII inhibitor at 8 μM for duration, and anti-IFNAR2 antibody was added to the media once at 10 μg/ml. “Nuc” in Table 2 and FIGS. 22A-22D denotes the condition where exogenous DNA was added to be inserted into cell genome at 20 μg (“+20 μg), 35 μg (“+35 μg), or 50 μg (“+50 μg”).

TABLE 5
Supplemental Conditions

| | | Akt VIII | NAC + Akt | IFNAR2 |
|---|---|---|---|---|
| Control | NAC | inhibitor | VIII | Antibody |
| Pulse | Pulse | Pulse | Pulse | Pulse |
| Cas9, gRNA | Cas9, gRNA | Cas9, gRNA | Cas9, gRNA | Cas9, gRNA |
| Nuc + 20 μg | Nuc + 20 μg | Nuc + 20 μg | Nuc + 20 μg | Nuc + 20 μg |
| Nuc + 30 μg | Nuc + 30 μg | Nuc + 30 μg | Nuc + 30 μg | Nuc + 30 μg |
| Nuc + 50 μg | Nuc + 50 μg | Nuc + 50 μg | Nuc + 50 μg | Nuc + 50 μg |

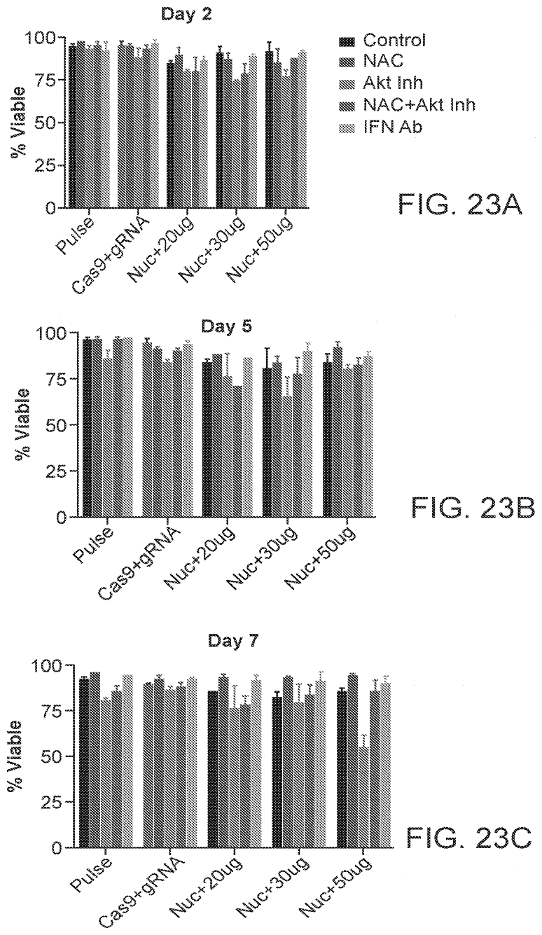

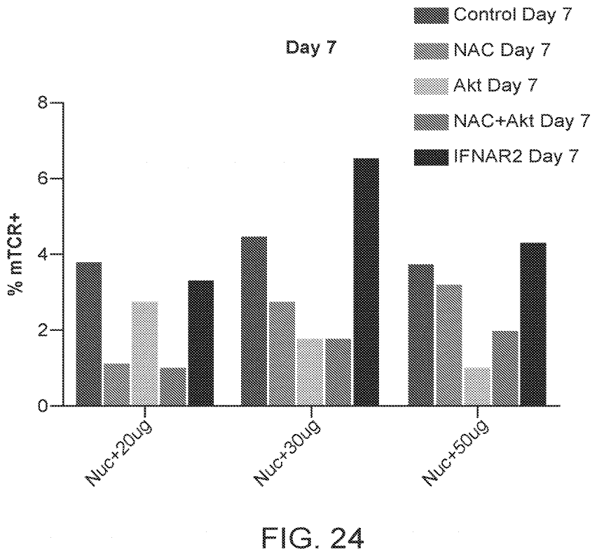

FIG. 23A-FIG. 23C show graphs of viable cell count in each condition on day 2, 5, and 7 post-electroporation, respectively. FIG. 23D-FIG. 23F show graphs of percentage of viable cells in each condition on day 2, 5, and 7 post-electroporation, respectively. As shown in FIG. 23B and FIG. 23C, under the experimental conditions, NAC treatment as well as IFNAR2 antibody treatment increased cell viability on day 7 post-electroporation. FIG. 24 shows a graph of percentage of mTCR positive cells on day 7 post-electroporation, it was found that when exogenous DNA was added at 30 μg and 50 μg, treatment with IFNAR2 antibody increased the percentage of mTCR expressing cells, suggesting an increase in integration efficiency.

### Example 12. Evaluation of DNA Repair Proteins in Donor Transgene Expression

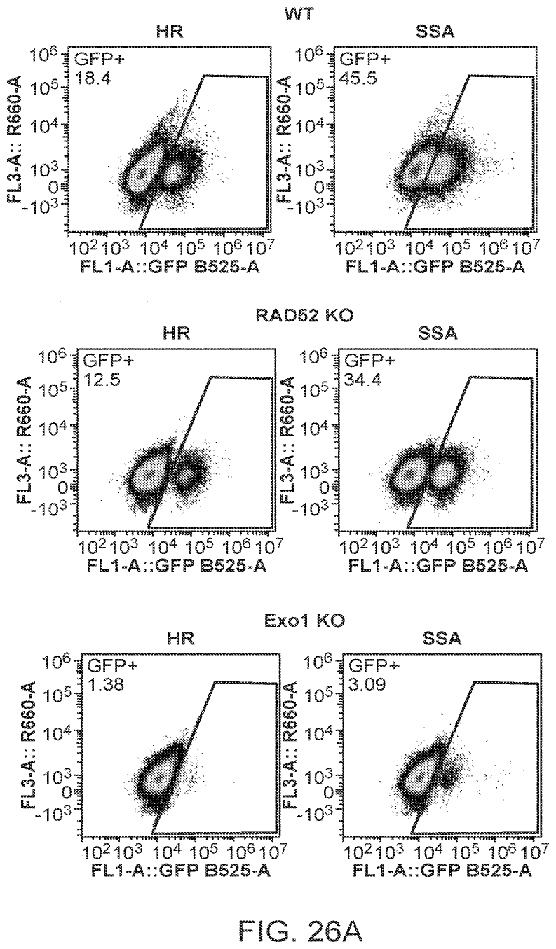

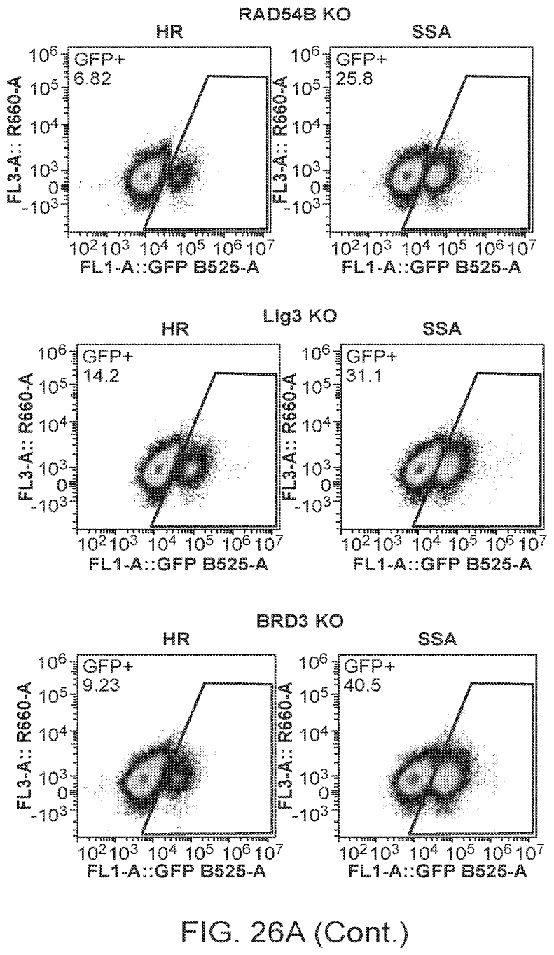

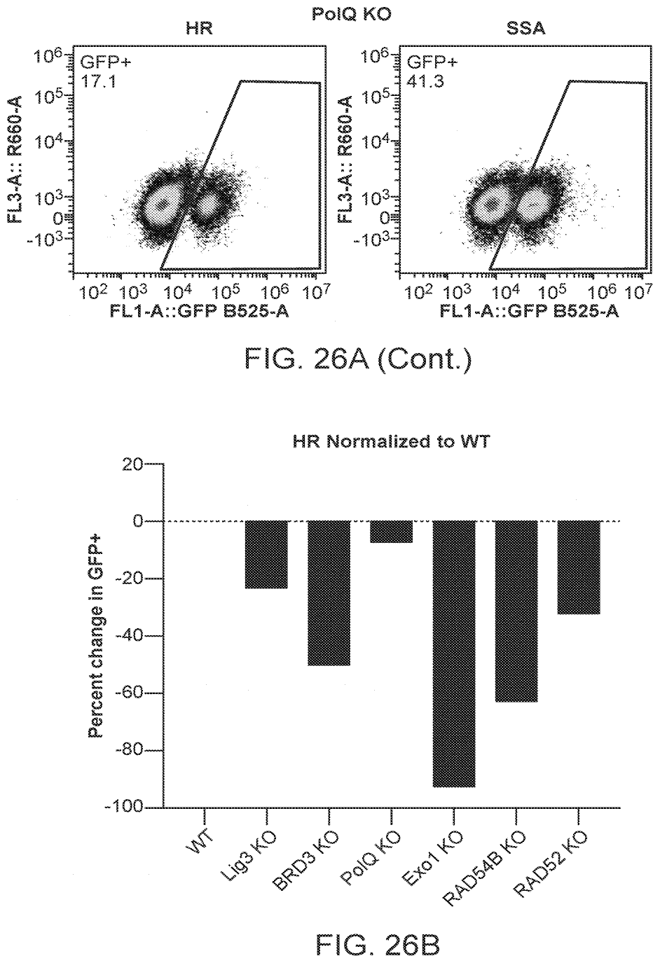

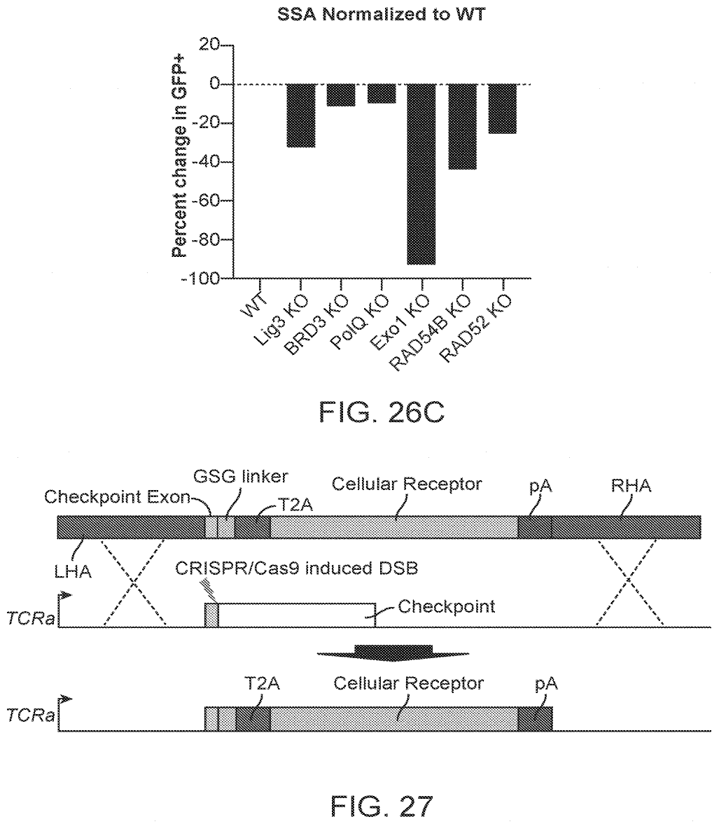

Exemplary DNA repair proteins, for example those implicated in repair mechanisms such as SSA or HR, were knocked out in the HCT116 cell line. Modified cells were utilized in an in vitro assay to determine if any repair protein has an effect on the expression of a donor, such as a cellular receptor, in a cell that has undergone transfection. Cells having knock outs in RAD52, Exo1, PolQ, BRD3, Lig3, RAD54B, or none (WT) were electroporated with an AAVS1 splice acceptor (SA)-GFP donor. Flow cytometry results measured on day 10 post electroporation are shown in FIG. 26A and charted in FIG. 26B and FIG. 26C.

### Example 13. Timing of Delivery of Transgene Donor and Knock-In Efficiency Electroporation Timing

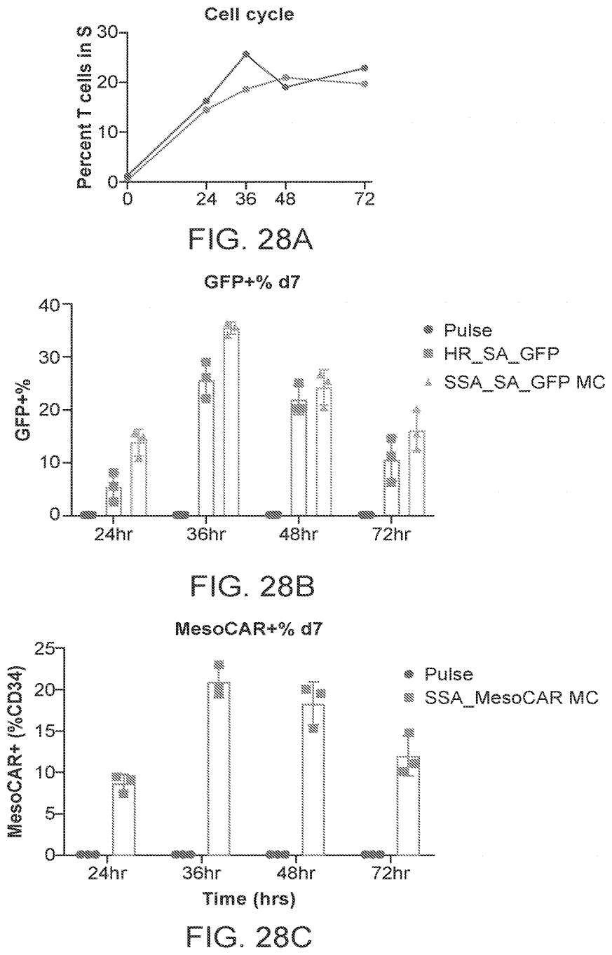

To determine if delivery timing of a transgene donor to cells plays any role in transgene expression, cells were transfected with 1 ug of an exemplary splice acceptor GFP donor (HR or SSA donor) with homology arms specific to AAVS1 (left homology arm from the adeno-associated virus integration site (AAVS1) within intron 1 of the human PPP1R12C gene) or 1 ug of an exemplary chimeric antigen receptor delivered via minicircle vector (anti-mesothelin CAR SSA donor) transgene at 24 hrs., 36 hr., 48 hrs., and 72 hrs. points post stimulation. Cells were evaluated for expression of GFP or CAR 7 days post electroporation. FIG. 28A shows the perfect T cells in S phase of control cells vs cells delivered an HR donor. FIG. 28B shows percent GFP on day 7 post electroporation and FIG. 28C shows percent CD34 (CAR) on day 7 post electroporation.

For reporting purposes enhanced GFP was utilized. The mammalian codon-optimized sequence comprises:

(SEQ ID NO: 87)
MVSKGEELFTGVVPILVELDGDVNGHKFSVSGEGEG
DATYGKLTLKFICTTGKLPVPWPTLVTTLTYGVQCF
SRYPDHMKQHDFFKSAMPEGYVQERTIFFKDDGNYK
TRAEVKFEGDTLVNRIELKGIDFKEDGNILGHKLEY
NYNSHNVYIMADKQKNGIKVNFKIRHNIEDGSVQLA
DHYQQNTPIGDGPVLLPDNHYLSTQSALSKDPNEKR
DHMVLLEFVTAAGITLGMDELYK.

TABLE 6
Exemplary polynucleic acid constructs

| SEQ | | |
|---|---|---|
| ID | | |
| NO | Identity | Sequence |
| 88 | pMC-HR- | acattaccctgttatccctagatgacattaccctg |
| | AAVS1- | ttatcccagatgacattaccctgttatccctagat |
| | SA-GFP | gacattaccctgttatccctagatgacatttaccc |
| | (HR). | tgttatccctagatgacattaccctgttatcccag |
| | Forward | atgacattaccctgttatccctagatacattaccc |
| | homology | tgttatcccagatgacataccctgttatccctaga |
| | arm | tgacattaccctgttatcccagatgacattaccct |
| | (735- | gttatccctagatacattaccctgttatcccagat |
| | 779; 45 | gacataccctgttatccctagatgacattaccctg |
| | bp in | ttatcccagatgacattaccctgttatccctagat |
| | size); | acattaccctgttatcccagatgacataccctgtt |
| | Reverse | atccctagatgacattaccctgttatcccagatga |
| | homology | cattaccctgttatccctagatacattaccctgtt |
| | arm | atcccagatgacataccctgttatccctagatgac |
| | (3612- | attaccctgttatcccagatgacattaccctgtta |
| | 3651; 40 | tccctagatacattaccctgttatcccagatgaca |
| | bp in | taccctgttatccctagatgacattaccctgttat |
| | size) | cccagatgacattaccctgttatccctagatacat |
| | | taccctgttatcccagatgacataccctgttatcc |
| | | ctagatgacattaccctgttatcccagataaactc |
| | | aatgatgatgatgatgatggtcgagactcagcggc |
| | | cgcggtgccagggcgtgcccttgggctccccgggc |
| | | gcgactagtgaattctgctttctctgacctgcatt |
| | | ctctcccctgggcctgtgccgctttctgtctgcag |
| | | cttgtggcctgggtcacctctacggctggcccaga |
| | | tccttccctgccgcctccttcaggttccgtcttcc |
| | | tccactccctcttccccttgctctctgctgtgttg |
| | | ctgcccaaggatgctctttccggagcacttccttc |
| | | tcggcgctgcaccacgtgatgtcctctgagcggat |
| | | cctccccgtgtctgggtcctctccgggcatctctc |
| | | ctccctcacccaaccccatgccgtcttcactcgct |
| | | gggttcccttttccttctccttctggggcctgtgc |
| | | catctctcgtttcttaggatggccttctccgacgg |
| | | atgtctcccttgcgtcccgcctccccttcttgtag |
| | | gcctgcatcatcaccgtttttctggacaaccccaa |
| | | agtaccccgtctccctggctttagccacctctcca |
| | | tcctcttgctttctttgcctggacaccccgttctc |
| | | ctgtggattcgggtcacctctcactcctttcattt |
| | | gggcagctcccctaccccccttacctctctagtct |
| | | gtgctagctcttccagccccctgtcatggcatctt |
| | | ccaggggtccgagagctcagctagtcttcttcctc |
| | | caacccgggcccctatgtccacttcaggacagcat |
| | | gtttgctgcctccagggatcctgtgtccccgagct |
| | | gggaccaccttatattcccagggccggttaatgtg |
| | | gctctggttctgggtacttttatctgtcccctcca |
| | | ccccacagtggggccactagggacagcgatcgggt |
| | | acatcgatcgcaggcgcaatcttcgcatttctttt |
| | | ttccagatggtgagcaagggcgaggagctgttcac |
| | | cggggtggtgcccatcctggtcgagctggacggcg |
| | | acgtaaacggccacaagttcagcgtgtccggcgag |
| | | ggcgagggcgatgccacctacggcaagctgaccct |
| | | gaagttcatctgcaccaccggcaagctgcccgtgc |
| | | cctggcccaccctcgtgaccaccctgacctacggc |
| | | gtgcagtgcttcagccgctaccccgaccacatgaa |
| | | gcagcacgacttcttcaagtccgccatgcccgaag |
| | | gctacgtccaggagcgcaccatcttcttcaaggac |
| | | gacggcaactacaagacccgcgccgaggtgaagtt |
| | | cgagggcgacaccctggtgaaccgcatcgagctga |
| | | agggcatcgacttcaaggaggacggcaacatcctg |
| | | gggcacaagctggagtacaactacaacagccacaa |
| | | cgtctatatcatggccgacaagcagaagaacggca |
| | | tcaaggtgaacttcaagatccgccacaacatcgag |
| | | gacggcagcgtgcagctcgccgaccactaccagca |
| | | gaacacccccatcggcgacggccccgtgctgctgc |
| | | ccgacaaccactacctgagcacccagtccgccctg |
| | | agcaaagaccccaacgagaagcgcgatcacatggt |
| | | cctgctggagttcgtgaccgccgccgggatcactc |
| | | tcggcatggacgagctgtacaagtaacgcggccgc |
| | | ctgtgccttctagttgccagccatctgttgtttgc |
| | | ccctcccccgtgccttccttgaccctggaaggtgc |
| | | cactcccactgtcctttcctaataaaatgaggaaa |
| | | ttgcatcgcattgtctgagtaggtgtcattctatt |
| | | ctggggggtggggtggggcaggacagcaaggggga |
| | | ggattgggaagacaatagcaggcatgctggggatg |
| | | cggtgggctctatgggattggtgacagaaaagccc |
| | | catccttaggcctcctccttcctagtctcctgata |
| | | ttgggtctaacccccacctcctgttaggcagattc |
| | | cttatctggtgacacacccccatttcctggagcca |
| | | tctctctccttgccagaacctctaaggtttgctta |
| | | cgatggagccagagaggatcctgggagggagagct |
| | | tggcagggggtgggagggaagggggggatgcgtga |
| | | cctgcccggttctcagtggccaccctgcgctaccc |
| | | tctcccagaacctgagctgctctgacgcggctgtc |
| | | tggtgcgtttcactgatcctggtgctgcagcttcc |
| | | ttacacttcccaagaggagaagcagtttggaaaaa |
| | | caaaatcagaataagttggtcctgagttctaactt |
| | | tggctcttcacctttctagtccccaatttatattg |
| | | ttcctccgtgcgtcagttttacctgtgagataagg |
| | | ccagtagccagccccgtcctggcagggctgtggtg |
| | | aggaggggggtgtccgtgtggaaaactccctttgt |
| | | gagaatggtgcgtcctaggtgttcaccaggtcgtg |
| | | gccgcctctactccctttctctttctccatccttc |
| | | tttccttaaagagtccccagtgctatctgggacat |
| | | attcctccgcccagagcagggtcccgcttccctaa |
| | | ggccctgctctgggcttctgggtttgagtccttgg |
| | | caagcccaggagaggcgctcaggcttccctgtccc |
| | | ccttcctcgtccaccatctcatgcccctggctctc |
| | | ctgccccttcxctacaggggttcctggctctgctc |
| | | ttcagactgagccccgttcccctgcatccccgttc |
| | | ccctgcatcccccttcccctgcatcccccagaggc |
| | | cccaggccacctacttggcctggaccccacgagag |
| | | gccaccccagccctgtctaccaggctgccttttgg |
| | | gtggattctcctccaactgtggggtgactgcttgg |
| | | gatatctctagagtcgacccatgggggcccgcccc |
| | | aactggggtaacctttgagttctctcagttggggg |
| | | taatcagcatcatgatgtggtaccacatcatgatg |
| | | ctgattataagaatgcggccgccacactctagtgg |
| | | atctcgagttaataat |
| | | tcagaagaactcgtcaagaaggcgatagaaggcga |
| | | tgcgctgcgaatcgggagcggcgataccgtaaagc |
| | | acgaggaagcggtcagcccattcgccgccaagctc |
| | | ttcagcaatatcacgggtagccaacgctatgtcct |
| | | gatagcggtccgccacacccagccggccacagtcg |
| | | atgaatccagaaaagcggccattttccaccatgat |
| | | attcggcaagcaggcatcgccatgggtcacgacga |
| | | gatcctcgccgtcgggcatgctcgccttgagcctg |
| | | gcgaacagttcggctggcgcgagcccctgatgctc |
| | | ttcgtccagatcatcctgatcgacaagaccggctt |
| | | ccatccgagtacgtgctcgctcgatgcgatgtttc |
| | | gcttggtggtcgaatgggcaggtagccggatcaag |
| | | cgtatgcagccgccgcattgcatcagccatgatgg |
| | | atactttctcggcaggagcaaggtgtagatgacat |
| | | ggagatcctgccccggcacttcgcccaatagcagc |
| | | cagtcccttcccgcttcagtgacaacgtcgagcac |
| | | agctgcgcaaggaacgcccgtcgtggccagccacg |
| | | atagccgcgctgcctcgtcttgcagttcattcagg |
| | | gcaccggacaggtcggtcttgacaaaaagaaccgg |
| | | gcgcccctgcgctgacagccggaacacggcggcat |
| | | cagagcagccgattgtctgttgtgcccagtcatag |
| | | ccgaatagcctctccacccaagcggccggagaacc |
| | | tgcgtgcaatccatcttgttcaatcatgcgaaacg |
| | | atcctcatcctgtctcttgatcagagcttgatccc |
| | | ctgcgccatcagatccttggcggcgagaaagccat |
| | | ccagtttactttgcagggcttcccaaccttaccag |
| | | agggcgccccagctggcaattccggttcgcttgct |
| | | gtccataaaaccgcccagtctagctatcgccatgt |
| | | aagcccactgcaagctacctgctttctctttgcgc |
| | | ttgcgttttcccttgtccagatagcccagtagctg |
| | | acattcatccggggtcagcaccgtttctgcggact |
| | | ggctttctacgtgctcgaggggggccaaacggtct |
| | | ccagcttggctgttttggcggatgagagaagattt |
| | | tcagcctgatacagattaaatcagaacgcagaagc |
| | | ggtctgataaaacagaatttgcctggcggcagtag |
| | | cgcggtggtcccacctgaccccatgccgaactcag |
| | | aagtgaaacgccgtagcgccgatggtagtgtgggg |
| | | tctccccatgcgagagtagggaactgccaggcatc |
| | | aaataaaacgaaaggctcagtcgaaagactgggcc |
| | | tttcgttttatctgttgtttgtcggtgaacgctct |
| | | cctgagtaggacaaatccgccgggagcggatttga |
| | | acgttgcgaagcaacggcccggagggtggcgggca |
| | | ggacgcccgccataaactgccaggcatcaaattaa |
| | | gcagaaggccatcctgacggatggcctttttgcgt |
| | | ttctacaaactcttttgtttatttttctaaataca |
| | | ttcaaatatgtatccgctcatgaccaaaatccctt |
| | | aacgtgagttttcgttccactgagcgtcagacccc |
| | | gtagaaaagatcaaaggatcttcttgagatccttt |
| | | ttttctgcgcgtaatctgctgcttgcaaacaaaaa |
| | | aaccaccgctaccagcggtggtttgtttgccggat |
| | | caagagctaccaactctttttccgaaggtaactgg |
| | | cttcagcagagcgcagataccaaatactgtccttc |
| | | tagtgtagccgtagttaggccaccacttcaagaac |
| | | tctgtagcaccgcctacatacctcgctctgctaat |
| | | cctgttaccagtggctgctgccagtggcgataagt |
| | | cgtgtcttaccgggttggactcaagacgatagtta |
| | | ccggataaggcgcagcggtcgggctgaacgggggg |
| | | ttcgtgcacacagcccagcttggagcgaacgacct |
| | | acaccgaactgagatacctacagcgtgagctatga |
| | | gaaagcgccacgcttcccgaagggagaaaggcgga |
| | | caggtatccggtaagcggcagggtcggaacaggag |
| | | agcgcacgagggagcttccagggggaaacgcctgg |
| | | tatctttatagtcctgtcgggtttcgccacctctg |
| | | acttgagcgtcgatttttgtgatgctcgtcagggg |
| | | ggcggagcctatggaaaaacgccagcaacgcggcc |
| | | tttttacggttcctggccttttgctggccttttgc |
| | | tcacatgttctttcctgcgttatcccctgattctg |
| | | tggataaccgtattaccgcctttgagtgagctgat |
| | | accgctcgccgcagccgaacgaccgagcgcagcga |
| | | gtcagtgagcgaggaagcggaagagcgcctgatgc |
| | | ggtattttctccttacgcatctgtgcggtatttca |
| | | caccgcatatggtgcactctcagtacaatctgctc |
| | | tgatgccgcatagttaagccagtatacactccgct |
| | | atcgctacgtgactgggtcatggctgcgccccgac |
| | | acccgccaacacccgctgacgcgccctgacgggct |
| | | tgtctgctcccggcatccgcttacagacaagctgt |
| | | gaccgtctccgggagctgcatgtgtcagaggtttt |
| | | caccgtcatcaccgaaacgcgcgaggcagcagatc |
| | | aattcgcgcgcgaaggcgaagcggcatgcataatg |
| | | tgcctgtcaaatggacgaagcagggattctgcaaa |
| | | ccctatgctactccgtcaagccgtcaattgtctga |
| | | ttcgttaccaattatgacaacttgacggctacatc |
| | | attcactttttcttcacaaccggcacggaactcgc |
| | | tcgggctggccccggtgcattttttaaatacccgc |
| | | gagaaatagagttgatcgtcaaaaccaacattgcg |
| | | accgacggtggcgataggcatccgggtggtgctca |
| | | aaagcagcttcgcctggctgatacgttggtcctcg |
| | | cgccagcttaagacgctaatccctaactgctggcg |
| | | gaaaagatgtgacagacgcgacggcgacaagcaaa |
| | | catgctgtgcgacgctggcgat |
| 89 | pMC- | acattaccctgttatccctagatgacattaccctg |
| | AAVS1- | ttatcccagatgacattaccctgttatccctagat |
| | SSA48v | gacattaccctgttatccctagatgacatttaccc |
| | 3-SA- | tgttatccctagatgacattaccctgttatcccag |
| | GFP- | atgacattaccctgttatccctagatacattaccc |
| | 2cut_ | tgttatcccagatgacataccctgttatccctaga |
| | universal | tgacattaccctgttatcccagatgacattaccct |
| | gRNA | gttatccctagatacattaccctgttatcccagat |
| | (SSA/H | gacataccctgttatccctagatgacattaccctg |
| | MEJ) | ttatcccagatgacattaccctgttatccctagat |
| | Forward | acattaccctgttatcccagatgacat |
| | homology | accctgttatccctagatgacattaccctgttatc |
| | arm | ccagatgacattaccctgttatccctagatacatt |
| | (735- | accctgttatcccagatgacataccctgttatccc |
| | 779; 45 | tagatgacattaccctgttatcccagatgacatta |
| | bp in | ccctgttatccctagatacattaccctgttatccc |
| | length) | agatgacataccctgttatccctagatgacattac |
| | 5′ | cctgttatcccagatgacattaccctgttatccct |
| | universal | agatacattaccctgttatcccagatgacataccc |
| | guide | tgttatccctagatgacattaccctgttatcccag |
| | (780- | ataaactcaatgatgatgatgatgatggtcgagac |
| | 802; 23 | tcagcggccgcggtgccagggcgtgcccttgggct |
| | bp in | ccccgggcgcgactagtgggaggcgttcgggccac |
| | length) | agcggcccgttctgggtacttttatctgtcccctc |
| | Universal | caccccacagtggggccactacgatcgatcgatcg |
| | guide | caggcgcaatcttcgcatttcttttttccagatgg |
| | 3′ | tgagcaagggcgaggagctgttcaccggggtggtg |
| | (1891- | cccatcctggtcgagctggacggcgacgtaaacgg |
| | 1913; 23 | ccacaagttcagcgtgtccggcgagggcgagggcg |
| | bp in | atgccacctacggcaagctgaccctgaagttcatc |
| | length) | tgcaccaccggcaagctgcccgtgccctggcccac |
| | Reverse | cctcgtgaccaccctgacctacggcgtgcagtgct |
| | homology | tcagccgctaccccgaccacatgaagcagcacgac |
| | arm | ttcttcaagtccgccatgcccgaaggctacgtcca |
| | (1914- | ggagcgcaccatcttcttcaaggacgacggcaact |
| | 1947; | acaagacccgcgccgaggtgaagttcgagggcgac |
| | 34 | accctggtgaaccgcatcgagctgaagggcatcga |
| | bp in | cttcaaggaggacggcaacatcctggggcacaagc |
| | length) | tggagtacaactacaacagccacaacgtctatatc |
| | | atggccgacaagcagaagaacggcatcaaggtgaa |
| | | cttcaagatccgccacaacatcgaggacggcagcg |
| | | tgcagctcgccgaccactaccagcagaacaccccc |
| | | atcggcgacggccccgtgctgctgcccgacaacca |
| | | ctacctgagcacccagtccgccctgagcaaagacc |
| | | ccaacgagaagcgcgatcacatggtcctgctggag |
| | | ttcgtgaccgccgccgggatcactctcggcatgga |
| | | cgagctgtacaagtaattaattaatgagcggccgc |
| | | gtttcagacatgataagatacattgatgagtttgg |
| | | acaaaccacaactagaatgcagtgaaaaaaatgct |
| | | ttatttgtgaaatttgtgatgctattgctttattt |
| | | gtaaccattataagctgcaataaacaagttaacaa |
| | | caacaattgcattcattttatgtttcaggttcagg |
| | | gggaggtgtgggaggttttttgacgtcgggacagg |
| | | attggtgacagaaaagccccatccttaggcctcct |
| | | ccttcaaaccgctgtggcccgaacgcctcccgtcg |
| | | acccatgggggcccgccccaactggggtaaccttt |
| | | gagttctctcagttgggggtaatcagcatcatgat |
| | | gtggtaccacatcatgatgctgattataagaatgc |
| | | ggccgccacactctagtggatctcgagttaataat |
| | | tcagaagaactcgtcaagaaggcgatagaaggcga |
| | | tgcgctgcgaatcgggagcggcgataccgtaaagc |
| | | acgaggaagcggtcagcccattcgccgccaagctc |
| | | ttcagcaatatcacgggtagccaacgctatgtcct |
| | | gatagcggtccgccacacccagccggccacagtcg |
| | | atgaatccagaaaagcggccattttccaccatgat |
| | | attcggcaagcaggcatcgccatgggtcacgacga |
| | | gatcctcgccgtcgggcatgctcgccttgagcctg |
| | | gcgaacagttcggctggcgcgagcccctgatgctc |
| | | ttcgtccagatcatcctgatcgacaagaccggctt |
| | | ccatccgagtacgtgctcgctcgatgcgatgtttc |
| | | gcttggtggtcgaatgggcaggtagccggatcaag |
| | | cgtatgcagccgccgcattgcatcagccatgatgg |
| | | atactttctcggcaggagcaaggtgtagatgacat |
| | | ggagatcctgccccggcacttcgcccaatagcagc |
| | | cagtcccttcccgcttcagtgacaacgtcgagcac |
| | | agctgcgcaaggaacgcccgtcgtggccagccacg |
| | | atagccgcgctgcctcgtcttgcagttcattcagg |
| | | gcaccggacaggtcggtcttgacaaaaagaaccgg |
| | | gcgcccctgcgctgacagccggaacacggcggcat |
| | | cagagcagccgattgtctgttgtgcccagtcatag |
| | | ccgaatagcctctccacccaagcggccggagaacc |
| | | tgcgtgcaatccatcttgttcaatcatgcgaaacg |
| | | atcctcatcctgtctcttgatcagagcttgatccc |
| | | ctgcgccatcagatccttggcggcgagaaagccat |
| | | ccagtttactttgcagggcttcccaaccttaccag |
| | | agggcgccccagctggcaattccggttcgcttgct |
| | | gtccataaaaccgcccagtctagctatcgccatgt |
| | | aagcccactgcaagctacctgctttctctttgcgc |
| | | ttgcgttttcccttgtccagatagcccagtagctg |
| | | acattcatccggggtcagcaccgtttctgcggact |
| | | ggctttctacgtgctcgaggggggccaaacggtct |
| | | ccagcttggctgttttggcggatgagagaagattt |
| | | tcagcctgatacagattaaatcagaacgcagaagc |
| | | ggtctgataaaacagaatttgcctggcggcagtag |
| | | cgcggtggtcccacctgaccccatgccgaactcag |
| | | aagtgaaacgccgtagcgccgatggtagtgtgggg |
| | | tctccccatgcgagagtagggaactgccaggcatc |
| | | aaataaaacgaaaggctcagtcgaaagactgggcc |
| | | tttcgttttatctgttgtttgtcggtgaacgctct |
| | | cctgagtaggacaaatccgccgggagcggatttga |
| | | acgttgcgaagcaacggcccggagggtggcgggca |
| | | ggacgcccgccataaactgccaggcatcaaattaa |
| | | gcagaaggccatcctgacggatggcctttttgcgt |
| | | ttctacaaactcttttgtttatttttctaaataca |
| | | ttcaaatatgtatccgctcatgaccaaaatccctt |
| | | aacgtgagttttcgttccactgagcgtcagacccc |
| | | gtagaaaagatcaaaggatcttcttgagatccttt |
| | | ttttctgcgcgtaatctgctgcttgcaaacaaaaa |
| | | aaccaccgctaccagcggtggtttgtttgccggat |
| | | caagagctaccaactctttttccgaaggtaactgg |
| | | cttcagcagagcgcagataccaaatactgtccttc |
| | | tagtgtagccgtagttaggccaccacttcaagaac |
| | | tctgtagcaccgcctacatacctcgctc |
| | | accctgttatccctagatgacattaccctgttatc |
| | | ccagatgacattaccctgttatccctagatacatt |
| | | accctgttatcccagatgacataccctgttatccc |
| | | tagatgacattaccctgttatcccagatgacatta |
| | | ccctgttatccctagatacattaccctgttatccc |
| | | agatgacataccctgttatccctagatgacattac |
| | | cctgttatcccagatgacattaccctgttatccct |
| | | agatacattaccctgttatcccagatgacataccc |
| | | tgttatccctagatgacattaccctgttatcccag |
| | | ataaactcaatgatgatgatgatgatggtcgagac |
| | | tcagcggccgcggtgccagggcgtgcccttgggct |
| | | ccccgggcgcgactagtgggaggcgttcgggccac |
| | | agcggcccgttctgggtacttttatctgtcccctc |
| | | caccccacagtggggccactacgatcgatcgatcg |
| | | caggcgcaatcttcgcatttcttttttccagatgg |
| | | tgagcaagggcgaggagctgttcaccggggtggtg |
| | | cccatcctggtcgagctggacggcgacgtaaacgg |
| | | ccacaagttcagcgtgtccggcgagggcgagggcg |
| | | atgccacctacggcaagctgaccctgaagttcatc |
| | | tgcaccaccggcaagctgcccgtgccctggcccac |
| | | cctcgtgaccaccctgacctacggcgtgcagtgct |
| | | tcagccgctaccccgaccacatgaagcagcacgac |
| | | ttcttcaagtccgccatgcccgaaggctacgtcca |
| | | ggagcgcaccatcttcttcaaggacgacggcaact |
| | | acaagacccgcgccgaggtgaagttcgagggcgac |
| | | accctggtgaaccgcatcgagctgaagggcatcga |
| | | cttcaaggaggacggcaacatcctggggcacaagc |
| | | tggagtacaactacaacagccacaacgtctatatc |
| | | atggccgacaagcagaagaacggcatcaaggtgaa |
| | | cttcaagatccgccacaacatcgaggacggcagcg |
| | | tgcagctcgccgaccactaccagcagaacaccccc |
| | | atcggcgacggccccgtgctgctgcccgacaacca |
| | | ctacctgagcacccagtccgccctgagcaaagacc |
| | | ccaacgagaagcgcgatcacatggtcctgctggag |
| | | ttcgtgaccgccgccgggatcactctcggcatgga |
| | | cgagctgtacaagtaattaattaatgagcggccgc |
| | | gtttcagacatgataagatacattgatgagtttgg |
| | | acaaaccacaactagaatgcagtgaaaaaaatgct |
| | | ttatttgtgaaatttgtgatgctattgctttattt |
| | | gtaaccattataagctgcaataaacaagttaacaa |
| | | caacaattgcattcattttatgtttcaggttcagg |
| | | gggaggtgtgggaggttttttgacgtcgggacagg |
| | | attggtgacagaaaagccccatccttaggcctcct |
| | | ccttcaaaccgctgtggcccgaacgcctcccgtcg |
| | | acccatgggggcccgccccaactggggtaaccttt |
| | | gagttctctcagttgggggtaatcagcatcatgat |
| | | gtggtaccacatcatgatgctgattataagaatgc |
| | | ggccgccacactctagtggatctcgagttaataat |
| | | tcagaagaactcgtcaagaaggcgatagaaggcga |
| | | tgcgctgcgaatcgggagcggcgataccgtaaagc |
| | | acgaggaagcggtcagcccattcgccgccaagctc |
| | | ttcagcaatatcacgggtagccaacgctatgtcct |
| | | gatagcggtccgccacacccagccggccacagtcg |
| | | atgaatccagaaaagcggccattttccaccatgat |
| | | attcggcaagcaggcatcgccatgggtcacgacga |
| | | gatcctcgccgtcgggcatgctcgccttgagcctg |
| | | gcgaacagttcggctggcgcgagcccctgatgctc |
| | | ttcgtccagatcatcctgatcgacaagaccggctt |
| | | ccatccgagtacgtgctcgctcgatgcgatgtttc |
| | | gcttggtggtcgaatgggcaggtagccggatcaag |
| | | cgtatgcagccgccgcattgcatcagccatgatgg |
| | | atactttctcggcaggagcaaggtgtagatgacat |
| | | ggagatcctgccccggcacttcgcccaatagcagc |
| | | cagtcccttcccgcttcagtgacaacgtcgagcac |
| | | agctgcgcaaggaacgcccgtcgtggccagccacg |
| | | atagccgcgctgcctcgtcttgcagttcattcagg |
| | | gcaccggacaggtcggtcttgacaaaaagaaccgg |
| | | gcgcccctgcgctgacagccggaacacggcggcat |
| | | cagagcagccgattgtctgttgtgcccagtcatag |
| | | ccgaatagcctctccacccaagcggccggagaacc |
| | | tgcgtgcaatccatcttgttcaatcatgcgaaacg |
| | | atcctcatcctgtctcttgatcagagcttgatccc |
| | | ctgcgccatcagatccttggcggcgagaaagccat |
| | | ccagtttactttgcagggcttcccaaccttaccag |
| | | agggcgccccagctggcaattccggttcgcttgct |
| | | gtccataaaaccgcccagtctagctatcgccatgt |
| | | aagcccactgcaagctacctgctttctctttgcgc |
| | | ttgcgttttcccttgtccagatagcccagtagctg |
| | | acattcatccggggtcagcaccgtttctgcggact |
| | | ggctttctacgtgctcgaggggggccaaacggtct |
| | | ccagcttggctgttttggcggatgagagaagattt |
| | | tcagcctgatacagattaaatcagaacgcagaagc |
| | | ggtctgataaaacagaatttgcctggcggcagtag |
| | | cgcggtggtcccacctgaccccatgccgaactcag |
| | | aagtgaaacgccgtagcgccgatggtagtgtgggg |
| | | tctccccatgcgagagtagggaactgccaggcatc |
| | | aaataaaacgaaaggctcagtcgaaagactgggcc |
| | | tttcgttttatctgttgtttgtcggtgaacgctct |
| | | cctgagtaggacaaatccgccgggagcggatttga |
| | | acgttgcgaagcaacggcccggagggtggcgggca |
| | | ggacgcccgccataaactgccaggcatcaaattaa |
| | | gcagaaggccatcctgacggatggcctttttgcgt |
| | | ttctacaaactcttttgtttatttttctaaataca |
| | | ttcaaatatgtatccgctcatgaccaaaatccctt |
| | | aacgtgagttttcgttccactgagcgtcagacccc |
| | | gtagaaaagatcaaaggatcttcttgagatccttt |
| | | ttttctgcgcgtaatctgctgcttgcaaacaaaaa |
| | | aaccaccgctaccagcggtggtttgtttgccggat |
| | | caagagctaccaactctttttccgaaggtaactgg |
| | | cttcagcagagcgcagataccaaatactgtccttc |
| | | tagtgtagccgtagttaggccaccacttcaagaac |
| | | tctgtagcaccgcctacatacctcgctctgc |
| | | taatcctgttaccagtggctgctgccagtggcg |
| | | ataagtcgtgtcttaccgggttggactcaagacga |
| | | tagttaccggataaggcgcagcggtcgggctgaac |
| | | ggggggttcgtgcacacagcccagcttggagcgaa |
| | | cgacctacaccgaactgagatacctacagcgtgag |
| | | ctatgagaaagcgccacgcttcccgaagggagaaa |
| | | ggcggacaggtatccggtaagcggcagggtcggaa |
| | | caggagagcgcacgagggagcttccagggggaaac |
| | | gcctggtatctttatagtcctgtcgggtttcgcca |
| | | cctctgacttgagcgtcgatttttgtgatgctcgt |
| | | caggggggcggagcctatggaaaaacgccagcaac |
| | | gcggcctttttacggttcctggccttttgctggcc |
| | | ttttgctcacatgttctttcctgcgttatcccctg |
| | | attctgtggataaccgtattaccgcctttgagtga |
| | | gctgataccgctcgccgcagccgaacgaccgagcg |
| | | cagcgagtcagtgagcgaggaagcggaagagcgcc |
| | | tgatgcggtattttctccttacgcatctgtgcggt |
| | | atttcacaccgcatatggtgcactctcagtacaat |
| | | ctgctctgatgccgcatagttaagccagtatacac |
| | | tccgctatcgctacgtgactgggtcatggctgcgc |
| | | cccgacacccgccaacacccgctgacgcgccctga |
| | | cgggcttgtctgctcccggcatccgcttacagaca |
| | | agctgtgaccgtctccgggagctgcatgtgtcaga |
| | | ggttttcaccgtcatcaccgaaacgcgcgaggcag |
| | | cagatcaattcgcgcgcgaaggcgaagcggcatgc |
| | | ataatgtgcctgtcaaatggacgaagcagggattc |
| | | tgcaaaccctatgctactccgtcaagccgtcaatt |
| | | gtctgattcgttaccaattatgacaacttgacggc |
| | | tacatcattcactttttcttcacaaccggcacgga |
| | | actcgctcgggctggccccggtgcattttttaaat |
| | | acccgcgagaaatagagttgatcgtcaaaaccaac |
| | | attgcgaccgacggtggcgataggcatccgggtgg |
| | | tgctcaaaagcagcttcgcctggctgatacgttgg |
| | | tcctcgcgccagcttaagacgctaatccctaactg |
| | | ctggcggaaaagatgtgacagacgcgacggcgaca |
| | | agcaaacatgctgtgcgacgctggcgat |
| 90 | pMC- | acattaccctgttatccctagatgacattaccctg |
| | L + R- | ttatcccagatgacattaccctgttatccctagat |
| | TRAC1- | gacattaccctgttatccctagatgacatttaccc |
| | SSA48- | tgttatccctagatgacattaccctgttatcccag |
| | Anti- | atgacattaccctgttatccctagatacattaccc |
| | meso- | tgttatcccagatgacataccctgttatccctaga |
| | thelin | tgacattaccctgttatcccagatgacattaccct |
| | CAR | gttatccctagatacattaccctgttatcccagat |
| | (SSA/H | gacataccctgttatccctagatgacattaccctg |
| | MEJ) | ttatcccagatgacattaccctgttatccctagat |
| | Forward | acattaccctgttatcccagatgacataccctgtt |
| | homology | atccctagatgacattaccctgttatcccagatga |
| | arm | cattaccctgttatccctagatacattaccctgtt |
| | (735- | atcccagatgacataccctgttatccctagatgac |
| | 779; 45 | attaccctgttatcccagatgacattaccctgtta |
| | bp in | tccctagatacattaccctgttatcccagatgaca |
| | length) | taccctgttatccctagatgacattaccctgttat |
| | 5′ | cccagatgacattaccctgttatccctagatacat |
| | universal | taccctgttatcccagatgacataccctgttatcc |
| | guide | ctagatgacattaccctgttatcccagataaactc |
| | (786- | aatgatgatgatgatgatggtcgagactcagcggc |
| | 808; 23 | cgcggtgccagggcgtgcccttgggctccccgggc |
| | bp in | gcgactagtgaattcgggaggcgttcgggccacag |
| | length) | cggccctaaccctgatcctcttgtcccacagatat |
| | Reverse | ccagaaccctgaccctgccggttctggcgagggca |
| | homology | ggggttccctccttacatgcggagatgtagaagaa |
| | arm | aatccagggcctatggccctgcccgtcaccgctct |
| | (3200- | gctgctgcctctggctctgctgctgcatgccgctc |
| | 3233; 34 | gccccggaagtcaggtccagctgcagcagagcgga |
| | bp in | cctgagctggagaagccaggagcatccgtgaagat |
| | length) | ctcttgcaaggcctctggctacagcttcaccggct |
| | 3′ | atacaatgaactgggtgaagcagagccacggcaag |
| | universa1 | tccctggagtggatcggcctgatcaccccctacaa |
| | guide | cggcgccagctcctataatcagaagtttcgcggca |
| | (3171- | aggccaccctgacagtggacaagtctagctccacc |
| | 3193; 23 | gcctatatggacctgctgtccctgacatctgagga |
| | bp) | tagcgccgtgtacttctgcgcaaggggaggatatg |
| | | acggaaggggctttgattactggggccagggcacc |
| | | acagtgaccgtgtctagcggaggaggaggatccgg |
| | | aggaggaggatcctctggcggcggcagcgacatcg |
| | | agctgacacagtccccagcaatcatgtctgccagc |
| | | ccaggagagaaggtgaccatgacatgttctgccag |
| | | ctcctctgtgagctacatgcactggtatcagcaga |
| | | agtccggcacctctcccaagcggtggatctatgat |
| | | acatctaagctggcaagcggagtgcctggccggtt |
| | | ctccggctctggcagcggcaattcctactctctga |
| | | ccatcagctccgtggaggccgaggacgatgccaca |
| | | tactattgccagcagtggtccaagcaccctctgac |
| | | ctacggcgccggcacaaagctggagatcaaggcct |
| | | ctaccacaaccccagcacccagaccccctacccct |
| | | gcaccaacaatcgcatcccagccactgagcctgcg |
| | | gcccgaggcctgtaggccagcagcaggaggagcag |
| | | tgcacaccaggggcctggacttcgcctgcgatttt |
| | | tgggtgctggtggtggtgggaggcgtgctggcctg |
| | | ttatagcctgctggtgacagtggccttcatcatct |
| | | tttgggtgagaagcaagagatccaggctgctgcac |
| | | tccgactacatgaacatgacccctagacggcccgg |
| | | ccctacaaggaagcactaccagccatatgccccac |
| | | ccagagattttgccgcctataggagcaagcgcggc |
| | | cggaagaagctgctgtacatcttcaagcagccctt |
| | | catgcggcccgtgcagacaacccaggaggaggacg |
| | | gctgctcctgtaggttcccagaagaggaggaggga |
| | | ggatgcgagctgagggtgaagtttagccggtccgc |
| | | cgatgcaccagcatataagcagggacagaatcagc |
| | | tgtacaacgagctgaatctgggcaggcgcgaggag |
| | | tacgacgtgctggataagaggagaggacgggaccc |
| | | cgagatgggaggcaagcccaggcgcaagaaccctc |
| | | aggagggcctgtataatgagctgcagaaggacaag |
| | | atggccgaggcctactctgagatcggcatgaaggg |
| | | agagcggagaaggggcaagggacacgatggcctgt |
| | | atcagggcctgtccaccgccacaaaggacacctac |
| | | gatgccctgcacatgcaggccctgcctccaaggag |
| | | ggcaaagaggggatccggagagggacggggctctc |
| | | tgctgacctgcggcgatgtggaggagaacccaggc |
| | | cccatgggcacaagcctgctgtgctggatggcact |
| | | gtgcctgctgggagcagaccacgccgatgcctgcc |
| | | cctattctaatcccagcctgtgctccggaggagga |
| | | ggatctgagctgcctacccagggcacattctctaa |
| | | cgtgagcaccaatgtgagcccagccaagcccacaa |
| | | ccacagcctgcccatactccaaccccagcctgtgc |
| | | agcggcggaggaggcagccctgcaccaagaccccc |
| | | taccccagcacctacaatcgcaagtcagcctctga |
| | | gcctgcggcccgaggcctgtcgccctgccgccggc |
| | | ggcgccgtccatactaggggcctggactttgcctg |
| | | cgatatctacatctgggcaccactggcaggaacct |
| | | gtggcgtgctgctgctgagcctggtcatcacactg |
| | | tattgtaatcataggaatcggaggagagtgtgcaa |
| | | atgcccccgccctgtcgtctaaaccggtaataaaa |
| | | gatccttattttcattggatctgtgtgttggtttt |
| | | ttgtgtgagcgctagctgagactctaaatccagtg |
| | | acaagtctgtctgcctaaaaccgctgtggcccgaa |
| | | cgcctcccgatatcgtcgacccatgggggcccgcc |
| | | ccaactggggtaacctttgagttctctcagttggg |
| | | ggtaatcagcatcatgatgtggtaccacatcatga |
| | | tgctgattataagaatgcggccgccacactctagt |
| | | ggatctcgagttaataattcagaagaactcgtcaa |
| | | gaaggcgatagaaggcgatgcgctgcgaatcggga |
| | | gcggcgataccgtaaagcacgaggaagcggtcagc |
| | | ccattcgccgccaagctcttcagcaatatcacggg |
| | | tagccaacgctatgtcctgatagcggtccgccaca |
| | | cccagccggccacagtcgatgaatccagaaaagcg |
| | | gccattttccaccatgatattcggcaagcaggcat |
| | | cgccatgggtcacgacgagatcctcgccgtcgggc |
| | | atgctcgccttgagcctggcgaacagttcggctgg |
| | | cgcgagcccctgatgctcttcgtccagatcatcct |
| | | gatcgacaagaccggcttccatccgagtacgtgct |
| | | cgctcgatgcgatgtttcgcttggtggtcgaatgg |
| | | gcaggtagccggatcaagcgtatgcagccgccgca |
| | | ttgcatcagccatgatggatactttctcggcagga |
| | | gcaaggtgtagatgacatggagatcctgccccggc |
| | | acttcgcccaatagcagccagtcccttcccgcttc |
| | | agtgacaacgtcgagcacagctgcgcaaggaacgc |
| | | ccgtcgtggccagccacgatagccgcgctgcctcg |
| | | tcttgcagttcattcagggcaccggacaggtcggt |
| | | cttgacaaaaagaaccgggcgcccctgcgctgaca |
| | | gccggaacacggcggcatcagagcagccgattgtc |
| | | tgttgtgcccagtcatagccgaatagcctctccac |
| | | ccaagcggccggagaacctgcgtgcaatccatctt |
| | | gttcaatcatgcgaaacgatcctcatcctgtctct |
| | | tgatcagagcttgatcccctgcgccatcagatcct |
| | | tggcggcgagaaagccatccagtttactttgcagg |
| | | gcttcccaaccttaccagagggcgccccagctggc |
| | | aattccggttcgcttgctgtccataaaaccgccca |
| | | gtctagctatcgccatgtaagcccactgcaagcta |
| | | cctgctttctctttgcgcttgcgttttcccttgtc |
| | | cagatagcccagtagctgacattcatccggggtca |
| | | gcaccgtttctgcggactggctttctacgtgctcg |
| | | aggggggccaaacggtctccagcttggctgttttg |
| | | gcggatgagagaagattttcagcctgatacagatt |
| | | aaatcagaacgcagaagcggtctgataaaacagaa |
| | | tttgcctggcggcagtagcgcggtggtcccacctg |
| | | accccatgccgaactcagaagtgaaacgccgtagc |
| | | gccgatggtagtgtggggtctccccatgcgagagt |
| | | agggaactgccaggcatcaaataaaacgaaaggct |
| | | cagtcgaaagactgggcctttcgttttatctgttg |
| | | tttgtcggtgaacgctctcctgagtaggacaaatc |
| | | cgccgggagcggatttgaacgttgcgaagcaacgg |
| | | cccggagggtggcgggcaggacgcccgccataaac |
| | | tgccaggcatcaaattaagcagaaggccatcctga |
| | | cggatggcctttttgcgtttctacaaactcttttg |
| | | tttatttttctaaatacattcaaatatgtatccgc |
| | | tcatgaccaaaatcccttaacgtgagttttcgttc |
| | | cactgagcgtcagaccccgtagaaaagatcaaagg |
| | | atcttcttgagatcctttttttctgcgcgtaatct |
| | | gctgcttgcaaacaaaaaaaccaccgctaccagcg |
| | | gtggtttgtttgccggatcaagagctaccaactct |
| | | ttttccgaaggtaactggcttcagcagagcgcaga |
| | | taccaaatactgtccttctagtgtagccgtagtta |
| | | ggccaccacttcaagaactctgtagcaccgcctac |
| | | atacctcgctctgctaatcctgttaccagtggctg |
| | | ctgccagtggcgataagtcgtgtcttaccgggttg |
| | | gactcaagacgatagttaccggataaggcgcagcg |
| | | gtcgggctgaacggggggttcgtgcacacagccca |
| | | gcttggagcgaacgacctacaccgaactgagatac |
| | | ctacagcgtgagctatgagaaagcgccacgcttcc |
| | | cgaagggagaaaggcggacaggtatccggtaagcg |
| | | gcagggtcggaacaggagagcgcacgagggagctt |
| | | ccagggggaaacgcctggtatctttatagtcctgt |
| | | cgggtttcgccacctctgacttgagcgtcgatttt |
| | | tgtgatgctcgtcaggggggcggagcctatggaaa |
| | | aacgccagcaacgcggcctttttacggttcctggc |
| | | cttttgctggccttttgctcacatgttctttcctg |
| | | cgttatcccctgattctgtggataaccgtattacc |
| | | gcctttgagtgagctgataccgctcgccgcagccg |
| | | aacgaccgagcgcagcgagtcagtgagcgaggaag |
| | | cggaagagcgcctgatgcggtattttctccttacg |
| | | catctgtgcggtatttcacaccgcatatggtgcac |
| | | tctcagtacaatctgctctgatgccgcatagttaa |
| | | gccagtatacactccgctatcgctacgtgactggg |
| | | tcatggctgcgccccgacacccgccaacacccgct |
| | | gacgcgccctgacgggcttgtctgctcccggcatc |
| | | cgcttacagacaagctgtgaccgtctccgggagct |
| | | gcatgtgtcagaggttttcaccgtcatcaccgaaa |
| | | cgcgcgaggcagcagatcaattcgcgcgcgaaggc |
| | | gaagcggcalgcataatgtgcctglcaaatggacg |
| | | aagcagggatlctgcaaaccctatgctactccgtc |
| | | aagccgtcaattgtctgattcgttaccaattatga |
| | | caacttgacggctacatcattcactttttcttcac |
| | | aaccggcacggaactcgctcgggctggccccggtg |
| | | cattttttaaatacccgcgagaaatagagttgatc |
| | | gtcaaaaccaacattgcgaccgacggtggcgatag |
| | | gcatccgggtggtgctcaaaagcagcttcgcctgg |
| | | ctgatacgttggtcctcgcgccagcttaagacgct |
| | | aatccctaactgctggcggaaaagatgtgacagac |
| | | gcgacggcgacaagcaaacatgctgtgcgacgctg |
| | | gcgat |
| 91 | Anti- | MALPVTALLLPLALLLHAARPGSQVQLQQSGPELE |
| | meso- | KPGASVKISCKASGYSFTGYTMNWVKQSHGKSLEW |
| | thelin | IGLITPYNGASSYNQKFRGKATLTVDKSSSTAYMD |
| | CAR | LLSLTSEDSAVYFCARGGYDGRGFDYWGQGTTVTV |
| | CoDing | SSGGGGSGGGGSSGGGSDIELTQSPA1MSASPGEK |
| | Sequence | VTMTCSASSSVSYMHWYQQKSGTSPKRWIYDTSKL |
| | (CDS) | ASGVPGRFSGSGSGNSYSLTISSVEAEDDATYYCQ |
| | | QWSKHPLTYGAGTKLEIKASTTTPAPRPPTPAPTI |
| | | ASQPLSLRPEACRPAAGGAVHTRGLDFACDFWVLV |
| | | VVGGVLACYSLLVTVAFIIFWVRSKRSRLLHSDYM |
| | | NMTPRRPGPTRKHYQPYAPPRDFAAYRSKRGRKKL |
| | | LYIFKQPFMRPVQTTQEEDGCSCRFPEEEEGGCEL |
| | | RVKFSRSADAPAYKQGQNQLYNELNLGRREEYDVL |
| | | DKRRGRDPEMGGKPRRKNPQEGLYNELQKDKMAEA |
| | | YSEIGMKGERRRGKGHDGLYQGLSTATKDTYDALH |
| | | MQALPPR |

### Intersection of DNA Sensor Expression and Electroporation Timing

To evaluate timing of expression of DNA sensors (RIG-1, STING, 1F116, and AIM2) T cells were stimulated with anti-CD3 and anti-CD28 beads at a ratio of 1:2.5 (bead:cell). Stimulated cells were electroporated with the SA-GFP plasmid alone (plasmid control) or the SA-Donor in combination with Cas9 and AAVS1 gRNA (HR) at 12 hrs., 24 hrs., 30 hrs., 36 hrs., 48 hrs., and 72 hrs. post stimulation. Expression of the DNA sensors was evaluated after electroporation, FIG. 29A. A determination of the cell cycle phase was also determined and charted at the same time points, FIG. 29B. Percent GFP expression was quantified post electroporation, FIG. 29C.

### Example 14. Integration Mechanism Influences Expression of Insert Cargo

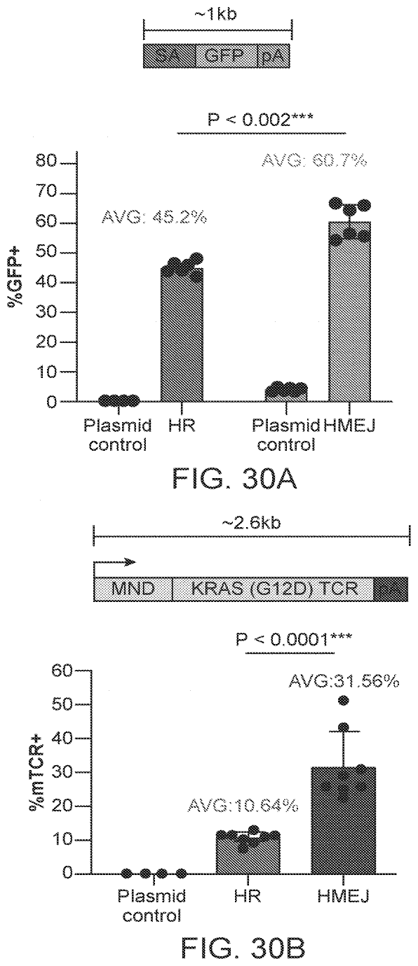

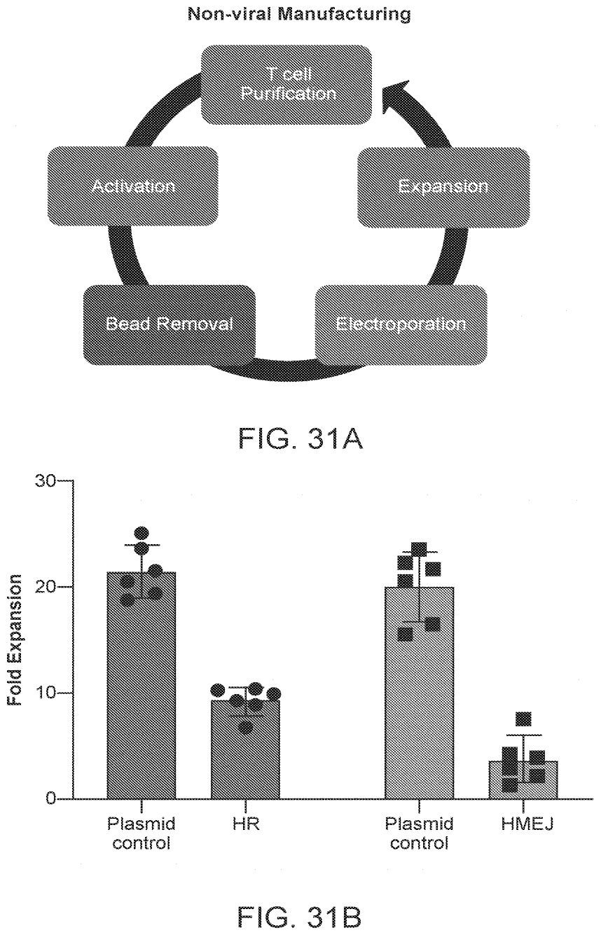

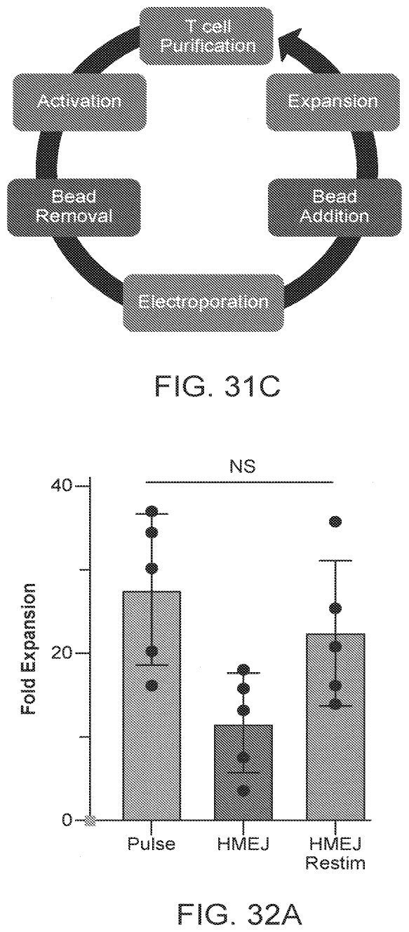

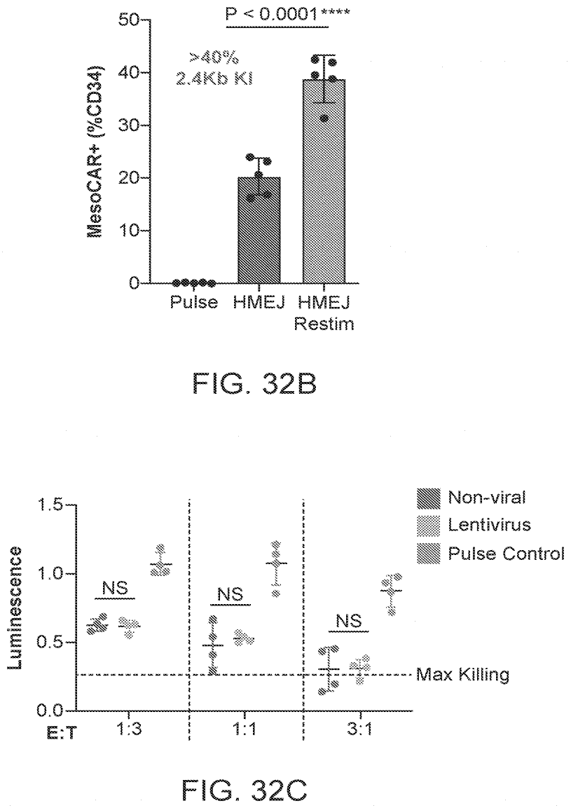

T cells were stimulated using anti-CD3 and anti-CD28 coated beads for 36 hours and electroporated with 1 ug donor plasmid having an anti-KRAS TCR alone (control), or donor plasmid having the anti-KRAS TCR in combination with 1.5 ug Cas9 mRNA and 1 ug AAVS1 gRNA (HR), or for HMEJ, the plasmid containing the anti-KRAS TCR, Cas9 mRNA, anti-AAVS1 gRNA, and Universal gRNA. Both the HR and HMEJ cargo is the SA-GFP construct integrated at AAVS1. Both the HR and HMEJ cargo is the MND-KRAS TCR with 1 kb homology (for HR) and with 48 bp homology (HMEJ). 7 days after electroporation percent GFP was analyzed, FIG. 30A (1 Kb) and FIG. 30B (2.6 kb). Results show that at least for larger cargo, the HMEJ construct is the preferred delivery mechanism.

### Example 15. Effect of Homology Arm Length on Integration by HR and HMEJ

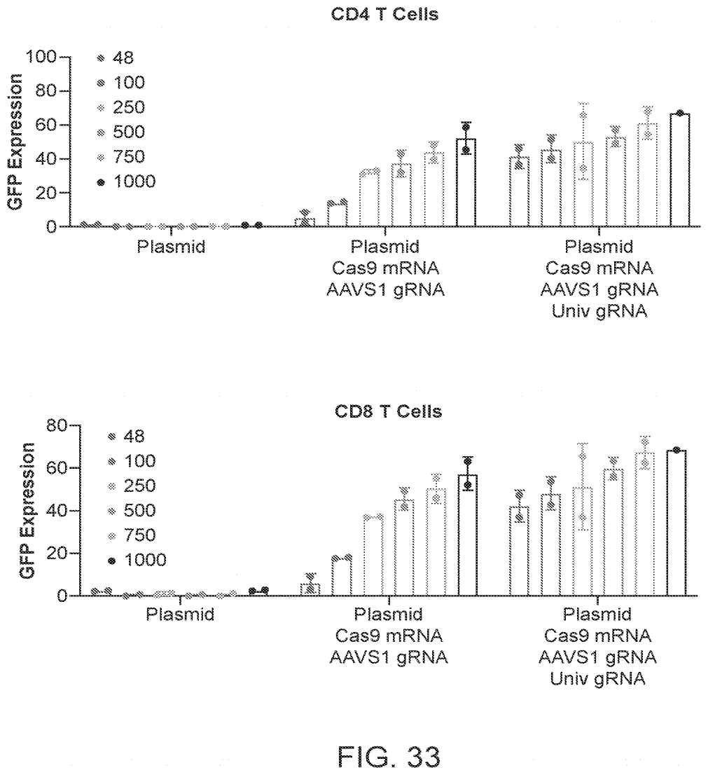

Donor transgenes with varying homology arm lengths (48, 100, 250, 500, 750, and 1000 bases) flanked by “universal” gRNA cut sites were generated and used to transfect cells along with Cas9 and AAVS1 gRNA post stimulation. FIG. 33 shows expression of GFP in both CD4 and CD8 cells after knock in. Plasmid only (donor transgene with no CRISPR reagents-episomal expression control). Data indicates that as the length of homology arms increase, the donor insert expression increased.

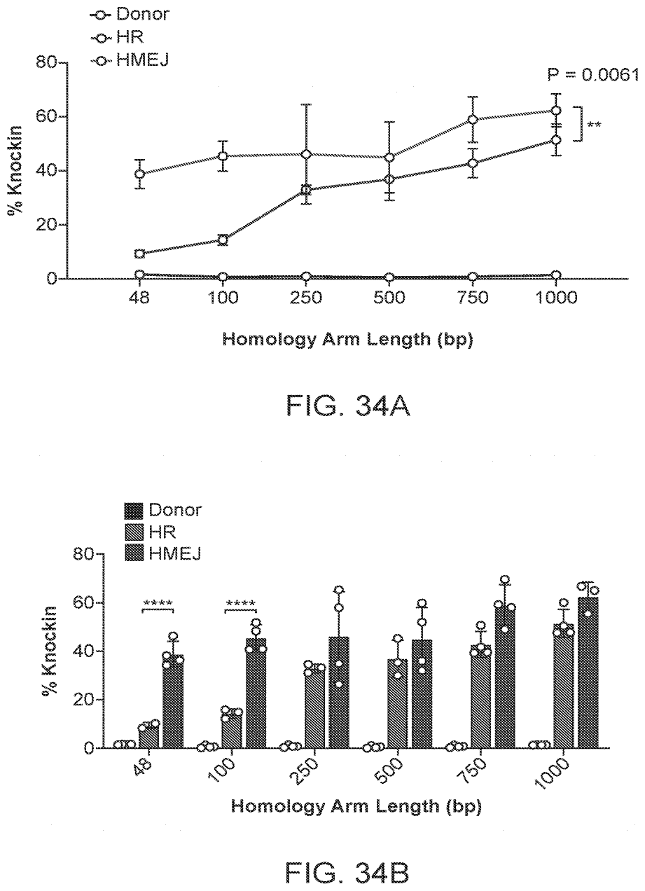

In a second experiment, T cells were stimulated and later electroporated with 1 ug donor only (control), SA-eGFP-pA (HR), or SA-eGFP-pA (HMEJ) constructs, each independently comprising homology arms of length 48, 100, 250, 500, 750, and 100 base pairs. Cells underwent a second stimulation and on day 7 were evaluated for percent knock in via flow cytometry, FIG. 34A. The same data is tabulated in FIG. 34B. Results show that the HMEJ construct has higher knock-in efficiency as compared to the comparable HR donor particularly at the lower homology arm lengths of 48 and 100 base pairs.

### Example 16. Additional Stimulation

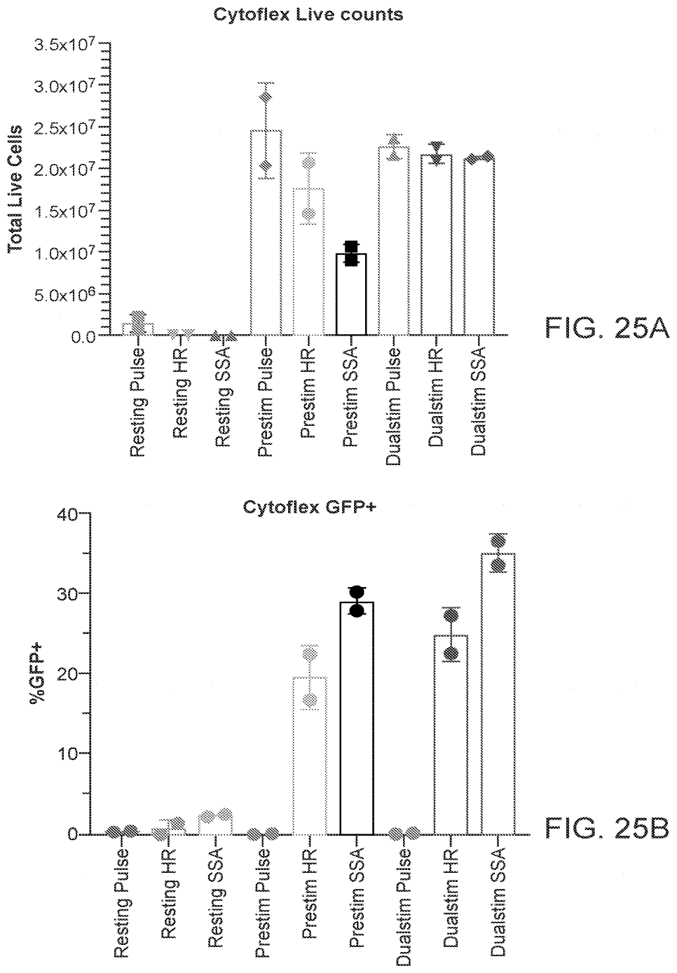

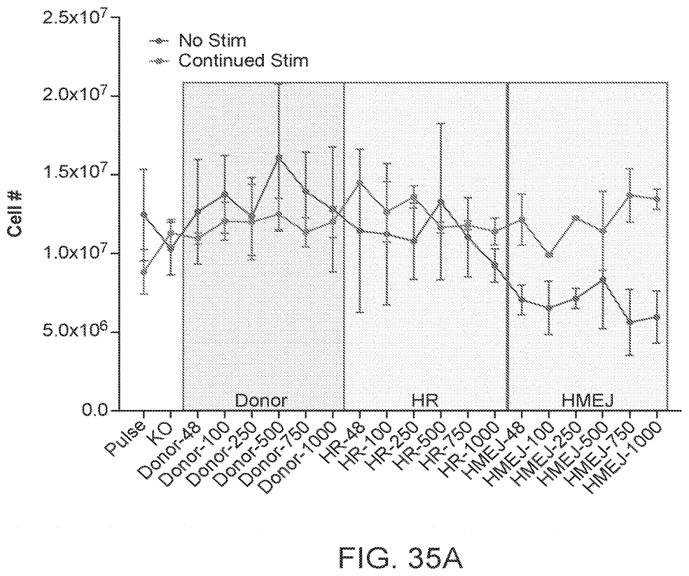

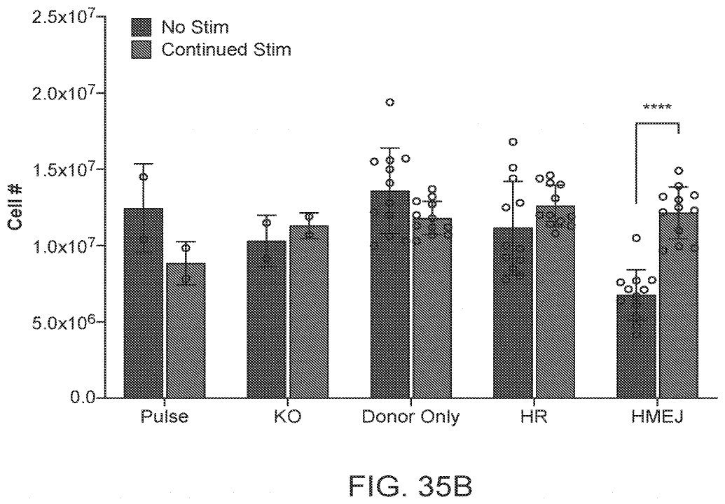

To evaluate any benefit in performing an additional stimulation as described in protocol 2, T cells were activated and stimulated and electroporated with constructs comprising an SA-eGFP-pA (HR), or SA-eGFP-pA (HMEJ) donor comprising homology arms (HR) or an HMEJ donor (denoted as SSA) that target AAVS1, electroporation method previously described herein. Cells were exposed to an additional stimulation, about 30 minutes after electroporation. GFP was measured at day 7 post electroporation. The additional stimulation assists in overcoming any cell expansion deficits in cells post-electroporation, see for example FIG. 25A and FIG. 25B. Additionally, results show that the additional stimulation increases the fold-expansion of SA-EGFP-pA (HMEJ) modified T cells, see FIG. 35A and FIG. 35B.

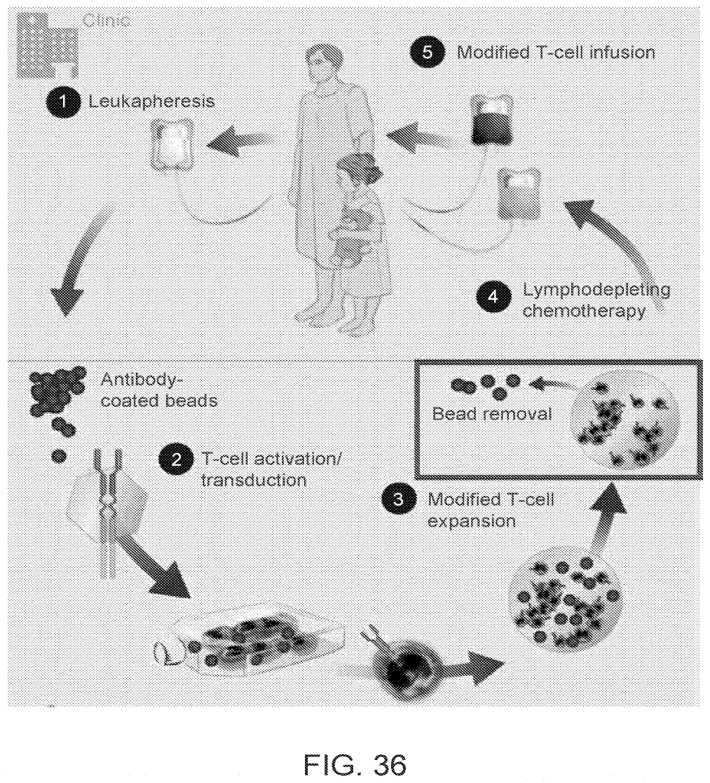

Additional stimulations can be introduced into a clinical workflow as outlined in FIG. 36. For example, an additional stimulation can be performed after step (2) and/or after step (3).

### Example 17. Treatment of Cancer Patient Using TCR-Modified T Cells

CRISPR-Cas9 system can be designed to transfer a TCR gene into autologous primary T cells from a patient of cancer. The TCR gene can be designed to have a high affinity to a target antigen expressed by the cancer cell identified in the patient. The TCR gene can be driven by a strong promoter to compete with endogenous TCR expressed by the primary T cells, for example, cytomegalovirus (CMV), murine stem cell virus (MSCV) U3, phosphoglycerate kinase (PGK), O-actin, ubiquitin, and a simian virus 40 (SV40)/CD43 composite promoter. The patient will be administered with the TCR-modified T cells.

Autologous CD3+ T cells will be obtained from peripheral blood of the patient according to the protocols described in Example 6. The isolated T cells will be cultured under standard conditions according to GMP guidance.

At least 30 min before electroporation, CD3+ T cells will be stimulated using anti-CD3 and anti-CD28 coated beads. Beads can be plated at ratios of 2 beads per cell or 1 bead for every 2.5 cells. Electroporation will be performed in two steps: first, the CD3+ T cells will be electroporated in the presence of Cas9 mRNA; and 6-24 hr. later, the cells will be subject to electroporation with the TCR gene-containing minicircle construct and gRNA. gRNA will be designed to target a safe harbor site of human genome, like AAVS1 site. Stable expression of the TCR gene will be validated by next-generation sequencing 2 weeks post-transfection. The cell viability, transfection efficiency and transgene load in the electroporated T cells will be assessed. Certain measure will also be taken to minimize any safety concern.

After validation, the TCR modified T cells will be infused to the cancer patient. The infused TCR modified T cells is expected to expand in vitro to a clinically desirable level, including the number of TCR modified T cells in the peripheral blood stream of the patient, and the expression level of the transplanted TCR gene. The infusion regimen will also be determined based on clinical evaluations, for instance, the stage of the cancer, the treatment history of the patient, the CBC (complete blood cell count) and vital signs of the patient on the day of treatment. Infusion dose may be escalated or deescalated depending on the progression of the disease, the repulsion reaction of the patient, and many other medical factors.

1.-113. (canceled)

114. A method of generating a population of engineered mammalian cells, the method comprising:
(a) contacting a plurality of mammalian cells with a polynucleic acid construct comprising an insert sequence flanked by homology arms, wherein each of the homology arms comprises a sequence homologous to at most 400 consecutive nucleotides of a sequence adjacent to a target site in the genome of the plurality of mammalian cells;
(b) cleaving the polynucleic acid construct;
(c) generating a first double stranded break in the genome of the plurality of mammalian cells at the target site and generating a second double stranded break in the genome of the plurality of mammalian cells at a second site; and
(d) inserting the insert sequence in the target site, to thereby generate a population of engineered mammalian cells.

115. The method of claim 114, further comprising expanding the population of engineered mammalian cells.

116. The method of claim 114, further comprising contacting the plurality of mammalian cells with a DNase.

117. The method of claim 116, wherein the DNase is selected from the group consisting of: DNase I, Benzonase, Exonuclease I, Exonuclease III, Mung Bean Nuclease, Nuclease BAL 31, RNase I, S1 Nuclease, Lambda Exonuclease, RecJ, T7 exonuclease, restriction enzymes, and any combination thereof.

118. The method of claim 114, further comprising contacting the plurality of mammalian cells with an exogenous immunostimulatory agent.

119. The method of claim 118, wherein the exogenous immunostimulatory agent is B7, CD80, CD83, CD86, CD32, CD64, 4-1BBL, anti-CD3, anti-CD3 mAb, S-2-hydroxyglutarate, anti-CD28, anti-CD28 mAb, CD1d, anti-CD2, IL-15, IL-17, IL-21, IL-2, IL-7, or truncated CD19.

120. The method of claim 118, wherein the exogenous immunostimulatory agent stimulates expansion of at least a portion of the plurality of mammalian cells.

121. The method of claim 118, wherein the concentration of the exogenous immunostimulatory agent is from about 50 IU/ml to about 1000 IU/ml.

122. The method of claim 118, the contacting of (a) occurs from about 30 hours up to 36 hours after the contacting with the exogenous immunostimulatory agent.

123. The method of claim 114, further comprising contacting the plurality of mammalian cells with an exogenous agent that modulates DNA double strand break repair.

124. The method of claim 123, wherein the exogenous agent that modulates DNA double strand break repair comprises a protein.

125. The method of claim 124, wherein the protein is selected from the group consisting of: Ku70, Ku80, BRCA1, BRCA2, RAD51, RS-1, PALB2, Nap1, p400 ATPase, EVL, NAC, MRE11, RAD50, RAD52, RAD55, RAD57, RAD54, RAD54B, Srs2, NBS1, H2AX, PARP-1, RAD18, DNA-PKcs, XRCC4, XLF, Artemis, TdT, pol μ and pol λ, ATM, AKT1, AKT2, AKT3, Nibrin, CtIP, EXO1, BLM, E4 orf6, E1b55K, and Scr7.

126. The method of claim 114, wherein the plurality of mammalian cells are cultured in vitro or ex vivo in a culture medium, wherein the culture medium is substantially antibiotic free.

127. The method of claim 114, wherein the insert sequence is introduced into the plurality of mammalian cells using a plasmid, a minicircle vector, a linearized double stranded DNA construct, or a viral vector.

128. The method of claim 114, wherein the insert sequence comprises a sequence encoding an exogenous receptor.

129. The method of claim 128, wherein the exogenous receptor is a T cell receptor (TCR), a chimeric antigen receptor (CAR), a B cell receptor (BCR), a natural killer cell (NK cell) receptor, a cytokine receptor, or a chemokine receptor.

130. The method of claim 128, wherein the exogenous receptor is an immune receptor with specificity for a disease-associated antigen.

131. The method of claim 128, wherein the exogenous receptor is an immune receptor that specifically binds to a cancer antigen.

132. The method of claim 128, wherein the exogenous receptor is an immune receptor that specifically binds an autoimmune antigen.

133. The method of claim 128, wherein the exogenous receptor is a TCR.

134. A method of making an engineered T cell, the method comprising:
(a) providing a primary T cell from a human subject;
(b) introducing, ex vivo, into the primary T cell:
(i) a nuclease or a polynucleic acid encoding the nuclease, wherein the nuclease is a CRISPR-associated nuclease;
(ii) a first guide RNA or polynucleic acid encoding the first guide RNA, wherein the first guide RNA targets a sequence in a TRAC or TCRB locus of the primary T cell;
(iii) a second guide RNA or a polynucleic acid encoding the second guide RNA; and
(iv) a polynucleic acid construct comprising a sequence for insertion, wherein the sequence for insertion comprises a sequence encoding an exogenous T cell receptor or chimeric antigen receptor, wherein the polynucleic acid construct comprises a first short homology arm and a second short homology arm that flank the sequence for insertion, wherein the first short homology arm and the second short homology arm comprise sequences homologous to sequences in the TRAC or TCRB locus of the primary T cell, wherein the first short homology arm is less than 50 base pairs and the second short homology arm is less than 50 base pairs, wherein the first short homology arm and the second short homology arm are flanked by sequences targeted by the second guide RNA;
(c) producing a double stranded break in the TRAC or TCRB locus of the genome of the primary T cell, wherein double stranded break in the TRAC or TCRB locus is produced by the CRISPR-associated nuclease and the first guide RNA, wherein the double stranded break is between a first sequence homologous to the first short homology arm and a second sequence homologous to the second short homology arm; and
(d) producing two double stranded breaks in the polynucleic acid construct, thereby generating a cleaved polynucleic acid construct, wherein the cleaved polynucleic acid construct comprises the first short homology arm at a first end and the second short homology arm at a second end, wherein the two double stranded breaks are produced by the CRISPR-associated nuclease and the second guide RNA;
(e) inserting the sequence encoding the exogenous T cell receptor into the primary T cell genome at the site of the double stranded break in the TRAC or TCRB locus by homology mediated end joining.
